(19) United States
(12) Patent Application Publication
(10) Pub. No.: US 2023/0124616 A1
(43) Pub. Date: Apr. 20, 2023

(54) COMPOUNDS AND METHODS FOR MODULATING KCNQ2

(71) Applicant: Ionis Pharmaceuticals, Inc., Carlsbad, CA (US)

(72) Inventors: Paymaan Jafar-nejad, Carlsbad, CA (US); Huynh-Hoa Bui, San Diego, CA (US)

(73) Assignee: Ionis Pharmaceuticals, Inc., Carlsbad, CA (US)

(21) Appl. No.: 17/908,135

(22) PCT Filed: Mar. 5, 2021
(86) PCT No.: PCT/US2021/021039

§ 371 Date: Aug. 30, 2022

Related U.S. Application Data
(60) Provisional application No. 62/986,553, filed on Mar. 6, 2020.

(51) Int. Cl. C12N 15/113; A61P 43/00

(52) U.S. Cl. CPC C12N 15/113; A61P 43/00; C12N 2310/11; C12N 2310/315; C12N 2310/322; C12N 2310/341; C12N 2310/345

(57) ABSTRACT

Provided are compounds, methods, and pharmaceutical compositions for reducing the amount or activity of KCNQ2 RNA in a cell or subject, and in certain instances reducing the amount of Kᵥ7.2 protein in a cell or subject. Such compounds, methods, and pharmaceutical compositions are useful to ameliorate at least one symptom or hallmark of an epileptic encephalopathy. Such symptoms and hallmarks include infantile spasms or seizures, EEC abnormalities, brain MRI abnormalities in the infant, and an associated developmental impairment. Such epileptic encephalopathies include those associated with gain-of-function and dominant negative mutations in KCNQ2.

### SEQUENCE LISTING

The present application is being filed along with a Sequence Listing in electronic format. The Sequence Listing is provided as a file entitled BIOL0375WOSEQ_ST25.txt, created on Mar. 3, 2021, which is 295 KB in size. The information in the electronic format of the sequence listing is incorporated herein by reference in its entirety.

### FIELD

Provided are compounds, methods, and pharmaceutical compositions for reducing the amount of KCNQ2 RNA in a cell or subject, and in certain instances reducing the amount of Kᵥ7.2 protein in a cell or subject. Such compounds, methods, and pharmaceutical compositions are useful to ameliorate at least one symptom or hallmark of an epileptic encephalopathy. Such symptoms and hallmarks include infantile spasms or seizures, EEG abnormalities, brain MRI abnormalities in the infant, and developmental impairment. Such epileptic encephalopathies include KCNQ2-related neonatal epileptic encephalopathy.

### BACKGROUND

KCNQ2-related neonatal epileptic encephalopathy is caused by gain-of-function or dominant negative mutations in the KCNQ2 gene. This disorder causes severe developmental impairment in affected infants. Symptoms and hallmarks include infantile spasms or seizures, EEG abnormalities, brain MRI abnormalities in the infant, and an associated developmental impairment. There are no specific therapies for epileptic encephalopathy caused by mutations in the KCNQ2 gene.

KCNQ2-related neonatal epileptic encephalopathy can be caused by a number of different mutations in the KCNQ2 gene. Certain such genetic mutations cause mutations in the KCNQ2-encoded neuronal voltage-gated potassium channel (Kᵥ7.2 protein), including gain-of-function mutations and dominant-negative mutations of the Kᵥ7.2 protein. Gain-of-function mutations at position 201 of the Kᵥ7.2 protein have been identified to cause some cases of neonatal epileptic encephalopathy (e.g., R201H, R201Q, R201C; Miceli, et al., “Early-Onset Epileptic Encephalopathy Caused by Gain-of-Function Mutations in the Voltage Sensor of Kᵥ7.2 and Kᵥ7.3 Potassium Channel Subunits”, J Neuroscience, 2015, 35(9):3782-3793; Mulkey, et al, “Neonatal Non-Epileptic Myoclonus is a Prominent Clinical Feature of KCNQ2 Gain-of-Function Variants R201C and R201H”, Epilepsia, 2017, 58(3): 436-445; Millichap, et al., “KCNQ2 encephalopathy: Features, mutational hot spots, and ezogabine treatment of 11 patients”, Neurology Genetics, 2016, 2:e96). Dominant-negative mutations at various positions of the protein have been shown to reduce not only the function of the mutated protein, but to also reduce the function of the wild-type protein expressed by the alternative allele, thus exacerbating the disease (Orhan, et al., “Dominant-negative effects of KCNQ2 mutations are associated with epileptic encephalopathy”, Annals of Neurology, 2014, 75:382-394).

Currently there is a lack of acceptable options for treating epileptic encephalopathies such as KCNQ2-related neonatal epileptic encephalopathies. It is therefore an object herein to provide compounds, methods, and pharmaceutical compositions for the treatment of such diseases.

### SUMMARY OF THE INVENTION

Provided herein are compounds, methods and pharmaceutical compositions for reducing the amount of KCNQ2 RNA, and in certain embodiments reducing the amount of Kᵥ7.2 protein in a cell or subject. In certain embodiments, the subject has an epileptic encepholapathy. In certain embodiments, the subject has a gain-of-function or dominant negative mutation in the KCNQ2 gene. In certain embodiments, compounds useful for reducing the amount of KCNQ2 RNA and/or Kᵥ7.2 protein are oligomeric compounds. In certain embodiments, oligomeric compounds comprise modified oligonucleotides.

Also provided are methods useful for ameliorating at least one symptom or hallmark of an epileptic encepholapathy. In certain embodiments, the epileptic encepholapathy is caused by a gain-of-function or dominant negative mutation in the Kᵥ7.2 protein encoded by a mutated KCNQ gene. In certain embodiments, the symptom or hallmark is infantile spasms or seizures, EEG abnormalities, brain MRI abnormalities in the infant, and an associated developmental impairment.

### DETAILED DESCRIPTION OF THE INVENTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings:

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings:

As used herein, “2′-deoxyribonucleoside” means a nucleoside comprising a 2′-H(H) deoxyribosyl sugar moiety. In certain embodiments, a 2′-deoxyribonucleoside is a 2′-β-D deoxyribonucleoside and comprises a 2′-β-D-deoxyribosyl sugar moiety, which has the β-D configuration as found in naturally occurring deoxyribonucleic acids (DNA). In certain embodiments, a 2′-deoxyribonucleoside may comprise a modified nucleobase or may comprise an RNA nucleobase (uracil).

As used herein, “2′-MOE” means a 2′-OCH₂CH₂OCH₃ group in place of the 2′-OH group of a ribosyl sugar moiety. A “2′-MOE sugar moiety” is a sugar moiety with a 2′-OCH₂CH₂OCH₃ group in place of the 2′-OH group of a ribosyl sugar moiety. Unless otherwise indicated, a 2′-MOE sugar moiety is in the β-D configuration. “MOE” means O-methoxyethyl.

As used herein, “2′-MOE nucleoside” means a nucleoside comprising a 2′-MOE sugar moiety.

As used herein, “2′-OMe” means a 2′-OCH₃ group in place of the 2′-OH group of a ribosyl sugar moiety. A “2′-OMe sugar moiety” is a sugar moiety with a 2′-OCH₃ group in place of the 2′-OH group of a ribosyl sugar moiety. Unless otherwise indicated, a 2′-OMe sugar moiety is in the β-D configuration. “OMe” means O-methyl.

As used herein, “2′-OMe nucleoside” means a nucleoside comprising a 2′-OMe sugar moiety.

As used herein, “2′-substituted nucleoside” means a nucleoside comprising a 2′-substituted sugar moiety. As used herein, “2′-substituted” in reference to a sugar moiety means a sugar moiety comprising at least one 2′-substituent group other than H or OH. As used herein, “2′-substituted nucleoside” means a nucleoside comprising a 2′-substituted sugar moiety. As used herein, “2′-substituted” in reference to a sugar moiety means a sugar moiety comprising at least one 2′-substituent group other than H or OH.

As used herein, “5-methyl cytosine” means a cytosine modified with a methyl group attached to the 5 position. A 5-methyl cytosine is a modified nucleobase.

As used herein, “administering” means providing a pharmaceutical agent to a subject.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound.

As used herein, “antisense compound” means an oligomeric compound or oligomeric duplex capable of achieving at least one antisense activity.

As used herein, “ameliorate” in reference to a treatment means improvement in at least one symptom relative to the same symptom in the absence of the treatment. In certain embodiments, amelioration is the reduction in the severity or frequency of a symptom or the delayed onset or slowing of progression in the severity or frequency of a symptom. In certain embodiments, the symptom or hallmark is infantile spasms or seizures, EEG abnormalities, brain MRI abnormalities in the infant, and an associated developmental impairment.

As used herein, “bicyclic nucleoside” or “BNA” means a nucleoside comprising a bicyclic sugar moiety.

As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the furanosyl moiety is a ribosyl moiety. In certain embodiments, the bicyclic sugar moiety does not comprise a furanosyl moiety.

As used herein, “cleavable moiety” means a bond or group of atoms that is cleaved under physiological conditions, for example, inside a cell, a subject, or a human.

As used herein, “complementary” in reference to an oligonucleotide means that at least 70% of the nucleobases of the oligonucleotide or one or more portions thereof and the nucleobases of another nucleic acid or one or more portions thereof are capable of hydrogen bonding with one another when the nucleobase sequence of the oligonucleotide and the other nucleic acid are aligned in opposing directions. Complementary nucleobases means nucleobases that are capable of forming hydrogen bonds with one another. Complementary nucleobase pairs include adenine (A) with thymine (T), adenine (A) with uracil (U), cytosine (C) with guanine (G), and 5-methyl cytosine (mC) with guanine (G). Complementary oligonucleotides and/or target nucleic acids need not have nucleobase complementarity at each nucleoside. Rather, some mismatches are tolerated. As used herein, “fully complementary” or “100% complementary” in reference to an oligonucleotide, or a portion thereof, means that the oligonucleotide, or portion thereof, is complementary to another oligonucleotide or target nucleic acid at each nucleobase of the shorter of the two oligonucleotides, or at each nucleoside if the oligonucleotides are the same length.

As used herein, “conjugate group” means a group of atoms that is directly or indirectly attached to an oligonucleotide. Conjugate groups include a conjugate moiety and a conjugate linker that attaches the conjugate moiety to the oligonucleotide.

As used herein, “conjugate linker” means a single bond or a group of atoms comprising at least one bond that connects a conjugate moiety to an oligonucleotide.

As used herein, “conjugate moiety” means a group of atoms that is attached to an oligonucleotide via a conjugate linker.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “cEt” means a 4′ to 2′ bridge in place of the 2′OH-group of a ribosyl sugar moiety, wherein the bridge has the formula of 4′-CH(CH3)¬-O-2′, and wherein the methyl group of the bridge is in the S configuration. A “cEt sugar moiety” is a bicyclic sugar moiety with a 4′ to 2′ bridge in place of the 2′OH-group of a ribosyl sugar moiety, wherein the bridge has the formula of 4′-CH(CH3)¬-O-2′, and wherein the methyl group of the bridge is in the S configuration. “cEt” means constrained ethyl.

As used herein, “cEt nucleoside” means a nucleoside comprising a cEt sugar moiety.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more stereorandom chiral centers. In certain embodiments, the molecules are modified oligonucleotides. In certain embodiments, the molecules are compounds comprising modified oligonucleotides.

As used herein, “deoxy region” means a region of 5-12 contiguous nucleotides, wherein at least 70% of the nucleosides are 2′-β-D-deoxynucleosides. In certain embodiments, each nucleoside is selected from a 2′-β-D-deoxynucleoside, a bicyclic nucleoside, and a 2′-substituted nucleoside. In certain embodiments, a deoxy region supports RNase H activity. In certain embodiments, a deoxy region is the gap or internal region of a gapmer.

As used herein, “gapmer” means a modified oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.” The internal region is a deoxy region. The positions of the internal region or gap refer to the order of the nucleosides of the internal region and are counted starting from the 5′-end of the internal region. Unless otherwise indicated, “gapmer” refers to a sugar motif. In certain embodiments, each nucleoside of the gap is a 2′-β-D-deoxynucleoside. In certain embodiments, the gap comprises one 2′-substituted nucleoside at position 1, 2, 3, 4, or 5 of the gap, and the remainder of the nucleosides of the gap are 2′-β-D-deoxynucleosides. As used herein, the term “MOE gapmer” indicates a gapmer having a gap comprising 2′-β-D-deoxynucleosides and wings comprising 2′-MOE nucleosides. As used herein, the term “mixed wing gapmer” indicates a gapmer having wings comprising modified nucleosides comprising at least two different sugar modifications. Unless otherwise indicated, a gapmer may comprise one or more modified internucleoside linkages and/or modified nucleobases and such modifications do not necessarily follow the gapmer pattern of the sugar modifications.

As used herein, “hotspot region” is a range of nucleobases on a target nucleic acid that is amenable to oligomeric compound-mediated reduction of the amount or activity of the target nucleic acid.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, “internucleoside linkage” means the covalent linkage between contiguous nucleosides in an oligonucleotide. As used herein, “modified internucleoside linkage” means any internucleoside linkage other than a phosphodiester internucleoside linkage. “Phosphorothioate internucleoside linkage” is a modified internucleoside linkage in which one of the non-bridging oxygen atoms of a phosphodiester internucleoside linkage is replaced with a sulfur atom.

As used herein, “linker-nucleoside” means a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

As used herein, “non-bicyclic modified sugar moiety” means a modified sugar moiety that comprises a modification, such as a substituent, that does not form a bridge between two atoms of the sugar to form a second ring.

As used herein, “mismatch” or “non-complementary” means a nucleobase of a first oligonucleotide that is not complementary with the corresponding nucleobase of a second oligonucleotide or target nucleic acid when the first and second oligonucleotide are aligned.

As used herein, “motif” means the pattern of unmodified and/or modified sugar moieties, nucleobases, and/or internucleoside linkages, in an oligonucleotide.

As used herein, “nucleobase” means an unmodified nucleobase or a modified nucleobase. As used herein an “unmodified nucleobase” is adenine (A), thymine (T), cytosine (C), uracil (U), or guanine (G). As used herein, a “modified nucleobase” is a group of atoms other than unmodified A, T, C, U, or G capable of pairing with at least one unmodified nucleobase. A “5-methyl cytosine” is a modified nucleobase. A universal base is a modified nucleobase that can pair with any one of the five unmodified nucleobases. As used herein, “nucleobase sequence” means the order of contiguous nucleobases in a target nucleic acid or oligonucleotide independent of any sugar or internucleoside linkage modification.

As used herein, “nucleoside” means a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. “Linked nucleosides” are nucleosides that are connected in a contiguous sequence (i.e., no additional nucleosides are presented between those that are linked).

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound. The term “oligomeric duplex” means a duplex formed by two oligomeric compounds having complementary nucleobase sequences. Each oligomeric compound of an oligomeric duplex may be referred to as a “duplexed oligomeric compound.”

As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “pharmaceutically acceptable carrier or diluent” means any substance suitable for use in administering to a subject. Certain such carriers enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable carrier or diluent is sterile water, sterile saline, sterile buffer solution or sterile artificial cerebrospinal fluid.

As used herein, “pharmaceutically acceptable salts” means physiologically and pharmaceutically acceptable salts of compounds. Pharmaceutically acceptable salts retain the desired biological activity of the parent compound and do not impart undesired toxicological effects thereto.

As used herein, “pharmaceutical composition” means a mixture of substances suitable for administering to a subject. For example, a pharmaceutical composition may comprise an oligomeric compound and a sterile aqueous solution.

As used herein, “reducing the amount” refers to a reduction of the transcriptional expression or activity relative to the transcriptional expression in an untreated or control sample and does not necessarily indicate a total elimination of transcriptional expression.

As used herein, “RNA” means an RNA transcript and includes pre-mRNA and mature mRNA unless otherwise specified.

As used herein, “self-complementary” in reference to an oligonucleotide means an oligonucleotide that at least partially hybridizes to itself.

As used herein, “standard cell assay” means the assay described in Example 1 and reasonable variations thereof.

As used herein, “stereorandom chiral center” in the context of a population of molecules of identical molecular formula means a chiral center having a random stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center. The stereochemical configuration of a chiral center is considered random when it is the result of a synthetic method that is not designed to control the stereochemical configuration. In certain embodiments, a stereorandom chiral center is a stereorandom phosphorothioate internucleoside linkage.

As used herein, “subject” means a human or non-human animal.

As used herein, “sugar moiety” means an unmodified sugar moiety or a modified sugar moiety. As used herein, “unmodified sugar moiety” means a 2′-OH(H) β-D-ribosyl moiety, as found in RNA (an “unmodified RNA sugar moiety”), or a 2′-H(H) β-D-deoxyribosyl moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate.

As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or target nucleic acids.

As used herein, “symptom or hallmark” means any physical feature or test result that indicates the existence or extent of a disease or disorder. In certain embodiments, a symptom is apparent to a subject or to a medical professional examining or testing the subject. In certain embodiments, a hallmark is apparent upon invasive diagnostic testing, including, but not limited to, post-mortem tests. In certain embodiments, a hallmark is apparent on a brain MRI scan.

As used herein, “target nucleic acid” mean a nucleic acid that an antisense compound is designed to affect.

As used herein, “target region” means a portion of a target nucleic acid to which an oligomeric compound is designed to hybridize.

As used herein, “terminal group” means a chemical group or group of atoms that is covalently linked to a terminus of an oligonucleotide.

As used herein, “therapeutically effective amount” means an amount of a pharmaceutical agent that provides a therapeutic benefit to a subject. For example, a therapeutically effective amount improves a symptom or hallmark of a disease.

### Certain Embodiments

The present disclosure provides the following non-limiting numbered embodiments:
Embodiment 1. An oligomeric compound, comprising a modified oligonucleotide consisting of 12 to 30 linked nucleosides wherein the nucleobase sequence of the modified oligonucleotide is at least 90% complementary to an equal length portion of a KCNQ2 nucleic acid, and wherein the modified oligonucleotide comprises at least one modification selected from a modified sugar moiety and a modified internucleoside linkage.
Embodiment 2. The oligomeric compound of embodiment 1, wherein the modified oligonucleotide comprises an at least 8 nucleobase portion, at least 9 nucleobase portion, at least 10 nucleobase portion, at least 11 nucleobase portion, at least 12 nucleobase portion, at least 13 nucleobase portion, at least 14 nucleobase portion, at least 15 nucleobase portion, at least 16 nucleobase portion, at least 17 nucleobase portion, at least 18 nucleobase portion, at least 19 nucleobase portion, or an at least 20 nucleobase portion of any of SEQ ID NO: 21-98.
Embodiment 3. An oligomeric compound comprising a modified oligonucleotide consisting of 12 to 30 linked nucleosides and having a nucleobase sequence comprising at least 12, at least 13, at least 14, at least 15, at least 16, at least 17, at least 18, at least 19 or 20 contiguous nucleobases of any of SEQ ID NO: 21-98.
Embodiment 4. An oligomeric compound comprising a modified oligonucleotide consisting of 12 to 30 linked nucleosides and having a nucleobase sequence having at least 8, at least 9, at least 10, at least 11, at least 12, at least 13, at least 14, at least 15, at least 16, at least 17, at least 18, at least 19, or at least 20 contiguous nucleobases at least 90%, at least 95%, or 100% complementary to:
   an equal length portion of nucleobases 4,600-4,624 of SEQ ID NO: 2;
   an equal length portion of nucleobases 8,970-8,990 of SEQ ID NO: 2;
   an equal length portion of nucleobases 23,730-23,752 of SEQ ID NO: 2;
   an equal length portion of nucleobases 24,439-24,775 of SEQ ID NO: 2;
   an equal length portion of nucleobases 27,275-27,306 of SEQ ID NO: 2;
   an equal length portion of nucleobases 33,048-33,083 of SEQ ID NO: 2;
   an equal length portion of nucleobases 33,054-33,083 of SEQ ID NO: 2;
   an equal length portion of nucleobases 34,198-34,232 of SEQ ID NO: 2;
   an equal length portion of nucleobases 34,543-34,563 of SEQ ID NO: 2;
   an equal length portion of nucleobases 35,323-35,343 of SEQ ID NO: 2;
   an equal length portion of nucleobases 41,334-41,357 of SEQ ID NO: 2;
   an equal length portion of nucleobases 57,517-57,552 of SEQ ID NO: 2;
   an equal length portion of nucleobases 57,523-57,544 of SEQ ID NO: 2;
   an equal length portion of nucleobases 65,636-65,671 of SEQ ID NO: 2; or
   an equal length portion of nucleobases 65,745-65,772 of SEQ ID NO: 2.
Embodiment 5. An oligomeric compound comprising a modified oligonucleotide consisting of 12 to 30 linked nucleosides and having a nucleobase sequence comprising at least 8, at least 9, at least 10, at least 11, at least 12, at least 13, at least 14, at least 15, at least 16, at least 17, at least 18, at least 19, or at least 20 contiguous nucleobases of a sequence selected from:
   SEQ ID NOs: 67, 82;
   SEQ ID NOs: 34, 83;
   SEQ ID NOs: 61, 97;
   SEQ ID NOs: 40, 42, 88;
   SEQ ID NOs: 21, 24, 32, 41, 44, 71, 96;
   SEQ ID NOs: 23, 25, 30, 50, 57, 68, 89, 91, 95;
   SEQ ID NOs: 25, 30, 50, 68, 89, 91, 95;
   SEQ ID NOs: 35, 56;
   SEQ ID NOs: 58, 84;
   SEQ ID NOs: 37, 74;
   SEQ ID NOs: 31, 98;
   SEQ ID NOs: 59, 76;
   SEQ ID NOs: 22, 36, 52, 55, 64, 69, 70, 86, 90;
   SEQ ID NOs: 55, 86;
   SEQ ID NOs: 26-29, 49, 60, 80, 87, 92; or
   SEQ ID NOs: 43, 45, 54, 63, 78.
Embodiment 6. The oligomeric compound of any of embodiments 3-5, wherein the modified oligonucleotide has a nucleobase sequence that is at least 70%, at least 75%, at least 80%, at least 85%, at least 90%, at least 95%, or is 100% complementary to the nucleobase sequence of SEQ ID NO: 1 or SEQ ID NO: 2 when measured across the entire nucleobase sequence of the modified oligonucleotide.
Embodiment 7. The oligomeric compound of any of embodiments 1-6, wherein the modified oligonucleotide comprises at least one modified nucleoside.
Embodiment 8. The oligomeric compound of embodiment 7, wherein the modified oligonucleotide comprises at least one modified nucleoside comprising a modified sugar moiety.
Embodiment 9. The oligomeric compound of embodiment 8, wherein the modified oligonucleotide comprises at least one modified nucleoside comprising a bicyclic sugar moiety.
Embodiment 10. The oligomeric compound of embodiment 9, wherein the modified oligonucleotide comprises at least one modified nucleoside comprising a bicyclic sugar moiety having a 2′-4′ bridge, wherein the 2′-4′ bridge is selected from —O—CH₂—; and —O—CH(CH₃)—.
Embodiment 11. The oligomeric compound of any of embodiments 8-10, wherein the modified oligonucleotide comprises at least one modified nucleoside comprising a non-bicyclic modified sugar moiety.
Embodiment 12. The oligomeric compound of embodiment 11, wherein the non-bicyclic modified sugar moiety is a 2′-MOE sugar moiety or 2′-OMe modified sugar moiety.
Embodiment 13. The oligomeric compound of any of embodiments 8-13, wherein the modified oligonucleotide comprises at least one modified nucleoside comprising a sugar surrogate.
Embodiment 14. The oligomeric compound of embodiment 13, wherein the modified oligonucleotide comprises at least one modified nucleoside comprising a sugar surrogate selected from morpholino and PNA.
Embodiment 15. The oligomeric compound of any of embodiments 1-14, wherein the modified oligonucleotide is a gapmer.
Embodiment 16. The oligomeric compound of any of embodiments 1-15, wherein the modified oligonucleotide has a sugar motif comprising:
   a 5′-region consisting of 1-6 linked 5′-region nucleosides;
   a central region consisting of 6-10 linked central region nucleosides; and
   a 3′-region consisting of 1-6 linked 3′-region nucleosides; wherein each of the 5′-region nucleosides and each of the 3′-region nucleosides comprises a modified sugar moiety and the central region is a deoxy region.
Embodiment 17. A pharmaceutical composition comprising the oligomeric compound of any of embodiments 1-16 and a pharmaceutically acceptable diluent or carrier.
Embodiment 18. A method of treating a disease associated with KCNQ2 comprising administering to a subject having or at risk for developing a disease associated with KCNQ2 a therapeutically effective amount of a pharmaceutical composition of embodiment 17; thereby treating the disease associated with KCNQ2.
Embodiment 19. The method of embodiment 18, further comprising identifying a subject having or at risk for developing a disease associate with KCNQ2.
Embodiment 20. The method of embodiment 18 or 19, further comprising genetically testing the subject for a mutation in a KCNQ2 gene.
Embodiment 21. The method of embodiment 18, wherein the disease associated with KCNQ2 is an epileptic encephalopathy.
Embodiment 22. The method of embodiment 21, wherein the epileptic encephalopathy is KCNQ2-associated neonatal epileptic encephalopathy.
Embodiment 23. The method of embodiment 22, wherein at least one symptom or hallmark of the epileptic encephalopathy is ameliorated.
Embodiment 24. The method of embodiment 23, wherein the symptom or hallmark is any of infantile spasms or seizures, EEG abnormalities, brain MRI abnormalities, or developmental impairment.
Embodiment 25. An oligomeric compound, comprising a modified oligonucleotide consisting of 12 to 30 linked nucleosides wherein the nucleobase sequence of the modified oligonucleotide is at least 90% complementary to an equal length portion of a KCNQ2 nucleic acid, and wherein the modified oligonucleotide comprises at least one modification selected from a modified sugar moiety and a modified internucleoside linkage.
Embodiment 26. The oligomeric compound of embodiment 25, wherein the modified oligonucleotide comprises an at least 8 nucleobase portion, at least 9 nucleobase portion, at least 10 nucleobase portion, at least 11 nucleobase portion, at least 12 nucleobase portion, at least 13 nucleobase portion, at least 14 nucleobase portion, at least 15 nucleobase portion, at least 16 nucleobase portion, at least 17 nucleobase portion, at least 18 nucleobase portion, at least 19 nucleobase portion, or a 20 nucleobase portion of any of SEQ ID NO: 21-1029.
Embodiment 27. An oligomeric compound comprising a modified oligonucleotide consisting of 12 to 30 linked nucleosides and having a nucleobase sequence comprising at least 12, at least 13, at least 14, at least 15, at least 16, at least 17, at least 18, at least 19 or 20 contiguous nucleobases of any of SEQ ID NO: 21-1029.
Embodiment 28. An oligomeric compound comprising a modified oligonucleotide consisting of 12 to 30 linked nucleosides and having a nucleobase sequence having at least 8, at least 9, at least 10, at least 11, at least 12, at least 13, at least 14, at least 15, at least 16, at least 17, at least 18, at least 19, or at least 20 contiguous nucleobases at least 90%, at least 95%, or 100% complementary to:
   an equal length portion of nucleobases 4,600-4,624 of SEQ ID NO: 2;
   an equal length portion of nucleobases 8,970-8,990 of SEQ ID NO: 2;
   an equal length portion of nucleobases 23,730-23,752 of SEQ ID NO: 2;
   an equal length portion of nucleobases 24,439-24,775 of SEQ ID NO: 2;
   an equal length portion of nucleobases 27,275-27,306 of SEQ ID NO: 2;
   an equal length portion of nucleobases 33,048-33,083 of SEQ ID NO: 2;
   an equal length portion of nucleobases 33,054-33,083 of SEQ ID NO: 2;
   an equal length portion of nucleobases 34,198-34,232 of SEQ ID NO: 2;
   an equal length portion of nucleobases 34,543-34,563 of SEQ ID NO: 2;
   an equal length portion of nucleobases 35,323-35,343 of SEQ ID NO: 2;
   an equal length portion of nucleobases 41,334-41,357 of SEQ ID NO: 2;
   an equal length portion of nucleobases 57,517-57,552 of SEQ ID NO: 2;
   an equal length portion of nucleobases 57,523-57,544 of SEQ ID NO: 2;
   an equal length portion of nucleobases 65,636-65,671 of SEQ ID NO: 2;
   an equal length portion of nucleobases 65,745-65,772 of SEQ ID NO: 2;
   an equal length portion of nucleobases 1,615-1,642 of SEQ ID NO: 2;
   an equal length portion of nucleobases 3,758-3,780 of SEQ ID NO: 2;
   an equal length portion of nucleobases 4,631-4,650 of SEQ ID NO: 2;
   an equal length portion of nucleobases 6,025-6,050 of SEQ ID NO: 2;
   an equal length portion of nucleobases 6,033-6,055 of SEQ ID NO: 2;
   an equal length portion of nucleobases 6,547-6,573 of SEQ ID NO: 2;
   an equal length portion of nucleobases 7,371-7,394 of SEQ ID NO: 2;
   an equal length portion of nucleobases 9,932-9,955 of SEQ ID NO: 2;
   an equal length portion of nucleobases 10,324-10,347 of SEQ ID NO: 2;
   an equal length portion of nucleobases 10,439-10,462 of SEQ ID NO: 2;
   an equal length portion of nucleobases 13,863-13,889 of SEQ ID NO: 2;
   an equal length portion of nucleobases 14,261-14,284 of SEQ ID NO: 2;
   an equal length portion of nucleobases 16,110-16,137 of SEQ ID NO: 2;
   an equal length portion of nucleobases 16,142-16,167 of SEQ ID NO: 2;
   an equal length portion of nucleobases 16,506-16,533 of SEQ ID NO: 2;
   an equal length portion of nucleobases 18,835-18,858 of SEQ ID NO: 2;
   an equal length portion of nucleobases 20,464-20,486 of SEQ ID NO: 2;
   an equal length portion of nucleobases 24,161-24,186 of SEQ ID NO: 2;
   an equal length portion of nucleobases 25,662-25,685 of SEQ ID NO: 2;
   an equal length portion of nucleobases 26,622-26,647 of SEQ ID NO: 2;
   an equal length portion of nucleobases 26,709-26,741 of SEQ ID NO: 2;
   an equal length portion of nucleobases 27,035-27,059 of SEQ ID NO: 2;
   an equal length portion of nucleobases 28,752-28,774 of SEQ ID NO: 2;
   an equal length portion of nucleobases 29,184-29,211 of SEQ ID NO: 2;
   an equal length portion of nucleobases 29,405-29,431 of SEQ ID NO: 2;
   an equal length portion of nucleobases 29,938-29,960 of SEQ ID NO: 2;
   an equal length portion of nucleobases 30,968-30,991 of SEQ ID NO: 2;
   an equal length portion of nucleobases 31,860-31,886 of SEQ ID NO: 2;
   an equal length portion of nucleobases 32,962-32,984 of SEQ ID NO: 2;
   an equal length portion of nucleobases 37,062-37,091 of SEQ ID NO: 2; or
   an equal length portion of nucleobases 39,847-39,870 of SEQ ID NO: 2.
Embodiment 29. An oligomeric compound comprising a modified oligonucleotide consisting of 12 to 30 linked nucleosides and having a nucleobase sequence comprising at least 8, at least 9, at least 10, at least 11, at least 12, at least 13, at least 14, at least 15, at least 16, at least 17, at least 18, at least 19, or 20 contiguous nucleobases of a sequence selected from:
   SEQ ID NOs: 67, 82;
   SEQ ID NOs: 34, 83;
   SEQ ID NOs: 61, 97;
   SEQ ID NOs: 40, 42, 88;
   SEQ ID NOs: 21, 24, 32, 41, 44, 71, 96;
   SEQ ID NOs: 23, 25, 30, 50, 57, 68, 89, 91, 95;
   SEQ ID NOs: 25, 30, 50, 68, 89, 91, 95;
   SEQ ID NOs: 35, 56;
   SEQ ID NOs: 58, 84;
   SEQ ID NOs: 37, 74;
   SEQ ID NOs: 31, 98;
   SEQ ID NOs: 59, 76;
   SEQ ID NOs: 22, 36, 52, 55, 64, 69, 70, 86, 90;
   SEQ ID NOs: 55, 86;
   SEQ ID NOs: 26-29, 49, 60, 80, 87, 92;
   SEQ ID NOs: 43, 45, 54, 63, 78;
   SEQ ID NOs: 132-137;
   SEQ ID NOs: 725, 846, 903, 1005;
   SEQ ID NOs: 99-105;
   SEQ ID NOs: 641, 679, 771, 804, 949, 987;
   SEQ ID NOs: 714, 772, 816, 946;
   SEQ ID NOs: 240-244, 246;
   SEQ ID NOs: 223-227;
   SEQ ID NOs: 644, 715, 760, 866, 991;
   SEQ ID NOs: 807, 886, 913, 960, 971;
   SEQ ID NOs: 654, 655, 754, 796, 817;
   SEQ ID NOs: 575, 590, 684, 750, 869, 881, 974, 1029;
   SEQ ID NOs: 722, 769, 849, 945, 992;
   SEQ ID NOs: 597, 680, 777, 785, 800, 863, 889, 951, 955;
   SEQ ID NOs: 207-212, 214;
   SEQ ID NOs: 577, 592, 666, 720, 778, 834, 938, 1021, 1026;
   SEQ ID NOs: 326-329;
   SEQ ID NOs: 314, 315, 317;
   SEQ ID NOs: 302-306;
   SEQ ID NOs: 286-289;
   SEQ ID NOs: 617, 671, 746, 801, 809, 896, 1027;
   SEQ ID NOs: 579, 643, 653, 676, 748, 773, 838, 868, 898, 911, 918, 957, 969, 1003;
   SEQ ID NOs: 572, 673, 789, 850, 887, 997;
   SEQ ID NOs: 266, 267, 718, 739;
   SEQ ID NOs: 255-259, 410;
   SEQ ID NOs: 636, 694, 752, 828, 952, 970, 1020;
   SEQ ID NOs: 626, 630, 1004;
   SEQ ID NOs: 602, 765, 812, 932, 972;
   SEQ ID NOs: 397-401, 692, 729, 832;
   SEQ ID NOs: 638, 658, 853, 897;
   SEQ ID NOs: 615, 627, 645, 747, 788, 815, 862, 915, 917, 1024, 1028; or
   SEQ ID NOs: 591, 650, 731, 743, 996.
Embodiment 30. The oligomeric compound of any of embodiments 27-29, wherein the modified oligonucleotide has a nucleobase sequence that is at least 70%, at least 75%, at least 80%, at least 85%, at least 90%, at least 95%, or is 100% complementary to the nucleobase sequence of SEQ ID NO: 1 or SEQ ID NO: 2 when measured across the entire nucleobase sequence of the modified oligonucleotide.
Embodiment 31. The oligomeric compound of any of embodiments 25-30, wherein the modified oligonucleotide comprises at least one modified nucleoside.
Embodiment 32. The oligomeric compound of embodiment 31, wherein the modified oligonucleotide comprises at least one modified nucleoside comprising a modified sugar moiety.
Embodiment 33. The oligomeric compound of embodiment 32, wherein the modified oligonucleotide comprises at least one modified nucleoside comprising a bicyclic sugar moiety.
Embodiment 34. The oligomeric compound of embodiment 33, wherein the modified oligonucleotide comprises at least one modified nucleoside comprising a bicyclic sugar moiety having a 2′-4′ bridge, wherein the 2′-4′ bridge is selected from —O—CH₂—; and —O—CH(CH₃)—.
Embodiment 35. The oligomeric compound of any of embodiments 32-34, wherein the modified oligonucleotide comprises at least one modified nucleoside comprising a non-bicyclic modified sugar moiety.
Embodiment 36. The oligomeric compound of embodiment 35, wherein the non-bicyclic modified sugar moiety is a 2′-MOE sugar moiety or 2′-OMe modified sugar moiety.
Embodiment 37. The oligomeric compound of any of embodiments 32-36, wherein the modified oligonucleotide comprises at least one modified nucleoside comprising a sugar surrogate.
Embodiment 38. The oligomeric compound of embodiment 37, wherein the modified oligonucleotide comprises at least one modified nucleoside comprising a sugar surrogate selected from morpholino and PNA.
Embodiment 39. The oligomeric compound of any of embodiments 25-38, wherein the modified oligonucleotide is a gapmer.
Embodiment 40. The oligomeric compound of any of embodiments 25-39, wherein the modified oligonucleotide has a sugar motif comprising:
   a 5′-region consisting of 1-6 linked 5′-region nucleosides;
   a central region consisting of 6-10 linked central region nucleosides; and
   a 3′-region consisting of 1-6 linked 3′-region nucleosides; wherein each of the 5′-region nucleosides and each of the 3′-region nucleosides comprises a modified sugar moiety and the central region is a deoxy region.
Embodiment 41. The oligomeric compound of any of embodiments 25-40, wherein the oligomeric compound consists of the modified oligonucleotide.
Embodiment 42. The oligomeric compound of any of embodiments 25-41, wherein the oligomeric compound is single stranded.
Embodiment 43. An oligomeric duplex, comprising the oligomeric compound of any of embodiments 25-42.
Embodiment 44. A pharmaceutical composition comprising the oligomeric compound of any of embodiments 25-42 or the oligomeric duplex of embodiment 43 and a pharmaceutically acceptable diluent or carrier.
Embodiment 45. A method of treating a disease associated with KCNQ2 comprising administering to a subject having or at risk for developing a disease associated with KCNQ2 a therapeutically effective amount of a pharmaceutical composition of embodiment 44; thereby treating the disease associated with KCNQ2.
Embodiment 46. The method of embodiment 45, further comprising identifying a subject having or at risk for developing a disease associate with KCNQ2.
Embodiment 47. The method of embodiment 45 or 46, further comprising genetically testing the subject for a mutation in a KCNQ2 gene.
Embodiment 48. The method of embodiment 47, wherein the disease associated with KCNQ2 is an epileptic encephalopathy.
Embodiment 49. The method of embodiment 48, wherein the epileptic encephalopathy is KCNQ2-associated neonatal epileptic encephalopathy.
Embodiment 50. The method of embodiment 49, wherein at least one symptom or hallmark of the epileptic encephalopathy is ameliorated.
Embodiment 51. The method of embodiment 50, wherein the symptom or hallmark is any of infantile spasms or seizures, EEG abnormalities, brain MRI abnormalities, or developmental impairment.

I. Certain Oligonucleotides

In certain embodiments, provided herein are oligomeric compounds comprising oligonucleotides, which consist of linked nucleosides. Oligonucleotides may be unmodified oligonucleotides (RNA or DNA) or may be modified oligonucleotides. Modified oligonucleotides comprise at least one modification relative to unmodified RNA or DNA. That is, modified oligonucleotides comprise at least one modified nucleoside (comprising a modified sugar moiety and/or a modified nucleobase) and/or at least one modified internucleoside linkage.

A. Certain Modified Nucleosides

Modified nucleosides comprise a modified sugar moiety or a modified nucleobase or both a modified sugar moiety and a modified nucleobase.

1. Certain Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more substituent groups none of which bridges two atoms of the furanosyl ring to form a bicyclic structure. Such non bridging substituents may be at any position of the furanosyl, including but not limited to substituents at the 2′, 4′, and/or 5′ positions. In certain embodiments one or more non-bridging substituent of non-bicyclic modified sugar moieties is branched. Examples of 2′-substituent groups suitable for non-bicyclic modified sugar moieties include but are not limited to: 2′-F, 2′-OCH₃ (“OMe” or “O-methyl”), and 2′-O(CH₂)₂OCH₃ (“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5-methyl (R or S), 5′-vinyl, and 5′-methoxy. In certain embodiments, non-bicyclic modified sugar moieties comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836.

In certain embodiments, a 2′-substituted non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted nucleoside non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCH₃, and OCH₂CH₂OCH₃.

Certain modified sugar moieties comprise a substituent that bridges two atoms of the furanosyl ring to form a second ring, resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt”), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O- 2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;
wherein:

x is 0, 1, or 2;

n is 1, 2, 3, or 4;

each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and

each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129, 8362-8379; Wengel et al., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

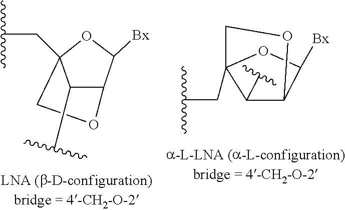

α-L-methyleneoxy (4′-CH₂—O-2′) or α-L-LNA bicyclic nucleosides have been incorporated into oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, C J. Bioorg. & Med. Chem. 2002, 10, 841-854), fluoro HNA:

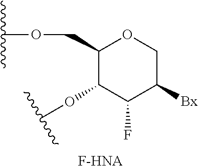

(“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

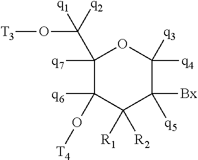

wherein, independently, for each of said modified THP nucleoside:

Bx is a nucleobase moiety;

T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group;

q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

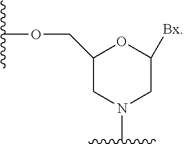

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides.

2. Certain Modified Nucleobases

In certain embodiments, modified oligonucleotides comprise one or more nucleosides comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside.

In certain embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

3. Certain Modified Internucleoside Linkages

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphodiesters, which contain a phosphodiester bond (“P(O₂)═O”) (also referred to as unmodified or naturally occurring linkages), phosphotriesters, methylphosphonates, phosphoramidates, and phosphorothioates (“P(O₂)═S”), and phosphorodithioates (“HS—P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphodiester internucleoside linkages, can be used to alter, typically increase, nuclease resistance of the oligonucleotide. In certain embodiments, internucleoside linkages having a chiral atom can be prepared as a racemic mixture, or as separate enantiomers. Methods of preparation of phosphorous-containing and non-phosphorous-containing internucleoside linkages are well known to those skilled in the art.

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkage in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al., JACCS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

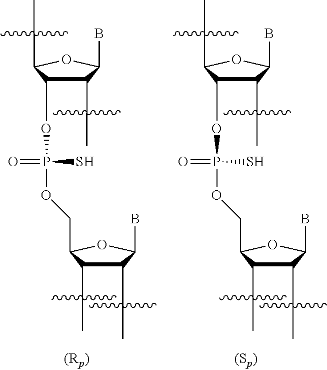

Unless otherwise indicated, chiral internucleoside linkages of modified oligonucleotides described herein can be stereorandom or in a particular stereochemical configuration.

Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl (MOP), and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

B. Certain Motifs

In certain embodiments, modified oligonucleotides comprise one or more modified nucleosides comprising a modified sugar moiety. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

1. Certain Sugar Motifs

In certain embodiments, oligonucleotides comprise one or more type of modified sugar and/or unmodified sugar moiety arranged along the oligonucleotide or portion thereof in a defined pattern or sugar motif. In certain instances, such sugar motifs include but are not limited to any of the sugar modifications discussed herein.

In certain embodiments, modified oligonucleotides have a gapmer motif, which is defined by two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer). In certain embodiments, the wings of a gapmer comprise 1-6 nucleosides. In certain embodiments, each nucleoside of each wing of a gapmer comprises a modified sugar moiety. In certain embodiments, at least one nucleoside of each wing of a gapmer comprises a modified sugar moiety. In certain embodiments, at least two nucleosides of each wing of a gapmer comprises a modified sugar moiety. In certain embodiments, at least three nucleosides of each wing of a gapmer comprises a modified sugar moiety. In certain embodiments, at least four nucleosides of each wing of a gapmer comprises a modified sugar moiety. In certain embodiments, at least five nucleosides of each wing of a gapmer comprises a modified sugar moiety.

In certain embodiments, the gap of a gapmer comprises 7-12 nucleosides. In certain embodiments, each nucleoside of the gap of a gapmer comprises a 2′-deoxyribosyl sugar moiety. In certain embodiments, each nucleoside of the gap of a gapmer comprises a 2′-β-D-deoxyribosyl sugar moiety. In certain embodiments, at least one nucleoside of the gap of a gapmer comprises a modified sugar moiety. In certain embodiments, at least one nucleoside of the gap of a gapmer comprises a 2′-OMe sugar moiety.

In certain embodiments, the gapmer is a deoxy gapmer. In certain embodiments, the nucleosides on the gap side of each wing/gap junction comprise 2′-deoxyribosyl sugar moieties and the nucleosides on the wing sides of each wing/gap junction comprise modified sugar moieties. In certain embodiments, each nucleoside of the gap comprises a 2′-deoxyribosyl sugar moiety. In certain embodiments, each nucleoside of each wing of a gapmer comprises a modified sugar moiety. In certain embodiments, one nucleoside of the gap comprises a modified sugar moiety and each remaining nucleoside of the gap comprises a 2′-deoxyribosyl sugar moiety.

In certain embodiments, modified oligonucleotides comprise or consist of a portion having a fully modified sugar motif. In such embodiments, each nucleoside of the fully modified portion of the modified oligonucleotide comprises a modified sugar moiety. In certain embodiments, each nucleoside of the entire modified oligonucleotide comprises a modified sugar moiety. In certain embodiments, modified oligonucleotides comprise or consist of a portion having a fully modified sugar motif, wherein each nucleoside within the fully modified portion comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified oligonucleotide comprises the same 2′-modification.

Herein, the lengths (number of nucleosides) of the three regions of a gapmer may be provided using the notation [# of nucleosides in the 5′-wing]-[# of nucleosides in the gap]-[# of nucleosides in the 3′-wing]. Thus, a 5-10-5 gapmer consists of 5 linked nucleosides in each wing and 10 linked nucleosides in the gap. Where such nomenclature is followed by a specific modification, that modification is the modification in each sugar moiety of each wing and the gap nucleosides comprises a 2′-β-D-deoxyribosyl sugar moiety. Thus, a 5-10-5 MOE gapmer consists of 5 linked 2′-MOE nucleosides in the 5′-wing, 10 linked a 2′-β-D-deoxynucleosides in the gap, and 5 linked 2′-MOE nucleosides in the 3′-wing.

In certain embodiments, modified oligonucleotides are 5-10-5 MOE gapmers. In certain embodiments, modified oligonucleotides are X-Y-Z MOE gapmers, wherein X and Z are independently selected from 1, 2, 3, 4, 5, or 6 2′-MOE modified nucleosides and Y is 7, 8, 9, 10, or 11 2′-deoxynucleosides. In certain embodiments, modified oligonucleotides are X-Y-Z mixed wing gapmers, wherein X and Z are independently selected from 1, 2, 3, 4, 5, or 6 and Y is 7, 8, 9, 10, or 11.

2. Certain Nucleobase Motifs

In certain embodiments, oligonucleotides comprise modified and/or unmodified nucleobases arranged along the oligonucleotide or portion thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methyl cytosines. In certain embodiments, all of the cytosine nucleobases are 5-methyl cytosines and all of the other nucleobases of the modified oligonucleotide are unmodified nucleobases.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-deoxyribosyl sugar moiety. In certain embodiments, the modified nucleobase is selected from: a 2-thiopyrimidine and a 5-propynepyrimidine.

3. Certain Internucleoside Linkage Motifs

In certain embodiments, oligonucleotides comprise modified and/or unmodified internucleoside linkages arranged along the oligonucleotide or portion thereof in a defined pattern or motif. In certain embodiments, each internucleoside linking group is a phosphodiester internucleoside linkage (P(O₂)═0). In certain embodiments, each internucleoside linking group of a modified oligonucleotide is a phosphorothioate internucleoside linkage (P(O₂)═S). In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate, a (Sp) phosphorothioate, and a (Rp) phosphorothioate. In certain embodiments, the sugar motif of a modified oligonucleotide is a gapmer and the internucleoside linkages within the gap are all modified. In certain such embodiments, some or all of the internucleoside linkages in the wings are unmodified phosphodiester internucleoside linkages. In certain embodiments, the terminal internucleoside linkages are modified. In certain embodiments, the sugar motif of a modified oligonucleotide is a gapmer, and the internucleoside linkage motif comprises at least one phosphodiester internucleoside linkage in at least one wing, wherein the at least one phosphodiester linkage is not a terminal internucleoside linkage, and the remaining internucleoside linkages are phosphorothioate internucleoside linkages. In certain such embodiments, all of the phosphorothioate linkages are stereorandom. In certain embodiments, all of the phosphorothioate linkages in the wings are (Sp) phosphorothioates, and the gap comprises at least one Sp, Sp, Rp motif. In certain embodiments, all of the internucleoside linkages are either phosphodiester internucleoside linkages or phosphorothioate internucleoside linkages, and the chiral motif is (5′ to 3′): Sp-o-o-o-Sp-Sp-Sp-Rp-Sp-Sp-Rp-Sp-Sp-Sp-Sp-Sp-Sp-Sp-Sp or Sp-o-o-o-Sp-Sp-Sp-Rp-Sp-Sp-Sp-Sp-Sp-Sp-Sp-Sp-Sp-Sp-Sp, wherein each ‘Sp’ represents a (Sp) phosphorothioate internucleoside linkage, each ‘Rp’ is a Rp internucleoside linkage, and each ‘o’ represents a phosphodiester internucleoside linkage. In certain embodiments, populations of modified oligonucleotides are enriched for modified oligonucleotides comprising such internucleoside linkage motifs.

In certain embodiments, modified oligonucleotides have an internucleoside linkage motif of soooossssssssssooss, wherein each “s” represents a phosphorothioate internucleoside linkage and each “o” represents a phosphodiester internucleoside linkage. In certain embodiments, modified oligonucleotides have an internucleoside linkage motif of (5′ to 3′): sooooossssssssssoss, wherein each “s” represents a phosphorothioate internucleoside linkage and each “o” represents a phosphodiester internucleoside linkage. In certain embodiments, modified oligonucleotides have an internucleoside linkage motif of (5′ to 3′): sssosssssssssssosss, wherein each “s” represents a phosphorothioate internucleoside linkage and each “o” represents a phosphodiester internucleoside linkage. In certain embodiments, modified oligonucleotides have an internucleoside linkage motif of (5′ to 3′): sssosssssssssoss, wherein each “s” represents a phosphorothioate internucleoside linkage and each “o” represents a phosphodiester internucleoside linkage.

C. Certain Lengths

It is possible to increase or decrease the length of an oligonucleotide without eliminating activity. For example, in Woolf et al. (Proc. Natl. Acad. Sci. USA 89:7305-7309, 1992), a series of oligonucleotides 13-25 nucleobases in length were tested for their ability to induce cleavage of a target RNA in an oocyte injection model. Oligonucleotides 25 nucleobases in length with 8 or 11 mismatch bases near the ends of the oligonucleotides were able to direct specific cleavage of the target RNA, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

D. Certain Modified Oligonucleotides

In certain embodiments, the above modifications (sugar, nucleobase, internucleoside linkage) are incorporated into a modified oligonucleotide. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such sugar gapmer oligonucleotides may comprise one or more modified nucleobase independent of the gapmer pattern of the sugar modifications. Unless otherwise indicated, all modifications are independent of nucleobase sequence.

E. Certain Populations of Modified Oligonucleotides

Populations of modified oligonucleotides in which all of the modified oligonucleotides of the population have the same molecular formula can be stereorandom populations or chirally enriched populations. All of the chiral centers of all of the modified oligonucleotides are stereorandom in a stereorandom population. In a chirally enriched population, at least one particular chiral center is not stereorandom in the modified oligonucleotides of the population. In certain embodiments, the modified oligonucleotides of a chirally enriched population are enriched for β-D ribosyl sugar moieties, and all of the phosphorothioate internucleoside linkages are stereorandom. In certain embodiments, the modified oligonucleotides of a chirally enriched population are enriched for both β-D ribosyl sugar moieties and at least one, particular phosphorothioate internucleoside linkage in a particular stereochemical configuration.

F. Nucleobase Sequence

In certain embodiments, oligonucleotides (unmodified or modified oligonucleotides) are further described by their nucleobase sequence. In certain embodiments oligonucleotides have a nucleobase sequence that is complementary to a second oligonucleotide or an identified reference nucleic acid, such as a target nucleic acid. In certain such embodiments, a portion of an oligonucleotide has a nucleobase sequence that is complementary to a second oligonucleotide or an identified reference nucleic acid, such as a target nucleic acid. In certain embodiments, the nucleobase sequence of a portion or entire length of an oligonucleotide is at least 50%, at least 60%, at least 70%, at least 80%, at least 85%, at least 90%, at least 95%, or 100% complementary to the second oligonucleotide or nucleic acid, such as a target nucleic acid.

I. Certain Oligomeric Compounds

In certain embodiments, provided herein are oligomeric compounds, which consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance. In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N. Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

1. Conjugate Moieties

Conjugate moieties include, without limitation, intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

2. Conjugate Linkers

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methyl cytosine, 4-N-benzoyl-5-methyl cytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate or phosphodiester linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxynucleoside that is attached to either the 3′ or 5′-terminal nucleoside of an oligonucleotide by a phosphodiester internucleoside linkage and covalently attached to the remainder of the conjugate linker or conjugate moiety by a phosphate or phosphorothioate linkage. In certain such embodiments, the cleavable moiety is 2′-deoxyadenosine.

3. Cell-Targeting Moieties

In certain embodiments, a conjugate group comprises a cell-targeting moiety. In certain embodiments, a conjugate group has the general formula:

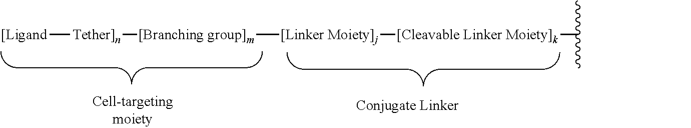

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

B. Certain Terminal Groups

In certain embodiments, oligomeric compounds comprise one or more terminal groups. In certain such embodiments, oligomeric compounds comprise a stabilized 5′-phosphate. Stabilized 5′-phosphates include, but are not limited to 5′-phosphanates, including, but not limited to 5′-vinylphosphonates. In certain embodiments, terminal groups comprise one or more abasic nucleosides and/or inverted nucleosides. In certain embodiments, terminal groups comprise one or more 2′-linked nucleosides. In certain such embodiments, the 2′-linked nucleoside is an abasic nucleoside.

III. Oligomeric Duplexes

In certain embodiments, oligomeric compounds described herein comprise an oligonucleotide, having a nucleobase sequence complementary to that of a target nucleic acid. In certain embodiments, an oligomeric compound is paired with a second oligomeric compound to form an oligomeric duplex. Such oligomeric duplexes comprise a first oligomeric compound having a portion complementary to a target nucleic acid and a second oligomeric compound having a portion complementary to the first oligomeric compound. In certain embodiments, the first oligomeric compound of an oligomeric duplex comprises or consists of (1) a modified or unmodified oligonucleotide and optionally a conjugate group and (2) a second modified or unmodified oligonucleotide and optionally a conjugate group. Either or both oligomeric compounds of an oligomeric duplex may comprise a conjugate group. The oligonucleotides of each oligomeric compound of an oligomeric duplex may include non-complementary overhanging nucleosides.

IV. Antisense Activity

In certain embodiments, oligomeric compounds and oligomeric duplexes are capable of hybridizing to a target nucleic acid, resulting in at least one antisense activity; such oligomeric compounds and oligomeric duplexes are antisense compounds. In certain embodiments, antisense compounds have antisense activity when they reduce the amount or activity of a target nucleic acid by 25% or more in the standard cell assay. In certain embodiments, antisense compounds selectively affect one or more target nucleic acid. Such antisense compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, described herein are antisense compounds that are sufficiently “DNA-like” to elicit RNase H activity. In certain embodiments, one or more non-DNA-like nucleoside in the gap of a gapmer is tolerated.

In certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute. Antisense compounds that are loaded into RISC are RNAi compounds. RNAi compounds may be double-stranded (siRNA) or single-stranded (ssRNA).

In certain embodiments, hybridization of an antisense compound to a target nucleic acid does not result in recruitment of a protein that cleaves that target nucleic acid. In certain embodiments, hybridization of the antisense compound to the target nucleic acid results in alteration of splicing of the target nucleic acid. In certain embodiments, hybridization of an antisense compound to a target nucleic acid results in inhibition of a binding interaction between the target nucleic acid and a protein or other nucleic acid. In certain embodiments, hybridization of an antisense compound to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein and/or a phenotypic change in a cell or subject.

V. Certain Target Nucleic Acids

In certain embodiments, oligomeric compounds comprise or consist of an oligonucleotide comprising a portion that is complementary to a target nucleic acid. In certain embodiments, the target nucleic acid is an endogenous RNA molecule. In certain embodiments, the target nucleic acid encodes a protein. In certain such embodiments, the target nucleic acid is selected from: a mature mRNA and a pre-mRNA, including intronic, exonic and untranslated regions. In certain embodiments, the target RNA is a mature mRNA. In certain embodiments, the target nucleic acid is a pre-mRNA. In certain such embodiments, the target region is entirely within an intron. In certain embodiments, the target region spans an intron/exon junction. In certain embodiments, the target region is at least 50% within an intron.

A. Complementarity/Mismatches to the Target Nucleic Acid

It is possible to introduce mismatch bases without eliminating activity. For example, Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo. Maher and Dolnick (Nuc. Acid. Res. 16:3341-3358, 1988) tested a series of tandem 14 nucleobase oligonucleotides, and 28 and 42 nucleobase oligonucleotides comprised of the sequence of two or three of the tandem oligonucleotides, respectively, for their ability to arrest translation of human DHFR in a rabbit reticulocyte assay. Each of the three 14 nucleobase oligonucleotides alone was able to inhibit translation, albeit at a more modest level than the 28 or 42 nucleobase oligonucleotides.

In certain embodiments, oligonucleotides are complementary to the target nucleic acid over the entire length of the oligonucleotide. In certain embodiments, oligonucleotides are 99%, 95%, 90%, 85%, or 80% complementary to the target nucleic acid. In certain embodiments, oligonucleotides are at least 80% complementary to the target nucleic acid over the entire length of the oligonucleotide and comprise a portion that is 100% or fully complementary to a target nucleic acid. In certain embodiments, the portion of full complementarity is 6, 7, 8, 9, 10, 11, 12, 13, 14, 15, 16, 17, 18, 19, 20, 21, 22, 23, or 24 nucleobases in length.

In certain embodiments, oligonucleotides comprise one or more mismatched nucleobases relative to the target nucleic acid. In certain embodiments, antisense activity against the target is reduced by such mismatch, but activity against a non-target is reduced by a greater amount. Thus, in certain embodiments selectivity of the oligonucleotide is improved. In certain embodiments, the mismatch is specifically positioned within an oligonucleotide having a gapmer motif. In certain embodiments, the mismatch is at position 1, 2, 3, 4, 5, 6, 7, 8, 9, 10, or 11 from the 5′-end of the gap region. In certain embodiments, the mismatch is at position 1, 2, 3, 4, 5, or 6 from the 5′-end of the 5′ wing region or the 3′ wing region.

B. KCNQ2

In certain embodiments, oligomeric compounds comprise or consist of an oligonucleotide that is complementary to a target nucleic acid, wherein the target nucleic acid is a KCNQ2 nucleic acid. In certain embodiments, the KCNQ2 nucleic acid has the sequence set forth in SEQ ID NO: 1 (cDNA of ENSEMBL Accession No. ENST00000359125.6 from ENSEMBL version 98: September 2019, human reference assembly version GRCh38.p13 located on the reverse strand of chromosome 20 (CM000682.2) from positions 63,406,137 to 63,472,590) or SEQ ID NO: 2 (ENSEMBL Accession No. ENSG00000075043.18 from ENSEMBL version 98: September 2019, human reference assembly version GRCh38.p13 located on the reverse strand of chromosome 20 (CM000682.2) from positions 63,400,210 to 63,472,677).

In certain embodiments, contacting a cell with an oligomeric compound complementary to SEQ ID NO: 1 or SEQ ID NO: 2 reduces the amount of KCNQ2 RNA in a cell. In certain embodiments, contacting a cell with an oligomeric compound complementary to SEQ ID NO: 1 or SEQ ID NO: 2 reduces the amount of Kᵥ7.2 protein in a cell. In certain embodiments, the cell is in vitro. In certain embodiments, the cell is in a subject. In certain embodiments, the oligomeric compound consists of a modified oligonucleotide. In certain embodiments, contacting a cell in a subject with an oligomeric compound complementary to SEQ ID NO: 1 or SEQ ID NO: 2 ameliorates one or more symptoms or hallmarks of an epileptic encephalopathy. In certain embodiments, the epileptic encephalopathy is associated with a gain-of-function or dominant negative mutation in KCNQ2. In certain embodiments, the symptom or hallmark is selected from infantile spasms or seizures, EEG abnormalities, brain MRI abnormalities in the infant, and developmental impairment.

In certain embodiments, an oligomeric compound complementary to SEQ ID NO: 1 or SEQ ID NO: 2 is capable of reducing the detectable amount of KCNQ2 RNA in vitro by at least 10%, at least 20%, at least 30%, at least 40%, at least 50%, at least 60%, at least 70%, at least 80%, or at least 90% when administered according to the standard cell assay. In certain embodiments, an oligomeric compound complementary to SEQ ID NO: 1 or SEQ ID NO: 2 is capable of decreasing the amount of Kᵥ7.2 protein in vitro by at least 10%, at least 20%, at least 30%, at least 40%, at least 50%, at least 60%, at least 70%, at least 80%, or at least 90% when administered according to the standard cell assay. In certain embodiments, an oligomeric compound complementary to SEQ ID NO: 1 or SEQ ID NO: 2 is capable of reducing the detectable amount of KCNQ2 RNA in the CSF of a subject by at least 10%, at least 20%, at least 30%, at least 40%, at least 50%, at least 60%, at least 70%, at least 80%, or at least 90%. In certain embodiments, an oligomeric compound complementary to SEQ ID NO: 1 or SEQ ID NO: 2 is capable of decreasing the detectable amount of Kᵥ7.2 protein in the CSF of a subject by at least 10%, at least 20%, at least 30%, at least 40%, at least 50%, at least 60%, at least 70%, at least 80%, or at least 90%.

C. Certain Target Nucleic Acids in Certain Tissues

In certain embodiments, oligomeric compounds comprise or consist of an oligonucleotide comprising a portion that is complementary to a target nucleic acid, wherein the target nucleic acid is expressed in a pharmacologically relevant tissue. In certain embodiments, the pharmacologically relevant tissues are the cells and tissues that comprise the central nervous system. Such tissues include the cortex, hippocampus, and spinal cord.

VI. Certain Pharmaceutical Compositions

In certain embodiments, described herein are pharmaceutical compositions comprising one or more oligomeric compounds. In certain embodiments, the one or more oligomeric compounds each consists of a modified oligonucleotide. In certain embodiments, the pharmaceutical composition comprises a pharmaceutically acceptable diluent or carrier. In certain embodiments, a pharmaceutical composition comprises or consists of a sterile saline solution and one or more oligomeric compound. In certain embodiments, the sterile saline is pharmaceutical grade saline. In certain embodiments, a pharmaceutical composition comprises or consists of one or more oligomeric compound and sterile water. In certain embodiments, the sterile water is pharmaceutical grade water. In certain embodiments, a pharmaceutical composition comprises or consists of one or more oligomeric compound and phosphate-buffered saline (PBS). In certain embodiments, the sterile PBS is pharmaceutical grade PBS. In certain embodiments, a pharmaceutical composition comprises or consists of one or more oligomeric compound and artificial cerebrospinal fluid. In certain embodiments, the artificial cerebrospinal fluid is pharmaceutical grade.

In certain embodiments, a pharmaceutical composition comprises a modified oligonucleotide and artificial cerebrospinal fluid. In certain embodiments, a pharmaceutical composition consists of a modified oligonucleotide and artificial cerebrospinal fluid. In certain embodiments, a pharmaceutical composition consists essentially of a modified oligonucleotide and artificial cerebrospinal fluid. In certain embodiments, the artificial cerebrospinal fluid is pharmaceutical grade.

In certain embodiments, pharmaceutical compositions comprise one or more oligomeric compound and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, oligomeric compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an oligomeric compound encompass any pharmaceutically acceptable salts of the oligomeric compound, esters of the oligomeric compound, or salts of such esters. In certain embodiments, pharmaceutical compositions comprising oligomeric compounds comprising one or more oligonucleotide, upon administration to a subject, including a human, are capable of providing (directly or indirectly) the biologically active metabolite or residue thereof. Accordingly, for example, the disclosure is also drawn to pharmaceutically acceptable salts of oligomeric compounds, prodrugs, pharmaceutically acceptable salts of such prodrugs, and other bioequivalents. Suitable pharmaceutically acceptable salts include, but are not limited to, sodium and potassium salts. In certain embodiments, prodrugs comprise one or more conjugate group attached to an oligonucleotide, wherein the conjugate group is cleaved by endogenous nucleases within the body.

Lipid moieties have been used in nucleic acid therapies in a variety of methods. In certain such methods, the nucleic acid, such as an oligomeric compound, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV), etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

VI. Certain Hotspot Regions

In certain embodiments, nucleobases in the ranges specified below comprise a hotspot region of a KCNQ2 nucleic acid.

In certain embodiments, the ranges described in the Table below comprise hotspot regions. Each hotspot region begins with the nucleobase of SEQ ID NO:2 identified in the “Start Site SEQ ID NO: 2” column and ends with the nucleobase of SEQ ID NO: 2 identified in the “Stop Site SEQ ID NO: 2” column. In certain embodiments, modified oligonucleotides are complementary within any of the hotspot regions 1-43, as defined in the table below. In certain embodiments, modified oligonucleotides are 20 nucleobases in length.

In certain embodiments, the modified oligonucleotides are gapmers. In certain embodiments, the gapmers are 5-10-5 MOE gapmers.

In certain embodiments, the internucleoside linkages of the modified oligonucleotides are phosphorothioate internucleoside linkages and phosphodiester internucleoside linkages. In certain embodiments, the phosphodiester (“o”) and phosphorothioate (“s”) internucleoside linkages are arranged in order from 5′ to 3′: soooossssssssssooss, wherein each “s” represents a phosphorothioate internucleoside linkage and each “o” represents a phosphodiester internucleoside linkage.

The nucleobase sequence of compounds listed in the “Compound No. in range” column in the table below are complementary to SEQ ID NO: 2 within the specified hotspot region. The nucleobase sequence of the oligonucleotides listed in the “SEQ ID NO: in range” column in the table below are complementary to the target sequence, SEQ ID NO: 2, within the specified hotspot region.

In certain embodiments, modified oligonucleotides complementary to nucleobases within the hotspot region achieve an average of “Avg. % Red. in vitro” (average % reduction, relative to untreated control cells) of KCNQ2 RNA in vitro in the standard cell assay, as indicated in the table below.

TABLE 1
KCNQ2 Hotspots

| | SEQ ID | SEQ ID | Avg. % | Min. % | Max. % | | |
|---|---|---|---|---|---|---|---|
| Hotspot | NO: 2 | NO: 2 | Red. in | Red. in | Red. in | | SEQ ID NO |
| ID | Start Site | Stop Site | vitro | vitro | vitro | Compound No. in range | in range |
| 1 | 4600 | 4624 | 87.5 | 85 | 90 | 1375651, 1375636 | 67, 82 |
| 2 | 8970 | 8990 | 66 | 60 | 71 | 1375603, 1375652 | 34, 83 |
| 3 | 23730 | 23752 | 72 | 65 | 78 | 1375630, 1375666 | 61, 97 |
| 4 | 24439 | 24459 | 80 | 79 | 80 | 1375609, 1375611 | 40, 42 |
| 5 | 33048 | 33083 | 49 | 11 | 75 | 1375626, 1375592, 1375599, | 23, 25, 30, 50, |
| | | | | | | 1375664, 1375658, 1375660, | 57, 68, 89, 91, |
| | | | | | | 1375619, 1375637, 1375594 | 95 |
| 6 | 33054 | 33083 | 60 | 48 | 75 | 1375664, 1375658, 1375660, | 25, 50, 68, |
| | | | | | | 1375619, 1375637, 1375594 | 89, 91, 95 |
| 7 | 34198 | 34232 | 86 | 84 | 88 | 1375604, 1375625 | 35, 56 |
| 8 | 34543 | 34563 | 89 | 89 | 89 | 1375653, 1375627 | 58, 84 |
| 9 | 35323 | 35343 | 87 | 84 | 90 | 1375606, 1375643 | 37, 74 |
| 10 | 36653 | 36674 | 88 | 86 | 90 | 1375600, 1375667 | 31, 98 |
| 11 | 41334 | 41357 | 49 | 44 | 54 | 1375645, 1375628 | 59, 76 |
| 12 | 57523 | 57544 | 38 | 32 | 43 | 1375624, 1375655 | 55, 86 |
| 13 | 1615 | 1642 | 74 | 56 | 79 | 1499068-1499073 | 132-137 |
| 14 | 3758 | 3780 | 68 | 54 | 81 | 1577101, 1577249, 1577375, | 725, 846, 903, |
| | | | | | | 1577429 | 1005 |
| 15 | 4631 | 4650 | 71 | 57 | 79 | 1499035-1499041 | 99-105 |
| 16 | 6025 | 6050 | 65 | 54 | 75 | 1577113, 1577274, 1577296, | 641, 679, 771, |
| | | | | | | 1577349, 1577504, 1577524 | 804, 949, 987 |
| 17 | 6033 | 6055 | 65 | 61 | 74 | 1577180, 1577352, 1577498, | 714, 772, 816, |
| | | | | | | 1577528 | 946 |
| 18 | 6547 | 6573 | 71 | 68 | 74 | 1499020-1499024, 1499026 | 240-244, 246 |
| 19 | 7371 | 7394 | 71 | 66 | 79 | 1499003-1499007 | 223-227 |
| 20 | 9932 | 9955 | 61 | 47 | 84 | 1577289, 1577326, 1577479, | 644, 715, 760, |
| | | | | | | 1577531, 1577542, | 866, 991 |
| 21 | 10324 | 10347 | 86 | 77 | 90 | 1577118, 1577136, 1577138, | 807, 886, 913, |
| | | | | | | 1577205, 1577313 | 960, 971 |
| 22 | 10439 | 10462 | 79 | 72 | 89 | 1577150, 1577179, 1577185, | 654, 655, 754, |
| | | | | | | 1577262, 1577516 | 796, 817 |
| 23 | 13863 | 13889 | 62 | 52 | 73 | 1577120, 1577140, 1577222, | 575, 590, 684, |
| | | | | | | 1577227, 1577238, 1577310 | 750, 869, 881, |
| | | | | | | 1577519, 1577541 | 974, 1029 |
| 24 | 14261 | 14284 | 67 | 54 | 78 | 1577327, 1577335, 1577386, | 722, 769, 849, |
| | | | | | | 1577488, 1577548 | 945, 992 |
| 25 | 16110 | 16137 | 63 | 55 | 83 | 1577090, 1577105, 1577144, | 597, 680, 777, |
| | | | | | | 1577288, 1577295, 1577395, | 785, 800, 863, |
| | | | | | | 1577448, 1577460, 1577518 | 889, 951, 955 |
| 26 | 16142 | 16167 | 61 | 47 | 69 | 1498987-1498992, 1498994 | 207-212,214 |
| 27 | 16506 | 16533 | 77 | 61 | 89 | 1577160, 1577233, 1577245, | 577, 592, 666, |
| | | | | | | 1577311, 1577398, 1577442, | 720, 778, 834, |
| | | | | | | 1577492, 1577512, 1577540 | 938, 1021, |
| | | | | | | | 1026 |
| 28 | 18835 | 18858 | 67 | 53 | 75 | 1498950-1498953 | 326-329 |
| 29 | 20464 | 20486 | 71 | 66 | 74 | 1498938, 1498939, 1498941 | 314, 315, 317 |
| 30 | 24161 | 24186 | 63 | 55 | 66 | 1498926-1498930 | 302-306 |
| 31 | 25662 | 25685 | 67 | 65 | 72 | 1498910-1498913 | 286-289 |
| 32 | 26622 | 26647 | 74 | 62 | 86 | 1577093, 1577147, 1577201, | 617, 671,746, |
| | | | | | | 1577202, 1577248, 1577366, | 801, 809, 896, |
| | | | | | | 1577532 | 1027 |
| 33 | 26709 | 26741 | 82 | 63 | 95 | 1577111, 1577149, 1577169, | 579, 643, 653, |
| | | | | | | 1577173, 1577212, 1577214, | 676, 748, 773, |
| | | | | | | 1577269, 1577304, 1577334, | 838, 868, 898, |
| | | | | | | 1577341, 1577358, 1577421, | 911, 918, 957, |
| | | | | | | 1577489, 1577530 | 969, 1003 |
| 34 | 27035 | 27059 | 68 | 56 | 78 | 1577108, 1577137, 1577260, | 572, 673, 789, |
| | | | | | | 1577387, 1577388, 1577501 | 850, 887, 997 |
| 35 | 28752 | 28774 | 61 | 55 | 68 | 1498890-1498891, 1577171, | 266, 267, 718, |
| | | | | | | 1577538 | 739 |
| 36 | 29184 | 29211 | 77 | 67 | 84 | 1498878-1498883 | 255-259, 410 |
| 37 | 29405 | 29431 | 62 | 49 | 78 | 1577189, 1577235, 1577251, | 636, 694, 752, |
| | | | | | | 1577399, 1577480, 1577491, | 828, 952, 970, |
| | | | | | | 1577529 | 1020 |
| 38 | 29938 | 29960 | 75 | 72 | 81 | 1577181, 1577422, 1577432 | 626, 630, |
| | | | | | | | 1004 |
| 39 | 30968 | 30991 | 60 | 45 | 67 | 1577161, 1577209, 1577308, | 602, 765, 812, |
| | | | | | | 1577317, 1577408 | 932, 972 |
| 40 | 31860 | 31886 | 59 | 49 | 70 | 1498865-1498869, 1577121, | 397-401, 692, |
| | | | | | | 1577266, 1577379 | 729, 832 |
| 41 | 32962 | 32984 | 59 | 51 | 74 | 1577191, 1577204, 1577400, | 638, 658, 853, |
| | | | | | | 1577497 | 897 |
| 42 | 37062 | 37091 | 67 | 46 | 84 | 1577087, 1577174, 1577211, | 615, 627, 645, |
| | | | | | | 1577321, 1577329, 1577362, | 747, 788, 815, |
| | | | | | | 1577423, 1577459, 1577494, | 862, 915,917, |
| | | | | | | 1577503, 1577536 | 1024, 1028 |
| 43 | 39847 | 39870 | 64 | 51 | 76 | 1577123, 1577126, 1577186, | 591, 650, 731, |
| | | | | | | 1577241, 1577361 | 743, 996 |

### Nonlimiting Disclosure and Incorporation by Reference

Each of the literature and patent publications listed herein is incorporated by reference in its entirety.

While certain compounds, compositions and methods described herein have been described with specificity in accordance with certain embodiments, the following examples serve only to illustrate the compounds described herein and are not intended to limit the same. Each of the references, GenBlank accession numbers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of an uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein ^{m}C indicates a cytosine base comprising a methyl group at the 5-position.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as α or β such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms, unless specified otherwise. Likewise, tautomeric forms of the compounds herein are also included unless otherwise indicated. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or 3H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXAMPLES

### Example 1: Effect of 5-10-5 MOE Gapmer Modified Oligonucleotides on Human KCNQ2 RNA In Vitro, Single Dose

Modified oligonucleotides complementary to a human KCNQ2 nucleic acid were designed and tested for their single dose effects on KCNQ2 RNA in vitro. The modified oligonucleotides were tested in a series of experiments that had similar culture conditions.

The modified oligonucleotides in the tables below are 5-10-5 MOE gapmers with mixed PO/PS internucleoside linkages. The gapmers are 20 nucleosides in length, wherein the deoxy region consists of ten 2′-β-D-deoxynucleosides and the 3′ and 5′ wings each consist of five 2′-MOE modified nucleosides. The sugar motif of the gapmers is (from 5′ to 3′): eeeeeddddddddddeeeee; wherein “d” represents a 2′-β-D-deoxyribosyl sugar moiety, and ‘e’ represents a 2′-MOE sugar moiety. The internucleoside linkage motif of the gapmers is (from 5′ to 3′): soooossssssssssooss; wherein ‘o’ represents a phosphodiester internucleoside linkage and ‘s’ represents a phosphorothioate internucleoside linkage. Each cytosine residue is a 5-methyl cytosine.

“Start site” indicates the 5′-most nucleoside to which the modified oligonucleotide is complementary in the human gene sequence. “Stop site” indicates the 3′-most nucleoside to which the modified oligonucleotide is complementary in the human gene sequence. Each modified oligonucleotide listed in the Tables below is 100% complementary to SEQ ID NO: 1 (cDNA of ENSEMBL Accession No. ENST00000359125.6, version 98: September 2019) or SEQ ID NO: 2 (ENSEMBL Accession No. ENG00000075043, the reverse strand of chromosome 20 from genome assembly GRCh38 truncated from 63/400,210 to 63/472,677). ‘N/A’ indicates that the modified oligonucleotide is not 100% complementary to that particular gene sequence.

Cultured SH-SY5Y cells were treated with modified oligonucleotide at a concentration of 4,000 nM by electroporation at a density of 20,000 cells per well for a treatment period of 24 hours. At the end of their treatment period, total RNA was isolated from the cells and KCNQ2 RNA levels were measured by quantitative real-time RTPCR. KCNQ2 RNA levels were measured by human KCNQ2 primer probe set RTS49037 (forward sequence GGTTTGCCCTGAAGGTTC, designated herein as SEQ ID NO: 11; reverse sequence GAGGTTGGTGGCGTAGAATC, designated herein as SEQ ID NO: 12; probe sequence TCTCAAAGTGCTTCTGCCTGTGCT, designated herein as SEQ ID NO: 13). KCNQ2 RNA levels were normalized to total RNA content, as measured by RIBOGREEN®. Results are presented in the tables below as percent KCNQ2 RNA levels relative to untreated control cells (% control). Each table represents results from an individual assay plate. The Compound Nos. marked with an asterisk (*) indicate that the modified oligonucleotide is complementary to the amplicon region of the primer probe set. Additional assays may be used to measure the potency and efficacy of the modified oligonucleotide complementary to the amplicon region.

TABLE 2
Reduction of KCNQ2 RNA by 5-10-5 MOE gapmers with mixed PO/PS linkages in SH-SY5Y Cells

| | SEQ ID | SEQ ID | SEQ ID | SEQ ID | | | |
|---|---|---|---|---|---|---|---|
| | NO: 1 | NO: 1 | NO: 2 | NO: 2 | | | |
| Compound | Start | Stop | Start | Stop | | KCNQ2 | SEQ |
| Number | Site | Site | Site | Site | Sequence (5′ to 3′) | (%UTC) | ID NO |
| 1375590 | N/A | N/A | 27287 | 27306 | GGAGGAAAGCTGAGGCCACC | 80 | 21 |
| 1375591 | N/A | N/A | 57527 | 57546 | CCCACGGGAACCGACAGACA | 84 | 22 |
| 1375592 | 1024 | 1043 | 33050 | 33069 | CGAAGAAGGAGACACCGATG | 74 | 23 |
| 1375593 | N/A | N/A | 27275 | 27294 | AGGCCACCTTGAGGCCTGGG | 65 | 24 |
| 1375594 | N/A | N/A | 33064 | 33083 | TACTGCAGGCAGCGCGAAGA | 47 | 25 |
| 1375595 | 2360 | 2379 | 65648 | 65667 | GACCGCTCGTGGGCAGGCGG | 81 | 26 |
| 1375596 | 2364 | 2383 | 65652 | 65671 | CAGCGACCGCTCGTGGGCAG | 73 | 27 |
| 1375597 | 2348 | 2367 | 65636 | 65655 | GCAGGCGGCGGCGGGATGCG | 84 | 28 |
| 1375598 | 2354 | 2373 | 65642 | 65661 | TCGTGGGCAGGCGGCGGCGG | 82 | 29 |
| 1375599 | 1026 | 1045 | 33052 | 33071 | CGCGAAGAAGGAGACACCGA | 63 | 30 |
| 1375600 | N/A | N/A | 36653 | 36672 | ACAGAATCTACTCCAGGCAA | 10 | 31 |
| 1375601 | N/A | N/A | 27281 | 27300 | AAGCTGAGGCCACCTTGAGG | 76 | 32 |
| 1375602 | N/A | N/A | 41287 | 41306 | TTCAAGTGTTTCCACACACA | 58 | 33 |
| 1375603 | N/A | N/A | 8970 | 8989 | TCATGTTTTTTCCAAACCTA | 29 | 34 |
| 1375604 | N/A | N/A | 34198 | 34217 | ACACACACTTCACATCCTCG | 12 | 35 |
| 1375605 | N/A | N/A | 57517 | 57536 | CCGACAGACAGACAGAAAAA | 99 | 36 |
| 1375606 | N/A | N/A | 35323 | 35342 | GTCACTTTTTAAACCGTCAA | 16 | 37 |
| 1375607 | N/A | N/A | 71222 | 71241 | CCGGAGTCCACCATCCCACA | 80 | 38 |
| 1375608 | N/A | N/A | 36727 | 36746 | TGGTTATAATCCTTTCTTCT | 20 | 39 |
| 1375609 | N/A | N/A | 24439 | 24458 | TCTCATTTACATTTTTCGCA | 20 | 40 |
| 1375610 | N/A | N/A | 27285 | 27304 | AGGAAAGCTGAGGCCACCTT | 71 | 41 |
| 1375611 | N/A | N/A | 24440 | 24459 | GTCTCATTTACATTTTTCGC | 21 | 42 |
| 1375612 | 2463 | 2482 | 65751 | 65770 | GTCGCTGTCCCGCAGGTTCC | 70 | 43 |
| 1375613 | N/A | N/A | 27279 | 27298 | GCTGAGGCCACCTTGAGGCC | 39 | 44 |
| 1375614 | 2461 | 2480 | 65749 | 65768 | CGCTGTCCCGCAGGTTCCCC | 68 | 45 |
| 1375615 | N/A | N/A | 48045 | 48064 | TCCGACTCTTTTTTCCACGC | 36 | 46 |
| 1375616 | N/A | N/A | 39243 | 39262 | GTACTGGTTCCACTCTTGAA | 58 | 47 |
| 1375617 | N/A | N/A | 25195 | 25214 | CCATGATTGCTCTTCCCATT | 55 | 48 |
| 1375618 | 2352 | 2371 | 65640 | 65659 | GTGGGCAGGCGGCGGCGGGA | 85 | 49 |
| 1375619 | 1034 | 1053 | 33060 | 33079 | GCAGGCAGCGCGAAGAAGGA | 25 | 50 |
| 1375620 | N/A | N/A | 25167 | 25186 | ACGCGATTACCTCACTCACT | 38 | 51 |
| 1375621 | N/A | N/A | 57529 | 57548 | CTCCCACGGGAACCGACAGA | 83 | 52 |
| 1375622 | N/A | N/A | 59468 | 59487 | ACACACATTTTCAGGGACCC | 92 | 53 |
| 1375623 | 2457 | 2476 | 65745 | 65764 | GTCCCGCAGGTTCCCCTCGG | 64 | 54 |
| 1375624 | N/A | N/A | 57523 | 57542 | CGGGAACCGACAGACAGACA | 57 | 55 |
| 1375625 | N/A | N/A | 34213 | 34232 | GAGCCATTTCTCAACACACA | 16 | 56 |
| 1375626 | 1022 | 1041 | 33048 | 33067 | AAGAAGGAGACACCGATGAG | 89 | 57 |
| 1375627 | N/A | N/A | 34544 | 34563 | GTCCGTATTTTCAAGGTGGC | 11 | 58 |
| 1375628 | N/A | N/A | 41338 | 41357 | CTGTCCATGCATTTCCTACC | 56 | 59 |
| 1375629 | 2356 | 2375 | 65644 | 65663 | GCTCGTGGGCAGGCGGCGGC | 58 | 60 |
| 1375630 | N/A | N/A | 23730 | 23749 | TTGCAATTTCCTTTCCAGTC | 22 | 61 |
| 1375631 | N/A | N/A | 47332 | 47351 | TCATGTCCATCTTACATCGA | 61 | 62 |
| 1375632 | 2459 | 2478 | 65747 | 65766 | CTGTCCCGCAGGTTCCCCTC | 83 | 63 |
| 1375633 | N/A | N/A | 57521 | 57540 | GGAACCGACAGACAGACAGA | 88 | 64 |
| 1375634 | N/A | N/A | 49301 | 49320 | GCCAGATTTACTCTGCAACA | 57 | 65 |
| 1375635 | N/A | N/A | 57909 | 57928 | GCACAAGTCTCACCTCAATT | 65 | 66 |
| 1375636 | N/A | N/A | 4605 | 4624 | GTACAATGTCTTATCACTCC | 15 | 67 |
| 1375637 | 1036 | 1055 | 33062 | 33081 | CTGCAGGCAGCGCGAAGAAG | 29* | 68 |
| 1375638 | N/A | N/A | 57533 | 57552 | GCTGCTCCCACGGGAACCGA | 76 | 69 |
| 1375639 | N/A | N/A | 57519 | 57538 | AACCGACAGACAGACAGAAA | 89 | 70 |
| 1375640 | N/A | N/A | 27277 | 27296 | TGAGGCCACCTTGAGGCCTG | 97 | 71 |
| 1375641 | N/A | N/A | 8652 | 8671 | CAACCATGCTCTCCTATGCA | 45 | 72 |
| 1375642 | N/A | N/A | 48017 | 48036 | ATGGAATGATTCTCTTCCGC | 77 | 73 |
| 1375643 | N/A | N/A | 35324 | 35343 | GGTCACTTTTTAAACCGTCA | 10 | 74 |
| 1375644 | N/A | N/A | 5353 | 5372 | GTAGCAGATTCATCTCCCCA | 12 | 75 |
| 1375645 | N/A | N/A | 41334 | 41353 | CCATGCATTTCCTACCTGGA | 46 | 76 |
| 1375646 | N/A | N/A | 42976 | 42995 | GCTGACTCCATTGTCCCTCA | 51 | 77 |
| 1375647 | 2465 | 2484 | 65753 | 65772 | GTGTCGCTGTCCCGCAGGTT | 81 | 78 |
| 1375648 | N/A | N/A | 35013 | 35032 | CAGGATTATCTCCATCTCAA | 13 | 79 |
| 1375649 | 2358 | 2377 | 65646 | 65665 | CCGCTCGTGGGCAGGCGGCG | 90 | 80 |
| 1375650 | N/A | N/A | 25154 | 25173 | ACTCACTGATCCTTCTTCAA | 65 | 81 |
| 1375651 | N/A | N/A | 4600 | 4619 | ATGTCTTATCACTCCAGAGT | 10 | 82 |
| 1375652 | N/A | N/A | 8971 | 8990 | CTCATGTTTTTTCCAAACCT | 40 | 83 |
| 1375653 | N/A | N/A | 34543 | 34562 | TCCGTATTTTCAAGGTGGCT | 11 | 84 |
| 1375654 | N/A | N/A | 47377 | 47396 | TGGAGACCAGCATTCAACCA | 65 | 85 |
| 1375655 | N/A | N/A | 57525 | 57544 | CACGGGAACCGACAGACAGA | 68 | 86 |
| 1375656 | 2350 | 2369 | 65638 | 65657 | GGGCAGGCGGCGGCGGGATG | 112 | 87 |
| 1375657 | N/A | N/A | 24756 | 24775 | CGTGACTTTTTCTCAGCCCT | 27 | 88 |
| 1375658 | 1030 | 1049 | 33056 | 33075 | GCAGCGCGAAGAAGGAGACA | 52 | 89 |
| 1375659 | N/A | N/A | 57531 | 57550 | TGCTCCCACGGGAACCGACA | 87 | 90 |
| 1375660 | 1032 | 1051 | 33058 | 33077 | AGGCAGCGCGAAGAAGGAGA | 48 | 91 |
| 1375661 | 2362 | 2381 | 65650 | 65669 | GCGACCGCTCGTGGGCAGGC | 92 | 92 |
| 1375662 | 1436 | 1455 | 57559 | 57578 | ACACGATCTTTCAAACTGAC | 70 | 93 |
| 1375663 | N/A | N/A | 10031 | 10050 | TGAGAACATTTTTACAGCCA | 23 | 94 |
| 1375664 | 1028 | 1047 | 33054 | 33073 | AGCGCGAAGAAGGAGACACC | 37 | 95 |
| 1375665 | N/A | N/A | 27283 | 27302 | GAAAGCTGAGGCCACCTTGA | 80 | 96 |
| 1375666 | N/A | N/A | 23733 | 23752 | GCCTTGCAATTTCCTTTCCA | 35 | 97 |
| 1375667 | N/A | N/A | 36655 | 36674 | CGACAGAATCTACTCCAGGC | 14 | 98 |

### Example 2: Effect of 5-10-5 MOE Modified Oligonucleotides with Mixed PO/PS Linkages on Human KCNQ2 RNA In Vitro, Single Dose

Modified oligonucleotides complementary to a human KCNQ2 nucleic acid were designed and tested for their single dose effects on KCNQ2 RNA in vitro. The modified oligonucleotides were tested in a series of experiments that had the same culture conditions.

“Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the table below is 100% complementary to SEQ ID NO: 2 (described herein above).

Each separate experimental analysis described in this example is identified by a letter ID in the table column labeled “AID” (Analysis ID). Cultured SH-SY5Y cells were treated with modified oligonucleotide at a concentration of 4,000 nM by electroporation at a density of 20,000 cells per well. After a treatment period of approximately 24 hours, total RNA was isolated from the cells, and KCNQ2 RNA levels were measured by quantitative real-time RTPCR. KCNQ2 RNA levels were measured by human primer-probe set RTS49037 (described herein above). KCNQ2 RNA levels were normalized to total RNA content, as measured by RIBOGREEN® Reduction of KCNQ2 RNA is presented in the table below as percent KCNQ2 RNA relative to the amount in untreated control cells (% UTC).

The modified oligonucleotides in the table below are 5-10-5 MOE modified oligonucleotides with mixed PO/PS internucleoside linkages. The modified oligonucleotides are 20 nucleosides in length, wherein the central gap segment consists of ten 2′-β-D-deoxynucleosides and wherein the 5′ and 3′ wings each consist of five 2′-MOE modified nucleosides. The sugar motif of the modified oligonucleotides is (from 5′ to 3′): eeeeeddddddddddeeeee; wherein each “d” represents a 2′-β-D-deoxyribosyl sugar moiety, and each “e” represents a 2′-MOE sugar moiety. The internucleoside linkage motif of the modified oligonucleotides is (from 5′ to 3′): soooossssssssssooss; wherein each “o” represents a phosphodiester internucleoside linkage and each “s” represents a phosphorothioate internucleoside linkage. Each cytosine residue is a 5-methyl cytosine.

TABLE 3
Reduction of KCNQ2 RNA by 5-10-5 MOE modified oligonucleotides
with mixed PO/PS linkages in SH-SY5Y cells

| | SEQ ID | SEQ ID | | | | |
|---|---|---|---|---|---|---|
| | No: 2 | No: 2 | | KCNQ2 | | |
| Compound | Start | Stop | | (% | | SEQ ID |
| Number | Site | Site | Sequence (5′ to 3′) | UTC) | AID | NO |
| 1499035 | 4638 | 4657 | GATTGACTCCACTGTGTAAC | 36 | A | 99 |
| 1499036 | 4637 | 4656 | ATTGACTCCACTGTGTAACT | 43 | A | 100 |
| 1499037 | 4636 | 4655 | TTGACTCCACTGTGTAACTA | 21 | A | 101 |
| 1499038 | 4634 | 4653 | GACTCCACTGTGTAACTAAG | 21 | A | 102 |
| 1499039 | 4632 | 4651 | CTCCACTGTGTAACTAAGCA | 28 | A | 103 |
| 1499040 | 57264 | 57283 | GGAGGGGGGGAGGCTACCGG | 121 | A | 104 |
| 1499041 | 4631 | 4650 | TCCACTGTGTAACTAAGCAG | 25 | A | 105 |
| 1499042 | 3999 | 4018 | GGCCAGAGGCCCCTAAAACC | 48 | A | 106 |
| 1499043 | 3998 | 4017 | GCCAGAGGCCCCTAAAACCC | 67 | A | 107 |
| 1499044 | 3997 | 4016 | CCAGAGGCCCCTAAAACCCT | 92 | A | 108 |
| 1499045 | 3996 | 4015 | CAGAGGCCCCTAAAACCCTG | 56 | A | 109 |
| 1499046 | 3995 | 4014 | AGAGGCCCCTAAAACCCTGG | 47 | A | 110 |
| 1499047 | 3994 | 4013 | GAGGCCCCTAAAACCCTGGA | 45 | A | 111 |
| 1499048 | 3993 | 4012 | AGGCCCCTAAAACCCTGGAC | 55 | A | 112 |
| 1499049 | 3992 | 4011 | GGCCCCTAAAACCCTGGACC | 71 | A | 113 |
| 1499050 | 3991 | 4010 | GCCCCTAAAACCCTGGACCC | 59 | A | 114 |
| 1499051 | 57263 | 57282 | GAGGGGGGGAGGCTACCGGG | 135 | A | 115 |
| 1499052 | 3990 | 4009 | CCCCTAAAACCCTGGACCCC | 62 | A | 116 |
| 1499053 | 3960 | 3979 | TGGCGCAAAAGCCAGGATGG | 71 | A | 117 |
| 1499054 | 3958 | 3977 | GCGCAAAAGCCAGGATGGCA | 59 | A | 118 |
| 1499055 | 3957 | 3976 | CGCAAAAGCCAGGATGGCAC | 68 | A | 119 |
| 1499056 | 3956 | 3975 | GCAAAAGCCAGGATGGCACA | 64 | A | 120 |
| 1499057 | 3954 | 3973 | AAAAGCCAGGATGGCACAGC | 68 | A | 121 |
| 1499058 | 3952 | 3971 | AAGCCAGGATGGCACAGCGA | 61 | A | 122 |
| 1499059 | 56101 | 56120 | AGGGCCCAGAGCACTGTTGC | 62 | A | 123 |
| 1499060 | 3951 | 3970 | AGCCAGGATGGCACAGCGAG | 57 | A | 124 |
| 1499061 | 2853 | 2872 | CGGGCCAGAGGACATGGGGC | 45 | A | 125 |
| 1499062 | 2851 | 2870 | GGCCAGAGGACATGGGGCCA | 72 | A | 126 |
| 1499063 | 2850 | 2869 | GCCAGAGGACATGGGGCCAT | 33 | A | 127 |
| 1499064 | 2849 | 2868 | CCAGAGGACATGGGGCCATC | 64 | A | 128 |
| 1499065 | 2847 | 2866 | AGAGGACATGGGGCCATCAG | 65 | A | 129 |
| 1499066 | 2845 | 2864 | AGGACATGGGGCCATCAGGC | 40 | A | 130 |
| 1499067 | 2844 | 2863 | GGACATGGGGCCATCAGGCC | 32 | A | 131 |
| 1499068 | 1623 | 1642 | ACGGAGGGTCTGCTAAGGGC | 30 | A | 132 |
| 1499069 | 1621 | 1640 | GGAGGGTCTGCTAAGGGCAC | 29 | A | 133 |
| 1499070 | 1620 | 1639 | GAGGGTCTGCTAAGGGCACA | 21 | A | 134 |
| 1499071 | 1619 | 1638 | AGGGTCTGCTAAGGGCACAC | 27 | A | 135 |
| 1499072 | 1617 | 1636 | GGTCTGCTAAGGGCACACTC | 24 | A | 136 |
| 1499073 | 1615 | 1634 | TCTGCTAAGGGCACACTCAA | 44 | A | 137 |
| 1499074 | 56099 | 56118 | GGCCCAGAGCACTGTTGCTG | 58 | A | 138 |
| 1499075 | 1614 | 1633 | CTGCTAAGGGCACACTCAAG | 56 | A | 139 |
| 1499076 | 1291 | 1310 | CAGGAGGCAGCCGTTCCCTG | 67 | A | 140 |
| 1499077 | 1289 | 1308 | GGAGGCAGCCGTTCCCTGAG | 71 | A | 141 |
| 1499078 | 1288 | 1307 | GAGGCAGCCGTTCCCTGAGC | 58 | A | 142 |
| 1499079 | 1287 | 1306 | AGGCAGCCGTTCCCTGAGCC | 45 | A | 143 |
| 1499080 | 1286 | 1305 | GGCAGCCGTTCCCTGAGCCT | 71 | A | 144 |
| 1499081 | 1285 | 1304 | GCAGCCGTTCCCTGAGCCTC | 30 | A | 145 |
| 1499082 | 1283 | 1302 | AGCCGTTCCCTGAGCCTCGC | 37 | A | 146 |
| 1499083 | 56098 | 56117 | GCCCAGAGCACTGTTGCTGG | 48 | A | 147 |
| 1499084 | 1282 | 1301 | GCCGTTCCCTGAGCCTCGCC | 41 | A | 148 |
| 1499085 | 56097 | 56116 | CCCAGAGCACTGTTGCTGGA | 58 | A | 149 |
| 1499086 | 56095 | 56114 | CAGAGCACTGTTGCTGGAGC | 79 | A | 150 |
| 1499087 | 56093 | 56112 | GAGCACTGTTGCTGGAGCCT | 59 | A | 151 |
| 1499088 | 65098 | 65117 | AAATGGGGGGGCCCAGGCTG | 103 | A | 152 |
| | 65210 | 65229 | | | | |
| 1499089 | 56092 | 56111 | AGCACTGTTGCTGGAGCCTG | 67 | A | 153 |
| 1499090 | 55372 | 55391 | CTCTCTTTCTCCACCGCACG | 69 | A | 154 |
| 1499091 | 55371 | 55390 | TCTCTTTCTCCACCGCACGA | 63 | A | 155 |
| 1499092 | 55370 | 55389 | CTCTTTCTCCACCGCACGAG | 89 | A | 156 |
| 1499093 | 55369 | 55388 | TCTTTCTCCACCGCACGAGC | 91 | A | 157 |
| 1499094 | 55368 | 55387 | CTTTCTCCACCGCACGAGCC | 78 | A | 158 |
| 1499095 | 55367 | 55386 | TTTCTCCACCGCACGAGCCA | 86 | A | 159 |
| 1499096 | 55366 | 55385 | TTCTCCACCGCACGAGCCAT | 65 | A | 160 |
| 1499097 | 55364 | 55383 | CTCCACCGCACGAGCCATTT | 76 | A | 161 |
| 1499098 | 55363 | 55382 | TCCACCGCACGAGCCATTTC | 97 | A | 162 |
| 1499099 | 55316 | 55335 | CCGCTCAGGCAGGCCTCAGG | 68 | A | 163 |
| 1499100 | 55314 | 55333 | GCTCAGGCAGGCCTCAGGGC | 46 | A | 164 |
| 1499101 | 55313 | 55332 | CTCAGGCAGGCCTCAGGGCA | 55 | A | 165 |
| 1499102 | 55312 | 55331 | TCAGGCAGGCCTCAGGGCAA | 53 | A | 166 |
| 1499103 | 55310 | 55329 | AGGCAGGCCTCAGGGCAAGT | 93 | A | 167 |
| 1499104 | 55308 | 55327 | GCAGGCCTCAGGGCAAGTGG | 70 | A | 168 |
| 1499105 | 65096 | 65115 | ATGGGGGGGCCCAGGCTGGT | 112 | A | 169 |
| | 65208 | 65227 | | | | |
| 1499106 | 55307 | 55326 | CAGGCCTCAGGGCAAGTGGG | 67 | A | 170 |
| 1499107 | 55079 | 55098 | CAGCTTGAGGAATGGGCATC | 54 | A | 171 |
| 1499108 | 55077 | 55096 | GCTTGAGGAATGGGCATCAG | 61 | A | 172 |
| 1499109 | 55076 | 55095 | CTTGAGGAATGGGCATCAGA | 67 | A | 173 |
| 1499110 | 55075 | 55094 | TTGAGGAATGGGCATCAGAT | 73 | A | 174 |
| 1499111 | 55073 | 55092 | GAGGAATGGGCATCAGATTC | 57 | A | 175 |
| 1499112 | 55071 | 55090 | GGAATGGGCATCAGATTCCC | 73 | A | 176 |
| 1498957 | 58309 | 58328 | TCTGCACCCAGCTGCTCTCG | 98 | B | 177 |
| 1498958 | 18830 | 18849 | TCTGCATCCCACCGAGGATG | 55 | B | 178 |
| 1498959 | 18637 | 18656 | TCCTAAGGTGGCTCCGACCA | 62 | B | 179 |
| 1498960 | 18635 | 18654 | CTAAGGTGGCTCCGACCAGG | 60 | B | 180 |
| 1498961 | 18634 | 18653 | TAAGGTGGCTCCGACCAGGC | 43 | B | 181 |
| 1498962 | 18633 | 18652 | AAGGTGGCTCCGACCAGGCA | 37 | B | 182 |
| 1498963 | 18631 | 18650 | GGTGGCTCCGACCAGGCATC | 46 | B | 183 |
| 1498964 | 18629 | 18648 | TGGCTCCGACCAGGCATCTC | 71 | B | 184 |
| 1498965 | 65100 | 65119 | GGAAATGGGGGGGCCCAGGC | 81 | B | 185 |
| | 65212 | 65231 | | | | |
| 1498966 | 58308 | 58327 | CTGCACCCAGCTGCTCTCGA | 77 | B | 186 |
| 1498967 | 18628 | 18647 | GGCTCCGACCAGGCATCTCA | 52 | B | 187 |
| 1498968 | 18048 | 18067 | TCCAGGCCTCCGCCTACAAA | 55 | B | 188 |
| 1498969 | 18047 | 18066 | CCAGGCCTCCGCCTACAAAC | 84 | B | 189 |
| 1498970 | 18046 | 18065 | CAGGCCTCCGCCTACAAACC | 69 | B | 190 |
| 1498971 | 18045 | 18064 | AGGCCTCCGCCTACAAACCG | 59 | B | 191 |
| 1498972 | 18044 | 18063 | GGCCTCCGCCTACAAACCGT | 71 | B | 192 |
| 1498973 | 18042 | 18061 | CCTCCGCCTACAAACCGTGG | 63 | B | 193 |
| 1498974 | 18041 | 18060 | CTCCGCCTACAAACCGTGGT | 90 | B | 194 |
| 1498975 | 18040 | 18059 | TCCGCCTACAAACCGTGGTG | 85 | B | 195 |
| 1498976 | 57272 | 57291 | GGCGGGAGGGAGGGGGGGAG | 88 | B | 196 |
| 1498977 | 18039 | 18058 | CCGCCTACAAACCGTGGTGT | 74 | B | 197 |
| 1498978 | 16343 | 16362 | GGGAAGAGGACTCGACGCAG | 54 | B | 198 |
| 1498979 | 16341 | 16360 | GAAGAGGACTCGACGCAGGA | 64 | B | 199 |
| 1498980 | 16340 | 16359 | AAGAGGACTCGACGCAGGAG | 78 | B | 200 |
| 1498981 | 16339 | 16358 | AGAGGACTCGACGCAGGAGG | 58 | B | 201 |
| 1498982 | 16337 | 16356 | AGGACTCGACGCAGGAGGGG | 73 | B | 202 |
| 1498983 | 16335 | 16354 | GACTCGACGCAGGAGGGGTC | 76 | B | 203 |
| 1498984 | 16334 | 16353 | ACTCGACGCAGGAGGGGTCT | 78 | B | 204 |
| 1498985 | 16151 | 16170 | GGAGGGAAGGCGTCACCACC | 46 | B | 205 |
| 1498986 | 16149 | 16168 | AGGGAAGGCGTCACCACCTC | 61 | B | 206 |
| 1498987 | 16148 | 16167 | GGGAAGGCGTCACCACCTCA | 44 | B | 207 |
| 1498988 | 16147 | 16166 | GGAAGGCGTCACCACCTCAC | 37 | B | 208 |
| 1498989 | 16146 | 16165 | GAAGGCGTCACCACCTCACC | 38 | B | 209 |
| 1498990 | 16145 | 16164 | AAGGCGTCACCACCTCACCG | 34 | B | 210 |
| 1498991 | 16144 | 16163 | AGGCGTCACCACCTCACCGC | 31 | B | 211 |
| 1498992 | 16143 | 16162 | GGCGTCACCACCTCACCGCC | 53 | B | 212 |
| 1498993 | 57270 | 57289 | CGGGAGGGAGGGGGGGAGGC | 163 | B | 213 |
| 1498994 | 16142 | 16161 | GCGTCACCACCTCACCGCCA | 37 | B | 214 |
| 1498995 | 14330 | 14349 | CTGGCCCTCGAGGCCGCTGC | 57 | B | 215 |
| 1498996 | 14328 | 14347 | GGCCCTCGAGGCCGCTGCAG | 50 | B | 216 |
| 1498997 | 14327 | 14346 | GCCCTCGAGGCCGCTGCAGT | 51 | B | 217 |
| 1498998 | 14326 | 14345 | CCCTCGAGGCCGCTGCAGTG | 73 | B | 218 |
| 1498999 | 14324 | 14343 | CTCGAGGCCGCTGCAGTGCC | 84 | B | 219 |
| 1499000 | 14322 | 14341 | CGAGGCCGCTGCAGTGCCTC | 105 | B | 220 |
| 1499001 | 57269 | 57288 | GGGAGGGAGGGGGGGAGGCT | 130 | B | 221 |
| 1499002 | 14321 | 14340 | GAGGCCGCTGCAGTGCCTCA | 80 | B | 222 |
| 1499003 | 7375 | 7394 | AGCAGCATCCTGAGCCACTC | 26 | B | 223 |
| 1499004 | 7374 | 7393 | GCAGCATCCTGAGCCACTCC | 21 | B | 224 |
| 1499005 | 7373 | 7392 | CAGCATCCTGAGCCACTCCC | 34 | B | 225 |
| 1499006 | 7372 | 7391 | AGCATCCTGAGCCACTCCCT | 30 | B | 226 |
| 1499007 | 7371 | 7390 | GCATCCTGAGCCACTCCCTC | 34 | B | 227 |
| 1499008 | 7369 | 7388 | ATCCTGAGCCACTCCCTCCT | 60 | B | 228 |
| 1499009 | 7367 | 7386 | CCTGAGCCACTCCCTCCTCC | 48 | B | 229 |
| 1499010 | 57268 | 57287 | GGAGGGAGGGGGGGAGGCTA | 139 | B | 230 |
| 1499011 | 7366 | 7385 | CTGAGCCACTCCCTCCTCCA | 54 | B | 231 |
| 1499012 | 7285 | 7304 | CGCCTTGTGCTGGCCGCCTC | 69 | B | 232 |
| 1499013 | 7283 | 7302 | CCTTGTGCTGGCCGCCTCCC | 65 | B | 233 |
| 1499014 | 7282 | 7301 | CTTGTGCTGGCCGCCTCCCT | 67 | B | 234 |
| 1499015 | 7281 | 7300 | TTGTGCTGGCCGCCTCCCTC | 52 | B | 235 |
| 1499016 | 7279 | 7298 | GTGCTGGCCGCCTCCCTCAC | 56 | B | 236 |
| 1499017 | 7277 | 7296 | GCTGGCCGCCTCCCTCACAC | 53 | B | 237 |
| 1499018 | 7276 | 7295 | CTGGCCGCCTCCCTCACACC | 76 | B | 238 |
| 1499019 | 6556 | 6575 | GGATGCTGGGAACACGCCGG | 47 | B | 239 |
| 1499020 | 6554 | 6573 | ATGCTGGGAACACGCCGGAA | 30 | B | 240 |
| 1499021 | 6553 | 6572 | TGCTGGGAACACGCCGGAAG | 32 | B | 241 |
| 1499022 | 6552 | 6571 | GCTGGGAACACGCCGGAAGC | 26 | B | 242 |
| 1499023 | 6550 | 6569 | TGGGAACACGCCGGAAGCCA | 27 | B | 243 |
| 1499024 | 6548 | 6567 | GGAACACGCCGGAAGCCACG | 30 | B | 244 |
| 1499025 | 57266 | 57285 | AGGGAGGGGGGGAGGCTACC | 106 | B | 245 |
| 1499026 | 6547 | 6566 | GAACACGCCGGAAGCCACGA | 28 | B | 246 |
| 1499027 | 4806 | 4825 | ATATCCAGGGCGCCGGCACC | 61 | B | 247 |
| 1499028 | 4804 | 4823 | ATCCAGGGCGCCGGCACCTC | 47 | B | 248 |
| 1499029 | 4803 | 4822 | TCCAGGGCGCCGGCACCTCT | 88 | B | 249 |
| 1499030 | 4802 | 4821 | CCAGGGCGCCGGCACCTCTG | 42 | B | 250 |
| 1499031 | 4800 | 4819 | AGGGCGCCGGCACCTCTGGA | 63 | B | 251 |
| 1499032 | 4798 | 4817 | GGCGCCGGCACCTCTGGAGA | 96 | B | 252 |
| 1499033 | 4797 | 4816 | GCGCCGGCACCTCTGGAGAG | 86 | B | 253 |
| 1499034 | 4640 | 4659 | GAGATTGACTCCACTGTGTA | 52 | B | 254 |
| 1498879 | 29190 | 29209 | CCACCATCATCACCACCACC | 18 | C | 255 |
| | 29286 | 29305 | | | | |
| | 29530 | 29549 | | | | |
| 1498880 | 29189 | 29208 | CACCATCATCACCACCACCA | 16 | C | 256 |
| | 29285 | 29304 | | | | |
| | 29354 | 29373 | | | | |
| | 29375 | 29394 | | | | |
| | 29529 | 29548 | | | | |
| 1498881 | 29188 | 29207 | ACCATCATCACCACCACCAT | 26 | C | 257 |
| | 29284 | 29303 | | | | |
| | 29374 | 29393 | | | | |
| | 29528 | 29547 | | | | |
| 1498882 | 29186 | 29205 | CATCATCACCACCACCATCA | 33 | C | 258 |
| | 29282 | 29301 | | | | |
| | 29372 | 29391 | | | | |
| | 29526 | 29545 | | | | |
| 1498883 | 29184 | 29203 | TCATCACCACCACCATCACC | 30 | C | 259 |
| | 29280 | 29299 | | | | |
| | 29370 | 29389 | | | | |
| | 29524 | 29543 | | | | |
| 1498884 | 65101 | 65120 | AGGAAATGGGGGGGCCCAGG | 111 | C | 260 |
| | 65213 | 65232 | | | | |
| 1498885 | 58717 | 58736 | CACCCGTGTCTTAGCCCTTT | 62 | C | 261 |
| 1498886 | 29523 | 29542 | CATCACCACCACCATCACCG | 23 | C | 262 |
| 1498887 | 28759 | 28778 | AGCAGGGCTGGAGAATCTTG | 67 | C | 263 |
| 1498888 | 28757 | 28776 | CAGGGCTGGAGAATCTTGGG | 47 | C | 264 |
| 1498889 | 28756 | 28775 | AGGGCTGGAGAATCTTGGGA | 57 | C | 265 |
| 1498890 | 28755 | 28774 | GGGCTGGAGAATCTTGGGAC | 45 | C | 266 |
| 1498891 | 28753 | 28772 | GCTGGAGAATCTTGGGACTG | 37 | C | 267 |
| 1498892 | 28751 | 28770 | TGGAGAATCTTGGGACTGTT | 61 | C | 268 |
| 1498893 | 58317 | 58336 | CCCACCCATCTGCACCCAGC | 76 | C | 269 |
| 1498894 | 28750 | 28769 | GGAGAATCTTGGGACTGTTG | 53 | C | 270 |
| 1498895 | 27688 | 27707 | TCAGGCCCCAGGAAGGACAT | 74 | C | 271 |
| 1498896 | 27686 | 27705 | AGGCCCCAGGAAGGACATTA | 57 | C | 272 |
| 1498897 | 27685 | 27704 | GGCCCCAGGAAGGACATTAC | 68 | C | 273 |
| 1498898 | 27684 | 27703 | GCCCCAGGAAGGACATTACT | 39 | C | 274 |
| 1498899 | 27682 | 27701 | CCCAGGAAGGACATTACTAT | 57 | C | 275 |
| 1498900 | 27680 | 27699 | CAGGAAGGACATTACTATCG | 58 | C | 276 |
| 1498901 | 27679 | 27698 | AGGAAGGACATTACTATCGT | 65 | C | 277 |
| 1498902 | 26496 | 26515 | GCCCCAGGTAACTGCAAAGG | 48 | C | 278 |
| 1498903 | 26494 | 26513 | CCCAGGTAACTGCAAAGGGG | 58 | C | 279 |
| 1498904 | 26493 | 26512 | CCAGGTAACTGCAAAGGGGG | 60 | C | 280 |
| 1498905 | 26492 | 26511 | CAGGTAACTGCAAAGGGGGC | 74 | C | 281 |
| 1498906 | 26490 | 26509 | GGTAACTGCAAAGGGGGCCA | 29 | C | 282 |
| 1498907 | 26488 | 26507 | TAACTGCAAAGGGGGCCACA | 73 | C | 283 |
| 1498908 | 58315 | 58334 | CACCCATCTGCACCCAGCTG | 71 | C | 284 |
| 1498909 | 26487 | 26506 | AACTGCAAAGGGGGCCACAG | 94 | C | 285 |
| 1498910 | 25666 | 25685 | ACCAGAGCTCGCTCGGGCCA | 28 | C | 286 |
| 1498911 | 25664 | 25683 | CAGAGCTCGCTCGGGCCAGA | 35 | C | 287 |
| 1498912 | 25663 | 25682 | AGAGCTCGCTCGGGCCAGAG | 32 | C | 288 |
| 1498913 | 25662 | 25681 | GAGCTCGCTCGGGCCAGAGG | 36 | C | 289 |
| 1498914 | 25660 | 25679 | GCTCGCTCGGGCCAGAGGGC | 45 | C | 290 |
| 1498915 | 25658 | 25677 | TCGCTCGGGCCAGAGGGCAC | 47 | C | 291 |
| 1498916 | 58314 | 58333 | ACCCATCTGCACCCAGCTGC | 71 | C | 292 |
| 1498917 | 25657 | 25676 | CGCTCGGGCCAGAGGGCACC | 41 | C | 293 |
| 1498918 | 24287 | 24306 | CTCTCGTTCCAACCGTGCTC | 33 | C | 294 |
| 1498919 | 24285 | 24304 | CTCGTTCCAACCGTGCTCAG | 69 | C | 295 |
| 1498920 | 24284 | 24303 | TCGTTCCAACCGTGCTCAGA | 49 | C | 296 |
| 1498921 | 24283 | 24302 | CGTTCCAACCGTGCTCAGAA | 35 | C | 297 |
| 1498922 | 24281 | 24300 | TTCCAACCGTGCTCAGAACC | 62 | C | 298 |
| 1498923 | 24279 | 24298 | CCAACCGTGCTCAGAACCAG | 36 | C | 299 |
| 1498924 | 58313 | 58332 | CCCATCTGCACCCAGCTGCT | 82 | C | 300 |
| 1498925 | 24278 | 24297 | CAACCGTGCTCAGAACCAGG | 64 | C | 301 |
| 1498926 | 24167 | 24186 | GGCACCACACGACCGCCACG | 34 | C | 302 |
| 1498927 | 24165 | 24184 | CACCACACGACCGCCACGGA | 45 | C | 303 |
| 1498928 | 24164 | 24183 | ACCACACGACCGCCACGGAG | 35 | C | 304 |
| 1498929 | 24163 | 24182 | CCACACGACCGCCACGGAGC | 38 | C | 305 |
| 1498930 | 24161 | 24180 | ACACGACCGCCACGGAGCCT | 34 | C | 306 |
| 1498931 | 24159 | 24178 | ACGACCGCCACGGAGCCTGT | 59 | C | 307 |
| 1498932 | 58312 | 58331 | CCATCTGCACCCAGCTGCTC | 81 | C | 308 |
| 1498933 | 24158 | 24177 | CGACCGCCACGGAGCCTGTG | 55 | C | 309 |
| 1498934 | 20473 | 20492 | TTTTAAAGCGTCTCATGTGA | 56 | C | 310 |
| 1498935 | 20471 | 20490 | TTAAAGCGTCTCATGTGAAA | 98 | C | 311 |
| 1498936 | 20470 | 20489 | TAAAGCGTCTCATGTGAAAA | 63 | C | 312 |
| 1498937 | 20469 | 20488 | AAAGCGTCTCATGTGAAAAC | 63 | C | 313 |
| 1498938 | 20467 | 20486 | AGCGTCTCATGTGAAAACTG | 28 | C | 314 |
| 1498939 | 20465 | 20484 | CGTCTCATGTGAAAACTGTC | 26 | C | 315 |
| 1498940 | 58311 | 58330 | CATCTGCACCCAGCTGCTCT | 91 | C | 316 |
| 1498941 | 20464 | 20483 | GTCTCATGTGAAAACTGTCA | 34 | C | 317 |
| 1498942 | 18909 | 18928 | CACAGGGGGAGAAGGGGAAG | 101 | C | 318 |
| 1498943 | 18907 | 18926 | CAGGGGGAGAAGGGGAAGGG | 114 | C | 319 |
| 1498944 | 18906 | 18925 | AGGGGGAGAAGGGGAAGGGC | 108 | C | 320 |
| 1498945 | 18905 | 18924 | GGGGGAGAAGGGGAAGGGCA | 72 | C | 321 |
| 1498946 | 18903 | 18922 | GGGAGAAGGGGAAGGGCATT | 108 | C | 322 |
| 1498947 | 18901 | 18920 | GAGAAGGGGAAGGGCATTCC | 69 | C | 323 |
| 1498948 | 58310 | 58329 | ATCTGCACCCAGCTGCTCTC | 67 | C | 324 |
| 1498949 | 18900 | 18919 | AGAAGGGGAAGGGCATTCCC | 67 | C | 325 |
| 1498950 | 18839 | 18858 | GCAGGCGTATCTGCATCCCA | 32 | C | 326 |
| 1498951 | 18837 | 18856 | AGGCGTATCTGCATCCCACC | 47 | C | 327 |
| 1498952 | 18836 | 18855 | GGCGTATCTGCATCCCACCG | 25 | C | 328 |
| 1498953 | 18835 | 18854 | GCGTATCTGCATCCCACCGA | 28 | C | 329 |
| 1498954 | 18833 | 18852 | GTATCTGCATCCCACCGAGG | 64 | C | 330 |
| 1498955 | 18832 | 18851 | TATCTGCATCCCACCGAGGA | 58 | C | 331 |
| 1498956 | 18831 | 18850 | ATCTGCATCCCACCGAGGAT | 66 | C | 332 |
| 1498801 | 45035 | 45054 | TGGCCGCATCACTTCAGTCT | 80 | D | 333 |
| 1498802 | 45033 | 45052 | GCCGCATCACTTCAGTCTCT | 60 | D | 334 |
| 1498803 | 45031 | 45050 | CGCATCACTTCAGTCTCTCT | 53 | D | 335 |
| 1498804 | 65102 | 65121 | CAGGAAATGGGGGGGCCCAG | 91 | D | 336 |
| | 65214 | 65233 | | | | |
| 1498805 | 63995 | 64014 | GGCAGTGGGTGCCAGGACAG | 78 | D | 337 |
| 1498806 | 45030 | 45049 | GCATCACTTCAGTCTCTCTC | 62 | D | 338 |
| 1498807 | 44604 | 44623 | TGGGACACTTTGTCGTACGA | 29 | D | 339 |
| 1498808 | 44602 | 44621 | GGACACTTTGTCGTACGACC | 34 | D | 340 |
| 1498809 | 44601 | 44620 | GACACTTTGTCGTACGACCC | 72 | D | 341 |
| 1498810 | 44600 | 44619 | ACACTTTGTCGTACGACCCA | 44 | D | 342 |
| 1498811 | 44598 | 44617 | ACTTTGTCGTACGACCCACG | 52 | D | 343 |
| 1498812 | 44596 | 44615 | TTTGTCGTACGACCCACGCG | 61 | D | 344 |
| 1498813 | 58726 | 58745 | AGCCAGGTCCACCCGTGTCT | 51 | D | 345 |
| 1498814 | 44595 | 44614 | TTGTCGTACGACCCACGCGC | 66 | D | 346 |
| 1498815 | 43262 | 43281 | GGCCACGTCCTGGTCTCTCC | 81 | D | 347 |
| 1498816 | 43260 | 43279 | CCACGTCCTGGTCTCTCCTC | 68 | D | 348 |
| 1498817 | 43259 | 43278 | CACGTCCTGGTCTCTCCTCT | 63 | D | 349 |
| 1498818 | 43258 | 43277 | ACGTCCTGGTCTCTCCTCTG | 53 | D | 350 |
| 1498819 | 43256 | 43275 | GTCCTGGTCTCTCCTCTGCG | 49 | D | 351 |
| 1498820 | 43255 | 43274 | TCCTGGTCTCTCCTCTGCGT | 57 | D | 352 |
| 1498821 | 43254 | 43273 | CCTGGTCTCTCCTCTGCGTT | 61 | D | 353 |
| 1498822 | 58725 | 58744 | GCCAGGTCCACCCGTGTCTT | 71 | D | 354 |
| 1498823 | 43253 | 43272 | CTGGTCTCTCCTCTGCGTTG | 62 | D | 355 |
| 1498824 | 42381 | 42400 | CTTCAGCGCCAGCCTCCTGG | 64 | D | 356 |
| 1498825 | 42379 | 42398 | TCAGCGCCAGCCTCCTGGCC | 63 | D | 357 |
| 1498826 | 42378 | 42397 | CAGCGCCAGCCTCCTGGCCC | 51 | D | 358 |
| 1498827 | 42377 | 42396 | AGCGCCAGCCTCCTGGCCCC | 61 | D | 359 |
| 1498828 | 42375 | 42394 | CGCCAGCCTCCTGGCCCCAG | 52 | D | 360 |
| 1498829 | 42373 | 42392 | CCAGCCTCCTGGCCCCAGGG | 49 | D | 361 |
| 1498830 | 58724 | 58743 | CCAGGTCCACCCGTGTCTTA | 77 | D | 362 |
| 1498831 | 42372 | 42391 | CAGCCTCCTGGCCCCAGGGG | 60 | D | 363 |
| 1498832 | 41143 | 41162 | CTGCCCTGGGCTGAGAGAGG | 62 | D | 364 |
| 1498833 | 41141 | 41160 | GCCCTGGGCTGAGAGAGGTC | 74 | D | 365 |
| 1498834 | 41140 | 41159 | CCCTGGGCTGAGAGAGGTCA | 72 | D | 366 |
| 1498835 | 41139 | 41158 | CCTGGGCTGAGAGAGGTCAC | 75 | D | 367 |
| 1498836 | 41137 | 41156 | TGGGCTGAGAGAGGTCACTT | 45 | D | 368 |
| 1498837 | 41135 | 41154 | GGCTGAGAGAGGTCACTTTG | 46 | D | 369 |
| 1498838 | 58723 | 58742 | CAGGTCCACCCGTGTCTTAG | 54 | D | 370 |
| 1498839 | 41134 | 41153 | GCTGAGAGAGGTCACTTTGC | 75 | D | 371 |
| 1498840 | 39464 | 39483 | TCACAGGACCTGCTAGCACT | 55 | D | 372 |
| 1498841 | 39462 | 39481 | ACAGGACCTGCTAGCACTGC | 52 | D | 373 |
| 1498842 | 39461 | 39480 | CAGGACCTGCTAGCACTGCC | 55 | D | 374 |
| 1498843 | 39460 | 39479 | AGGACCTGCTAGCACTGCCT | 50 | D | 375 |
| 1498844 | 39458 | 39477 | GACCTGCTAGCACTGCCTCG | 73 | D | 376 |
| 1498845 | 39456 | 39475 | CCTGCTAGCACTGCCTCGAC | 75 | D | 377 |
| 1498846 | 58722 | 58741 | AGGTCCACCCGTGTCTTAGC | 68 | D | 378 |
| 1498847 | 39455 | 39474 | CTGCTAGCACTGCCTCGACA | 68 | D | 379 |
| 1498848 | 37505 | 37524 | TGAAGCAGGTGGATCGCCTG | 72 | D | 380 |
| 1498849 | 37503 | 37522 | AAGCAGGTGGATCGCCTGAG | 84 | D | 381 |
| 1498850 | 37502 | 37521 | AGCAGGTGGATCGCCTGAGC | 65 | D | 382 |
| 1498851 | 37501 | 37520 | GCAGGTGGATCGCCTGAGCC | 67 | D | 383 |
| 1498852 | 37499 | 37518 | AGGTGGATCGCCTGAGCCCA | 68 | D | 384 |
| 1498853 | 37497 | 37516 | GTGGATCGCCTGAGCCCAGG | 57 | D | 385 |
| 1498854 | 37496 | 37515 | TGGATCGCCTGAGCCCAGGA | 57 | D | 386 |
| 1498855 | 32368 | 32387 | CCAAGGGTCCCGGTGAGCCC | 58 | D | 387 |
| 1498856 | 32366 | 32385 | AAGGGTCCCGGTGAGCCCAG | 94 | D | 388 |
| 1498857 | 32365 | 32384 | AGGGTCCCGGTGAGCCCAGG | 39 | D | 389 |
| 1498858 | 32364 | 32383 | GGGTCCCGGTGAGCCCAGGA | 50 | D | 390 |
| 1498859 | 32362 | 32381 | GTCCCGGTGAGCCCAGGAGC | 49 | D | 391 |
| 1498860 | 32360 | 32379 | CCCGGTGAGCCCAGGAGCAC | 44 | D | 392 |
| 1498861 | 58720 | 58739 | GTCCACCCGTGTCTTAGCCC | 73 | D | 393 |
| 1498862 | 32359 | 32378 | CCGGTGAGCCCAGGAGCACC | 43 | D | 394 |
| 1498863 | 31870 | 31889 | TGGGGGCCTTGGCTGCCACC | 41 | D | 395 |
| 1498864 | 31868 | 31887 | GGGGCCTTGGCTGCCACCAC | 71 | D | 396 |
| 1498865 | 31867 | 31886 | GGGCCTTGGCTGCCACCACC | 30 | D | 397 |
| 1498866 | 31866 | 31885 | GGCCTTGGCTGCCACCACCA | 34 | D | 398 |
| 1498867 | 31864 | 31883 | CCTTGGCTGCCACCACCACG | 44 | D | 399 |
| 1498868 | 31862 | 31881 | TTGGCTGCCACCACCACGCG | 51 | D | 400 |
| 1498869 | 31861 | 31880 | TGGCTGCCACCACCACGCGG | 40 | D | 401 |
| 1498870 | 30792 | 30811 | GCGGGGCTAGGCCCGGGGTC | 101 | D | 402 |
| 1498871 | 30790 | 30809 | GGGGCTAGGCCCGGGGTCTG | 66 | D | 403 |
| 1498872 | 30789 | 30808 | GGGCTAGGCCCGGGGTCTGG | 67 | D | 404 |
| 1498873 | 30788 | 30807 | GGCTAGGCCCGGGGTCTGGT | 53 | D | 405 |
| 1498874 | 30786 | 30805 | CTAGGCCCGGGGTCTGGTCT | 61 | D | 406 |
| 1498875 | 30784 | 30803 | AGGCCCGGGGTCTGGTCTCA | 56 | D | 407 |
| 1498876 | 58718 | 58737 | CCACCCGTGTCTTAGCCCTT | 72 | D | 408 |
| 1498877 | 30783 | 30802 | GGCCCGGGGTCTGGTCTCAG | 33 | D | 409 |
| 1498878 | 29192 | 29211 | CACCACCATCATCACCACCA | 16 | D | 410 |
| | 29288 | 29307 | | | | |
| | 29532 | 29551 | | | | |
| 1498723 | 51256 | 51275 | GGCGCTGCAGGCACAGCCCC | 64 | E | 411 |
| 1498724 | 51254 | 51273 | CGCTGCAGGCACAGCCCCCC | 39 | E | 412 |
| 1498725 | 51252 | 51271 | CTGCAGGCACAGCCCCCCAA | 60 | E | 413 |
| 1498726 | 64401 | 64420 | TGCACAGAGCAGCTGTCAGT | 95 | E | 414 |
| 1498727 | 51251 | 51270 | TGCAGGCACAGCCCCCCAAG | 56 | E | 415 |
| 1498728 | 50991 | 51010 | GGAGGGGGAGCCCGAACAGG | 107 | E | 416 |
| 1498729 | 50989 | 51008 | AGGGGGAGCCCGAACAGGCA | 95 | E | 417 |
| 1498730 | 50988 | 51007 | GGGGGAGCCCGAACAGGCAC | 86 | E | 418 |
| 1498731 | 50987 | 51006 | GGGGAGCCCGAACAGGCACG | 66 | E | 419 |
| 1498732 | 50985 | 51004 | GGAGCCCGAACAGGCACGGG | 60 | E | 420 |
| 1498733 | 50983 | 51002 | AGCCCGAACAGGCACGGGGG | 71 | E | 421 |
| 1498734 | 64004 | 64023 | CGACCAGGGGGCAGTGGGTG | 72 | E | 422 |
| 1498735 | 50982 | 51001 | GCCCGAACAGGCACGGGGGA | 94 | E | 423 |
| 1498736 | 50909 | 50928 | GGGGCAGGAGGCAGTGAGAT | 80 | E | 424 |
| 1498737 | 50907 | 50926 | GGCAGGAGGCAGTGAGATGG | 69 | E | 425 |
| 1498738 | 50906 | 50925 | GCAGGAGGCAGTGAGATGGG | 44 | E | 426 |
| 1498739 | 50905 | 50924 | CAGGAGGCAGTGAGATGGGG | 115 | E | 427 |
| 1498740 | 50903 | 50922 | GGAGGCAGTGAGATGGGGTG | 96 | E | 428 |
| 1498741 | 50901 | 50920 | AGGCAGTGAGATGGGGTGTC | 90 | E | 429 |
| 1498742 | 50900 | 50919 | GGCAGTGAGATGGGGTGTCG | 110 | E | 430 |
| 1498743 | 50272 | 50291 | CTCCTCACTCCCACCGCACC | 66 | E | 431 |
| 1498744 | 50270 | 50289 | CCTCACTCCCACCGCACCCC | 57 | E | 432 |
| 1498745 | 50269 | 50288 | CTCACTCCCACCGCACCCCA | 55 | E | 433 |
| 1498746 | 50268 | 50287 | TCACTCCCACCGCACCCCAG | 96 | E | 434 |
| 1498747 | 50266 | 50285 | ACTCCCACCGCACCCCAGGC | 64 | E | 435 |
| 1498748 | 50264 | 50283 | TCCCACCGCACCCCAGGCAC | 86 | E | 436 |
| 1498749 | 64002 | 64021 | ACCAGGGGGCAGTGGGTGCC | 90 | E | 437 |
| 1498750 | 50263 | 50282 | CCCACCGCACCCCAGGCACC | 73 | E | 438 |
| 1498751 | 47097 | 47116 | ATACAAAAATTAGCCGGGTG | 120 | E | 439 |
| 1498752 | 47095 | 47114 | ACAAAAATTAGCCGGGTGTG | 95 | E | 440 |
| 1498753 | 47094 | 47113 | CAAAAATTAGCCGGGTGTGG | 118 | E | 441 |
| 1498754 | 47093 | 47112 | AAAAATTAGCCGGGTGTGGT | 96 | E | 442 |
| 1498755 | 47091 | 47110 | AAATTAGCCGGGTGTGGTGG | 110 | E | 443 |
| 1498756 | 47089 | 47108 | ATTAGCCGGGTGTGGTGGCA | 133 | E | 444 |
| 1498757 | 64001 | 64020 | CCAGGGGGCAGTGGGTGCCA | 106 | E | 445 |
| 1498758 | 47088 | 47107 | TTAGCCGGGTGTGGTGGCAG | 79 | E | 446 |
| 1498759 | 46172 | 46191 | CTGCCCTGAATGCCTTTTTT | 62 | E | 447 |
| 1498760 | 46170 | 46189 | GCCCTGAATGCCTTTTTTTT | 66 | E | 448 |
| 1498761 | 46169 | 46188 | CCCTGAATGCCTTTTTTTTT | 65 | E | 449 |
| 1498762 | 46168 | 46187 | CCTGAATGCCTTTTTTTTTT | 57 | E | 450 |
| 1498763 | 46166 | 46185 | TGAATGCCTTTTTTTTTTTT | 78 | E | 451 |
| 1498764 | 46164 | 46183 | AATGCCTTTTTTTTTTTTTT | 95 | E | 452 |
| 1498765 | 64000 | 64019 | CAGGGGGCAGTGGGTGCCAG | 92 | E | 453 |
| 1498766 | 46163 | 46182 | ATGCCTTTTTTTTTTTTTTT | 67 | E | 454 |
| 1498767 | 45654 | 45673 | GAGGTCAGGAGCTTCAGACC | 55 | E | 455 |
| 1498768 | 45652 | 45671 | GGTCAGGAGCTTCAGACCAG | 97 | E | 456 |
| 1498769 | 45651 | 45670 | GTCAGGAGCTTCAGACCAGC | 76 | E | 457 |
| 1498770 | 45650 | 45669 | TCAGGAGCTTCAGACCAGCC | 105 | E | 458 |
| 1498771 | 45648 | 45667 | AGGAGCTTCAGACCAGCCTG | 87 | E | 459 |
| 1498772 | 45646 | 45665 | GAGCTTCAGACCAGCCTGGC | 63 | E | 460 |
| 1498773 | 45645 | 45664 | AGCTTCAGACCAGCCTGGCC | 66 | E | 461 |
| 1498774 | 45346 | 45365 | GTCCTCCACGCATGAACCTG | 36 | E | 462 |
| 1498775 | 45344 | 45363 | CCTCCACGCATGAACCTGTG | 79 | E | 463 |
| 1498776 | 45343 | 45362 | CTCCACGCATGAACCTGTGA | 53 | E | 464 |
| 1498777 | 45342 | 45361 | TCCACGCATGAACCTGTGAT | 80 | E | 465 |
| 1498778 | 45341 | 45360 | CCACGCATGAACCTGTGATT | 79 | E | 466 |
| 1498779 | 45340 | 45359 | CACGCATGAACCTGTGATTC | 64 | E | 467 |
| 1498780 | 45338 | 45357 | CGCATGAACCTGTGATTCTA | 60 | E | 468 |
| 1498781 | 63998 | 64017 | GGGGGCAGTGGGTGCCAGGA | 100 | E | 469 |
| 1498782 | 45337 | 45356 | GCATGAACCTGTGATTCTAG | 48 | E | 470 |
| 1498783 | 45295 | 45314 | GGTTCTGATGTCACACCCGC | 52 | E | 471 |
| 1498784 | 45293 | 45312 | TTCTGATGTCACACCCGCGC | 74 | E | 472 |
| 1498785 | 45292 | 45311 | TCTGATGTCACACCCGCGCT | 74 | E | 473 |
| 1498786 | 45291 | 45310 | CTGATGTCACACCCGCGCTT | 62 | E | 474 |
| 1498787 | 45289 | 45308 | GATGTCACACCCGCGCTTGT | 68 | E | 475 |
| 1498788 | 45287 | 45306 | TGTCACACCCGCGCTTGTAG | 52 | E | 476 |
| 1498789 | 45286 | 45305 | GTCACACCCGCGCTTGTAGG | 53 | E | 477 |
| 1498790 | 45274 | 45293 | CTTGTAGGCGTTTGCCACAG | 64 | E | 478 |
| 1498791 | 45272 | 45291 | TGTAGGCGTTTGCCACAGCC | 78 | E | 479 |
| 1498792 | 45271 | 45290 | GTAGGCGTTTGCCACAGCCG | 70 | E | 480 |
| 1498793 | 45270 | 45289 | TAGGCGTTTGCCACAGCCGC | 52 | E | 481 |
| 1498794 | 45268 | 45287 | GGCGTTTGCCACAGCCGCAC | 65 | E | 482 |
| 1498795 | 45266 | 45285 | CGTTTGCCACAGCCGCACGC | 65 | E | 483 |
| 1498796 | 63996 | 64015 | GGGCAGTGGGTGCCAGGACA | 88 | E | 484 |
| 1498797 | 45265 | 45284 | GTTTGCCACAGCCGCACGCG | 79 | E | 485 |
| 1498798 | 45039 | 45058 | CTTGTGGCCGCATCACTTCA | 79 | E | 486 |
| 1498799 | 45037 | 45056 | TGTGGCCGCATCACTTCAGT | 64 | E | 487 |
| 1498800 | 45036 | 45055 | GTGGCCGCATCACTTCAGTC | 80 | E | 488 |
| 1498645 | 65104 | 65123 | CCCAGGAAATGGGGGGGCCC | 57 | F | 489 |
| | 65216 | 65235 | | | | |
| 1498646 | 65095 | 65114 | TGGGGGGGCCCAGGCTGGTT | 105 | F | 490 |
| | 65207 | 65226 | | | | |
| 1498647 | 55070 | 55089 | GAATGGGCATCAGATTCCCA | 61 | F | 491 |
| 1498648 | 54339 | 54358 | GCACCTGAGAGCGCGCCATC | 56 | F | 492 |
| 1498649 | 54337 | 54356 | ACCTGAGAGCGCGCCATCCC | 81 | F | 493 |
| 1498650 | 54336 | 54355 | CCTGAGAGCGCGCCATCCCC | 65 | F | 494 |
| 1498651 | 54335 | 54354 | CTGAGAGCGCGCCATCCCCA | 67 | F | 495 |
| 1498652 | 54333 | 54352 | GAGAGCGCGCCATCCCCACC | 70 | F | 496 |
| 1498653 | 54331 | 54350 | GAGCGCGCCATCCCCACCCG | 85 | F | 497 |
| 1498654 | 64410 | 64429 | TGAGGGGTCTGCACAGAGCA | 83 | F | 498 |
| 1498655 | 54332 | 54351 | AGAGCGCGCCATCCCCACCC | 74 | F | 499 |
| 1498656 | 54330 | 54349 | AGCGCGCCATCCCCACCCGG | 75 | F | 500 |
| 1498657 | 54233 | 54252 | CCCCTGCCCAGCTGGTGTCC | 42 | F | 501 |
| 1498658 | 54231 | 54250 | CCTGCCCAGCTGGTGTCCGG | 61 | F | 502 |
| 1498659 | 54230 | 54249 | CTGCCCAGCTGGTGTCCGGA | 71 | F | 503 |
| 1498660 | 54229 | 54248 | TGCCCAGCTGGTGTCCGGAC | 67 | F | 504 |
| 1498661 | 54227 | 54246 | CCCAGCTGGTGTCCGGACCA | 56 | F | 505 |
| 1498662 | 54225 | 54244 | CAGCTGGTGTCCGGACCAAC | 63 | F | 506 |
| 1498663 | 54224 | 54243 | AGCTGGTGTCCGGACCAACG | 70 | F | 507 |
| 1498664 | 54021 | 54040 | TAGATGCTCCGGACACGCAG | 41 | F | 508 |
| 1498665 | 54019 | 54038 | GATGCTCCGGACACGCAGGA | 78 | F | 509 |
| 1498666 | 54018 | 54037 | ATGCTCCGGACACGCAGGAC | 84 | F | 510 |
| 1498667 | 54017 | 54036 | TGCTCCGGACACGCAGGACA | 72 | F | 511 |
| 1498668 | 54015 | 54034 | CTCCGGACACGCAGGACAGC | 76 | F | 512 |
| 1498669 | 54013 | 54032 | CCGGACACGCAGGACAGCTG | 57 | F | 513 |
| 1498670 | 64408 | 64427 | AGGGGTCTGCACAGAGCAGC | 83 | F | 514 |
| 1498671 | 54012 | 54031 | CGGACACGCAGGACAGCTGC | 103 | F | 515 |
| 1498672 | 53982 | 54001 | GCAGGCCATGCCCCAGGCAG | 59 | F | 516 |
| 1498673 | 53980 | 53999 | AGGCCATGCCCCAGGCAGCC | 44 | F | 517 |
| 1498674 | 53979 | 53998 | GGCCATGCCCCAGGCAGCCC | 55 | F | 518 |
| 1498675 | 53978 | 53997 | GCCATGCCCCAGGCAGCCCA | 45 | F | 519 |
| 1498676 | 53976 | 53995 | CATGCCCCAGGCAGCCCACA | 40 | F | 520 |
| 1498677 | 53974 | 53993 | TGCCCCAGGCAGCCCACACG | 61 | F | 521 |
| 1498678 | 64407 | 64426 | GGGGTCTGCACAGAGCAGCT | 97 | F | 522 |
| 1498679 | 53973 | 53992 | GCCCCAGGCAGCCCACACGC | 62 | F | 523 |
| 1498680 | 53963 | 53982 | GCCCACACGCCCGTCTCCCT | 35 | F | 524 |
| 1498681 | 53961 | 53980 | CCACACGCCCGTCTCCCTCT | 59 | F | 525 |
| 1498682 | 53960 | 53979 | CACACGCCCGTCTCCCTCTC | 58 | F | 526 |
| 1498683 | 53959 | 53978 | ACACGCCCGTCTCCCTCTCT | 79 | F | 527 |
| 1498684 | 53957 | 53976 | ACGCCCGTCTCCCTCTCTGG | 51 | F | 528 |
| 1498685 | 53956 | 53975 | CGCCCGTCTCCCTCTCTGGA | 77 | F | 529 |
| 1498686 | 53955 | 53974 | GCCCGTCTCCCTCTCTGGAG | 54 | F | 530 |
| 1498687 | 64406 | 64425 | GGGTCTGCACAGAGCAGCTG | 129 | F | 531 |
| 1498688 | 53954 | 53973 | CCCGTCTCCCTCTCTGGAGC | 102 | F | 532 |
| 1498689 | 53443 | 53462 | GCTGCCTCCCGCGGTCGAGG | 47 | F | 533 |
| 1498690 | 53441 | 53460 | TGCCTCCCGCGGTCGAGGCA | 107 | F | 534 |
| 1498691 | 53440 | 53459 | GCCTCCCGCGGTCGAGGCAG | 83 | F | 535 |
| 1498692 | 53439 | 53458 | CCTCCCGCGGTCGAGGCAGC | 65 | F | 536 |
| 1498693 | 53437 | 53456 | TCCCGCGGTCGAGGCAGCCA | 65 | F | 537 |
| 1498694 | 53435 | 53454 | CCGCGGTCGAGGCAGCCAGG | 74 | F | 538 |
| 1498695 | 53434 | 53453 | CGCGGTCGAGGCAGCCAGGA | 84 | F | 539 |
| 1498696 | 52161 | 52180 | CACTTCTCAAACTCGGTGCC | 49 | F | 540 |
| 1498697 | 52160 | 52179 | ACTTCTCAAACTCGGTGCCC | 83 | F | 541 |
| 1498698 | 52159 | 52178 | CTTCTCAAACTCGGTGCCCC | 75 | F | 542 |
| 1498699 | 52158 | 52177 | TTCTCAAACTCGGTGCCCCA | 91 | F | 543 |
| 1498700 | 52157 | 52176 | TCTCAAACTCGGTGCCCCAG | 66 | F | 544 |
| 1498701 | 52155 | 52174 | TCAAACTCGGTGCCCCAGGA | 63 | F | 545 |
| 1498702 | 52153 | 52172 | AAACTCGGTGCCCCAGGAAC | 83 | F | 546 |
| 1498703 | 64404 | 64423 | GTCTGCACAGAGCAGCTGTC | 70 | F | 547 |
| 1498704 | 52152 | 52171 | AACTCGGTGCCCCAGGAACC | 102 | F | 548 |
| 1498705 | 51772 | 51791 | CACGAACGCGACGGTGGGAC | 57 | F | 549 |
| 1498706 | 51770 | 51789 | CGAACGCGACGGTGGGACGA | 62 | F | 550 |
| 1498707 | 51769 | 51788 | GAACGCGACGGTGGGACGAG | 59 | F | 551 |
| 1498708 | 51768 | 51787 | AACGCGACGGTGGGACGAGG | 81 | F | 552 |
| 1498709 | 51766 | 51785 | CGCGACGGTGGGACGAGGCA | 93 | F | 553 |
| 1498710 | 51764 | 51783 | CGACGGTGGGACGAGGCAGG | 68 | F | 554 |
| 1498711 | 51763 | 51782 | GACGGTGGGACGAGGCAGGA | 71 | F | 555 |
| 1498712 | 51331 | 51350 | TTTTAACAGCCACATGTGGC | 90 | F | 556 |
| 1498713 | 51329 | 51348 | TTAACAGCCACATGTGGCTG | 105 | F | 557 |
| 1498714 | 51328 | 51347 | TAACAGCCACATGTGGCTGG | 69 | F | 558 |
| 1498715 | 51327 | 51346 | AACAGCCACATGTGGCTGGC | 87 | F | 559 |
| 1498716 | 51325 | 51344 | CAGCCACATGTGGCTGGCAG | 111 | F | 560 |
| 1498717 | 51323 | 51342 | GCCACATGTGGCTGGCAGCT | 56 | F | 561 |
| 1498718 | 64402 | 64421 | CTGCACAGAGCAGCTGTCAG | 63 | F | 562 |
| 1498719 | 51322 | 51341 | CCACATGTGGCTGGCAGCTC | 49 | F | 563 |
| 1498720 | 51260 | 51279 | GGGAGGCGCTGCAGGCACAG | 61 | F | 564 |
| 1498721 | 51258 | 51277 | GAGGCGCTGCAGGCACAGCC | 44 | F | 565 |
| 1498722 | 51257 | 51276 | AGGCGCTGCAGGCACAGCCC | 80 | F | 566 |

### Example 3: Effect of 5-10-5 MOE Modified Oligonucleotides with Mixed PO/PS Linkages on Human KCNQ2 RNA In Vitro, Single Dose

Modified oligonucleotides complementary to a human KCNQ2 nucleic acid were designed and tested for their single dose effects on KCNQ2 RNA in vitro. The modified oligonucleotides were tested in a series of experiments that had the same culture conditions.

“Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the tables below is 100% complementary to SEQ ID NO: 2 (described herein above), or to both.

Each separate experimental analysis described in this example is identified by a letter ID in the table column labeled “AID” (Analysis ID). Cultured SH-SY5Y cells were treated with modified oligonucleotide at a concentration of 4000 nM by electroporation at a density of 20,000-35,000 cells per well. After a treatment period of approximately 24 hours, total RNA was isolated from the cells, and KCNQ2 RNA levels were measured by quantitative real-time RTPCR. KCNQ2 RNA levels were measured by human primer-probe set RTS49037 (described herein above). KCNQ2 RNA levels were normalized to total RNA content, as measured by RIBOGREEN®. Reduction of KCNQ2 RNA is presented in Tables 4-5 below as percent KCNQ2 RNA relative to the amount in untreated control cells (% UTC).

The modified oligonucleotides in the table below are 5-10-5 MOE modified oligonucleotides with mixed PO/PS internucleoside linkages. The modified oligonucleotides are 20 nucleosides in length, wherein the central gap segment consists of ten 2′-β-D-deoxynucleosides and wherein the 5′ and 3′ wings each consist of five 2′-MOE modified nucleosides. The sugar motif of the modified oligonucleotides is (from 5′ to 3′): eeeeeddddddddddeeeee; wherein each “d” represents a 2′-β-D-deoxyribosyl sugar moiety, and each “e” represents a 2′-MOE sugar moiety. The internucleoside linkage motif of the modified oligonucleotides is (from 5′ to 3′): soooossssssssssooss; wherein each “o” represents a phosphodiester internucleoside linkage and each “s” represents a phosphorothioate internucleoside linkage. Each cytosine residue is a 5-methyl cytosine.

TABLE 4
Reduction of KCNQ2 RNA by 5-10-5 MOE modified oligonucleotides
with mixed PO/PS linkages in SH-SY5Y cells

| | SEQ ID | SEQ ID | | | | |
|---|---|---|---|---|---|---|
| | No: 2 | No: 2 | | KCNQ2 | | |
| Compound | Start | Stop | | (% | | SEQ ID |
| Number | Site | Site | Sequence (5′ to 3′) | UTC) | AID | NO |
| 1577094 | 71981 | 72000 | ATGGCAGACTGCAATGGCGT | 81 | H | 567 |
| 1577095 | 27047 | 27066 | CTGTCTGAGCTGGGGACTCT | 49 | H | 568 |
| 1577098 | 32961 | 32980 | GTCAGCGTGATCTGTGGGAC | 65 | H | 569 |
| 1577100 | 65742 | 65761 | CCGCAGGTTCCCCTCGGGGG | 82 | H | 570 |
| 1577104 | 71086 | 71105 | CTCCCCAGGCGGCCATTCCG | 72 | H | 571 |
| 1577108 | 27035 | 27054 | GGGACTCTATCTGGGCTAGG | 44 | H | 572 |
| 1577125 | 68319 | 68338 | GCCACACCTTGGGGGGGGAG | 67 | H | 573 |
| 1577135 | 26489 | 26508 | GTAACTGCAAAGGGGGCCAC | 57 | H | 574 |
| 1577140 | 13864 | 13883 | GCAGCTAGGCCTGGGGCCCA | 31 | H | 575 |
| 1577153 | 29866 | 29885 | ATTACCACCACCATTACCAC | 62 | H | 576 |
| 1577160 | 16508 | 16527 | CTCCGGGAGACCCCTCTGCT | 20 | H | 577 |
| | 16816 | 16835 | | | | |
| 1577162 | 27007 | 27026 | TCTGAGCTGGCAGGGCTGCC | 56 | H | 578 |
| 1577169 | 26722 | 26741 | CCTGTCTGAGCTGGGAGGCC | 32 | H | 579 |
| | 26779 | 26798 | | | | |
| | 26836 | 26855 | | | | |
| | 26874 | 26893 | | | | |
| | 26931 | 26950 | | | | |
| | 26950 | 26969 | | | | |
| 1577178 | 29413 | 29432 | CACATCACCATCGCCACCAT | 62 | H | 580 |
| 1577182 | 27041 | 27060 | GAGCTGGGGACTCTATCTGG | 48 | H | 581 |
| 1577192 | 70474 | 70493 | CACCCTCCACAGCAGGTCCA | 53 | H | 582 |
| | 70880 | 70899 | | | | |
| 1577197 | 5510 | 5529 | GGACCTTCGGAGGGCCCAGG | 67 | H | 583 |
| 1577200 | 17560 | 17579 | GGATGAGGGGAGGACGGCGG | 88 | H | 584 |
| 1577203 | 9939 | 9958 | ACCATTTGCAGCGGCAAAGT | 38 | H | 585 |
| 1577213 | 6032 | 6051 | CGTTGAATCCCGGCGGCCAT | 87 | H | 586 |
| 1577219 | 30793 | 30812 | TGCGGGGCTAGGCCCGGGGT | 84 | H | 587 |
| 1577221 | 28749 | 28768 | GAGAATCTTGGGACTGTTGA | 58 | H | 588 |
| 1577236 | 40723 | 40742 | CTCCACCCTCAGGGTAGGAT | 58 | H | 589 |
| 1577238 | 13870 | 13889 | AGGCTGGCAGCTAGGCCTGG | 48 | H | 590 |
| 1577241 | 39848 | 39867 | GGGAAGGCTCCACCCTCTGG | 49 | H | 591 |
| | 40046 | 40065 | | | | |
| | 40136 | 40155 | | | | |
| | 40190 | 40209 | | | | |
| | 40298 | 40317 | | | | |
| | 40478 | 40497 | | | | |
| 1577245 | 16509 | 16528 | CCTCCGGGAGACCCCTCTGC | 23 | H | 592 |
| | 16817 | 16836 | | | | |
| 1577247 | 32073 | 32092 | AGACAGAGGGATCGAGGCCT | 31 | H | 593 |
| 1577264 | 71092 | 71111 | CACAGGCTCCCCAGGCGGCC | 49 | H | 594 |
| 1577268 | 71398 | 71417 | CCCCCGGCGATGTCACCTCC | 67 | H | 595 |
| 1577279 | 17551 | 17570 | GAGGACGGCGGGAAGACGAT | 83 | H | 596 |
| 1577295 | 16114 | 16133 | CCCTGCCTCAGACAGAGCCA | 37 | H | 597 |
| 1577298 | 3750 | 3769 | AACCTTCCTCTGCCTCAGTT | 45 | H | 598 |
| 1577302 | 16108 | 16127 | CTCAGACAGAGCCAGGGTTG | 67 | H | 599 |
| 1577303 | 40501 | 40520 | CCTCAGGGAAGGCCACACCC | 68 | H | 600 |
| 1577305 | 14260 | 14279 | CCCTCGGCAAACGCCGTCCC | 47 | H | 601 |
| 1577308 | 30972 | 30991 | CACAGCCTGGGCCCCGGGGC | 39 | H | 602 |
| 1577309 | 26619 | 26638 | CTGTCCAAGCTGGGGGACTC | 66 | H | 603 |
| 1577312 | 71987 | 72006 | CAGAGGATGGCAGACTGCAA | 78 | H | 604 |
| 1577322 | 29931 | 29950 | TCACCACCTCCACCATCACC | 20 | H | 605 |
| 1577330 | 68418 | 68437 | GCCTCACCTCGGGGGAGGAA | 79 | H | 606 |
| 1577332 | 43442 | 43461 | CTCACAGCATTCCAGCTCGC | 71 | H | 607 |
| 1577333 | 70478 | 70497 | TGGGCACCCTCCACAGCAGG | 54 | H | 608 |
| | 70884 | 70903 | | | | |
| 1577337 | 40654 | 40673 | AGGCCCCATCCACAGGGAAG | 58 | H | 609 |
| 1577340 | 14266 | 14285 | CTCCTGCCCTCGGCAAACGC | 52 | H | 610 |
| 1577343 | 70405 | 70424 | GGCAGGTCCGAGCTTTGTGA | 85 | H | 611 |
| 1577350 | 51175 | 51194 | CTGGCACTGGGCTTCCTTCC | 64 | H | 612 |
| 1577354 | 28814 | 28833 | TCGCACGGCCAGCCCACCGT | 56 | H | 613 |
| 1577356 | 28820 | 28839 | AGTGGCTCGCACGGCCAGCC | 59 | H | 614 |
| 1577362 | 37072 | 37091 | GCCAGAGAGTACACGGCGGC | 34 | H | 615 |
| 1577364 | 10329 | 10348 | GGGAGGAGGCTGCATCTACC | 46 | H | 616 |
| | 10449 | 10468 | | | | |
| | 10479 | 10498 | | | | |
| | 10630 | 10649 | | | | |
| | 10720 | 10739 | | | | |
| 1577366 | 26625 | 26644 | GCTGCCCTGTCCAAGCTGGG | 30 | H | 617 |
| 1577370 | 40495 | 40514 | GGAAGGCCACACCCACAGGG | 67 | H | 618 |
| 1577374 | 40717 | 40736 | CCTCAGGGTAGGATCCACCC | 54 | H | 619 |
| 1577383 | 40660 | 40679 | CAGGGAAGGCCCCATCCACA | 59 | H | 620 |
| 1577385 | 51181 | 51200 | CCGCAGCTGGCACTGGGCTT | 68 | H | 621 |
| 1577401 | 30966 | 30985 | CTGGGCCCCGGGGCACGCTC | 61 | H | 622 |
| 1577404 | 10330 | 10349 | AGGGAGGAGGCTGCATCTAC | 52 | H | 623 |
| | 10450 | 10469 | | | | |
| | 10480 | 10499 | | | | |
| | 10631 | 10650 | | | | |
| | 10721 | 10740 | | | | |
| 1577406 | 39308 | 39327 | ATCCGGGGTTACTTTCCTGC | 57 | H | 624 |
| 1577417 | 70411 | 70430 | CTCCACGGCAGGTCCGAGCT | 44 | H | 625 |
| 1577419 | 68310 | 68329 | TGGGGGGGGAGGAAAGAGCA | 93 | H | 626 |
| 1577423 | 37066 | 37085 | GAGTACACGGCGGCAGGGCT | 28 | H | 627 |
| 1577424 | 43436 | 43455 | GCATTCCAGCTCGCTTCAGG | 82 | H | 628 |
| 1577431 | 32361 | 32380 | TCCCGGTGAGCCCAGGAGCA | 57 | H | 629 |
| 1577432 | 29940 | 29959 | CCACCATCATCACCACCTCC | 27 | H | 630 |
| 1577450 | 71350 | 71369 | TTACAAGACGGCAAAGTTCA | 97 | H | 631 |
| 1577461 | 32079 | 32098 | ATAAACAGACAGAGGGATCG | 49 | H | 632 |
| 1577467 | 32969 | 32988 | CAATGGTGGTCAGCGTGATC | 45 | H | 633 |
| 1577468 | 68424 | 68443 | GGCGGGGCCTCACCTCGGGG | 63 | H | 634 |
| 1577473 | 5516 | 5535 | TCAAGTGGACCTTCGGAGGG | 82 | H | 635 |
| 1577480 | 29406 | 29425 | CCATCGCCACCATCATCACC | 41 | H | 636 |
| 1577485 | 71356 | 71375 | CCACCCTTACAAGACGGCAA | 92 | H | 637 |
| 1577497 | 32965 | 32984 | GGTGGTCAGCGTGATCTGTG | 42 | H | 638 |
| 1577510 | 3756 | 3775 | TCACTCAACCTTCCTCTGCC | 60 | H | 639 |
| 1577517 | 27001 | 27020 | CTGGCAGGGCTGCCCTGTCT | 75 | H | 640 |
| 1577524 | 6025 | 6044 | TCCCGGCGGCCATCACGTGA | 38 | H | 641 |
| 1577527 | 39302 | 39321 | GGTTACTTTCCTGCCCCCCA | 55 | H | 642 |
| 1577530 | 26716 | 26735 | TGAGCTGGGAGGCCCTGTCT | 15 | H | 643 |
| | 26773 | 26792 | | | | |
| | 26830 | 26849 | | | | |
| | 26868 | 26887 | | | | |
| | 26925 | 26944 | | | | |
| | 26944 | 26963 | | | | |
| 1577542 | 9933 | 9952 | TGCAGCGGCAAAGTCCTAGC | 49 | H | 644 |

The modified oligonucleotides in the table below are 5-10-5 MOE modified oligonucleotides with mixed PO/PS internucleoside linkages. The modified oligonucleotides are 20 nucleosides in length, wherein the central gap segment consists of ten 2′-β-D-deoxynucleosides and wherein the 5′ and 3′ wings each consist of five 2′-MOE modified nucleosides. The sugar motif of the modified oligonucleotides is (from 5′ to 3′): eeeeeddddddddddeeeee; wherein each “d” represents a 2′-β-D-deoxyribosyl sugar moiety, and each “e” represents a 2′-MOE sugar moiety. The internucleoside linkage motif of the modified oligonucleotides is (from 5′ to 3′): soooossssssssssooss; wherein each “o” represents a phosphodiester internucleoside linkage and each “s” represents a phosphorothioate internucleoside linkage. Each cytosine residue is a 5-methyl cytosine.

TABLE 5
Reduction of KCNQ2 RNA by 5-10-5 MOE modified oligonucleotides
with mixed PO/PS linkages in SH-SY5Y cells

| | SEQ ID | SEQ ID | | | | |
|---|---|---|---|---|---|---|
| | No: 2 | No: 2 | | KCNQ2 | | |
| Compound | Start | Stop | | (% | | SEQ ID |
| Number | Site | Site | Sequence (5′ to 3′) | UTC) | AID | NO |
| 1577087 | 37067 | 37086 | AGAGTACACGGCGGCAGGGC | 37 | I | 645 |
| 1577091 | 51176 | 51195 | GCTGGCACTGGGCTTCCTTC | 61 | I | 646 |
| 1577097 | 71399 | 71418 | TCCCCCGGCGATGTCACCTC | 47 | I | 647 |
| 1577109 | 26620 | 26639 | CCTGTCCAAGCTGGGGGACT | 62 | I | 648 |
| 1577117 | 71345 | 71364 | AGACGGCAAAGTTCAGCAAA | 63 | I | 649 |
| 1577123 | 39850 | 39869 | CAGGGAAGGCTCCACCCTCT | 34 | I | 650 |
| | 40048 | 40067 | | | | |
| | 40138 | 40157 | | | | |
| | 40192 | 40211 | | | | |
| | 40300 | 40319 | | | | |
| | 40480 | 40499 | | | | |
| 1577131 | 30967 | 30986 | CCTGGGCCCCGGGGCACGCT | 54 | I | 651 |
| 1577139 | 68419 | 68438 | GGCCTCACCTCGGGGGAGGA | 77 | I | 652 |
| 1577149 | 26717 | 26736 | CTGAGCTGGGAGGCCCTGTC | 37 | I | 653 |
| | 26774 | 26793 | | | | |
| | 26831 | 26850 | | | | |
| | 26869 | 26888 | | | | |
| | 26926 | 26945 | | | | |
| | 26945 | 26964 | | | | |
| 1577150 | 10440 | 10459 | CTGCATCTACCCCAGGCAGC | 28 | I | 654 |
| | 10711 | 10730 | | | | |
| 1577179 | 10439 | 10458 | TGCATCTACCCCAGGCAGCA | 27 | I | 655 |
| | 10710 | 10729 | | | | |
| 1577181 | 29941 | 29960 | ACCACCATCATCACCACCTC | 19 | I | 656 |
| 1577188 | 27678 | 27697 | GGAAGGACATTACTATCGTC | 38 | I | 657 |
| 1577191 | 32962 | 32981 | GGTCAGCGTGATCTGTGGGA | 26 | 1 | 658 |
| 1577198 | 71081 | 71100 | CAGGCGGCCATTCCGTCCTG | 101 | I | 659 |
| 1577199 | 5517 | 5536 | CTCAAGTGGACCTTCGGAGG | 46 | I | 660 |
| 1577207 | 13871 | 13890 | CAGGCTGGCAGCTAGGCCTG | 103 | I | 661 |
| 1577208 | 43443 | 43462 | CCTCACAGCATTCCAGCTCG | 73 | I | 662 |
| 1577217 | 27042 | 27061 | TGAGCTGGGGACTCTATCTG | 69 | I | 663 |
| 1577223 | 71393 | 71412 | GGCGATGTCACCTCCTCCCA | 61 | I | 664 |
| 1577231 | 16109 | 16128 | CCTCAGACAGAGCCAGGGTT | 62 | I | 665 |
| 1577233 | 16513 | 16532 | CCCACCTCCGGGAGACCCCT | 30 | I | 666 |
| | 16787 | 16806 | | | | |
| | 16821 | 16840 | | | | |
| | 16889 | 16908 | | | | |
| 1577234 | 32970 | 32989 | CCAATGGTGGTCAGCGTGAT | 48 | I | 667 |
| 1577237 | 40718 | 40737 | CCCTCAGGGTAGGATCCACC | 45 | I | 668 |
| 1577239 | 40496 | 40515 | GGGAAGGCCACACCCACAGG | 52 | I | 669 |
| 1577240 | 51182 | 51201 | TCCGCAGCTGGCACTGGGCT | 58 | I | 670 |
| 1577248 | 26626 | 26645 | AGCTGCCCTGTCCAAGCTGG | 16 | I | 671 |
| 1577255 | 40655 | 40674 | AAGGCCCCATCCACAGGGAA | 52 | I | 672 |
| 1577260 | 27036 | 27055 | GGGGACTCTATCTGGGCTAG | 32 | I | 673 |
| 1577261 | 29867 | 29886 | CATTACCACCACCATTACCA | 64 | I | 674 |
| 1577263 | 37073 | 37092 | GGCCAGAGAGTACACGGCGG | 47 | I | 675 |
| 1577269 | 26718 | 26737 | TCTGAGCTGGGAGGCCCTGT | 16 | I | 676 |
| | 26775 | 26794 | | | | |
| | 26832 | 26851 | | | | |
| | 26870 | 26889 | | | | |
| | 26927 | 26946 | | | | |
| | 26946 | 26965 | | | | |
| 1577271 | 70475 | 70494 | GCACCCTCCACAGCAGGTCC | 74 | I | 677 |
| | 70881 | 70900 | | | | |
| 1577273 | 70406 | 70425 | CGGCAGGTCCGAGCTTTGTG | 81 | I | 678 |
| 1577274 | 6027 | 6046 | AATCCCGGCGGCCATCACGT | 31 | I | 679 |
| 1577288 | 16115 | 16134 | CCCCTGCCTCAGACAGAGCC | 47 | I | 680 |
| 1577292 | 32074 | 32093 | CAGACAGAGGGATCGAGGCC | 49 | I | 681 |
| 1577294 | 40502 | 40521 | CCCTCAGGGAAGGCCACACC | 67 | I | 682 |
| 1577300 | 29414 | 29433 | TCACATCACCATCGCCACCA | 63 | I | 683 |
| 1577310 | 13865 | 13884 | GGCAGCTAGGCCTGGGGCCC | 27 | I | 684 |
| 1577318 | 32080 | 32099 | GATAAACAGACAGAGGGATC | 71 | I | 685 |
| 1577324 | 26491 | 26510 | AGGTAACTGCAAAGGGGGCC | 41 | I | 686 |
| 1577345 | 28821 | 28840 | CAGTGGCTCGCACGGCCAGC | 79 | I | 687 |
| 1577355 | 68311 | 68330 | TTGGGGGGGGAGGAAAGAGC | 104 | I | 688 |
| 1577363 | 27002 | 27021 | GCTGGCAGGGCTGCCCTGTC | 48 | I | 689 |
| 1577367 | 71351 | 71370 | CTTACAAGACGGCAAAGTTC | 88 | I | 690 |
| 1577376 | 29932 | 29951 | ATCACCACCTCCACCATCAC | 54 | I | 691 |
| 1577379 | 31860 | 31879 | GGCTGCCACCACCACGCGGA | 45 | 1 | 692 |
| 1577380 | 9940 | 9959 | CACCATTTGCAGCGGCAAAG | 47 | I | 693 |
| 1577399 | 29407 | 29426 | ACCATCGCCACCATCATCAC | 49 | I | 694 |
| 1577403 | 17561 | 17580 | AGGATGAGGGGAGGACGGCG | 73 | I | 695 |
| 1577418 | 40661 | 40680 | TCAGGGAAGGCCCCATCCAC | 45 | I | 696 |
| 1577420 | 70400 | 70419 | GTCCGAGCTTTGTGAACCGT | 51 | I | 697 |
| 1577425 | 43437 | 43456 | AGCATTCCAGCTCGCTTCAG | 57 | I | 698 |
| 1577443 | 32367 | 32386 | CAAGGGTCCCGGTGAGCCCA | 54 | I | 699 |
| 1577449 | 71982 | 72001 | GATGGCAGACTGCAATGGCG | 100 | I | 700 |
| 1577455 | 3751 | 3770 | CAACCTTCCTCTGCCTCAGT | 58 | I | 701 |
| 1577457 | 32966 | 32985 | TGGTGGTCAGCGTGATCTGT | 55 | I | 702 |
| 1577462 | 70469 | 70488 | TCCACAGCAGGTCCAAGCCT | 100 | I | 703 |
| | 70875 | 70894 | | | | |
| 1577463 | 5511 | 5530 | TGGACCTTCGGAGGGCCCAG | 80 | I | 704 |
| 1577465 | 30961 | 30980 | CCCCGGGGCACGCTCAGCTC | 59 | I | 705 |
| 1577466 | 71087 | 71106 | GCTCCCCAGGCGGCCATTCC | 79 | I | 706 |
| 1577472 | 14267 | 14286 | CCTCCTGCCCTCGGCAAACG | 60 | I | 707 |
| 1577477 | 27008 | 27027 | GTCTGAGCTGGCAGGGCTGC | 45 | I | 708 |
| 1577484 | 39303 | 39322 | GGGTTACTTTCCTGCCCCCC | 41 | I | 709 |
| 1577493 | 68320 | 68339 | GGCCACACCTTGGGGGGGGA | 76 | I | 710 |
| 1577505 | 40724 | 40743 | GCTCCACCCTCAGGGTAGGA | 44 | I | 711 |
| 1577507 | 71988 | 72007 | CCAGAGGATGGCAGACTGCA | 76 | I | 712 |
| 1577521 | 65743 | 65762 | CCCGCAGGTTCCCCTCGGGG | 65 | I | 713 |
| 1577528 | 6033 | 6052 | ACGTTGAATCCCGGCGGCCA | 39 | I | 714 |
| 1577531 | 9934 | 9953 | TTGCAGCGGCAAAGTCCTAG | 53 | I | 715 |
| 1577534 | 28815 | 28834 | CTCGCACGGCCAGCCCACCG | 83 | I | 716 |
| 1577535 | 3757 | 3776 | CTCACTCAACCTTCCTCTGC | 78 | I | 717 |
| 1577538 | 28752 | 28771 | CTGGAGAATCTTGGGACTGT | 43 | I | 718 |
| 1577539 | 17552 | 17571 | GGAGGACGGCGGGAAGACGA | 60 | I | 719 |
| 1577540 | 16510 | 16529 | ACCTCCGGGAGACCCCTCTG | 39 | I | 720 |
| | 16818 | 16837 | | | | |
| 1577545 | 39309 | 39328 | GATCCGGGGTTACTTTCCTG | 61 | I | 721 |
| 1577548 | 14261 | 14280 | GCCCTCGGCAAACGCCGTCC | 28 | I | 722 |
| 1577089 | 40650 | 40669 | CCCATCCACAGGGAAGGCTC | 51 | J | 723 |
| 1577092 | 40656 | 40675 | GAAGGCCCCATCCACAGGGA | 45 | J | 724 |
| 1577101 | 3758 | 3777 | GCTCACTCAACCTTCCTCTG | 24 | J | 725 |
| 1577102 | 27003 | 27022 | AGCTGGCAGGGCTGCCCTGT | 50 | J | 726 |
| 1577116 | 27681 | 27700 | CCAGGAAGGACATTACTATC | 69 | J | 727 |
| 1577119 | 26621 | 26640 | CCCTGTCCAAGCTGGGGGAC | 44 | J | 728 |
| 1577121 | 31863 | 31882 | CTTGGCTGCCACCACCACGC | 37 | J | 729 |
| 1577122 | 32075 | 32094 | ACAGACAGAGGGATCGAGGC | 40 | J | 730 |
| 1577126 | 39851 | 39870 | TCAGGGAAGGCTCCACCCTC | 42 | J | 731 |
| | 39941 | 39960 | | | | |
| | 40103 | 40122 | | | | |
| | 40733 | 40752 | | | | |
| 1577127 | 40719 | 40738 | ACCCTCAGGGTAGGATCCAC | 62 | J | 732 |
| 1577132 | 26495 | 26514 | CCCCAGGTAACTGCAAAGGG | 36 | J | 733 |
| 1577146 | 43444 | 43463 | CCCTCACAGCATTCCAGCTC | 49 | J | 734 |
| 1577155 | 13872 | 13891 | CCAGGCTGGCAGCTAGGCCT | 103 | J | 735 |
| 1577157 | 71401 | 71420 | CCTCCCCCGGCGATGTCACC | 66 | J | 736 |
| 1577164 | 71082 | 71101 | CCAGGCGGCCATTCCGTCCT | 60 | J | 737 |
| 1577167 | 70407 | 70426 | ACGGCAGGTCCGAGCTTTGT | 76 | J | 738 |
| 1577171 | 28754 | 28773 | GGCTGGAGAATCTTGGGACT | 32 | J | 739 |
| 1577172 | 71989 | 72008 | GCCAGAGGATGGCAGACTGC | 67 | J | 740 |
| 1577177 | 71394 | 71413 | CGGCGATGTCACCTCCTCCC | 113 | J | 741 |
| 1577184 | 65744 | 65763 | TCCCGCAGGTTCCCCTCGGG | 67 | J | 742 |
| 1577186 | 39849 | 39868 | AGGGAAGGCTCCACCCTCTG | 32 | J | 743 |
| | 40047 | 40066 | | | | |
| | 40137 | 40156 | | | | |
| | 40191 | 40210 | | | | |
| | 40299 | 40318 | | | | |
| | 40479 | 40498 | | | | |
| 1577187 | 28822 | 28841 | ACAGTGGCTCGCACGGCCAG | 78 | J | 744 |
| 1577190 | 71983 | 72002 | GGATGGCAGACTGCAATGGC | 113 | J | 745 |
| 1577202 | 26627 | 26646 | GAGCTGCCCTGTCCAAGCTG | 38 | J | 746 |
| 1577211 | 37068 | 37087 | GAGAGTACACGGCGGCAGGG | 49 | J | 747 |
| 1577212 | 26712 | 26731 | CTGGGAGGCCCTGTCTGAGC | 12 | J | 748 |
| | 26769 | 26788 | | | | |
| | 26826 | 26845 | | | | |
| | 26864 | 26883 | | | | |
| | 26921 | 26940 | | | | |
| | 26940 | 26959 | | | | |
| 1577224 | 40497 | 40516 | AGGGAAGGCCACACCCACAG | 66 | J | 749 |
| 1577227 | 13866 | 13885 | TGGCAGCTAGGCCTGGGGCC | 39 | J | 750 |
| 1577243 | 3752 | 3771 | TCAACCTTCCTCTGCCTCAG | 56 | J | 751 |
| 1577251 | 29408 | 29427 | CACCATCGCCACCATCATCA | 45 | J | 752 |
| 1577258 | 29415 | 29434 | ATCACATCACCATCGCCACC | 62 | J | 753 |
| 1577262 | 10441 | 10460 | GCTGCATCTACCCCAGGCAG | 23 | J | 754 |
| | 10712 | 10731 | | | | |
| 1577270 | 71352 | 71371 | CCTTACAAGACGGCAAAGTT | 85 | J | 755 |
| 1577272 | 43438 | 43457 | CAGCATTCCAGCTCGCTTCA | 61 | J | 756 |
| 1577276 | 70479 | 70498 | CTGGGCACCCTCCACAGCAG | 58 | J | 757 |
| | 70885 | 70904 | | | | |
| 1577283 | 68420 | 68439 | GGGCCTCACCTCGGGGGAGG | 105 | J | 758 |
| 1577285 | 29934 | 29953 | TCATCACCACCTCCACCATC | 72 | J | 759 |
| 1577289 | 9935 | 9954 | TTTGCAGCGGCAAAGTCCTA | 41 | J | 760 |
| 1577291 | 14268 | 14287 | CCCTCCTGCCCTCGGCAAAC | 63 | J | 761 |
| 1577293 | 30782 | 30801 | GCCCGGGGTCTGGTCTCAGC | 34 | J | 762 |
| 1577301 | 39304 | 39323 | GGGGTTACTTTCCTGCCCCC | 86 | J | 763 |
| 1577306 | 37279 | 37298 | CCTATCTCAACAACAACAAA | 49 | J | 764 |
| 1577317 | 30968 | 30987 | GCCTGGGCCCCGGGGCACGC | 33 | J | 765 |
| 1577319 | 70470 | 70489 | CTCCACAGCAGGTCCAAGCC | 61 | J | 766 |
| | 70876 | 70895 | | | | |
| 1577323 | 51177 | 51196 | AGCTGGCACTGGGCTTCCTT | 54 | J | 767 |
| 1577328 | 39310 | 39329 | TGATCCGGGGTTACTTTCCT | 69 | J | 768 |
| 1577335 | 14262 | 14281 | TGCCCTCGGCAAACGCCGTC | 39 | J | 769 |
| 1577344 | 28816 | 28835 | GCTCGCACGGCCAGCCCACC | 45 | J | 770 |
| 1577349 | 6028 | 6047 | GAATCCCGGCGGCCATCACG | 31 | J | 771 |
| 1577352 | 6034 | 6053 | CACGTTGAATCCCGGCGGCC | 37 | J | 772 |
| 1577358 | 26720 | 26739 | TGTCTGAGCTGGGAGGCCCT | 13 | J | 773 |
| | 26777 | 26796 | | | | |
| | 26834 | 26853 | | | | |
| | 26872 | 26891 | | | | |
| | 26929 | 26948 | | | | |
| | 26948 | 26967 | | | | |
| 1577365 | 40713 | 40732 | AGGGTAGGATCCACCCACAG | 59 | J | 774 |
| 1577382 | 51183 | 51202 | GTCCGCAGCTGGCACTGGGC | 59 | J | 775 |
| 1577392 | 29868 | 29887 | CCATTACCACCACCATTACC | 60 | J | 776 |
| 1577395 | 16110 | 16129 | GCCTCAGACAGAGCCAGGGT | 41 | J | 777 |
| 1577398 | 16511 | 16530 | CACCTCCGGGAGACCCCTCT | 18 | J | 778 |
| | 16819 | 16838 | | | | |
| 1577412 | 71088 | 71107 | GGCTCCCCAGGCGGCCATTC | 91 | J | 779 |
| 1577428 | 68321 | 68340 | GGGCCACACCTTGGGGGGGG | 65 | J | 780 |
| 1577433 | 30962 | 30981 | GCCCCGGGGCACGCTCAGCT | 51 | J | 781 |
| 1577436 | 71346 | 71365 | AAGACGGCAAAGTTCAGCAA | 68 | J | 782 |
| 1577438 | 17553 | 17572 | GGGAGGACGGCGGGAAGACG | 83 | J | 783 |
| 1577447 | 32081 | 32100 | AGATAAACAGACAGAGGGAT | 69 | J | 784 |
| 1577448 | 16116 | 16135 | GCCCCTGCCTCAGACAGAGC | 40 | J | 785 |
| 1577464 | 27009 | 27028 | TGTCTGAGCTGGCAGGGCTG | 62 | J | 786 |
| 1577476 | 16811 | 16830 | GGAGACCCCTCTGCTCACGG | 20 | J | 787 |
| 1577494 | 37062 | 37081 | ACACGGCGGCAGGGCTCACG | 22 | J | 788 |
| 1577501 | 27037 | 27056 | TGGGGACTCTATCTGGGCTA | 25 | J | 789 |
| 1577502 | 18038 | 18057 | CGCCTACAAACCGTGGTGTC | 73 | J | 790 |
| 1577506 | 9941 | 9960 | GCACCATTTGCAGCGGCAAA | 32 | J | 791 |
| 1577511 | 5512 | 5531 | GTGGACCTTCGGAGGGCCCA | 37 | J | 792 |
| 1577513 | 27043 | 27062 | CTGAGCTGGGGACTCTATCT | 53 | J | 793 |
| 1577514 | 5518 | 5537 | CCTCAAGTGGACCTTCGGAG | 47 | J | 794 |
| 1577515 | 68315 | 68334 | CACCTTGGGGGGGGAGGAAA | 61 | J | 795 |
| 1577516 | 10443 | 10462 | AGGCTGCATCTACCCCAGGC | 11 | J | 796 |
| | 10714 | 10733 | | | | |
| 1577520 | 32369 | 32388 | CCCAAGGGTCCCGGTGAGCC | 41 | J | 797 |
| 1577526 | 70401 | 70420 | GGTCCGAGCTTTGTGAACCG | 53 | J | 798 |
| 1577086 | 43433 | 43452 | TTCCAGCTCGCTTCAGGAAG | 49 | K | 799 |
| 1577090 | 16117 | 16136 | AGCCCCTGCCTCAGACAGAG | 17 | K | 800 |
| 1577093 | 26622 | 26641 | GCCCTGTCCAAGCTGGGGGA | 33 | K | 801 |
| 1577096 | 27044 | 27063 | TCTGAGCTGGGGACTCTATC | 62 | K | 802 |
| 1577106 | 5519 | 5538 | ACCTCAAGTGGACCTTCGGA | 58 | K | 803 |
| 1577113 | 6029 | 6048 | TGAATCCCGGCGGCCATCAC | 46 | K | 804 |
| 1577114 | 70471 | 70490 | CCTCCACAGCAGGTCCAAGC | 59 | K | 805 |
| | 70877 | 70896 | | | | |
| 1577115 | 40720 | 40739 | CACCCTCAGGGTAGGATCCA | 88 | K | 806 |
| 1577118 | 10327 | 10346 | GAGGAGGCTGCATCTACCCC | 23 | K | 807 |
| | 10447 | 10466 | | | | |
| | 10477 | 10496 | | | | |
| | 10628 | 10647 | | | | |
| | 10718 | 10737 | | | | |
| 1577133 | 70468 | 70487 | CCACAGCAGGTCCAAGCCTC | 79 | K | 808 |
| | 70874 | 70893 | | | | |
| 1577147 | 26628 | 26647 | GGAGCTGCCCTGTCCAAGCT | 26 | K | 809 |
| 1577151 | 27683 | 27702 | CCCCAGGAAGGACATTACTA | 47 | K | 810 |
| 1577154 | 32076 | 32095 | AACAGACAGAGGGATCGAGG | 54 | K | 811 |
| 1577161 | 30969 | 30988 | AGCCTGGGCCCCGGGGCACG | 55 | K | 812 |
| 1577163 | 28823 | 28842 | AACAGTGGCTCGCACGGCCA | N.D. | K | 813 |
| 1577168 | 43439 | 43458 | ACAGCATTCCAGCTCGCTTC | 52 | K | 814 |
| 1577174 | 37069 | 37088 | AGAGAGTACACGGCGGCAGG | 54 | K | 815 |
| 1577180 | 6035 | 6054 | ACACGTTGAATCCCGGCGGC | 36 | K | 816 |
| 1577185 | 10442 | 10461 | GGCTGCATCTACCCCAGGCA | 16 | K | 817 |
| | 10713 | 10732 | | | | |
| 1577193 | 39305 | 39324 | CGGGGTTACTTTCCTGCCCC | 63 | K | 818 |
| 1577194 | 40714 | 40733 | CAGGGTAGGATCCACCCACA | 56 | K | 819 |
| 1577195 | 28758 | 28777 | GCAGGGCTGGAGAATCTTGG | 69 | K | 820 |
| 1577196 | 68316 | 68335 | ACACCTTGGGGGGGGAGGAA | 97 | K | 821 |
| 1577206 | 68421 | 68440 | GGGGCCTCACCTCGGGGGAG | 105 | K | 822 |
| 1577210 | 37281 | 37300 | TCCCTATCTCAACAACAACA | 54 | K | 823 |
| 1577215 | 29416 | 29435 | CATCACATCACCATCGCCAC | 69 | K | 824 |
| 1577220 | 29935 | 29954 | ATCATCACCACCTCCACCAT | 54 | K | 825 |
| 1577225 | 51172 | 51191 | GCACTGGGCTTCCTTCCTGC | 55 | K | 826 |
| 1577232 | 71083 | 71102 | CCCAGGCGGCCATTCCGTCC | 68 | K | 827 |
| 1577235 | 29409 | 29428 | TCACCATCGCCACCATCATC | 51 | K | 828 |
| 1577246 | 17554 | 17573 | GGGGAGGACGGCGGGAAGAC | 58 | K | 829 |
| 1577253 | 32082 | 32101 | TAGATAAACAGACAGAGGGA | 56 | K | 830 |
| 1577254 | 30785 | 30804 | TAGGCCCGGGGTCTGGTCTC | 68 | K | 831 |
| 1577266 | 31865 | 31884 | GCCTTGGCTGCCACCACCAC | 45 | K | 832 |
| 1577275 | 13873 | 13892 | CCCAGGCTGGCAGCTAGGCC | 53 | K | 833 |
| 1577311 | 16512 | 16531 | CCACCTCCGGGAGACCCCTC | 32 | K | 834 |
| | 16820 | 16839 | | | | |
| 1577316 | 71347 | 71366 | CAAGACGGCAAAGTTCAGCA | 68 | K | 835 |
| 1577320 | 9942 | 9961 | AGCACCATTTGCAGCGGCAA | 28 | K | 836 |
| 1577331 | 27004 | 27023 | GAGCTGGCAGGGCTGCCCTG | 47 | K | 837 |
| 1577334 | 26719 | 26738 | GTCTGAGCTGGGAGGCCCTG | 14 | K | 838 |
| | 26776 | 26795 | | | | |
| | 26833 | 26852 | | | | |
| | 26871 | 26890 | | | | |
| | 26928 | 26947 | | | | |
| | 26947 | 26966 | | | | |
| 1577338 | 71353 | 71372 | CCCTTACAAGACGGCAAAGT | 104 | K | 839 |
| 1577339 | 68413 | 68432 | ACCTCGGGGGAGGAAAGAGC | 65 | K | 840 |
| 1577342 | 27010 | 27029 | ATGTCTGAGCTGGCAGGGCT | 49 | K | 841 |
| 1577346 | 32967 | 32986 | ATGGTGGTCAGCGTGATCTG | 63 | K | 842 |
| 1577347 | 70408 | 70427 | CACGGCAGGTCCGAGCTTTG | 52 | K | 843 |
| 1577368 | 71402 | 71421 | ACCTCCCCCGGCGATGTCAC | 67 | K | 844 |
| 1577372 | 71990 | 72009 | GGCCAGAGGATGGCAGACTG | 104 | K | 845 |
| 1577375 | 3759 | 3778 | AGCTCACTCAACCTTCCTCT | 40 | K | 846 |
| 1577378 | 16812 | 16831 | GGGAGACCCCTCTGCTCACG | 40 | K | 847 |
| 1577381 | 32959 | 32978 | CAGCGTGATCTGTGGGACCG | 32 | K | 848 |
| 1577386 | 14263 | 14282 | CTGCCCTCGGCAAACGCCGT | 46 | K | 849 |
| 1577387 | 27038 | 27057 | CTGGGGACTCTATCTGGGCT | 30 | K | 850 |
| 1577393 | 26497 | 26516 | GGCCCCAGGTAACTGCAAAG | 50 | K | 851 |
| 1577397 | 40491 | 40510 | GGCCACACCCACAGGGAAGG | 52 | K | 852 |
| 1577400 | 32963 | 32982 | TGGTCAGCGTGATCTGTGGG | 49 | K | 853 |
| 1577402 | 39311 | 39330 | GTGATCCGGGGTTACTTTCC | 50 | K | 854 |
| 1577409 | 65746 | 65765 | TGTCCCGCAGGTTCCCCTCG | 72 | K | 855 |
| 1577411 | 5513 | 5532 | AGTGGACCTTCGGAGGGCCC | 49 | K | 856 |
| 1577414 | 71984 | 72003 | AGGATGGCAGACTGCAATGG | 73 | K | 857 |
| 1577427 | 40657 | 40676 | GGAAGGCCCCATCCACAGGG | 47 | K | 858 |
| 1577434 | 71089 | 71108 | AGGCTCCCCAGGCGGCCATT | 49 | K | 859 |
| 1577444 | 14269 | 14288 | CCCCTCCTGCCCTCGGCAAA | 45 | K | 860 |
| 1577451 | 65739 | 65758 | CAGGTTCCCCTCGGGGGGCC | 88 | K | 861 |
| 1577459 | 37063 | 37082 | TACACGGCGGCAGGGCTCAC | 33 | K | 862 |
| 1577460 | 16111 | 16130 | TGCCTCAGACAGAGCCAGGG | 38 | K | 863 |
| 1577469 | 71395 | 71414 | CCGGCGATGTCACCTCCTCC | 99 | K | 864 |
| 1577475 | 30963 | 30982 | GGCCCCGGGGCACGCTCAGC | 63 | K | 865 |
| 1577479 | 9936 | 9955 | ATTTGCAGCGGCAAAGTCCT | 36 | K | 866 |
| 1577486 | 40651 | 40670 | CCCCATCCACAGGGAAGGCT | 45 | K | 867 |
| 1577489 | 26714 | 26733 | AGCTGGGAGGCCCTGTCTGA | 22 | K | 868 |
| | 26771 | 26790 | | | | |
| | 26828 | 26847 | | | | |
| | 26866 | 26885 | | | | |
| | 26923 | 26942 | | | | |
| | 26942 | 26961 | | | | |
| 1577519 | 13867 | 13886 | CTGGCAGCTAGGCCTGGGGC | 35 | K | 869 |
| 1577522 | 3753 | 3772 | CTCAACCTTCCTCTGCCTCA | 55 | K | 870 |
| 1577523 | 28817 | 28836 | GGCTCGCACGGCCAGCCCAC | 56 | K | 871 |
| 1577525 | 40498 | 40517 | CAGGGAAGGCCACACCCACA | 63 | K | 872 |
| 1577533 | 70402 | 70421 | AGGTCCGAGCTTTGTGAACC | 73 | K | 873 |
| 1577537 | 29869 | 29888 | ACCATTACCACCACCATTAC | 39 | K | 874 |
| 1577543 | 51178 | 51197 | CAGCTGGCACTGGGCTTCCT | 76 | K | 875 |
| 1577546 | 18043 | 18062 | GCCTCCGCCTACAAACCGTG | 53 | K | 876 |
| 1577088 | 26617 | 26636 | GTCCAAGCTGGGGGACTCTA | 58 | L | 877 |
| 1577099 | 70403 | 70422 | CAGGTCCGAGCTTTGTGAAC | 50 | L | 878 |
| 1577103 | 5514 | 5533 | AAGTGGACCTTCGGAGGGCC | 72 | L | 879 |
| 1577110 | 32077 | 32096 | AAACAGACAGAGGGATCGAG | 43 | L | 880 |
| 1577120 | 13868 | 13887 | GCTGGCAGCTAGGCCTGGGG | 46 | L | 881 |
| 1577124 | 43434 | 43453 | ATTCCAGCTCGCTTCAGGAA | 40 | L | 882 |
| 1577128 | 71403 | 71422 | AACCTCCCCCGGCGATGTCA | 48 | L | 883 |
| 1577129 | 14270 | 14289 | TCCCCTCCTGCCCTCGGCAA | 63 | L | 884 |
| 1577134 | 18049 | 18068 | TTCCAGGCCTCCGCCTACAA | 37 | L | 885 |
| 1577136 | 10326 | 10345 | AGGAGGCTGCATCTACCCCA | 14 | L | 886 |
| | 10446 | 10465 | | | | |
| | 10476 | 10495 | | | | |
| | 10627 | 10646 | | | | |
| | 10717 | 10736 | | | | |
| 1577137 | 27039 | 27058 | GCTGGGGACTCTATCTGGGC | 36 | L | 887 |
| 1577141 | 43440 | 43459 | CACAGCATTCCAGCTCGCTT | 39 | L | 888 |
| 1577144 | 16112 | 16131 | CTGCCTCAGACAGAGCCAGG | 30 | L | 889 |
| 1577158 | 32968 | 32987 | AATGGTGGTCAGCGTGATCT | 41 | L | 890 |
| 1577165 | 71354 | 71373 | ACCCTTACAAGACGGCAAAG | 58 | L | 891 |
| 1577170 | 29937 | 29956 | CCATCATCACCACCTCCACC | 65 | L | 892 |
| 1577175 | 40658 | 40677 | GGGAAGGCCCCATCCACAGG | 58 | L | 893 |
| 1577176 | 5520 | 5539 | AACCTCAAGTGGACCTTCGG | 70 | L | 894 |
| 1577183 | 71396 | 71415 | CCCGGCGATGTCACCTCCTC | 75 | L | 895 |
| 1577201 | 26623 | 26642 | TGCCCTGTCCAAGCTGGGGG | 14 | L | 896 |
| 1577204 | 32964 | 32983 | GTGGTCAGCGTGATCTGTGG | 48 | L | 897 |
| 1577214 | 26715 | 26734 | GAGCTGGGAGGCCCTGTCTG | 7 | L | 898 |
| | 26772 | 26791 | | | | |
| | 26829 | 26848 | | | | |
| | 26867 | 26886 | | | | |
| | 26924 | 26943 | | | | |
| | 26943 | 26962 | | | | |
| 1577216 | 71348 | 71367 | ACAAGACGGCAAAGTTCAGC | 62 | L | 899 |
| 1577229 | 65748 | 65767 | GCTGTCCCGCAGGTTCCCCT | 55 | L | 900 |
| 1577242 | 51173 | 51192 | GGCACTGGGCTTCCTTCCTG | 35 | L | 901 |
| 1577244 | 40652 | 40671 | GCCCCATCCACAGGGAAGGC | 43 | L | 902 |
| 1577249 | 3760 | 3779 | GAGCTCACTCAACCTTCCTC | 46 | L | 903 |
| 1577250 | 27011 | 27030 | CATGTCTGAGCTGGCAGGGC | 22 | L | 904 |
| 1577265 | 68416 | 68435 | CTCACCTCGGGGGAGGAAAG | 83 | L | 905 |
| 1577267 | 29864 | 29883 | TACCACCACCATTACCACCA | 13 | L | 906 |
| 1577282 | 40715 | 40734 | TCAGGGTAGGATCCACCCAC | 78 | L | 907 |
| 1577284 | 31869 | 31888 | GGGGGCCTTGGCTGCCACCA | 77 | L | 908 |
| 1577286 | 27005 | 27024 | TGAGCTGGCAGGGCTGCCCT | 45 | L | 909 |
| 1577287 | 40721 | 40740 | CCACCCTCAGGGTAGGATCC | 94 | L | 910 |
| 1577304 | 26710 | 26729 | GGGAGGCCCTGTCTGAGCTG | 24 | L | 911 |
| | 26767 | 26786 | | | | |
| | 26824 | 26843 | | | | |
| | 26862 | 26881 | | | | |
| | 26919 | 26938 | | | | |
| | 26938 | 26957 | | | | |
| 1577307 | 28818 | 28837 | TGGCTCGCACGGCCAGCCCA | 83 | L | 912 |
| 1577313 | 10328 | 10347 | GGAGGAGGCTGCATCTACCC | 10 | L | 913 |
| | 10448 | 10467 | | | | |
| | 10478 | 10497 | | | | |
| | 10629 | 10648 | | | | |
| | 10719 | 10738 | | | | |
| 1577315 | 65740 | 65759 | GCAGGTTCCCCTCGGGGGGC | 77 | L | 914 |
| 1577321 | 37070 | 37089 | CAGAGAGTACACGGCGGCAG | 37 | L | 915 |
| 1577325 | 39306 | 39325 | CCGGGGTTACTTTCCTGCCC | 32 | L | 916 |
| 1577329 | 37064 | 37083 | GTACACGGCGGCAGGGCTCA | 29 | L | 917 |
| 1577341 | 26709 | 26728 | GGAGGCCCTGTCTGAGCTGG | 22 | L | 918 |
| | 26766 | 26785 | | | | |
| | 26823 | 26842 | | | | |
| | 26861 | 26880 | | | | |
| | 26918 | 26937 | | | | |
| | 26937 | 26956 | | | | |
| 1577351 | 28824 | 28843 | CAACAGTGGCTCGCACGGCC | 81 | L | 919 |
| 1577353 | 27045 | 27064 | GTCTGAGCTGGGGACTCTAT | 56 | L | 920 |
| 1577357 | 68422 | 68441 | CGGGGCCTCACCTCGGGGGA | 110 | L | 921 |
| 1577359 | 70472 | 70491 | CCCTCCACAGCAGGTCCAAG | 73 | L | 922 |
| | 70878 | 70897 | | | | |
| 1577369 | 27687 | 27706 | CAGGCCCCAGGAAGGACATT | 37 | L | 923 |
| 1577371 | 70476 | 70495 | GGCACCCTCCACAGCAGGTC | 55 | L | 924 |
| | 70882 | 70901 | | | | |
| 1577373 | 71090 | 71109 | CAGGCTCCCCAGGCGGCCAT | 63 | L | 925 |
| 1577377 | 71991 | 72010 | CGGCCAGAGGATGGCAGACT | 72 | L | 926 |
| 1577384 | 71084 | 71103 | CCCCAGGCGGCCATTCCGTC | 66 | L | 927 |
| 1577389 | 70409 | 70428 | CCACGGCAGGTCCGAGCTTT | 45 | L | 928 |
| 1577390 | 32083 | 32102 | TTAGATAAACAGACAGAGGG | 52 | L | 929 |
| 1577396 | 71985 | 72004 | GAGGATGGCAGACTGCAATG | 49 | L | 930 |
| 1577405 | 39300 | 39319 | TTACTTTCCTGCCCCCCACG | 43 | L | 931 |
| 1577408 | 30970 | 30989 | CAGCCTGGGCCCCGGGGCAC | 37 | L | 932 |
| 1577410 | 39852 | 39871 | CTCAGGGAAGGCTCCACCCT | 75 | L | 933 |
| | 39942 | 39961 | | | | |
| | 40104 | 40123 | | | | |
| | 40734 | 40753 | | | | |
| 1577415 | 32960 | 32979 | TCAGCGTGATCTGTGGGACC | 50 | L | 934 |
| 1577426 | 30964 | 30983 | GGGCCCCGGGGCACGCTCAG | 65 | L | 935 |
| 1577430 | 30787 | 30806 | GCTAGGCCCGGGGTCTGGTC | 47 | L | 936 |
| 1577441 | 17557 | 17576 | TGAGGGGAGGACGGCGGGAA | 67 | L | 937 |
| 1577442 | 16514 | 16533 | CCCCACCTCCGGGAGACCCC | 20 | L | 938 |
| | 16788 | 16807 | | | | |
| | 16822 | 16841 | | | | |
| | 16890 | 16909 | | | | |
| 1577453 | 9943 | 9962 | CAGCACCATTTGCAGCGGCA | 56 | L | 939 |
| 1577456 | 51179 | 51198 | GCAGCTGGCACTGGGCTTCC | 42 | L | 940 |
| 1577470 | 16813 | 16832 | CGGGAGACCCCTCTGCTCAC | 28 | L | 941 |
| 1577481 | 40499 | 40518 | TCAGGGAAGGCCACACCCAC | 59 | L | 942 |
| 1577483 | 9937 | 9956 | CATTTGCAGCGGCAAAGTCC | 73 | L | 943 |
| 1577487 | 28760 | 28779 | CAGCAGGGCTGGAGAATCTT | 51 | L | 944 |
| 1577488 | 14264 | 14283 | CCTGCCCTCGGCAAACGCCG | 22 | L | 945 |
| 1577498 | 6036 | 6055 | CACACGTTGAATCCCGGCGG | 26 | L | 946 |
| 1577499 | 13874 | 13893 | GCCCAGGCTGGCAGCTAGGC | 32 | L | 947 |
| 1577500 | 68317 | 68336 | CACACCTTGGGGGGGGAGGA | 63 | L | 948 |
| 1577504 | 6030 | 6049 | TTGAATCCCGGCGGCCATCA | 25 | L | 949 |
| 1577508 | 29870 | 29889 | CACCATTACCACCACCATTA | 33 | L | 950 |
| 1577518 | 16118 | 16137 | CAGCCCCTGCCTCAGACAGA | 45 | L | 951 |
| 1577529 | 29410 | 29429 | ATCACCATCGCCACCATCAT | 29 | L | 952 |
| 1577544 | 3754 | 3773 | ACTCAACCTTCCTCTGCCTC | 45 | L | 953 |
| 1577547 | 40492 | 40511 | AGGCCACACCCACAGGGAAG | 51 | L | 954 |
| 1577105 | 16113 | 16132 | CCTGCCTCAGACAGAGCCAG | 33 | M | 955 |
| 1577107 | 68423 | 68442 | GCGGGGCCTCACCTCGGGGG | 58 | M | 956 |
| 1577111 | 26721 | 26740 | CTGTCTGAGCTGGGAGGCCC | 18 | M | 957 |
| | 26778 | 26797 | | | | |
| | 26835 | 26854 | | | | |
| | 26873 | 26892 | | | | |
| | 26930 | 26949 | | | | |
| | 26949 | 26968 | | | | |
| 1577112 | 40659 | 40678 | AGGGAAGGCCCCATCCACAG | 54 | M | 958 |
| 1577130 | 28819 | 28838 | GTGGCTCGCACGGCCAGCCC | 71 | M | 959 |
| 1577138 | 10324 | 10343 | GAGGCTGCATCTACCCCAGG | 11 | M | 960 |
| | 10444 | 10463 | | | | |
| | 10474 | 10493 | | | | |
| | 10625 | 10644 | | | | |
| | 10715 | 10734 | | | | |
| 1577142 | 26618 | 26637 | TGTCCAAGCTGGGGGACTCT | 85 | M | 961 |
| 1577143 | 40500 | 40519 | CTCAGGGAAGGCCACACCCA | 59 | M | 962 |
| 1577145 | 14259 | 14278 | CCTCGGCAAACGCCGTCCCT | 20 | M | 963 |
| 1577148 | 5521 | 5540 | AAACCTCAAGTGGACCTTCG | 51 | M | 964 |
| 1577152 | 9938 | 9957 | CCATTTGCAGCGGCAAAGTC | 33 | M | 965 |
| 1577156 | 16107 | 16126 | TCAGACAGAGCCAGGGTTGG | 30 | M | 966 |
| 1577159 | 51174 | 51193 | TGGCACTGGGCTTCCTTCCT | 50 | M | 967 |
| 1577166 | 40716 | 40735 | CTCAGGGTAGGATCCACCCA | 72 | M | 968 |
| 1577173 | 26713 | 26732 | GCTGGGAGGCCCTGTCTGAG | 5 | M | 969 |
| | 26770 | 26789 | | | | |
| | 26827 | 26846 | | | | |
| | 26865 | 26884 | | | | |
| | 26922 | 26941 | | | | |
| | 26941 | 26960 | | | | |
| 1577189 | 29405 | 29424 | CATCGCCACCATCATCACCA | 22 | M | 970 |
| 1577205 | 10325 | 10344 | GGAGGCTGCATCTACCCCAG | 14 | M | 971 |
| | 10445 | 10464 | | | | |
| | 10475 | 10494 | | | | |
| | 10626 | 10645 | | | | |
| | 10716 | 10735 | | | | |
| 1577209 | 30971 | 30990 | ACAGCCTGGGCCCCGGGGCA | 37 | M | 972 |
| 1577218 | 29871 | 29890 | TCACCATTACCACCACCATT | 28 | M | 973 |
| 1577222 | 13863 | 13882 | CAGCTAGGCCTGGGGCCCAC | 42 | M | 974 |
| 1577226 | 65750 | 65769 | TCGCTGTCCCGCAGGTTCCC | 65 | M | 975 |
| 1577228 | 70477 | 70496 | GGGCACCCTCCACAGCAGGT | 45 | M | 976 |
| | 70883 | 70902 | | | | |
| 1577230 | 68318 | 68337 | CCACACCTTGGGGGGGGAGG | 85 | M | 977 |
| 1577252 | 43441 | 43460 | TCACAGCATTCCAGCTCGCT | 67 | M | 978 |
| 1577256 | 30965 | 30984 | TGGGCCCCGGGGCACGCTCA | 43 | M | 979 |
| 1577257 | 71986 | 72005 | AGAGGATGGCAGACTGCAAT | 60 | M | 980 |
| 1577259 | 71085 | 71104 | TCCCCAGGCGGCCATTCCGT | 42 | M | 981 |
| 1577277 | 3755 | 3774 | CACTCAACCTTCCTCTGCCT | 41 | M | 982 |
| 1577278 | 71992 | 72011 | ACGGCCAGAGGATGGCAGAC | 76 | M | 983 |
| 1577280 | 71091 | 71110 | ACAGGCTCCCCAGGCGGCCA | 97 | M | 984 |
| 1577281 | 71408 | 71427 | CCCACAACCTCCCCCGGCGA | 50 | M | 985 |
| 1577290 | 27689 | 27708 | GTCAGGCCCCAGGAAGGACA | 39 | M | 986 |
| 1577296 | 6031 | 6050 | GTTGAATCCCGGCGGCCATC | 37 | M | 987 |
| 1577297 | 51180 | 51199 | CGCAGCTGGCACTGGGCTTC | 30 | M | 988 |
| 1577299 | 43435 | 43454 | CATTCCAGCTCGCTTCAGGA | 57 | M | 989 |
| 1577314 | 40494 | 40513 | GAAGGCCACACCCACAGGGA | 39 | M | 990 |
| 1577326 | 9932 | 9951 | GCAGCGGCAAAGTCCTAGCA | 16 | M | 991 |
| 1577327 | 14265 | 14284 | TCCTGCCCTCGGCAAACGCC | 32 | M | 992 |
| 1577336 | 40653 | 40672 | GGCCCCATCCACAGGGAAGG | 52 | M | 993 |
| 1577348 | 65741 | 65760 | CGCAGGTTCCCCTCGGGGGG | 72 | M | 994 |
| 1577360 | 27006 | 27025 | CTGAGCTGGCAGGGCTGCCC | 28 | M | 995 |
| 1577361 | 39847 | 39866 | GGAAGGCTCCACCCTCTGGG | 24 | M | 996 |
| | 40045 | 40064 | | | | |
| | 40135 | 40154 | | | | |
| | 40189 | 40208 | | | | |
| | 40297 | 40316 | | | | |
| | 40477 | 40496 | | | | |
| 1577388 | 27040 | 27059 | AGCTGGGGACTCTATCTGGG | 22 | M | 997 |
| 1577391 | 71397 | 71416 | CCCCGGCGATGTCACCTCCT | 34 | M | 998 |
| 1577394 | 32358 | 32377 | CGGTGAGCCCAGGAGCACCC | 40 | M | 999 |
| 1577407 | 26486 | 26505 | ACTGCAAAGGGGGCCACAGT | 120 | M | 1000 |
| 1577413 | 5515 | 5534 | CAAGTGGACCTTCGGAGGGC | 65 | M | 1001 |
| 1577416 | 68417 | 68436 | CCTCACCTCGGGGGAGGAAA | 88 | M | 1002 |
| 1577421 | 26711 | 26730 | TGGGAGGCCCTGTCTGAGCT | 16 | M | 1003 |
| | 26768 | 26787 | | | | |
| | 26825 | 26844 | | | | |
| | 26863 | 26882 | | | | |
| | 26920 | 26939 | | | | |
| | 26939 | 26958 | | | | |
| 1577422 | 29938 | 29957 | ACCATCATCACCACCTCCAC | 28 | M | 1004 |
| 1577429 | 3761 | 3780 | GGAGCTCACTCAACCTTCCT | 19 | M | 1005 |
| 1577435 | 70404 | 70423 | GCAGGTCCGAGCTTTGTGAA | 66 | M | 1006 |
| 1577437 | 17550 | 17569 | AGGACGGCGGGAAGACGATG | 58 | M | 1007 |
| 1577439 | 70410 | 70429 | TCCACGGCAGGTCCGAGCTT | 46 | M | 1008 |
| 1577440 | 39307 | 39326 | TCCGGGGTTACTTTCCTGCC | 34 | M | 1009 |
| 1577445 | 70473 | 70492 | ACCCTCCACAGCAGGTCCAA | 65 | M | 1010 |
| | 70879 | 70898 | | | | |
| 1577446 | 29865 | 29884 | TTACCACCACCATTACCACC | 31 | M | 1011 |
| 1577452 | 27012 | 27031 | CCATGTCTGAGCTGGCAGGG | 30 | M | 1012 |
| 1577454 | 27046 | 27065 | TGTCTGAGCTGGGGACTCTA | 34 | M | 1013 |
| 1577458 | 31871 | 31890 | CTGGGGGCCTTGGCTGCCAC | 36 | M | 1014 |
| 1577471 | 32078 | 32097 | TAAACAGACAGAGGGATCGA | 45 | M | 1015 |
| 1577474 | 40722 | 40741 | TCCACCCTCAGGGTAGGATC | 58 | M | 1016 |
| 1577478 | 28813 | 28832 | CGCACGGCCAGCCCACCGTG | 53 | M | 1017 |
| 1577482 | 30791 | 30810 | CGGGGCTAGGCCCGGGGTCT | 59 | M | 1018 |
| 1577490 | 17558 | 17577 | ATGAGGGGAGGACGGCGGGA | 61 | M | 1019 |
| 1577491 | 29412 | 29431 | ACATCACCATCGCCACCATC | 28 | M | 1020 |
| 1577492 | 16507 | 16526 | TCCGGGAGACCCCTCTGCTC | 11 | M | 1021 |
| | 16815 | 16834 | | | | |
| 1577495 | 71349 | 71368 | TACAAGACGGCAAAGTTCAG | 103 | M | 1022 |
| 1577496 | 39301 | 39320 | GTTACTTTCCTGCCCCCCAC | 44 | M | 1023 |
| 1577503 | 37065 | 37084 | AGTACACGGCGGCAGGGCTC | 23 | M | 1024 |
| 1577509 | 71355 | 71374 | CACCCTTACAAGACGGCAAA | 44 | M | 1025 |
| 1577512 | 16506 | 16525 | CCGGGAGACCCCTCTGCTCA | 12 | M | 1026 |
| | 16814 | 16833 | | | | |
| 1577532 | 26624 | 26643 | CTGCCCTGTCCAAGCTGGGG | 23 | M | 1027 |
| 1577536 | 37071 | 37090 | CCAGAGAGTACACGGCGGCA | 16 | M | 1028 |
| 1577541 | 13869 | 13888 | GGCTGGCAGCTAGGCCTGGG | 32 | M | 1029 |

### Example 4: Dose-Dependent Inhibition of Human KCNQ2 in SH-SY5Y Cells by Modified Oligonucleotides

Modified oligonucleotides selected from the examples above were tested at various doses in SH-SY5Y cells. Cultured SH-SY5Y cells at a density of 20,000 cells per well were treated by electroporation with various concentrations of modified oligonucleotide as specified in the tables below. After a treatment period of approximately 24 hours, total RNA was isolated from the cells, and KCNQ2 RNA levels were measured by quantitative real-time RTPCR. Human KCNQ2 primer-probe set RTS49037 (described herein above) was used to measure RNA levels as described above. KCNQ2 RNA levels were normalized to total RNA content, as measured by RIBOGREEN®. Reduction of KCNQ2 RNA is presented in the tables below as percent KCNQ2 RNA, relative to untreated control cells (% UTC).

The half maximal inhibitory concentration (IC₅₀) of each modified oligonucleotide was calculated using a linear regression on a log/linear plot of the data in Excel and is also presented in the tables below.

TABLE 6
Dose-dependent reduction of human KCNQ2 RNA
in SH-SY5Y cells by modified oligonucleotides

| Compound | KCNQ2 RNA (% UTC) | | | | IC₅₀ |
|---|---|---|---|---|---|
| No. | 109 nM | 438 nM | 1750 nM | 7000 nM | (μM) |
| 1498664 | 95 | 83 | 102 | 75 | >7.0 |
| 1498676 | 93 | 81 | 75 | 41 | 5.95 |
| 1498807 | 128 | 88 | 52 | 45 | 3.51 |
| 1498865 | 87 | 62 | 29 | 24 | 0.90 |
| 1498878 | 69 | 28 | 21 | 12 | 0.22 |
| 1498879 | 84 | 61 | 34 | 17 | 0.83 |
| 1498880 | 125 | 43 | 20 | 22 | 0.97 |
| 1498881 | 97 | 71 | 35 | 32 | 1.49 |
| 1498883 | 83 | 64 | 48 | 15 | 1.02 |
| 1498886 | 68 | 43 | 27 | 14 | 0.35 |
| 1498906 | 75 | 53 | 32 | 17 | 0.59 |
| 1498910 | 90 | 66 | 40 | 12 | 0.98 |
| 1498938 | 110 | 65 | 36 | 22 | 1.30 |
| 1498939 | 108 | 72 | 42 | 36 | 2.00 |
| 1498952 | 78 | 64 | 36 | 24 | 0.91 |
| 1498953 | 85 | 66 | 37 | 11 | 0.87 |
| 1504318 | 88 | 68 | 47 | 27 | 1.42 |
| 1504337 | 90 | 53 | 32 | 20 | 0.82 |
| 1504340 | 83 | 94 | 58 | 28 | 2.55 |

TABLE 7
Dose-dependent reduction of human KCNQ2 RNA
in SH-SY5Y cells by modified oligonucleotides

| Compound | KCNQ2 RNA (% UTC) | | | | IC₅₀ |
|---|---|---|---|---|---|
| No. | 109 nM | 438 nM | 1750 nM | 7000 nM | (μM) |
| 1498774 | 136 | 118 | 94 | 65 | >7.0 |
| 1498880 | 92 | 40 | 20 | 8 | 0.52 |
| 1499003 | 82 | 52 | 36 | 24 | 0.78 |
| 1499004 | 76 | 54 | 32 | 17 | 0.61 |
| 1499020 | 76 | 55 | 32 | 10 | 0.57 |
| 1499022 | 64 | 43 | 25 | 14 | 0.29 |
| 1499023 | 95 | 49 | 8 | 19 | 0.60 |
| 1499024 | 84 | 57 | 25 | 15 | 0.65 |
| 1499026 | 92 | 74 | 30 | 14 | 0.98 |
| 1499037 | 70 | 45 | 20 | 14 | 0.35 |
| 1499038 | 63 | 37 | 22 | 9 | 0.23 |
| 1499039 | 77 | 55 | 26 | 14 | 0.56 |
| 1499041 | 77 | 57 | 42 | 14 | 0.73 |
| 1499068 | 111 | 51 | 30 | 16 | 0.96 |
| 1499069 | 109 | 90 | 48 | 18 | 1.81 |
| 1499070 | 79 | 64 | 32 | 14 | 0.74 |
| 1499071 | 73 | 58 | 24 | 18 | 0.55 |
| 1499072 | 66 | 54 | 23 | 21 | 0.41 |
| 1499081 | 108 | 61 | 34 | 16 | 1.08 |

1. An oligomeric compound, comprising a modified oligonucleotide consisting of 12 to 30 linked nucleosides wherein the nucleobase sequence of the modified oligonucleotide is at least 90% complementary to an equal length portion of a KCNQ2 nucleic acid, and wherein the modified oligonucleotide comprises at least one modification selected from a modified sugar moiety and a modified internucleoside linkage.

2. The oligomeric compound of claim 1, wherein the modified oligonucleotide comprises an at least 8 nucleobase portion, at least 9 nucleobase portion, at least 10 nucleobase portion, at least 11 nucleobase portion, at least 12 nucleobase portion, at least 13 nucleobase portion, at least 14 nucleobase portion, at least 15 nucleobase portion, at least 16 nucleobase portion, at least 17 nucleobase portion, at least 18 nucleobase portion, at least 19 nucleobase portion, or a 20 nucleobase portion of any of SEQ ID NO: 21-1029.

3. An oligomeric compound comprising a modified oligonucleotide consisting of 12 to 30 linked nucleosides and having a nucleobase sequence comprising at least 12, at least 13, at least 14, at least 15, at least 16, at least 17, at least 18, at least 19 or 20 contiguous nucleobases of any of SEQ ID NO: 21-1029.

4. An oligomeric compound comprising a modified oligonucleotide consisting of 12 to 30 linked nucleosides and having a nucleobase sequence having at least 8, at least 9, at least 10, at least 11, at least 12, at least 13, at least 14, at least 15, at least 16, at least 17, at least 18, at least 19, or at least 20 contiguous nucleobases at least 90%, at least 95%, or 100% complementary to:
an equal length portion of nucleobases 4,600-4,624 of SEQ ID NO: 2;
an equal length portion of nucleobases 8,970-8,990 of SEQ ID NO: 2;
an equal length portion of nucleobases 23,730-23,752 of SEQ ID NO: 2;
an equal length portion of nucleobases 24,439-24,775 of SEQ ID NO: 2;
an equal length portion of nucleobases 27,275-27,306 of SEQ ID NO: 2;
an equal length portion of nucleobases 33,048-33,083 of SEQ ID NO: 2;
an equal length portion of nucleobases 33,054-33,083 of SEQ ID NO: 2;
an equal length portion of nucleobases 34,198-34,232 of SEQ ID NO: 2;
an equal length portion of nucleobases 34,543-34,563 of SEQ ID NO: 2;
an equal length portion of nucleobases 35,323-35,343 of SEQ ID NO: 2;
an equal length portion of nucleobases 41,334-41,357 of SEQ ID NO: 2;
an equal length portion of nucleobases 57,517-57,552 of SEQ ID NO: 2;
an equal length portion of nucleobases 57,523-57,544 of SEQ ID NO: 2;
an equal length portion of nucleobases 65,636-65,671 of SEQ ID NO: 2;
an equal length portion of nucleobases 65,745-65,772 of SEQ ID NO: 2;
an equal length portion of nucleobases 1,615-1,642 of SEQ ID NO: 2;
an equal length portion of nucleobases 3,758-3,780 of SEQ ID NO: 2;
an equal length portion of nucleobases 4,631-4,650 of SEQ ID NO: 2;
an equal length portion of nucleobases 6,025-6,050 of SEQ ID NO: 2;
an equal length portion of nucleobases 6,033-6,055 of SEQ ID NO: 2;
an equal length portion of nucleobases 6,547-6,573 of SEQ ID NO: 2;
an equal length portion of nucleobases 7,371-7,394 of SEQ ID NO: 2;
an equal length portion of nucleobases 9,932-9,955 of SEQ ID NO: 2;
an equal length portion of nucleobases 10,324-10,347 of SEQ ID NO: 2;
an equal length portion of nucleobases 10,439-10,462 of SEQ ID NO: 2;
an equal length portion of nucleobases 13,863-13,889 of SEQ ID NO: 2;
an equal length portion of nucleobases 14,261-14,284 of SEQ ID NO: 2;
an equal length portion of nucleobases 16,110-16,137 of SEQ ID NO: 2;
an equal length portion of nucleobases 16,142-16,167 of SEQ ID NO: 2;
an equal length portion of nucleobases 16,506-16,533 of SEQ ID NO: 2;
an equal length portion of nucleobases 18,835-18,858 of SEQ ID NO: 2;
an equal length portion of nucleobases 20,464-20,486 of SEQ ID NO: 2;
an equal length portion of nucleobases 24,161-24,186 of SEQ ID NO: 2;
an equal length portion of nucleobases 25,662-25,685 of SEQ ID NO: 2;
an equal length portion of nucleobases 26,622-26,647 of SEQ ID NO: 2;
an equal length portion of nucleobases 26,709-26,741 of SEQ ID NO: 2;
an equal length portion of nucleobases 27,035-27,059 of SEQ ID NO: 2;
an equal length portion of nucleobases 28,752-28,774 of SEQ ID NO: 2;
an equal length portion of nucleobases 29,184-29,211 of SEQ ID NO: 2;
an equal length portion of nucleobases 29,405-29,431 of SEQ ID NO: 2;
an equal length portion of nucleobases 29,938-29,960 of SEQ ID NO: 2;
an equal length portion of nucleobases 30,968-30,991 of SEQ ID NO: 2;
an equal length portion of nucleobases 31,860-31,886 of SEQ ID NO: 2;
an equal length portion of nucleobases 32,962-32,984 of SEQ ID NO: 2;
an equal length portion of nucleobases 37,062-37,091 of SEQ ID NO: 2; or
an equal length portion of nucleobases 39,847-39,870 of SEQ ID NO: 2.

5. An oligomeric compound comprising a modified oligonucleotide consisting of 12 to 30 linked nucleosides and having a nucleobase sequence comprising at least 8, at least 9, at least 10, at least 11, at least 12, at least 13, at least 14, at least 15, at least 16, at least 17, at least 18, at least 19, or 20 contiguous nucleobases of a sequence selected from:
SEQ ID NOs: 67, 82;
SEQ ID NOs: 34, 83;
SEQ ID NOs: 61, 97;
SEQ ID NOs: 40, 42, 88;
SEQ ID NOs: 21, 24, 32, 41, 44, 71, 96;
SEQ ID NOs: 23, 25, 30, 50, 57, 68, 89, 91, 95;
SEQ ID NOs: 25, 30, 50, 68, 89, 91, 95;
SEQ ID NOs: 35, 56;
SEQ ID NOs: 58, 84;
SEQ ID NOs: 37, 74;
SEQ ID NOs: 31, 98;
SEQ ID NOs: 59, 76;
SEQ ID NOs: 22, 36, 52, 55, 64, 69, 70, 86, 90;
SEQ ID NOs: 55, 86;
SEQ ID NOs: 26-29, 49, 60, 80, 87, 92;
SEQ ID NOs: 43, 45, 54, 63, 78;
SEQ ID NOs: 132-137;
SEQ ID NOs: 725, 846, 903, 1005;
SEQ ID NOs: 99-105;
SEQ ID NOs: 641, 679, 771, 804, 949, 987;
SEQ ID NOs: 714, 772, 816, 946;
SEQ ID NOs: 240-244, 246;
SEQ ID NOs: 223-227;
SEQ ID NOs: 644, 715, 760, 866, 991;
SEQ ID NOs: 807, 886, 913, 960, 971;
SEQ ID NOs: 654, 655, 754, 796, 817;
SEQ ID NOs: 575, 590, 684, 750, 869, 881, 974, 1029;
SEQ ID NOs: 722, 769, 849, 945, 992;
SEQ ID NOs: 597, 680, 777, 785, 800, 863, 889, 951, 955;
SEQ ID NOs: 207-212, 214;
SEQ ID NOs: 577, 592, 666, 720, 778, 834, 938, 1021, 1026;
SEQ ID NOs: 326-329;
SEQ ID NOs: 314, 315, 317;
SEQ ID NOs: 302-306;
SEQ ID NOs: 286-289;
SEQ ID NOs: 617, 671, 746, 801, 809, 896, 1027;
SEQ ID NOs: 579, 643, 653, 676, 748, 773, 838, 868, 898, 911, 918, 957, 969, 1003;
SEQ ID NOs: 572, 673, 789, 850, 887, 997;
SEQ ID NOs: 266, 267, 718, 739;
SEQ ID NOs: 255-259, 410;
SEQ ID NOs: 636, 694, 752, 828, 952, 970, 1020;
SEQ ID NOs: 626, 630, 1004;
SEQ ID NOs: 602, 765, 812, 932, 972;
SEQ ID NOs: 397-401, 692, 729, 832;
SEQ ID NOs: 638, 658, 853, 897;
SEQ ID NOs: 615, 627, 645, 747, 788, 815, 862, 915, 917, 1024, 1028; or
SEQ ID NOs: 591, 650, 731, 743, 996.

6. The oligomeric compound of any of claims 3-5, wherein the modified oligonucleotide has a nucleobase sequence that is at least 70%, at least 75%, at least 80%, at least 85%, at least 90%, at least 95%, or is 100% complementary to the nucleobase sequence of SEQ ID NO: 1 or SEQ ID NO: 2 when measured across the entire nucleobase sequence of the modified oligonucleotide.

7. The oligomeric compound of any of claims 1-6, wherein the modified oligonucleotide comprises at least one modified nucleoside.

8. The oligomeric compound of claim 7, wherein the modified oligonucleotide comprises at least one modified nucleoside comprising a modified sugar moiety.

9. The oligomeric compound of claim 8, wherein the modified oligonucleotide comprises at least one modified nucleoside comprising a bicyclic sugar moiety.

10. The oligomeric compound of claim 9, wherein the modified oligonucleotide comprises at least one modified nucleoside comprising a bicyclic sugar moiety having a 2′-4′ bridge, wherein the 2′-4′ bridge is selected from —O—CH₂—; and —O—CH(CH₃)—.

11. The oligomeric compound of any of claims 8-10, wherein the modified oligonucleotide comprises at least one modified nucleoside comprising a non-bicyclic modified sugar moiety.

12. The oligomeric compound of claim 11, wherein the non-bicyclic modified sugar moiety is a 2′-MOE sugar moiety or 2′-OMe modified sugar moiety.

13. The oligomeric compound of any of claims 8-12, wherein the modified oligonucleotide comprises at least one modified nucleoside comprising a sugar surrogate.

14. The oligomeric compound of claim 13, wherein the modified oligonucleotide comprises at least one modified nucleoside comprising a sugar surrogate selected from morpholino and PNA.

15. The oligomeric compound of any of claims 1-14, wherein the modified oligonucleotide is a gapmer.

16. The oligomeric compound of any of claims 1-15, wherein the modified oligonucleotide has a sugar motif comprising:
a 5′-region consisting of 1-6 linked 5′-region nucleosides;
a central region consisting of 6-10 linked central region nucleosides; and
a 3′-region consisting of 1-6 linked 3′-region nucleosides; wherein
each of the 5′-region nucleosides and each of the 3′-region nucleosides comprises a modified sugar moiety and the central region is a deoxy region.

17. The oligomeric compound of any of claims 1-16, wherein the oligomeric compound consists of the modified oligonucleotide.

18. The oligomeric compound of any of claims 1-17, wherein the oligomeric compound is single stranded.

19. An oligomeric duplex, comprising the oligomeric compound of any of claims 1-18.

20. A pharmaceutical composition comprising the oligomeric compound of any of claims 1-18 or the oligomeric duplex of claim 19, and a pharmaceutically acceptable diluent or carrier.

21. A method of treating a disease associated with KCNQ2 comprising administering to a subject having or at risk for developing a disease associated with KCNQ2 a therapeutically effective amount of a pharmaceutical composition of claim 20; thereby treating the disease associated with KCNQ2.

22. The method of claim 21, further comprising identifying a subject having or at risk for developing a disease associate with KCNQ2.

23. The method of claim 21 or 22, further comprising genetically testing the subject for a mutation in a KCNQ2 gene.

24. The method of claim 21, wherein the disease associated with KCNQ2 is an epileptic encephalopathy.

25. The method of claim 24, wherein the epileptic encephalopathy is KCNQ2-associated neonatal epileptic encephalopathy.

26. The method of claim 25, wherein at least one symptom or hallmark of the epileptic encephalopathy is ameliorated.

27. The method of claim 26, wherein the symptom or hallmark is any of infantile spasms or seizures, EEG abnormalities, brain MRI abnormalities, or developmental impairment.


SEQUENCE LISTING (1029 sequences)

SEQ ID NO: 1 - DNA, 3253 bp, Homo sapiens
gagtgcggaaccgccgcctcggccatgcggctcccggccggggggcctgggctggggcccgcgccgccccccgcgctccgcccccgctgagcctgagcccgacccggggcgcctcccgccaggcaccatggtgcagaagtcgcgcaacggcggcgtataccccggcccgagcggggagaagaagctgaaggtgggcttcgtggggctggaccccggcgcgcccgactccacccgggacggggcgctgctgatcgccggctccgaggcccccaagcgcggcagcatcctcagcaaacctcgcgcgggcggcgcgggcgccgggaagccccccaagcgcaacgccttctaccgcaagctgcagaatttcctctacaacgtgctggagcggccgcgcggctgggcgttcatctaccacgcctacgtgttcctcctggttttctcctgcctcgtgctgtctgtgttttccaccatcaaggagtatgagaagagctcggagggggccctctacatcctggaaatcgtgactatcgtggtgtttggcgtggagtacttcgtgcggatctgggccgcaggctgctgctgccggtaccgtggctggagggggcggctcaagtttgcccggaaaccgttctgtgtgattgacatcatggtgctcatcgcctccattgcggtgctggccgccggctcccagggcaacgtctttgccacatctgcgctccggagcctgcgcttcctgcagattctgcggatgatccgcatggaccggcggggaggcacctggaagctgctgggctctgtggtctatgcccacagcaaggagctggtcactgcctggtacatcggcttcctttgtctcatcctggcctcgttcctggtgtacttggcagagaagggggagaacgaccactttgacacctacgcggatgcactctggtggggcctgatcacgctgaccaccattggctacggggacaagtacccccagacctggaacggcaggctccttgcggcaaccttcaccctcatcggtgtctccttcttcgcgctgcctgcaggcatcttggggtctgggtttgccctgaaggttcaggagcagcacaggcagaagcactttgagaagaggcggaacccggcagcaggcctgatccagtcggcctggagattctacgccaccaacctctcgcgcacagacctgcactccacgtggcagtactacgagcgaacggtcaccgtgcccatgtacagttcgcaaactcaaacctacggggcctccagacttatccccccgctgaaccagctggagctgctgaggaacctcaagagtaaatctggactcgctttcaggaaggaccccccgccggagccgtctccaagtaaaggcagcccgtgcagagggcccctgtgtggatgctgccccggacgctctagccagaaggtcagtttgaaagatcgtgtcttctccagcccccgaggcgtggctgccaaggggaaggggtccccgcaggcccagactgtgaggcggtcacccagcgccgaccagagcctcgaggacagccccagcaaggtgcccaagagctggagcttcggggaccgcagccgggcacgccaggctttccgcatcaagggtgccgcgtcacggcagaactcagaagaagcaagcctccccggagaggacattgtggatgacaagagctgcccctgcgagtttgtgaccgaggacctgaccccgggcctcaaagtcagcatcagagccgtgtgtgtcatgcggttcctggtgtccaagcggaagttcaaggagagcctgcggccctacgacgtgatggacgtcatcgagcagtactcagccggccacctggacatgctgtcccgaattaagagcctgcagtccagagtggaccagatcgtggggcggggcccagcgatcacggacaaggaccgcaccaagggcccggccgaggcggagctgcccgaggaccccagcatgatgggacggctcgggaaggtggagaagcaggtcttgtccatggagaagaagctggacttcctggtgaatatctacatgcagcggatgggcatccccccgacagagaccgaggcctactttggggccaaagagccggagccggcgccgccgtaccacagcccggaagacagccgggagcatgtcgacaggcacggctgcattgtcaagatcgtgcgctccagcagctccacgggccagaagaacttctcggcgcccccggccgcgccccctgtccagtgtccgccctccacctcctggcagccacagagccacccgcgccagggccacggcacctcccccgtgggggaccacggctccctggtgcgcatcccgccgccgcctgcccacgagcggtcgctgtccgcctacggcgggggcaaccgcgccagcatggagttcctgcggcaggaggacaccccgggctgcaggccccccgaggggaacctgcgggacagcgacacgtccatctccatcccgtccgtggaccacgaggagctggagcgttccttcagcggcttcagcatctcccagtccaaggagaacctggatgctctcaacagctgctacgcggccgtggcgccttgtgccaaagtcaggccctacattgcggagggagagtcagacaccgactccgacctctgtaccccgtgcgggcccccgccacgctcggccaccggcgagggtccctttggtgacgtgggctgggccgggcccaggaagtgaggcggcgctgggccagtggacccgcccgcggccctcctcagcacggtgcctccgaggttttgaggcgggaaccctctggggcccttttcttacagtaactgagtgtggcgggaagggtgggccctggaggggcccatgtgggctgaaggatgggggctcctggcagtgaccttttacaaaagttattttccaacaggggctggagggctgggcagggccctgtggctccaggagcagcgtgcaggagcaaggctgccctgtccactctgctcagggccgcggccgacatcagcccggtgtgaggaggggcgggagtgatgacggggtgttgccagcgtggcaacaggcggggggttgtctcagccgagcccaggggaggcacaaagggcaggcctgttccctgaggacctgcgcaaagggcgggcctgtttggtgaggacctgcggccttgggtcccggtggggtttccgggcagctacaggcgggtgtggccggccgctgtgcgtg

SEQ ID NO: 2 - DNA, 72468 bp, Homo sapiens
ggagccggcgcggccgcgggccaggcgccgaggtgcgcgcggagcgaggtggccgcagcgtctccgcgcgcggcccaagcccggcaggagtgcggaaccgccgcctcggccatgcggctcccggccggggggcctgggctggggcccgcgccgccccccgcgctccgcccccgctgagcctgagcccgacccggggcgcctcccgccaggcaccatggtgcagaagtcgcgcaacggcggcgtataccccggcccgagcggggagaagaagctgaaggtgggcttcgtggggctggaccccggcgcgcccgactccacccgggacggggcgctgctgatcgccggctccgaggcccccaagcgcggcagcatcctcagcaaacctcgcgcgggcggcgcgggcgccgggaagccccccaagcgcaacgccttctaccgcaagctgcagaatttcctctacaacgtgctggagcggccgcgcggctgggcgttcatctaccacgcctacgtgtgagtggccggcggggcccccgtggcgacccccatggcgacccccatcggcgaccccatcggcgaccccggccaggctggctgcggcgggtttggctccctgcccctgggtctgggtccggggccggcgctccccggcgggagtgctgggccgagaccggagagcgattgtacagcgcgcgggcaggaaggattccggggtcggggccgctcaggtcggggtgggggctccaggcccgcaggacagagacgtgcggccgccccagccccctcctcagcctgggaggcccctccccgcggcctctgcgccgaacaaagggccggccggggagggacggcggcagcgctgggtctggactctccgggactctcggggacccgcggggctggggccgcccgaccaagcgagggcgggggggaggggtctggcctgtcctccgccatcctcccgagccgctctccctcagactagggtgagcttctgggttcctgagatgggggaggggccctcccatgacccccccaccccagcccgggctgagcgcagctgtctgtcctgccccctcggcgccgacgcccctccctggccccgcgaaccccctcagctcctgcgctcagctcctgcgctgtcccatctctgcagccgccggccgcccccgtgcacatcccgggctcaggacccctccttggcagagcgaggcccgggggaggggctgcggcagacctgggacccccagccctgggcctggaaggggctgtcccctcttcccgggcgaggctcagggaacggctgcctcctggggcagggctggcggggggcggccgaagcctgcgtccgtgtgcacctgggtgttcgcatgtggaattgcacggcccgctgcgtgtgcggcctgtgcggccggtgtgcacagcctccccgcgccagggcgcttgctctcccatcccagctgaacaagggacccgctgagctccgggaccggccattttgtgtgagaggcattttgggatgggagtgaggggcagactggtcacaagctggtcgggcgcccacctgggccgagaggggtgtgtgccccacgagtgtgccccagcctgcgtgtggccttgagtgtgcccttagcagaccctccgtggactgtgatatgagagggttctggggtagggagaagatcccagagggaaatgccaggccgcttacagggagggtgtctccgtggagggcccctgggcacccagcctgggactccccaagcagaagcttctccgacctcctgggtcctggaagccctgtggcccccaggaggtgagtctagcttttccagtccagggttcattcccgaatccccagatgagcttctgtgttctcaggaatggcctcccctgatggcccccacctggctgggggccaaaggggctcttggtgggccggtactggtcagccaggtccctgcccaaggtgactccccggaggagggtaggatagaacccacccccacaaccctcctgccctgacagcctggccagcgccccgctctgcgtctgacccagggttggttctgctttgagtccatgatccaagggcccagcgtggctgttcctgaggtttcccgttgggtcagcagctctccgcatggccgccccctgcctgggcctgcgtgggtcagcccctctgtctctccttcctgtctctgcctcctgagagcagcagtccagaactggggcaaagcatggggacacttgcacatgtggggtctgagccgtctcttcctgaccacccccagtccggacgggccccctctaagatagagcagcagcatgcgggggcttcaggcgagaccctcaacgtctgaacagccccgccttccagcctgcactaggcctctcctcagctcctttgctgtgggcacggagctccctgaacctccctgagctcccaggctctcctcagctcctttgctgtgggcaggggtggcttggactcagggaggcggcccctttgggagggggattttttctaagcggcggggctggagagcagggtttctggtcctgcctcgggttggacactgggggctggtctcagaacctctgcaaacctcagttttccgtcggtaacgtcggcacagctgcttctcaacccagggctttgtgaacaccactgctggggggcatcacactcttccaggccggcccagtgccctctgtgaaaggggatgtggctcagcactgctggtgcagctcaccgcccggctcccagacccctggtgtggtgtttgctgctgtccccgtggcccagcccatccttggagtgcagcccccacctccacttgggaggcctgatggccccatgtcctctggcccggcatggactggcccgtgtggcgtgttaggcagcatttggtctgtttgttcatcagctaaacggagacggagctcagttgggcctggggcagccgggagtggggggaaggccagggctccttgtgctgccgcacccagctgtggctgaatacaggtccccaggcccacctgccttttgcctattgccaccagtgtctggcttcggagaggtcaagttgtgagaacctctctgggcatccctgttggacccggggtagccactgtctggggtagagagggcggtggctgctgccccatggccccagggcttggtgggcgtcaggccaggcccctgctgcccccgctctggggaggtggggacgtccagcactgagttagagagggacttcatccactgttgctcctctgtgggagccgtggcctctgctcgcactgcctgggtgctttcccagaggctctggagacagggccttctctgagttagaagggcgtcagacctgctctctgcctggtcggggcggggtgctgggccacctctcccagacctcccaaagggagtgtgagctgcagaaatggcccgaactagcagatgatgtgcccctggggctgccgcaggggactctgggccagatcaccatggggggcccttcggagcctgaatcccgggctccagcctctctcctgcagcccatctgcgcataggactggagccgggcactgctccaccacggagttgaacctcctcgtccaggatccccgcgtgggttgctgggtgtgggaaagggagctccgctctgttcccgctgggtgcggacctcagttaccgcttagttacagcctgccagagggtcacgcagcctggttcctgtggatggggaaactgaggcagaggaaggttgagtgagctccagagttgtcctgcttaactgaaagggggggtcccagcctgagatgagagcttgggggtgcagattgcttgatcttgccagcagcccagccacgtggagtggccagagtgaagctgtgtttaaaataggccagatgggaaatctggcaccggggagggcctgtccctggggctcgctgtgccatcctggcttttgcgccaggcctcagctggggtccagggttttaggggcctctggccctgggctgcttgaccaggtgctgtgtcccatttcactgcagggaagggcctgggacggagggcctatgttcaagtcccccggtgccggggcccaggcgcttcccctcacagtcctggagaactcgggtggtgctgagcacaggccgctgggcctctttcccaccccttcattcagctctggggctggtgtgagccctcgaagggctggctggggtgggcaggcgcctggcacagtggtggggttcagacactctcttcttggtcaggctgctttgaggggtgagtgcaggcctgggctttacccagcacagagcggggccctggcaggaccagggtccagtctctgctgacggagcattgtgtgccgagaaagagactcgagtgcttgaacggccacattccccttcttcccacagcctcctgtgtaggaaatcttatggaaatttcccaaaaggtgaagaagcctcggagagttgtggtttctggggaaacctggatttctctggttcctggaccccttgggaggtctcgggcttcctcgcaggatttctgcccctcaccaaataatttaaagtgaaattactctggagtgataagacattgtacatgatgctgcttagttacacagtggagtcaatctctgtgcacattctgtgtctgtgagaggataggagctaaaggataagcagaaactctcctcctgaccattccacagtgagagcagagaaaatctgttttattagaaatcactgcaagccaatctctgcagagacgcggtctctccagaggtgccggcgccctggatatgggcactgctgggcactaaggattctcggcccccaggatgctgaccacgtgggaatcctgtctccagttttgaaaatgcttaaatcctggagtgtttcagaactggggtcacttctcacggcacaggctgagtccagtggtggagagtggcttaacatagtgattttcccagtttgttttagtcaataaacaaatgaatctatggatctccgttagtccctgtgctcctgacacgtccatggcccctgtgtgcacactcagcaagtccgtctggcagctgctcgcaagagcacggggctagcctctgccctgtaggagtgacattccatagcctcacaggagctgggctagcctcagccccataggaatgacattccacagccgtagaggagccaggctagcctcagccctgtaggagtgacgttccatagctgcacaggagccgggctagcctcagccctgtaggaatgacgttccgcagccgtagaggagccgtagaggagctgggcctgaagctgcttccagtgtggggagatgaatctgctacacagagtgggttccctcctaggaaaatgggcaagtctggaggaccaagggggacctgtgggggccttgaatatggggagaagggatatgggatggccgagctgccttgcccagaggcaggggaggggcagcctgtgccctgggccctccgaaggtccacttgaggtttgacgggaggctggggccagcggccaggccgtgtcctgttaaaggctcaggcgggagcaatgtgcccgttttgggaggatggggggcaaggatggtggggtgatgcctgcgatgtcccccgggggagaccttggccttgtgtccgacatcttgtcatcacagacagagcccaagcgtgttacctcacggggggctgtcttaactattttccagtgtgcatgccaggcagctctgagacgggaaactgctcgtgtgtgcgcggtctgtggacgcagccccgctcccctctgtgcttccgcccgtgtgagacgtgctcctgtgtggccgtgacctgtgaagaccctgcgtgacccgtggggcgtgaggctcggggagggcagggctggggctctggccgggggcgtcatcctgagactggccagcgtcgggctcctagacccgagtttccttgtctgcgtggtgggggtaacctgtctgcatctgtgtcacgtgatggccgccgggattcaacgtgtgagtcactccacacgatgcctgatgcgtgcgatgttccccaagctcgtagctgtcatcgccgtgaccgtgaggctccttcagcagagtggaaggcctcaccccggacggagcgtggaggccaggagggaggatggctggccggtccgggacggcgtcgctctgcccctccggccgattctcagtcccattggtcagcggctgggccgtgacggagaggcctgcgctgctgttgagggccaggtcgtgggttgaagtgcggggcccgggctcgagcgtcggaagcctgcgggggtcgtgggttgaagcgcggggccccggctcgagcgtgggaagcctgcgggggtcgtgggttgaagcgcggggcccgggctcgagcgtgggaagcttgcctgtttgtcgtgtgggaccggccgcggagatgccggctgttcacttggaacgaaggtgcctgccctgtgtgtgccgggtagtttgggggattgtcttcctgttcgtggcttccggcgtgttcccagcatcctgagggttgaggcttggaggacaggaatgatcttcggcttgagaaaccactgtgggcaggttttaccagggaaccccttaagctgtgaaagagctgcagtgcaagcttctagtttccacgcagaccgcgccagagaacccgccgggccgcctcgggacggcagcagaggacccgccgggacgcctcagcaccgacacgcgcccagccatgaggccgcctttagcacggagcgtcgagcggaaacggcggtgcccggaacgcggatgccctttgcagggctggagctcagctctgccttccgacaacacttggctgacagctctgggagtctcaaaacggtgcctttgtggaggggagggccctggacatcctcagaggaggaagcgcgtcctgggtagggtgggcggcacctgttcaacacgcctaacagatgcaaggtcgaaggaggaggggcctgcagatctgagcttgtcgccgaggaactctctggaaatggctcccgctggcccctgcgtgcgacccctacgctcaccagcccgtgtgccgagctttctctgttgatttcggagtgggttctggttcctgcctccctagtgggctctgagtggcagggccctgccgaccgctggcctcagcagcgggtgatggagtgtgggccctgcaggaggaggctctgtccttgatggctcctgggtgagggggtgtgagggaggcggccagcacaaggcgggtgggccctgagaagcccctctgtttcccctttagcctgagaaagctgtacccacagagctggaggagggagtggctcaggatgctgcttagcaaataggttggtctggagtctgggaaatggagggttagactttttgttttctgaagctgggatgggtcgtgcttttaattcgtccaggagaacatggggctgaagagctctctgctcttgtggcccctgatgggagtgctctgaggtctggcaggccacagagctgctgggacggcctcagccccaaacctgcgcctgtcgcttcccggggccaggccagaggtttcccagtgagagctttggggtcgacatttccaggaggagaaggcacttctgaaatagcgtgcccttggtgagcttgtgtggcttttctttctctgtaacctgctctgagttgtcccattgagtgttctggggatgtgggcatctggagcctgtggagaggacgggactgggggtgcgtgggggagacgcccctgccaaggacaggggagaccccctcagtcccacctgggagagtctgtgcgcggggctgtggttgattggctggcaagtttgaatgcggatcaatgtgccacttgtttctaagtcagcagggcccctgcccccacacactgagtggctggattctgggagccttttcctgtggagagtggagctggagtgtggggcgcagggccatggccctgttccagcgtcggggagctagtgtgcaggtgctttctcctgttagcagtgggcctgtgctgtgctccctgtggggttaggcggccccggtgggagcttcagtcttgaaaggggctcatagccctggggtgcaggctgctgtgcgtggggggcatcacacgcaggtccccaggatgatgtgtggtggaggacggggcgacgggagctctccacagagcttcatgtcccttctgttgtctaaaccacagaccccgtgccaggtgaggggctggggtgggtgaggctaaggtacctgccccgaggcctcggggcacttggcggcccctccacccccacaggagacatcggcttgagtggagaggccagcctctgagaaggcttctctctggccaccagctgcccagagctcccagcccagggccagcagcttgtgctggccccagacaggggatgctgggcggcctctcgcagggggagtggtggttttcagacagtggctgcctcgctttggggatgaggatgttttccagacgtgctctggaactgacattaggctctgctgcctacccagcttgaacaaaattaggtctttcatttttttgggttgctgggaatggctttgtggatgcataggagagcatggttgctccaggaagtccggcggggagtgggttgaacaatcggagtctcccatccctgaatcatctcagtgggggcggacgggcagctcctgggggtgtggcaggggctgggctccaggctgcccagcctgcagggagcctccttctagattcttcctctgtatctgtggcgtggaggcatctgctgcctctgtctgcccccgcctcatcctccaggtcttctctagttttgttcctccccatggagcccttacccgtgacgtaagctgaacacctgagaactttcgagcttgcttttctgtttaggtttggaaaaaacatgagaagggtgagtgggagcaaaaggccagtttctgcctggctccctgtcctgggaggaggtggggagaagcctcgggcagggatgagaggttgggatgtctgccctgaggggctgtgggcgggaggccggaggcgcctggttaatgtgtttctgcccctgtgtgcgggtgcagggttctaagctgggactcagggtgcagagggcacgtgtgtgggactttaaagtttgtcatggaggctgagccctggggaagggcagcagcactcggccggctgggcttccttctcctggagtagggtggtcagggaggcagggatatcggggagcgccaccccaggacggagcaggaggcagcctggagtcccctttgtagaccagataaggccaggcagggcctgatgtccgtattctattatttatttattttaagtgtgggggtctcgctctgtggcccaggctgagtgcagtggtgtgattgtagctcactgcagcccagaactcctgggcctgagcgatcctcctgccttgacctcctgagtagctgggagatgacccccttcttacccgttcttccagaataggtcaaattcacatgacatgtgcacacacacacacacacacacacacacacacagaaaagacacccacggaaaccctgaaatatataaaatgatgtccagccagtatgatttgccccctctgcgctgccccatctccgtaccgggtcagccatggcccatctcctggagccgttctctgcatgcgtgccacggtacagcatgtgtgtgtctgtgtgtgttcactcacttcttttcacatgtgtggagtcactccgtgtcctgccttgtgcgtgtggccccctctccctgcatgtccccctctaggctccgtcgggcgctcacgacttctctgaccggtttgcagtgatggaccttgaagcaggtgctaggactttgccgctgcaaatggtgctgcgtggggctccatgcacatcttcacgcaaggtgcaggtcggcaggatgaattcctagaagctgcctggtggctgtaaaaatgttctcatcccaaaggaccaaatatgcagagaagtttaacactcacttttcctgtcattgaaaagttacatttttgtggaaacccgtccaggctggagacagaaggtgtgtgcagggagaggaggctctggggcaccactctaggctgcctgctgctcaggtggttgatgtgggggcagatgccctgttccttggggtaggtgccacctcctccctgctccctggggtaggtgcattttcctccctgctgcctggggtaggtgccgcctcctccctgctccctggggtagatgcagcctcctccctgctgcctggggtaggtgccacctcctccctgctgcctggggtaggtgcagcctcctccctgctgcctggggtaggtgctgcctcctccctgctgcctggggtagatgcagcctcctccctgctccctggggtagatgcagcctcctccctgctccctggggtaggtgcagcctcctccctgctgccccggggtaggtgcagcctcttccctgctccctggggtgggtgcagcctcttccctgctccctggggtgggtgcagcctcctccctgctccctggggtagatgcagcctcctccctgctgcctggggtaggtgcagcctcctccctgctgcctggggtaggtgctgcctcctccctgctgcctggggtagatgcagcctcctccctgctccctggggtaggtgcagcctcctccctgctccctggggtaggtgcagcctcctccctgctgccccggggtaggtgctgcctcctccctgctgccccggggtaggtgcagcctcttccctgctccctggggtgggtgcagcctcttccctgctccctggggtaggtgcagcctcctccctgctccctggggtaggtgcagcctcctgcctgctgcccggggtaggtacagcctcctcctccctgctgcccggggtaggtgcagcctcctcctccctgctgcctggggtaggtgatatttctgccctcctacctgattaggctctggggccactcttggggatgggggaggagggccggcccccggggtgctggccatggtcctgaccaggagctgcagttgcggctcctcgtgggccagaggatccccttgtgtgattcttgtttggcatccgcccaggccatagagtcaggggcttggctcacctctgggcagctatggggaaggggctggtgtttgggaggtgcccgaggaatgatcggtggggggtggctgctggggcaaggtacacgaccggcttctctcctttacaattagcactgagccagcttatctgagaccatcccttcccacgggtgccatttaattagccaagagctgagttaagatcacgtgaagaaagttgggggggccttggcctgagcggcgggtggacagggctgcgtctgtcagggtgagggggtctggcggcccctgccgccctcgtttcttaaataaacgtttggtcgtggcttattttcttctgactgcaaagtaagcacatccaccgcaggagactgggaaagcgtggaaaacagggaaggacgtttgctcttcctggtttctttccaggtgtctgcgcccgcctgtgcgtttctaaacagaataggattcaggcggtcccgtcccgctgtggtgttaacggcatactgtccacacttctcatgggcaagatgagtctcgcgtgccaggtagttggcttgtctctgacctggggctgccgcagcacctgtccagccacctgtggacatggatgacggcagagtccctgtgttgtgtggtgtccctgcgtggatgtcgacaccacagctgtcctggggacgtgtccacatgaggcttggggaatccacatgtgtcccatccagggctgtcctgcaggccccgggtctgcctgaatgtgattaggtgatgaggccaccctggccaggaaggctggtgaggggagagggaggacgcaggctggcccaggaggggcttgtgttggaacgagattctttctgtggggtgagccctccgtcgccggaggtgtgcaagtggaggccgagagagagcactcactaggacgtttgtagggggccagggactggagctgtgagccgtggaccagctgagggcactggcctgcttgggagggggttaggcgtggctgtctcagggggctgtgggggggaaggcctgctgggagcctcggtcaggagtgccactgttggggtgccctcagcacctgaatgggtttcgggtggggctgacaggcagcttgagcccatgaagggagtgtgggatgtctcaggaggagcccccttgttgagggtggggggagcccctctccaccccagctcccctgggacctcatgaagcctttgctccccaaagctgctaagggcttgggtaaggccagggagggacctgcagggtcaggagcccaggccaggccaagcaggggtccccaaaggtgctggggatgggggtgtgcatgacagggaagctccggggctcgggtttggggaggagtaggctgggctgggatggggacactggccagggccagagccaagcttcagccacagaagaaggggcgattctacagctcacaaggtccccgggtgccgtctgcttcctgtagtgtaaacgcacctgtccacattccagccttcctaccgagacaacagcaccatggactggaggagtctggaggggacaaggtgtggtgagtgttaactctccccacccaaagctatcctattcagagagaaactgaggcacaggtgggtggacaccctaggcacagagccactgatgggcaggcaggttacctgtgtgcgcttgctgtcccagaccttgacaacacggtttattttgttttttaaaaatagaggttaaattatcctaacatgcagatagcattttaaagcgagtaattcagaggcgtttagagtggagaatttagtgatggtctgccgccaccagccctatttgaaaacttgtcaccccatgtggacactcttgccctctgccatcgtgtgccccaccctgtccctggcagttgccggtctcctgcctgtctctgtgacgtgcctgttattttattttatttaatttatttatttttgaggcagggtctcttttcgcccaggctggagtgcagtgtctcaatcacagctcactgcagcctggacctcctggactcgagccatcctccagcctccgtctcccgagtcgctgggactgcaggtgccccaccacatctggctaacttaaatcatttgtagagatggggtctcattgtgttacccaggctggtctcgaactcctaggcccaagctgtcctcctgcctcggcctcgccaagctctgggagtacaggcgtggccgccgggcctggcctgtttgcttcggatgtttcgtgtaagtggagttgtcatcgtggtgccgttttgtgcctggctttccttgccgagcctgccctcaggttccttcgtgtttagggtactccttcatcctctccgtgtctgggggccgctgcctcgtccttcctgtggccgcacggacggacctcagtttatctacccacccaccaactggatgttgggttgcgtctgccgtgaggctgctgtggacacgcggtgagtgtctctggacatttcgtcattcctcttgggacgaagcctgcgtcacatgtggccaggtgccctgggtgggccgtgccctcctcatctcagccccccagacccacacaggccccctcccggagccctcgccttgctcttcccgctgtgcggaaggtggtgcagtgacgcctgagtgtgggccccaggcctagctgccagcctgggctctcgagcaggttcaacttggccagggcagggtcttggctgccgcctgcctggatgtggggagacgcctctgtgcaggttctgggctgggcacttggggccactgtctgagcagaggatgtggggtcctgcctccgggagggcgagcagaccgctgctctggggggacagtgctcggtcaggctcagggccctggagagaatacaccagggagacttgggggctgcccggggaagggatggtcgggcagcctttcagagaaagtgacctgagattgaagcctgggggatgagggggcggctctgggacctgcgggtaggagaggattccaggcagcaggaagtgcacgtgcaaaggccctggggcagggacggcgtttgccgagggcaggaggggagcagggtggatgggtcgcgtggggtccaccatgaggcactgcagcggcctcgagggccaggccagggtggggtctatcttcagggcctggggctcgcctatgctggctgccattcccacccagcttctccacatgcttctgggatcaaagtggctggtacaggggcgcttctctgcgggctcagtgggttgggatggggcagtggagagtgggtagccggggcatgccgggaggaagagtaaagtccaggggaccccaggccaggggctgcgagcccagggaggttgcggggtcggggagggcgggccaggacctctgcatggggctgggctgggcactttggccagccctgcttggtgcaccggctgggtctctgtccagcacctgtggtctgggtgggacaggtgactaatggagaacaaatgagccctgcggagcttccgtctccagggaccgtgagcaggcgctgcgaagtcacgaggcagtgcccggggagctgatttaaccacgtgctgagccagcgggtttcctgccagggcctgggagtctgcccacagctgcagctcccagagcccctggggtgcatggcggatgccccctctttctgggccggggcgaaggccctggaaggacctgtggttgctgcagcaatgcagggggctgggggtccctgtgggccttgggcgtctgctgcgggagtggttagggagcaacgtgcaggtgggcaggacccccgcctccagctcctccactcctgttcccgagggagccacagctggctgaaggaagtggccatcctggggccatggtcagtgtggggcatcgagagccccctgagagcgcctccctgcccaggatgttggaaggagggctgtaggtgccaggtgggtgcaggcgggcataggcaggcccaggccgtttggaggccgtgagcatcaggctagcagctgggccttctgaggacctgagacttcctgacctgtgtcctgtcagggtccagttgagcacccagtgtttgggaggctcggtcccacctgggtcacctcccgtccctgccccatggccttaggctcaggtgggtgctgccggaagctccgaatgagggtggaccctggcctggcaggtacaagccacgcagtgctcagcatgaggcggctccacgcgagaggtctctgtgtgcagctctgcaggagcgtggcccttgtcccctccagccccggcggtctgcagtgatccatcacgcttggttcatgcccggttcacgcctggactctgtcagcggcgcccgcgcccgtcacttctgaggtgggaggtggcgtgtgtcctggaaacagagcagttcattagatggtgaaattagattccaggttgcagcgtggcggctgcctaatggacgaggcgagcggggcccccagggaggctcgagcagtcctggacctggtgcccactccggcctcacgctggctttctgctcatccccggggccctgctgggctcccaggtccccagccccggggaggtgcagcctcatcagcatggacattgttctggacagggaatttctgcctaaggaggctgcgtgggaccccccaggaccccctgcagtgggagaggggtgcatggtgggggccgtgcagccagccagctctggccaatccgcacgtgggcctctgagagtctccggagcctctgttttcccgactgtgaaatgggcacgtgactgtagggctgcttccctcctggcaggtcatgggtgtgctgagaagcttagcccgtggagtgggagtggggtcttgatgtcaggggcccctcctgacccaaccctggctctgtctgaggcaggggctgagggtggcggtgaggtggtgacgccttccctcctgcccgcccatccttcacgtgttccgttttcatttaactaaatacgcaagatgagatgaacagccctggccaacttggagtcagcctctggggaaggggcctgactggaggatccagagtcgcagagcgagggggctggctgtgggactgactcggggggcccagacccctcctgcgtcgagtcctcttccccaagtgcatgaagcgagtgaggagcggggtggcggcgtctgtgtcatgctttcccagaaaagaaatatccccttcaaccagaatggggaaggaaggcgggggagacctggaagcacaggagtctcccggaggtggggggcccatgagcagaggggtctcccggaggtggggggcctgtgggcagaggggcctcccggaggtggggggcccgtgggcagaggggcctcccggaggtggggggcccgtgaagcagaggggcctcccggaggtggggggcccgtgagcagaggggcctcccggaggtggggggcccgtgaagcagaggggcctcccggaggtggggggcccgtgagcagaggggcctcccggaggtggggggcccgtgagcagaggggcctcccggaggtggggggcccgtgagcacaggggtctcccggaggtggggggcccgtgagcagaggggtctcccggaggtggggggcccgtgagcagaggggcctcccggaggtggggggtccgtgagcacaggggtctcccggaggtgggggccccgtgagtagaggagtttttgcatctcaaggcctgagggtgccagcgtgggggccccaggactgtggtccggccttaactgggcggtgggggccccaggactgtggtccggccttaactgggcacccctctggcctggtgaccctttggtccctcccatgggcccctccctgccccaacttctcctctttgcacttgggtcccaggtcccaggaggagatcccccgctgctccatgctcagtctgtgactgcattcggggtgcctgatccccgccatggcctctcttgtgatctggggctgtctgatgggcacggggctgggctgtgcgtcagggacgtgcggtgagccccagccgacgcccgcggtccaggtgtccccctgcccctctgctcagtggtgggtgccagggctctgtggatcctccctgcccccaccttgactctttgcctgaatctggggctggggaccagctacttgccttatctccttggcaacggctgccagggaagctccggaagttgaacccattatcataattagcttcgaaaccctttgtgttgtaattctgggctgcagagaagctgctcggtcctcccatcgtcctcccagcgtcctcccaccgtcctcccatcctcccatcgtcttcccgccgtcctcccctcatcctcccccattctcccgtaggaagtctcttctttcttttcctgcagacttggccgctgcctgggcaggagggggcgggcagcccttgccgagccagcgagacctcagctgagtgccccgtggtgggctgtgtcccccagcttagtgcaggaggggctgtacccacactgtgggcctgggtctgaggtggtgatggccccgtcccgagaggcctctggcagctggccctcagggagtgtcccacatttggacgctcgtcgtgcctgggaccctgaggccacatgggcagggagtgtgtcccagtgttcccggggagcctcctctcccgaggcctgggggatgccatcaggggtctcctgccgccctctccctcccctggttcagaggcagaggccctcacctgcctcagtgagcctcagtttccccttctctaagatgggcatgagtgtggatgtcttggggacaccacggtttgtaggcggaggcctggaagcccctgtgtcttccccacggttcggccgaccggatggctggccagtgcgtttgggaactatccgcagtttggaggtttcttccctggtctgtcctggggcctggagccacggccgaggcaccgggatctttgcgtcccccagacgggcccagacgcaccccgtgccgcctcctccctcctccaaggctcttctcgcacgtccgtctctctgcctcccctgcaccggccgcccccggcagcgccatctgtgccatctgttccaaaaacttaaaaaacaaaagtcggagctgttgcctgcactttccgggtggggctgtggcaggaggggcagcggcgggaggggcagctgcttccttcatgcagggtctgcgggccggcgaggcgggggcagatgggcggaagctgagctggaaggagctggagccttctgagggcctggctgcgagattcatctccctgaattctggctttattcctcctggtccacgcctgctgggaggagccacagatctcggccgcatccggctgcattctgtctccagggtttctggaatgctctgagatgcctggtcggagccaccttaggaagcaggctgagtcctgaacatttgggacctgggcctcgttttttttagtttagaggaaggttccctgcagcccgaggagctgccttcctggatgcggcactgaggactcagaattcaagtctgtggggctgtcgttgtccccaccccgccctgttctcaagtctggtcagcctcatcctcggtgggatgcagatacgcctgcccttctctcgccaggcctccccagcggctcctccctctgctgggaatgcccttccccttctccccctgtggccaactcctctgtatcctgtagcccccccgcccctgtacacgcctcctccgggaagcccacaggattatgtgcgtggctctgctgggctgagctgggtccctgagcccctcctgtgatgtgtgatgccctctgtgtccccgcggcgcccctcctgcctcccaagcctctgggctcctgactgggaagccagggacccccgcgtcctcccctgctcctaccccgctgagagttcagagaaaaccccgttaggcagacagaggatcgcccgcagaaggcgcacgagatgtctgcgcatttttcccagggctcagttaattgaagaacttggccaacgagcacaggaaattaatcgcggcttggaaatgggacgtcccttgcgtatggtttgtctgcctgggcctcggcaccactgccagcgcccctgcctggagccgggagcttcggggtcctggcggaggcctctgggtcgcacacgtgggcggggccgagtgccccctggcgacggtgcctcagggccagtgccttcgagctggaagccgtgggcctgtggcggcgggcggaggactggtgtctcctggggtgcccgacacagggccttcctggtgctgctgtgccgacgtccaccgcggggcggcagctggatagggcaggacctggctgtggcttgtggtcttgggggtggcggggaggggtgcagcgtggggcaggctctggctcttggacccacccggcctccacacgcggtgctgcgctttgagcttctcactaggccctggaccctcccctcagcctgcagggactattcttcctggaagcccccgccccaacccctgcccctgcagctggagctccggctggcgcgtcgtcccgggctgaggcattctggctggcctgtgtggaggctcccagttcccacgaggagggtcccggtggggcctctctctgtggctgcaggccagctggtggggtcctgggcaggtgtgtggcctggacctgccctggagccgagctgccggcgagagacctggctctccccaccactgtccttcagcatcagtcagggtgttctgtgcctgcaccgctgaatcagcccgttcctgctgcagtcccaggcctctggccgaggcccctgtgtcctctcccagcccgtgatgcccactgtatgcatacactctgtatacacgcagctgcgccatggtgcccggctggtggccacgcacctgcctcgacgctgctgtgcaggtgtttttcggcatgaacacttagctcagcggacttcaagtgaagctagtgaccctctgccgtgggggtgggcgtcgtcccagcagctgaggccttgagagcaaagactgaggtttccggaagaaaaaggattttcctcaagaccgcaacatggaagtcgtgctgagtttccagcctgctgccctcagctcagaggcaggcactggattacagcttctcaatggcatggagctgggacgtcctcggggtgtacgtgggtagaatctgaacttgggctcacgctagagatgctgacagttttcacatgagacgctttaaaatgtggctttggcccctgcgccttcatctttggtttgggtgctgggccagagccttcacaggcacagtttctgccgccgctgcctgtggctgagcagtgccaggcctcagttggctctggtgctggaatgggcagttggtggggctgaccttggccgggccgcctgtgcccgaagcctccacctctgcttcagactgtgtggtcccgggtgcttctgaagcacggccgtggtccttcccggggcagatcttgggaggaccaccctgtgccccgccttccctgccctggacgccctgggccgtgcagcagaggccgctgctggggagtcggaaggtggagctatggggactgcgcctgcccaggccagaggactcaggctgcctggacttgggggtctccagcgagtgggaggggggctgcagggggccatgctggcctccccacatccagagggtgttcagtggggagatgactgggtccgggcagagggaagccgtgggggtgtccgtcgtgaatcgagcctggaacagcatggccaccctcttgttcttggctgaagggagcttgcccaagtccccaggtcctggaggcactttgcacacaggatgggcttgacacgaggtccacgcgggtttgtgtctttccacagggtcaggcttttattgatgctgtttcaatcacaaaagccacgagctacgtggagtccccaagcaggcaggttctccctagagcttctgggtcatgctgcggtcacggccactccatgttggtcagtgttgcagaccgtatattgccgtgtgctcatctggtcaaattccactttccacccaaagccaggtttaacatcatgaggaaaaagacacaagacagagggttaataccagcccagaggcctggcctgggctgctgcaggtgactgtgagacagcggcggtgaagacagaggcctccagggagaactgagcacggaggactggcttgtgtgtgtccttatctggttggacttggtcttcatttttttgtttgtttgtttcttttttttttttttttgggacagagtcttgctctattgcctaggctggagtgcagtggcatggtctcggctcactgcagcctccgcctcctgggttcaagcgattcttctgcctcagccttccaagtagctgggattacagacctgcgccaccatgcctggctaatttttgtatttttagtagagacagagtttcaccatattggccaggctggtttcgaactcctgatctcaagtgatctgcccgcctcggcctcctcccgaagtgctgatgcgtgagccgccatgcatggccaggttttcttcttttttgctttttggtttttaaacagacatcttatccttcttggtagagtgccctgtgagacaaaatggagtcttttcctaagatggagttagttatgccaagggctctctgcacaggtctgccctcccctgcttccaagcccccgcagcctcggctccacagacgctgctctgccagctctcacctgggcgcctgccactcttgttcaaccttgaccccaccttgaccctgtgtgacctctgcgctagggatctcctggcttcccccacccccagcaggtcttcccccacccccagcagtcttcccccaccccccagcaggtcttctggggctctgcccccaggggtgactgcagcgcccagtgctgcctcagggagtcctggcccttgcatggggcagccacagcgtcccacacctggggcagccccagggccctgggagcgggggcaggcatcagtgctgccttattcctggaagcctaaagccacttggggagggagatccagccctgacctcatgtgtggtggccttgctgggcctctgcggccgccctcgtccggcacgtgtgctgggccgtgacctctggaagagcagccagggtttcgtctaggtccctccggagcagtgggaagagggtttggggcacttcgtccctttgggtgagggaggggtgggggaggagtggtggtggaggtgaggggcctgtaagtgagggacggggtgaggcggggtgaggggcctgtaagtgagggagggggtgaggcggggtgaggggcctgcaggggcagaggcagtgagctcgtgtggtggcgggcgggttccaggggcgcaggcagcgcagggttgtgtgctgggtgtgggtgctggtgatggcactgatggtgcgggacggggtctgctctgagtgctgtgtgtcagatgtgggcccaattacgtgctccaattgctgccaccacccagtgggctggtcctggtttgggggctcagagcctgagcctggggtgatgggggccccgggtttgtgcccggatggcctggccctgtccttctgcctccgcggccaaaagtctgagaagtctccctgcaggccgcagacactggggcttccctcttccctcttctcccaggagctcagacgctggggcttttccctcttctcccgggctgtggagctcggcctttggtgagtccctcctctctctgcggtgctccccaggggctctcccacaagacagtgaagccgtgagcagagtggggtcctcttcccaccaggcaccacagccaatgctgacctctgctccccaaaccctccagaccctccaggcactgcggccaacgctgacctgtgctcactaaatagctccgcacatcgaggacttgttttttcctttttgatccgtggcagcttgtgtcacagccgcgtggcttgtctgcccagggaggagctgtgggtcagagtagcatgtggccacagcatggtggagtcaggcgtgggctcgcagcagggacacaccgatgtcagctgctcatagcgtgaaaggtcatggtcttttatttgcaaggggcagaaaacaaatgcaaagggttgtaaggaatgggcttacataacctggaagtccaaggggcttcaggcacagctggatccaggtgctcacattatgtgtcttaaggcctcttctcccatccttctgtgctgccttcctcatgacctcatcttcaggcaggttttcctgtgcccacccgtcctcctgccagtgccccaggcctgcctcctcctagttggcaacctttgtgggaagggggtgtcttattcctggtagttccagcagagattctggggccaccactcgctggccgtggttggccagcctcgatcccatgagcatccctgcacccccagactggaaaggaaattgcaaggctgagacaggaggggtgggtctagacaggtgctgtgatgctccgaagctccaatgatgagggggcgtgaccagtctggagagactcccccccaggatgcccatgcctgtggccagtgccccggcccagcaggtcaggggtgggcgtatccgggctggagggacttcccccaggatgcccgtgcctgtgcccagtctcggccctgcaggtcggggcatccagggaatatttcctctgtgggagtggggagaggggagcggggacactgcccggaacccaggctggactgcagcactgctttggggacagagggaaggagatcctgagtgtggggaatctccctcccttgacacccacctggatgtgggcgccggggaagggctcgggtttgagacacaggagggccccacaggctccgtggcggtcgtgtggtgccgcagggtgtgcaaggtgatggcgagtgtgaagacagtacaggggcgcggacacggggaagcggctgctctgtggctccgtccccgccggctcctggttctgagcacggttggaacgagaggcagcaacacggtcaaggtgaaggcgtctctcacattgcctggaatgagggcgtgtcccacgcatggcctcctttatggctgtgacgaaggacctggacgttggacacgtccccttcacatcccagaacacctgcgaaaaatgtaaatgagacaaagcatttgcttcctgcgaagtgggtttctctctggtgtgcattcagtgacttcagcacgcgggagttggctttcctgccaaagcggcagcttgaggctctgggttgcacagctttgctcccaggaagagctgggagatcttctatgtctcttctatgtgggtggccttgttgcagccacaagccgggttcccgcggcagggtcttcgggggctagcgggctcctcctgtctgcagcttcctggggtgggcaagggagcaagtgtagcagagtcgcttgtatcagaggagaatgcagggctgagaaaaagtcacgcatttgggccgagcatggtagctcatgactgtaatcccagccctttggggggccgaggcgggagcatcacttgagcccaggtgttcgagactagcctggccaatgtgatgaaaccccatctccactaaaaaaatacaaaaattagcctggtgtggtggcacatgcctgtaatcccagcaactctggaggctgaggcgggagaattgcttgaacccgggaggcggaggttgcagcgagccaagatcacgccattgcactccagcctgcgcgacaagtgaaactgtatctcaaaacaaaacaaaacaaacaaaaacaaaaatacccacatgcatttgggggaattttggtggggtgtgaacttgagcatctgaggggaaattgaagaaggatcagtgagtgaggtaatcgcgtgggacgtgaatgggaagagcaatcatggtggaagtcactggcgggaggaggtgcctgcaacccgggaggaaggagaaacggagcctcctggcagccaggacgaggctggaaagaaggtgatccgaggagctcctgttgcttggctccgggtttaactggcagagagaagccctctgcgttggggtctggaggaggtggccgagagtgaggagaggggaaggctgatcggtgccttctgccccaggggttttgcttgttcctgtgtttgtagcagttgatgggcaggccttgctgttcccatttgactgagagttttggagacgttgccaaaggatgaggctgccaggctgaaaccagacagaaggggcaggggccagagtggagaggggggtgcgtcctggagggggcagctgcgcatagagggggcagctgccactgtggagttcagggtcccgggaggtggctatgggggtgccctctggcccgagcgagctctggtggggtgggtggggagtggggtcctgcgttctggagacgcggccatgctggtgccctgctgcggcctggtggggagtggggtcctgagttctggagacacagccatgctgctgccctgctgcggcctgtctgagagcgcgcgttccctgcccccaggttcctcctggttttctcctgcctcgtgctgtctgtgttttccaccatcaaggagtatgagaagagctcggagggggccctctacatcctggtgagccccgagggagggcgggggctggaagtgcccaggaaggagctggagctgcctgggcgtctgtcttccgttcctgttactacagggcctctgacgcccccagccccagctctggccatgtctttgtgccctgtcagttcccatctgcccccagccccacagcccgggctatgccccagccccaccctggctgtaactcagccctcacctcacccctcctggtgacctcacagtggctgacagccccgcctccatcgcccccaccctgggtgggccaccctccctttcccatccccactttatctctggagccggagatgtggggcgtccccagcagacggcagacgtgggcgtcggggtgggggctgtgacgcttgcagcaaccctgtgccagggcagggtgggcgccatggcaacgggagtgcagttcctgtgttgctgttggtaacggggtggggggccctcaaggagactggtccccatccaaatgcacctccctctactgcccgccctgctcctctgttctggggcttcccaaggtctgggtggagactgtggccccctttgcagttacctggggccttccctcagctcagccctgctgtgcaccccccaactcaaggcaggtcccccactggctcagacagggtcccccagctcagacaggggcccccaactcagatagagtcccccagcttggacagggcagctccccaaccagctcagacagggtcccccagctcagacagggtcccccagctcagacagggtcccccagctcagacagggcctcccagctcagacagggtcccccagctcagacagggtcccccagctcagacagggcctcccagctcagacagggtcccccagctcagacagggtcccccagctcagacagggcctcccagctcagacagggtcccccagctcagacagggcctcccagctcagacagggtcccccagctcagacagggtcccccagctcagacagggcctcccagctcagacagggcctcccagctcagacagggttccctagcccagatagaaccccctagctcagacagggcagccctgccagctcagacatggtcccctagcccagatagagtccccagctcagacagggaggtggggagggtggccccatggtgttcatgctcagctgtgttcccggatgcagggtctccagagggcccaacccttcctgcccagaggctggagaggggtgggggggtcagcttgcagtttcctttgggggcctcctgccctgtggcttggtccctgggccagaactgctcctgtgggtgtccaggctgggcccccaggccctcccccaggcctcaaggtggcctcagctttcctcccctgcaggaaatcgtgactatcgtggtgtttggcgtggagtacttcgtgcggatctgggccgcaggctgctgctgccggtaccgtggctggagggggcggctcaagtttgcccggaaaccgttctgtgtgattggtgaggcctggtgggggtggtattgctagaatcagggccaggcacccagggacggactcagccctggggggagctggggcgtctgcgtgggccagagaggctgggcaggactggctccttctggaagtttcttttcacctgctgacttggagtcagaggctgtggttaactctgcctaaatgtcaggaagaggaatgtggcgctgggctgcccatcctgggccccacaggcagggtggacgatagtaatgtccttcctggggcctgacagagcccacaccaggcgctgggcatacaccttcccgcccctctgcacatcctcctggaacgcggggtggggagttttctccaagggggtgcggggagcggcccagcagctcaccagctccacgcccgctttgtagacatcatggtgctcatcgcctccattgcggtgctggccgccggctcccagggcaacgtctttgccacatctgcgctccggagcctgcgcttcctgcagattctgcggatgatccgcatggaccggcggggaggcacctggaagctgctgggctctgtggtctatgcccacagcaaggtgagtcacggccccaaggctggcggtgggcgcccccagccagcgagagtcctggcccagaccgggccccacccctgcctggggtttgcttcaagagccctgggtggagggatggagtcagtggtggctctggctggagcccatcaggtgtgaacgagcctccctcccctttcttctcggtgcttcttctcgtgacttgggccatctttgtcatctgtctccagaaagctgctttttcgaaaggccaggccaggccattttggcccctgccacccccaccttcctgccgcgaacccttggctctgtggttgggtgtcttctttcctgggtttttagtgcctggatcaatacaaagtgctagtcacctgcctggtggttgtttaagctgcggcttgcggcggccgctgtccgggggacccagagggtggctgtggccacacatgctgagttccaggggccccaggccccccagtgaccgagaccccatcctgccagactgtgctgtccctccaccttgctcacgggtgggctcatttgctcaaatggccccctccccattttcttccctcttgagccctggcagggtggtggctttgtctgcagaatgggcaggggtcagcccccggtggcccctctccctccctggacgctgtgggggttgagtgctgactgcagtgggcacccttttcacatctgtgcccggaggctcagggaggttgggggagggtctcaacagtcccaagattctccagccctgctggccgccagaatagacgctactgaccctggagggcacggtgggctggccgtgcgagccactgttgcccaggccaggaggctcaggggaatgggctgccgtagctaagatggtgaagccagcggcgcctcctggttcccctgtgccagccctgccaccgaccctggcgtggagcctctgctggcctcaggagacgggtccatccagcccctatatccctggagacagagcccaaagtgggctgactggcgaggctgcagtgctcctggaggcaggtggcgggtggtgatggtggtgagggtgatggtggtgatggtggtagtgatggtggtggtggtggtgatggtcatgatggtcatggtggtggtggtggtgatggtgatggtggtgatcgtggtggtggtgatggtgatggtggtggtgatgatggtggtgatggtggtgatggtgatggtggtggtgatggtgatggtgatgatggtggtgatggtgatggtggtgatggtgatggtggtggtgatgatggtggtgatgatggtgatggtggtggtggtggtgatggtggtgatggtgatggtggtggtggtgatgatggtgatggtggtggtgatgatggtgatggtgatggtggtgatgatggtggcgatggtgatgtgatggtggtgatggtgatggtggtgatcatggtggtggtggtgatggtggtggtggtggtgatggtgatggtgatgtgatggtggtggcgccggtgatggtggtggtgatgatggtggtggtggtgatagtggtggtggtgatggtgatggtggtgataatggtggtggtggtgataatggtgatggtgatgctgatgatggtgatggtggtggtgatgtgatggtggtggtgatggtgatggtggtgatggtgatggtaatggtggtgatggtggtggtggtggtgatggtgatgatggtgatggtggtggtgatggtgatggtggtgatggtgatggtggtggtgatggtggtgatggtggtggtgataatggtgatggtgatggtggtggtgatggtgatggtggtggtggtgatggtgatggtgatggtggtggtaatggtggtggtaatggtgatggtggtggtggtgatggtgatggtggtgatggtgatggtggtgatggtggaggtggtgatgatggtggtggtggtggtgatggtgatggtggtgatggtgatggtggtggtggtggtggtggtgatggtggtggtgatggttttggtggtggtgatggtggtcatggtgttgatgtgatggtggtggtgttggtggtggtgatggtgatgcttctggtgatggtggtcatgatggtggtgccgtctgcctctcaggagctggtcactgcctggtacatcggcttcctttgtctcatcctggcctcgttcctggtgtacttggcagagaagggggagaacgaccactttgacacctacgcggatgcactctggtggggcctggtgagttgtggtcattgtggttttccctttccctgctgatacacccctgtccctgtgctgggaccaggctctcactggctgagcctgctccatacatctctttggggccacctgctggctgcccgtggtcatgatggcttgtggtcgaggcggggtggtggttgccaagctggcgggggagtgggtggggagggctgggccccagagagcctgagctcagccgtggtgggcatcttccttcctcctgagctcagccgtggtgggcatcttccttcctcctgagtgaccctccacctgctccgtgtcccgattattgggggaaaggggagaagaggcatccctaggagatctgtcctgggcagccctgagtttttggagactaggtggctctgcaggccccactctgatcaggtctgaacagatctggcccctgggctcctcctcacctggggccacagacaccctcaggctgctggtgcacaggggcagggtctgtgctctcctgggaggctgagaccagaccccgggcctagccccgcacatgccggctgagtgggctcagccctgcggggaaggcatgggcagctccacctggtgcctaagccggatgcagctgatccgaagttattttattaatttcatgagacctggacactgtcttcccagtcggggcttgggtgggggttgagagctgagcgtgccccggggcccaggctgtggaccatacagatgacctggactcactgagccatcttgtgtggttcaggccccaccacagggtcagggtggggcccggggcgtcctgagctgtgactgctgggccctaggacttttgatggggacaagcccatcccccaggtcttttgtgggcaacagccttcttatctttacgattgggtgttttttcagagtaatgcttgtgtatatttttaaatgtcagagagttactaagccttatggtgagaatagagccccactctgtggccgcggtgccctcccctctgggtccctctgcagcggatgcggctgcttctgtgtttttctttggggtttacctgattagtcttaaatagcaaatagggatgctggtatttcctgattcaaacgcgctgactgtgtacctcctactgggatgagatctacccccagtttccctttcttgcagcctttgggggttttcagcagtgtggggggaaccccgccctcacctcccccgcaggccaggccccatctgccaggcctccggcttccttttccttgaatacaaaaaattagccgggtgtggtggcgggcgcctgtagtcccagctactcgggaggctgaggcaggagaatggcgtgaacctgggaggcggagcttgcagtgagccgggatcacgccactgcactccagcctgggggacagagcgagactctgtctcaaaacaaacaaacaaacaaacaaaaaaccacgcctcctttggtaacgaaattgtgtagagggctgaggccctggcttagtctccccaggagaggtttcttggtcaggcctcgcccagcctcccgcccactctgtgtggcagcctccttcccagagtctcctgtgcgctccgcgtggtggtggcagccaaggcccccaggtgctggcgctgctttgcaccaggccacagcttcctgtcggtgtggagcaggtgcggcccagccggcccaccctgtcctcagaccacacagcagcgtcctcggaacccagggtaaactccccaaggtggggctgcaggtcctggtgagaggtctccccttcccggccctgctgtctgcgctcaggcctcgatccctctgtctgtttatctaagactctgccctcacagggcctgtcctgggcccaccttctggtcctagccagagtggcctccgtgcggctgtcctgagcccgtggcttgtggtgcgtgcggttctggcaggcgggggtgggctgcggccccaacatgtccagcactgagtgcagggtgcctgcccctgccttcctcggccccggcacaggcatctcttgtcggagctgcttggagacgtgtccgtccttcgcttcccctcatccaggccccgagtcgggtgctcctgggctcaccgggacccttggggaggtccaacctggcctgacctcccccttgcccaccacccctagagaaactaagcacaacccctggggtccctgctccaggagcagcccaccccgccatggctcggtggagaccactcccccgcctcttgtggtggcctcctcctgcccacccctctcctctggcctcagctcagctcaggctcagcgagccccctgcacgccctgcccgttcctctgggtccctctgggcactcccttctccccagctgcactcttcacctggcggggatgcagtgggggctgctgtcctagccgagcctcgtgggagtcacgtcgctgggcctctggttctcgatgtgagatggggacacagcctttggctggaccccgcttctggcctgtaggggaagaggagagagggctcaggggatgaggggcggccgacaccgaccctggcctcactgggcctccgtgtggatggtgacacgggggtggccccgcatctgtcccatcccaagccccgagtcgccagcgggcgtccagcctgccctcaggggtgtgagcaggcccttcgtgtgactagagcctgcggtcccacagatcacgctgaccaccattggctacggggacaagtacccccagacctggaacggcaggctccttgcggcaaccttcaccctcatcggtgtctccttcttcgcgctgcctgcagtaagtccagctgcccctgcctgccttggagggggacgaggtcttgtaggctcccgaggtgaccacaggcccctgggcacagttccctaggtgggacctggggcaggagcagctctcagcaggtccacagcccccaggagctggaggtggcagctcagggtcagaggccttgttccccaaggactggagtgggggttccccagccccgacaggagcatgcccaaggctgcggctgtagcttcagggggctctgttagtcactggtggcccctcttagtctgagtggggctgagcaggaggtccttgtgaccaggagcagggcggctggtgacacaggtccctcatgggtctctaggcagtgaggcccacccagctcagagggaggtggaggggccctgtcaacccttgttgccccagggcagggcagggcagggcagctggtgcacggcaagaggctggccccggtgtccccttcactgtgtgcccttcaagatgggcctgcagactcttctgtgtggaagggaaagaggccactctgagttcagtgtgggtcctgccatgtctcctgcaagccagagggttccttcctcttgatgctgacaaattgtggggatatgggttcctggattagtctgaccttgatgaaggtgggggaggcatggatcggtggagacgggtctgaccctgatgaactggggggtgtgtgagtccctggggcgtggtctgaccctgatgaattggggggtgtgtgagtctctggagtgtggtctgaccctgatgaattggggggggtgtggggccccagagcatggtctgaccctgatgaattggggtgtggggggtccctggggtgtgacctgaccctgatgaattgcagggcatcttggggtctgggtttgccctgaaggttcaggagcagcacaggcagaagcactttgagaagaggcggaacccggcagcaggcctgatccaggtgagtccaggtgtcccccggggaccagcacagcccttgtcctggtcccaccttgttgaggagtggaggccgctggggctttggcattgccctgtctgtggatgggccacagcgctcaccctctgcaggaatctgtggaaggagcgaggatgtgaagtgtgtgtgttgagaaatggctccaacccctgaggctgcacaggggtggctcgtgtgaggacgctggggtggcccttgccttgtgtgggtgctggggcccggccatcaggtcctctctaccgcaccctgggaggtgagcacacccgttctgcagctgcagggccaaggctcctagagcccacacactgcagtgagtcagtgcctgacctcgtctgacccctgatgctggctgtgggcccccatgatgcccgcccggaccccacccagtgccgtcccaccatttgggggtgggtagcgtgcagtgaccactgtggcatccacaagtaattcctgagccaccttgaaaatacggacagacaatggccgggcacagtggctcacgcctgcaatcccagcactttgggaggccgaggtgggaggatcacgaggtcaggaaatcaagaccagcctggccaacatggtgaaaccctgtctctcctaaaaatacaaaaattagctgggcgtggtggcgtgcgtctgtaatcccagctactcgggaggctgaagcaggagaatcgcttgaaccagggaggcggaggttgcagtgagccgagatcacgccacagcactccagcctgggtgacagagcgagactccgtctcaaaaaaaaagagaaaatatggacagacagcaggacccccttgggaccagccattcttgcagctcctccccaaagtcttccatccactggacccgcagagctgaccaggatccctgccagcggaatgtggctataggctctctcctgtcttagacagcaaatcttgagatggagataatcctgggttaccccaggtgggccctaaatgcaatcacatgcatccttataagaaggagatttgacacagaggaggggaaagcagtgtgaccacaaaggcggagactggagacgtggccacaagccgcggagtgtcagtggccactgcaggggatgtgcctcttgtggtcttggctctgggtccagcctaccttaaggtgtgtatttatcttccactataaaaactacaaacagctatttatttccaggcaagagttttaagaagaaccaaaatgcccccaaatctcactccactattgacggtttaaaaagtgaccacgtacgtgtggtgaaaaggttacaatagtgccagaaaaagcagcgtctgagcacccagggtcccatgcgccctgcttccctcgaaggtcactcttagcagctggtgcacatttccagagcacagctgggcctctccacaggcgcacctcagaggacctgcagccgtggttccagaccactacggtagagatacggcaataaagcgagtcacacaaatttcgtggtttcccagtgcatatggaagttatgtttacaggctgggcaccgtggctcccacctgtaatcccagcactttaggaggctgaggcaggcagatctcttgaggtcaggagttcgagaccagcttagccaacatggtgaaaccccatctctactaaaaatacaaaaaaattagctgggcgtggtggcacacgcctgtaatcccagctactcaggaggctgaggcaggagaatcacttgaacccgggagacggaggttgcagtgagccgagattgcgccactgcattccagcctgggaaacagcccgagactccgtctcaaaaaaaaaaaaaaaaaaagttatgtttataccatgccatagcttactaagtgtgcagtagcattatgtctaaaaaacagtaaaacactgcacgtactttcacttacaaatacttaattgctaaaaaaatactaacaatcacctgagccttctgcgagttgcaatctttgtgctggtagggggtctcacctcgatgctggtggccactgctgatgaggtggggctgctgaaggctggggctgtggcgattttttttttttttttgagacggagtctcgctctgttgcccaggctggagtgcagtggtgcgatctcagctcactgcaagctccacctccctggttcctgccattctcctgcctcagcctcccaggtagctgggactacaggcacccaccaccacgcctggctaattttttgtatttttagtagagacggggtttcaccgtgttagccaggatggtctcgatctcctgacttcgtgatcctcccgccttggcctcccaaagtgctgggattacaggcgtgagccactgcgcccggctgtggtgatttcttaaaataaggcaagaatgaggtttgccgcatcgattcactcttcctttcacgaaagatttctctgcagcatgcgattctgttggatagcgttttacccagtggacctcctttcagcatcgcagtcagtcctctcaaatgctgccactgctgtatcaactaagtttatataatgttctcattccttgtcatttcaacacagttcacaacatctttgcctggagtagattctgtcgaccaaaaaaaaaaaaaaagaaaccacacacatgtcaaatgtatttgggaattagaagaaaggattataaccagagatgaaccactgtggccagagggtgggggactctcgagagatgagccagtgtggcgagccacccatgtgccggagagggaggggcagggaagctctccctggctgagaagttcacataagctgcttggaaacagagttcactggctctgaggctgaaaaccagagttggcagccgttcactggttgagatgccgctgctgggccagtgttcttcctagagcatcttgtctgaattgctgcattcctgataaggaattccttgtggggttattttagaatgtctttgacactgtcctcatctcagccatgcaggcgtgagccctgccgccgtgtactctctggcctcggttggtttggacctgacagaagtgaccttaccctgctgtctgcagtgttcaccgtccatcccaagaaaccacttactttgctcatccataaatgctgctcctcacctgttagagttttatcctgagatggcagcagttcagtcccgtcttcaggctccacgttaggatggccaactttttttttttgttgttgttgagatagggacttgctctcttgcccaggctggagtgcagttgtgtgatcttggctcactgcagcctcgacctgccaggttcaagcgatcctcccaccttagcctcctgagtaactgggactacaggtgtgcaccaccatatctggctaattttttgttttaagtaaaggcagggttttgccatgtttcccaggctgggcttgaactcctgggctcaggcgatccacctgcttcagcctccaaggtgttgtgagcctctgcgccaggccagggcggctgacatgttagcaaatggcaaatcagcgtagtgaggatgtgtggacaccgccccccctgctggtaggaatgtagcgatgcagtcaccctggaaagcagttcggttgtttctcagaaagttcgtaaacgtggagttgtcacgtgacccggccattctacctctaggtatagacagacacaaaagaattgaaaacagaccttcaaccaaaaacgtgcacacaaatttcacagcagcaccactcataggaaccaaaaagtggaaacagctgaaatgtctattaatggatgaacggaatgtgatttatccatacgatcctcctatcaggaggagcgggcgctgccccttgctacgacgcggatgggccgcggggacgtcacgctcagtggagggagccagacggggaaggccgtgcagcttgcgatgcccttggcaggaaatgcccggggcggggaagtccgtggagccccgtacaccctggcggtggccagggcctgggggaccaggaaccaggagcgcctgctgaacgtcgggggtaggagggggtttatttgggggcgacgaagaggtgtggagttagataggggcagtggtcgcacagcactgtgtgtgcaggcgctactgaagtgcacgctttaacatggctaacatggtgaacttcaggatatgtctatttcagcaccgcaaaacaaaacaaaggcatagagaacggaaatccagccaggcgaggttcagtctgacgtacaggctcatgccccgagccgggctgagtccccgccctgggaggttggaggctgaggtccctggccctacccatactcccagcgcctcaggttgcccagggcccaaccaggaggtcaggaaggtaacccccaggacaggtgatacccgccaggggccaggtaagagtcatgaaaaggaaagaacacagcctccctgcgagctcgctcagggccccgagtcaagccccaaccgctcccctccttcaggccccaccgtctgacaagcgggcttgcctgtctgtcctacagagggtaaactgaggctgtggggctgatgcagcactggggcctggcctgctggcccagagtcctgagtgcctggcctacagcatgggggtcccctctgccccgttccctccacgcccggccctcctgggcgcgcccctcgccgcctgcccctcgccaactgccttgtctttccctccgcagtcggcctggagattctacgccaccaacctctcgcgcacagacctgcactccacgtggcagtactacgagcgaacggtcaccgtgcccatgtacaggtaccgccgccgggcacctgccaccaagcaactgtttcattttttattttccatttgttcttaaaccccactttttgttgttcattattttgattgattttttttctttaaaatgtatttttcacaaaggagttctgtgtgtggtttatttcgaggcgttgcttctgccctttgtgttagagctgcggctttctcttcactttcatttcctcacccctttgctttatcgtcctggccgtgggtcgtgctgtgttttgtgcgttgcaaggacgctccccagggagctcaaggtaaatgccccccgccccgggctgctctggaccgacagctgctccccgggctgctctggaccggcagctcgaggtgcccaagcttcaagagtggaaccagtaccatgaccccgtcccccatcaaggacctagggctccggcgtggggggcaggaaagtaaccccggatcacagtgagactttcagaccaggaaactcaaattatcctgggggtcccaggtgttccccaacaggacagagcacacagacaaccttggagcccctcgggcagcagagaagggggcctagaaagtgcctgtcgaggcagtgctagcaggtcctgtgagcctgtgtggtcggagggcggctttcccgtgtttttttgttttacaaaagggcaggtggcagccagctgggattttcgttgcctggctcttgcttaaattaaggtctgagatgaggggttctgagggcctttctcctggtccacgcgggcctgacccctcgagtccgtctgtgtgggcaccaccttgcccgcccgccctctgctctgaggtggggaactgagccccgggacttcccagctcccgcgccccccgtcacctgctcctagggggtttctgtcccctggagccttccctaagggtggggccttccctgtgggtggagccttccctgagggtggatccttccctgggggtggggccttcccagagggtggagccttccctgagggtggggccttccctgagggtggggccttccctgagggtggagccttccctgtgggtggagccttccctgagggtggagccttccctgagggtggggccttcccagagggtggggccttccctgagggtggggccttctctgtgggtggatccttccctgagggtggggccttcccagagggtggagccttccctgtgggtggagccttccctgtgggtggatccttccctgagggtggagccttccctgagggtggggccttcccagagggtggagccttccctgtgggtggatccttccctgagggtggagccttcccagagggtggagccttccctgtgggtggagccttccctgtgggtggatccttccctgagggtggggccttccctgagggtggggccttccctgtgggtggggccttcccagagggtggagccttccctgtgggtggatccttccctgagggtggagccttcccagagggtggagccttctctgtgggtggagccttccctgagggtggggacttcccagaggctggggccttccctgtgggtggatccttccctgtgggtggagccttccctgtgggtggatccttcccagagggtggagccttccctgtgggtgtggccttccctgagggtggggccttccctgtgggtggatccttccctgagggtggggccttccctgagggtggggccttccctgtgggtggggccttccctgtgggtggagtcttccctgtgggtggatccttccctgagggtggagccttccctgtggatggggccttccctgagggtggggccttccctgcgggtggggccttccctgtgggtggatcctaccctgagggtggagccttccctgagggtggggccttccctgagggtggggccttccctgtgggcggggctttccctgagggtggggccttccctgagggtggggcaggtgcacagtgtggctgggaattctaagctggggtggggcccaggattcttcttgaggatcctgagttggccagggtggacagacccgcggcagtcctcagttgctacctgccttttcagggtgtcacttgggaggaggatgccagagcccttggcggtcctcgatgtttgcagtcgccctctggggtctgccccaacccctccctcagcaccatcccccctgcacctcctggaagcccgaccttaggagcctggccctgcttgtctttcctgggaatgtcggccatgcccttgtttctgcaaagtgacctctctcagcccagggcagggacagaaccagcccttgtgctaattttctttgttctgttgtttcccctactttcctcaactatttgtttcattttctttttcttatcccgttctttttgaaattttccgttcttttccctttgtgtgtgtggaaacacttgaaagttcgcaaactcaaacctacggggcctccaggtaggaaatgcatggacagaagccctgtgtgtgtgtgtggttctagttctgtgtctggaactgtgaccgggcaagcccctggagccccgtggcccacccggtctgcagagtgaccactgtccctgcccaccgagcatccctatgccccctgccctgcgtggccccatgctgggcggccagatcaccctgggccctcccctgcctggagcccacgtgccaaccctgccctcccagggcccatccacccttctcgtcctgcctccccccagccgctgcttcctgttgccagcccgcctgtgtcccccgtggtctcgttctgtcctgcttctggcctggctcccctcgtgcctccatccccaccccacaggccccatctgtgagcagagcaggaatgatggatggggggcttcccctgccctggcgctgggcacccatctggggccaagggtgctcccaggccaggacccccagaccagccacagtccttgtcccaccgtggctcacagggcagcctctgcgggccagctagcctcaggggtctgcctcagggtctcctgccctggactcaagtttctcctgagtcttgccctggcatcacaggggaaccccagggctccaggccgtcccacccctgtccccactgctgagagcaggaaacaaggctgtttgttcagactggagagcgctgagctgctatgggaagcctcacccccatacagcacaaagggcctttacctgtcgggtgggcaggcttgtgctctgtgatgacactgccctgcagagcctggggccagccactccccacatagaccagaagggcttcctgcacagaagcccttgatgactcctccaggggccagtcacccgtcctggaggagagacctggctgcccttcctgggaactgttagaatgttgactctccattcccagaaaggaagtgggtgcagtgctgggcagtggtgggtccagggctcctctgcccccgacatgctcagccataagacatctccaatgtctgcagaggccctgctgcccctggggccaggaggctggcgctgaagccggctgcctcctccccagcctctgctctctcccagggccgccccctccccttgttgcaccctcgtcccacagctccgtcctgccctggccctgccgcccgcctgtctgccctggcgctgctgctctgtgtgttcgaggcatggcctcctatgtggctctgagggctgacgtgtgtgtctctcacctgggcctgcgtgcgtgtgggtcccatcaaggaggggtggggagccacctgtggcccccagacagactccggccaggctgggctctgctgcgcccacaacaccagctcaccagctgcctggttcctgctccatcccgacttggacagcggactgtcctctgcagggagggggctcacaggagccccactcctgccagcattttgggggcacctctggggctgcagagggaacaaagaggggccaggcacggctttaccagctcacacctgcctagcctgtgggggggcaccagaggctggtggggtgcagggaggggcttctggacagctctggagggaggagtgtggctggacgaagtgggagtcggcagtagggtgatcaccaagccctgagggacaatggagtcagccatccttttgctttggaggcagtggggagccattgagtgtttggtcaggagagtggcaaggatgcccatctggaggaccaagtaagcctgggtgggcagaggcagggcaggtgaagagtctggaggatgtgggccacggcccagcctggggcagagggaggacacgggcaggggcatctccagggggtggccgtggccatagccgcgtagggagtcagctgggtgagggctgggctgggaggtggccgtcctgcctccaacgcagaggagagaccaggacgtggccccagtggggagtgctccacaggtgggtgtggggaggggtggaggccgtggatgggatggcagggcctgggagcagaggctgaggaggcggagtggagggggttcaggtgggtcagggagccgggtgggggaggccccagaggagggtgtctcttcctgaagcgagctggaatgctgtgagggcatgggtggtgaggaggagtgggcagagtgaggtgggtgggagtggggaggggtgaagagggggacgtcccacccagacgtgaggaggtgcctgcatacctgcctggggggcttcggcgggcccggacttggtcctgctggtggctgtgggcggcagggcaggaagacaggggtgggtatgaccaggactgggaagggggctcggagcagagctgggccacctgcggaggattcccaaggcatctgggcctgatgaggctgggctcatcctatagactccagaatgagctgctctttgccaagcagaatccttttctgtatccccacctcccaactcctccagctttggcagagaaagtggctgtgtagagaggggccggggccctaaatatattcgcgtgctgtccctctaggatgttcagggattagaagggggagtagcgcctccccctacctgcgcgccctcctccacctgctccaggctgcagggtcggggctgcatgggtggccaggcgccctcttccctcctccctgctgtccctgctggttgacaaggccccaggcacagccagagaatgaagatggctcccaggcctgcccaagagaggtgggcctgagcccagggcccagtgggcagctgcctgaaagcctggccacgtcacatgccgccgggtgtctgacactgcaggcgcctgcccatggagctgtgcaagcagagggaggtgtcccaggactcgggagggtgagacgctcactcccctctccttctcttgccccagacttatccccccgctgaaccagctggagctgctgaggaacctcaagagtaaatctggactcgctttcaggtcagctggggagctccaggtggggcgggtgggcgtctcagtcctcctgggggccccagctgcccacagaagacacgccaggacagtgccccagggactccagggagctgggacgcgcggggacgctggacttggtgatggtggtgctttctcttttcgcgtctgctgacctggtctgggctcccacccccagaggggcagctggttcgggggacgggcagagagggaggggcgaaggttgatatggggccgggggctctgcatggctcctaatggggcccaggcgcgtgggtcgtacgacaaagtgtcccagcccgtcgtcctctcgagtgcctggtggtaactcaggtgctgatggcgcctgctgcctctccttcaggcccaaagcccacggctgtcagagtgccggctgtcgaggtaggttgcagaagggcctgctgagcaggagaggtggaaggcgaatgcacctgccaggcctgcccaggcccagagccgtccccaagcagttccatgtctgaaagcccttggtggggcaggggcttccagtgggttggatgctggtttctgcctcctagagacactcttctgctctggaacctatcaggacgatctcagatcatcctggattttccgggcgggccctgagtccaacaacgagtgtccttacaaggagagggagatttggaagcacacagaggtgatagccagctaggatggagagagagactgaagtgatgcggccacaagccaagggctgcctggagccacagaagctggaagaggcagaaaggacccttccctggagctttcggagggagcaggccccgcccgcaccttgattttggactctggcgtctaggactgtgaaagaacaaacatctgttgttacacactccagcggcacttagttacaggagccgttgcacatgaatccagactttaataccgggagccgcgtgcggctgtggcaaacgcctacaagcgcgggtgtgacatcagaaccgggtgagccaagggctcgtttcctagaatcacaggttcatgcgtggaggactccagccccaggctgatgcacacctgcaatcacacacacctgatttaaatgattctgatgactttattattttatttattattattattttgagatggagtcttgctctttcgcccaggctggaatgcagtggtgcgatcttggctcactgcaacctccacttcccgggtacaagtgattctcctgcctcagcctcctgagtagctgggattacagacacctgctactacgcccggctaagttttgttatttttggtagagatggggtttcaccatgttggccaggctggtctgaagctcctgacctcaagggctctgcccgtctcggcctcccaaagtgctgggattacaggcgtgagccactgtgcccggcctctgatgacttcagatgatgaaatttgggactttttgaactgatgggatttggatgagattttggacgtgagtattttgaggtgcaggctggaagaaactgaggtgtcctgaagagatggtgggtggggacacggatgttgaaggtgcttctggtgcgggctcaggaggaagtgaggatgcaggtgttggaagctggagggaaggctgtccttgtgttgttggtggcagagactccactgggctgtgcgctactgttgggcagaggcagaatttttaagtgatgaactgggatatttagccaagaagatttccaagcaaagtgtggaaggtgcagcctaatttatttctgctgctcatagtaaagtatgagaggaaacagggagattcagagcagcacctggccgatccgccaaaagactattaaaaaaaaaaaaaaaggcattcagggcagaactgctgtaagaaaccagaacttgaaggtttgggaggtgctcagcctgtccagcttgcaaagggtcttaaattaggagattcactgttggcaaagcctgctttggagagaagcccacaggtgtggccagacagcctcatgctgaaggcaccaggtgtgcggctgcagcctccaccgtctccacagaggcctgaagatggagctatcgggaaggatgtgcagaggggcctcttgtctaacggcgttgcggcctgtggcatattcaggaggccctagaggttttggggagtttcataccagcagaaactagattggcagagacagcgttggacgaaggtaagagggcatcgaacttcgggagtctgcaggcaggaaacaggccggtggcgctcctcagctgtgaacacgagctgccctttaggaaacggggaacgatcccgaggctcccgcagaggccggcggggctgagagcccccgaggagcgtccccagagctgtgccacgtggagtttgccccgccggattggaaacgtggtggccggcggcccctctgtcattctccctttttgagtgggaagagctgtcctgctgttgatactttaagcagatggcttgttttctagttcacaagcccatgcatggaggactttagccccaggttggcgcacacccacggtctcacacatacctgatttaagtgattgtcatgacttcagaggatgggatttgggacgttttgaatgggtgggatttcttttttttttctctgagacggagtctccctgcgtcactcaggctggagtgcagtggcgcaatcacagcttactgcaacctctgcctcctgggttcaagtgattctcctccctcaacctcccgagtacctgggattacaggcacctgccaccacacccggctaatttttgtatttttagtagagacggggtttcaccatgttggtcaggctggtctcgaactcctgacctcaggtgatccacccgcctcggcctcccaaagtgctgggattacaggtgtgagccactgtgcctggccaacggatgggatttcgatgaggttttggacacacagtccatgctgcagagggtggagattttgggggatcttggcaggggtgagtgtattctcgatgtaagatggacatgaattttggggggtcgggggaatgcagtggttgaatgctggtctccaaaatgtcccagaatttgcaaatgtgacctgatttggagaaagggtccttgcagctgtaactgagcttgggatcttgagatgaggtcatcctggaacatcctgatgagcctggtgacagcttgagccctagcccggtgacagctgtccttacagaggtagaggaggagaagacacagagacacagagaatgtcgtgtagagacggaggcagagacggggcggtgcagccacaagccaaggacacctgggccaccgggagctgcaggaggtgatgagggaccctccccgagagcgtggaccatgttccacggcaacacggtccctgtcacatgaagtatttggaagtgcaccaaagagcctgcttgtaacacggcgtaccagccggggcaccagcccaggtgttcgaatgagatcgctggggcctccccatggatgagccaaatcacgatccgccgatctctcactcaatccataacgcctttacccgcggaaacagccccagatgagacgccggaaggcccagacagctttgcctgcaggacgaggccgggacagttggtcagcggtgcaaaccctgggctcactgactgtggcgcagtggagccacccctggaaagtcacgcggaagagaatcattccattaggatgagcgtggaaaaaagagtcggagcagctcactctgtctcgaaaattgattgtaaattaaactcccttccagagcttccttcagcaaacacgtgctttttggaactttattaatattggtgtggttctgattaaaatgagtgtctgtaaatgtggaggggtagagacccccaggtcagaagagcagaggcccccgccccatggagtccctcttccctctcggagctgcctccgtgggccctggggtgcccgttcctggggccgtgggctctctggggtgggtgggccagctccacccctgcccagccttttctgagacccccctcccctgcagcccctgacccatgggactgtggggggctggactggttgggggaccctcatgggtgctgagtgggcggccactaactctctgttgcttctgtttgaaggaaggaccccccgccggagccgtctccaaggtcagtgccccctgctgctaccctggtgtctgccgtgtgcacggccgtctggtctctctcccacacgtgtgcgcaacctgtcatggagatgtgagggccttgtgtgtgcttccgtgtgtgactgtgtgactgcgggtccagacccccgcctggcggtgatgtgggcccttaaatcactcttcctgctcaccccctccccagtgattcggttttactttgcagcactgtggatccgggcagctgggcggcttctcgggggtgggggatcccccaccccgcccacaagtctggccccagggttctcggaggcagggggtctctgttagtgcgctccctccagctgcaggcacatagcccgagctcacagctggcctgagtcgacgccggctggggtgaaagctccaagtgggcctctggccttcccgctgctctgggtccagagtgtctggagcatgtggcacagaccagggcccctcgtcctccgaggagggtgggacatcctctctgtctcacgcccctgggtggagattctggctggcctcctctccctgtttgccaaggtcaaagtgggccaagggtgcaggtgcttagcctggttccctctcccgggccccgaggttctgtgggtcgggcagattggagacaggactcgtgtaagggctctgctggggtgaaggatggagacagagaaaatcaagatcctttcacaagttaattctacgtctgctgagccccagcccccgacacatcaccctgaggaggtgctaggcttctctgggccccctgtgccccatccacatgttgcagagtaaatctggccccttggacctggggtccgagatggacgcctggctgcccctcctggactgcgggtgacagctggcgagacactgcggggcttgggtgcggggagatggagtggggctgagctgcatttttccagccaccccacatcccacagaaggggagtcatggtcagtgccttgagctggaaagacgggcaatgcttccggcccacaccaaccaagaaaaccaccaggggctcattcatcctctcaaagaggctaagaaaacggacgagggcccagaaagagctgggtcggcagcgtagcctctgctggtgccagcccccagtgctgccactgcctcccccgcccctgctgctggccctgaggctgggagtggctgtggggatgagcactgtgccccccacaatgggcacccttggctgtgatttgggcggaacctcggtgctgtcccaggtggacgcgaggcttcccgacggcttccgcgtggcagggccccacgcccttgctgggagcgacgccggaacccgccccgtgcctcacggcccctcgtgctcgctgccccagcccggcccgctccacgttctcgtctcagcctctctctttctctctctccctcccaccctccactccacccatctggacacccgctgcccccaagtgctttaatccctgttactgcccttctcgggcccctctctcgtctgccctctctttggggatgaggtgctggccctgaaggcgaactccaggtgaggacacgcgggagcctccgggctctccggtaagctctgtggctccccacccctcccccgtctcttccccatgtgtccggggggtccttgcctgtgcctcccacgccctcccccacgggcgagcacccacccgccccggccctcctcctgggacgcctgtggtgggggacgtggggctgctgggtctggggcttctggaaggtgcctggggtgcggtgggagtgaggagcagtctctgcccttgaagaccctccccgcccatcctggggtgctcacctggcaggtcacggggtggcatctctgttccctggcctggggcagccgcagcctgtccagtgtagggtcgtgcttaggaagttgttccgcaatcattgcctgccacgtccctgggctgctgggtgaggggcctgctctgcgaagcttgatggggagctcggctcctggcgcccctctgctgcctgacctgggctctgtgagtctggtgggacctgtgcctccgcctgccctttggaggtggagcccgcaccccgcatggcaggaccccgctggtgtcagctggtcttggcaggtggatggtgtggacacagggaccctcactcactgctgagacccccacccccatcctggctcagaacacaggcacgtggtggcaggcgacccggctggcgggggctcggttcccgtagggaccactggggctgtgctggagtcagaggaagcgtctacgttggggtcgggtggggagccaggcaggctgcggggctcgggccgtgctgccccaggggggtcccggggggtctctttggccacggcggttctgacttgtgctgtcttgtccgacaccccatctcactgcctcctgcccccgtgctggaggaggagcccccaacaccggctgccccctcatgcctgtgcgggctcccccgtgcctgttcgggctccccctcctcttctcaagcacacgtgtccgcgtgtttttggtgcatgtctgtcctgtcgggtctcggtcagtgtctgtgggtctcgagctcactgtgggcctgtccgcctgtccgacctcgggtcacagggccttctcacgcagtgtggagctccctgtggctgatggggcaggaaggaagcccagtgccagctgcggaccccagggggaggtgcagctgcacgggtcccccacaaagctttgggggtcttggggggctgtgcctgcagcgcctccctgccagctctccccggctctcagaccacgttgccccatacaggagctgccagccacatgtggctgttaaaataacatggattataacgggcacggttagagattcactctcgagtctcaggtgcctggtggcgacagccggagcttcagtgtggctacaggacgatcccattgcagaatcgggcggcagggccggcccgggaggggcctcttggtcaaggcgatcatgctggggtcaaaacaggagaccttgggaggtttggggaagcgggaccctgcctccttcatgctctttccctggggtggggaaggtctcctggggccctccaggcaggggctctgtgcttgggcctgggtggtgctggtgggagtctgaggtctttgcctgccaggaaggcctgggtgtcatcgtagctggggtggtcacagcgggagagactctgagcagccagaacctcaagccaggcgcagggagccggctccctcctgcctcgtcccaccgtcgcgttcgtggttaaccagcaaacagagtccagcctttgcactacatcccttcgtgcctggggaccccaaagtcgcttgtgtactaaaaagccggaccggcacgggggctggggtggaagccggccacccgcccttggagtcttgcaggctggtggggcttggggaagggtccagagccctcagcctacccgggcggctgagtgctgccgtgactggagctagccgtgtctgtgacgccgctcggagagctgcctgcgggtgcgtgcagggacatcggtgtctaaacacacgtgtgcgcaggaagacgggaggctgggtgctttcttcctagagcctgtgtgttctgagtggatgccgtcgccacgctggggttcctggggcaccgagtttgagaagtgctgtctcaggaagcagtggcaaggaggttgggggtggctggggtggggaaaaggaagagttcttgagttcttggtgattggtttcattttttgcttaatcatttgcacattgtgatgcagagctaaatcgccagggtttttggggagattcttgggaaaacgttccgtgttttctgagccttttctaaggaacgccccggcggggagctgcgctcctgaagggcttcacaatgaggggatccactgagccccaacgtgactgtcctcaggggagcccttgaacccccagatagccatgcctgcagtggcagccacgcctcctgggtaaaccgagacccacctccaactgggggactggctggcatggccaaacgtcgccgattggtttgcaaagcgggacaactgcacgttttcctctctcaaatgtgtgtgcttcacacccacgccctttcccggggtcacgtccttcccaagcagggcttctgcggcgtctgaggccacatgtattattgttttgtcagcatgaatactgcaaaaaataatacatcttttaagacatgaacccaaactgtggctttggaaggaaaaggtatttttggaggaaaaaaaaaacagggatttttttccttcagaaatagggaagtgttttcttctccggacggtggagacgatggtgcaccttcctcgctggccttgcttgggcgcaagctctgccgctggggactgcacacaacaccctgggtggttgcagtgcctcagtttccctgctcccggccgtgtgctgttgccggcgcccaggactaactgtgctctcctcatttccagtaaaggcagcccgtgcagagggcccctgtgtggatgctgccccggacgctctaggtaccgcggaacacgccgcacggactgacggctgctgcacggctcctctgcccctccctgcttactagaggttctggaggagccagggggaggctggcgccccaaccccaattctgacccaccgccctggcggtgcgggctcagctccctctattctagccgacagctgcgtgcagggcggagctgggggaaggtgcttcttcttgctccccacgccaggcgatgtcctgcgcacagccagaggggccagccaggggctctgaacccagcaaggcgtccgtgcctcctggggtccccgactgctcttcagggagcgtttcccctggggctgtcactctagcacctctgctgcgtacctgaggatgactgagccttcctggctgcctcgaccgcgggaggcagctccgtctgtgtgtgccgggctcctcagggtcctgatgcatcagaagccccagaaagccagaggcccgcggctccctctcccaacaacaggcctggtttagggcaggtggaaataggacgggggaggagccgccccctttgcaccgtgggctcctggcctgcaccgtgacccgcacctcccctgtccccgcgtcccctgtctgctgttgggtgtgtgcaggcctgcgctgggggcccctccctcctggccagcacagcccagcaaggtggaggtggctgtgtcgtgctcctgtggggctctccttggcatgtgcatgccgccggtggtgctggccttggagggctggtggggctgcatgctgcctatctgcatccgtgcatgcttcggaccctgctccatggggggcccgcgactccatgccaccctctccctgtgactttgctgcttgtgtgcttctgggggcgttggtgcttgcggggagactgttggcagctccagagagggagacgggcgtgtgggctgcctggggcatggcctgctggggcgctagcagctgtcctgcgtgtccggagcatctattcagggtgcttctggccgcaggctggtccctcagggcaggggctgaggtcaggctgcttggggaccagaggggtttgaaagtgggggtgggagcccaggccagagaacaacccagggcaggccctgtcctgctgcgagcagcccccacgggaggccccttcatcctcacgcttcctgggcgtcgttggtccggacaccagctgggcaggggtctggatgctcctcagcctcctgtttcctgcgggcaccgcacatctgaatttcaccattgggagggagggtggcaggccgggtggggatggcgcgctctcaggtgcttcttgagcctgccccggccccgtaagccccaagcgtaggtgccacggacgcccctcgcacacagagggcagctctggccgagggctcctcccaggtgggtggcggcagcttttcgcgtgcgcttgttcttggcttgtggccgagggagggcctgcgagaccacgcgtgctcctggcagctgaggcaggctggcggggtcgggtgtctgcagcatggagcctcaccagctgtgcttggagggtgcagagtttgggtcatggtggaggtccaggggagggaaggacatgtccaggtcgcgccaccctcctcttcctggcagagcctccctcgcagatctgaggtgggagcggagcccgagacccgggacagcagggccgcgccgaggccagggcaggctccttcccgccggctctgtctggggcagtacacgtgggctgcgtcccggcggcccctggggctgtgctgggcatcgggtccagcagggtgtgtctgccgctggcggggctgtggctccttgtcgtggtcagcggggggtcccccctgaaaaatgggaagcagacgtcggggctgggtcttctctcctggggcctgtggaatccacgccactcccttcctgcaactccctgcggactgtggggcctgacccttcctggcccgcacgtctgtgtggccgcctgggtatttgacccgttcccctccggggccgcaggtgggaatctgatgcccattcctcaagctgtgcctttcccactgcaagccctcagcaccgggctctgcccagagaccagccaagggcagccccgaagctacagttgggccgaccggctctgtcctccacccggtgctttgccagctgccgtgagaaaggaggtggctgcttctctctctggtgactggacctgcctcgggtggaaacgcagagagaggaattgagtcttccagtgactcccacttgccctgaggcctgcctgagcggggtcaggaggggcctattcttgcctttgaaatggctcgtgcggtggagaaagagagaacattccaggagtcccgctgagcctgtgcccatgtgctcccctggcccccgtccggttctccccgaccacctgtgcttttgttgttgggatggggaccagaggccgttgggacagaggcacccacagcaccaggactctgagcacccctggcccccatgggcacggtgtgttggcctcaggcagtgcaggggtgtggactcaggactggggtcctgagcgagcctgtaccatcggctcctgacgtccccaagcacgcgacgcctcgggactcgggggatctcggccactccgtctctgcttccccgagcgggcacaacacccaggggcttctttctttctctcagagacgcctgagtggggacagatggtctgctgagtgggcacagatggtctgggcggtcacctgctgcctgggggatggggtggggaggggcagccaagcgatccgcgtgtccaggccaggcagacgggcgcggtgggctctcatgtctgtggctggttatctctgtgccgtctggtgggcgtgtctctctggctgtgggtctggagtcggggctgatggtggctgctgaccggggcctcagtccctcgctgttggctggggagccgccgtgggatcagggagcccgtgaggcactgggcttcctctctgaagtgtgtcagggatctgcggggcctccccacccgaaggcttgtttcaggctccagcaacagtgctctgggccctgcccagaaccaccctgagaagttccgtggctgctaagcatggctgggcatgagcgggtccccagttcgtgagagccccactgtcctcaagggagccagggggttctgatgctttccagacctcaggaggccacatctgcctggcgtctcccaaccaccaaccacagtgaaccctcccctcccctcactgtcgcccggcggacgctggagttgaagaactcgggtgcaaacccatctccacggtcaccaaaaaactcgggggcaaggcaggcccaaggaccccccgagagtatggagctggagacccagccagacacagctgcaaatcctgcttcacgcctcgcaggccgagggggccgggccacagcgctgtggctttggaggctgcctccccctgccctggggcaggacttggatagtgaggcccagctggcggaggagccccgttatccattccttccttccagaagccggaaggagcagttctctgacacggacctggtgggaggggagcctgacaggggaggcctgagccctcctgaagcccatcccaccaggcaggcccccggggtgggtggaaaggccggagtgtggctgaacaagggtccaccgcagacttggcccccaatgtccttgttgtcccctggcccctcgactctggggcagaaggagagggacaggccgtgggaggggaggagggaggctgcttcctgggagcagttacgggtgtgaaaaggccacaggggaaaccacgggcggggccggggcagacagaggtgcccgaggaagagccacagccccggtgtttgggatgcccgttgccctgagtgtcactgggtgaggggctggcaggacagtgtgaggaaggctccggcacctcgtggtggtagcggaaagtgatttaagagtccacagacatcaactctccccacatggccaacctctggccacgcctgagccagccgggggagcttgctgcccacccagcctcccctccctccctcccgccgggtgctcagtgtctgtggcctcccctccctccctcccgccgggtgctcggtccccgtggcctcccctccctccctcccgccgggtgctcggtccccgtggcctcccctccctccctcccgccgggtgctcggccccggtagcctccccccctccctcccgccgggtgctcggtccccgtggcctcccctccctccctcccaccgggtgctcggtccccgtggcctcccctccctccctcccgccgggtgctcggtccccgtggcacctgctctgagccacccgtgtggctggctgttcttcagacctgtgtctccctccggcgcatgggcggcgtcggccatgagcagacacggcctgtgttctttgcagagtgacttctctccctgtttttctgtctgtctgtcggttcccgtgggagcagccagaaggtcagtttgaaagatcgtgtcttctccagcccccgaggcgtggctgccaaggggaaggggtccccgcaggcccagactgtgaggcggtcacccagcgccgaccagagcctcgaggacagccccagcaaggtgcccaagagctggagcttcggggaccgcagccgggcacgccaggctttccgcatcaagggtgccgcgtcacggcagaactcagaaggtgggtgtggccgcatcctctcctggtccatcctctccggggatggatgtggtggccacgctactgtggccaccctgtggcgtcgcagctgcttttgcttgctcttggagtgggaaccccctatcctttctaaaattgaggtgagacttgtgcaacctaaaattaactatttaaaagtgtaggaattcagtggcatgtagcacgctcacggtggagttgtcaccaccacctcagcctagttccagaacattctcatcaccacagaaggaaaccatgaccccatcagtagtcaccccattccccgcctcagccccagcacccgctgctcttgggccgtgcgtgtctgcggctcccacgtggggccgttgggtctggcttccttcactgagcctcagatgtcaggcctgcaccttccgccgttaacatctgtgcgtggatgcgccatgttctgttcatccattggccagaatgtcctttgacgccccatatcggccactccctgtggccacagcctgccggggaaggtcctgactcgagagcagctgggtgcagatgggtgggtttccagaaaagcaggtgcacccgaggggcaccacagggagcctcgaggggctccctggcgctctgtctagccggtgtgccctgcaggaccccgggaataggtctcccggccccctcggcccctcacggcctgtctccttccccccagaagcaagcctccccggagaggacattgtggatgacaagagctgcccctgcgagtttgtgaccgaggacctgaccccgggcctcaaagtcagcatcagagccgtgtggtgaggcccctgcccagccgggagcctgggggagtgaggaggggcctcccgctcggtggtcctgcctgctgccggtgctcatggtgctggacagagccgcccagagacagagtctactgggaaaagaaagggctaagacacgggtggacctggctgaatgtcctggagtgcactcctctgggactcaggcgggtttcgggtgggatgccggttcagcaagaggcctgaggccgggctccacctggcttaggaagcaactcagccaccttctgcctgctgtggcatggagtccaagggggcatggccaggcctgcaagccccacctgtcccgggcagagcagggccacataaaggcccttctgtgtgtggagctggtgggcggcagacaagaggggcaagtccagccatctccaggcgccgaggtctcagaggtgctaggaaggtcctggtgcctgtggccgggggtctctggcccagggctcacagccccacccacccccctgcagtgtcatgcggttcctggtgtccaagcggaagttcaaggagagcctgcggccctacgacgtgatggacgtcatcgagcagtactcagccggccacctggacatgctgtcccgaattaagagcctgcagtccaggcaagagccccgcctgcctgtccagcaggggacaagaacggggtgggcttctgggacaaagcccactgtggcccatggtgggagtgcagggggtgtgtgggcggggcctcctccccacccacgtcggcctctgtcagcttctgttgtgtcttcacaaagtctgttttaatatttctgctgtctctcccatctctcccccatccacaccgcggtgggcaagtgcgtgcacctgtgtgtgtgggtccctgaaaatgtgtgtgtgcatgtgtgtgtgtgtaagtcacgcatgcacacatgtatgcttctgcacaggtgtatgtgtgtggttgtatgcacgtgtggctctgctctgccatgtatgcacgtgtgtggggtgcatacatgtgtgtacacatgtgtgcatgtgtgttggggatggaataattctgttcagacatgaagctgcagggttcccgtgggccccaccaggcatcagttatgctacttggcaccttttcttctacctgccctccttggcataggtgtcccctaagtccacacacgtgcccacacatgtgcagcctcaggcacatacaaacaaggatatacacacgagtgggtgtttatgttgcctgtaatggggacacacggctgttgagagtctctcctgcagtaggggacatgctcctgcctatcagggtcccctccctctcccaggccccccaaccaaggctgtaccagtttctcagccctggaagcgaggctgggcccctgttacagggagaaggctgggatggtcagggacagcctgggggtcacagcccaactccgggccctccagtgggagtgaagctctcatctgaggaagtcgagagccccaccctggggctcctgcccagtcctgcttcagggggccagggaaggggcgcctccaggccttagcccactcctgctgtgcaggggccactgagccatccccttcaccccgctgggctccagctgtgaggcctctttcccagcgtcctctctggagcagcggccaaccctggctgcgtgtgcgtgtgcgtgtgtgtgcggaacgccctcctgtcttgacttctgtgctttgtctccacatctctctcacttcagctgtttttgttcaccaggatctcttgtcctaaattttccctttccttccctctctctctggaaaaacaaacaaaacaaagcaaacaccccaaggaccccaggagtcctcggccggctcctgacaatgcgtttcccgttgccccgccgcggcctgtctggtaccggctcctgctgcgggcacctcccctgcactccacgctctgggacgtggcacccgggtctccgcagtggggccgcccgccacccgctcccgagtcaaaagctttgcttgaaacacgggaaggacttggcaaatccaaggtccagcgaggcggcgtccacaccctctgtgggagagcacctgtttcgacggagcctccctgttgcttccgtttccgccagttcctggccagctcctcatttcctgtggctttacgatgcgtttcgccggtgtctcttttcttgtttactctgccaggatagatatgattgtgggtcccccgcccccttcaactccccggcacaagaagtaccccaccaaaggacccacggcccctccgagagagtcaccccagtactcacctaggttaggatgccagcgcctctccgacgtgtctgtggaatggccggccccttcgctccaggccacatgacggcagcaccttgtaccaccctttccccgggacagaggggacactcgggttttctctgttcctgaaaaagccacaagagggagaaggcccttcctccaccagggccccaggcaggctgtgcccctatcaggactgccaggggccccactgctcctctcgacaagtgccctggggaagccgggggctctgtggatgccctgtgagatttccctctggggttgggaggcccccgggcagcacctccaggatcagatgccatcagacctgcacagacatccctctggccctgcccgtgccccctggacaggcccctcctggctgagggctctccagcctccccaggcactgacctgcccctcagcacctgcccactgtgggggtgggcctggccacgtgcacggcccagacctgggccctgggtggctccgagcaggtggagccgcatctctgtgtctcttccctctcgtggaagatacagacggtcacagcatctaccccttaagccaccgggaagaagcgtgagaagaggcctgccaggtgcccggcacaggcgtgcgctccagcagctccggtgctggctttcaagctgccagcatcctaattagtgtttttgataagggagctggcgagagccccggctgatgggcagaacagaggtgcggcccagctccacgtgagctggcagaagccagttctttagtgattaactcagactttccaaggccttcttgcctcccagggactattcagatgacggtggttccctgtcttctggcaagtctgattggctgagggagggtagagcccctggggcggaggcctggaggctgagcatagacccctggccttccctgacagctcagggcagacagtttctgggtacagccaatgccagtgtctgggcaaatgaagcccaggcctgagctggtggggacggcgtctggggtgagtctgacccctcctccctctgtggtggggtttctgcctgtgaggccaagtgtctgtgcactggacatgtcagccccgtgctcctcccgtgagaacggagtttctgggagcaggcagggccgtgtctgttactgcccaacccagggtagccccggacggagtttgccgggttccctgctctgtggtgtcccccatgcccaagagtgctgtgccctggggctggtcctccgtcctcctgtgccttgccgcaagagacagcagctgggcatccacagctgtgagctcggacggtcttgtggctaggagacgtgcaggttcacacagggggccagggagacagttggagcgcttgccccacctcacggggcactgagcactcagggatcaagtctcctccagccaggggcccagggcaggaggggcacagggtccttggaggagcccccagcagcagctccaggctgagcccgtggccagggctcctattgggactgagcaggcgggcggcccgtcatgtggcaggggcgaaggtgctgagcatctgcctggactgtgtggatgccccgaacaccagggactcccatctcctccatcgccactggcgtctgctcggagccagagggtcccctcccgccgtcccctcccgccctcctggccctattgcatgagtccagcatgaagccccgcggagcatgaggagctgcactgtggccgacgcgagcctgaaaccctctctgtttctcgcaacagaaagaagataacacccctggcccaccctcccccctcccatcagaggcaggtagggcagcccagtggcaggaagtcccgcccccatcagaggcaggtagggcagcccagtggcaggaagtcccgcccccatcagaggcaggtagggcagcccagtggcaggaagtcccgcccccatcagaggcaggtagggcagcccagtggcaggaagtcccgcccccatcagaggcaggtagggcagcccagtggcaggaagtccctcccccaccatcagaggcaggtagggcagcccagtggcaggaagtgaggacttggccccgtcggctggtgtggggcagtcttcatgtaccaggccccagtccaggggagacttgggagcttgtggcctctcctgggtgggtcctgctcagccacaccccgtcttgagctgcccttgtccagctgggcttttgtagacttactgcagggtctttgtttccaagtgagttcccacgtagctgcaggcctcagcgcacacccaccgtggtggcccctgccagcccagcagtggccgtgactctgtggataccagactggtcagaagcccccgtccctgagggcaaaggagcctccctcccttcttattgtcatgagtgacctgtggcctcattggagatcagctcagctgaattctgcatcctacacacacacatgcacacacgcaaacgtgcacactcttgcacacatgaacacatgcacacgcgtgcacacagaaacttgtgcaatgcacatgagggcacacacatgcacatcgagtgcacacacacgtgcacacacagaccctcgcagctttgtctcccctcttctggtttgtgcgttggttcctgtgctgctctgggccttctctccttgtcctggtgcatggagccgcagctctcacgccttttctgcttctgttgtccccgccgcagacatttaactccgtccccatcagacccggtggggccgacggcttcagtggcacattacgtaggagtaaagctccggggtgggcacgggtagggggaggggcatcctctggagtgggctccaggccctgcagtgggagaccctccgtgggctgtggggccaataggccttgggcagggaagagggtgagcaggtggggctggctggagggggcggtatggagccgggagcccacggagaggagccgggcgactcggccctggtgggagcgtccttactttactcctcgctaatgtgccaggccccttggggacagagcgggcaggagcagaacctcacggtgccagaagacgggctgagaacggggcgcaccccgtccatctgtcctggcacccactgccccctggtcggctctgggctgagcctgcagcctagacccaggggcactgtggcctggtccaggaggagagggcaggggccctgctgcacccacccgaactcatgctttggggtctctgttcccggtagagtggaccagatcgtggggcggggcccagcgatcacggacaaggaccgcaccaagggcccggccgaggcggagctgcccgaggaccccagcatgatgggacggctcgggaaggtggagaagcaggtgcgagaggctgggcgggaggggtgcggggctggagggctgtggtgcctgccgtcaacctgtggcagtgtgggcttcaggggctgggtgccacggagggctcctcaagtctagggtttacatggccctgtcaccactgacagctgctctgtgcagacccctcacaccccaccgtgagcctcccacccccggcctgggtgccttccctcctgacagtacccaagggccaaagtccccaggagtcctgggtgggggctgctgagaggtggatccctcaggggttccttgtggaacgtgccggaagttgtacctgcctggccttcctcctctctgtcctccttgcccgacaccctggccgctgtcttctgggggtgcattctcaggaaccaccgcagaggacccttgttttggggcctgcttctgagagccgggcctgagcagtgagtaaggaactgccaggtcctgccctgtggggctttcctgaagggggttccacggtgggcagcaggagggcctggctctgaacacacgccccaaccgtggagctggaggctttgcccctaggggtctgggaaggaggaagagctgctcctgggcctcctcttcccgctcctccagcttccagggagcagggatcccccctccctgtacctctccagggagcaacccaggtccccccacctcctgggaccagcccaagtcgaccgacttcctgggagtggtccaggtccccccatctcctaggaccagcctgggtcccccccatttcctgggactagcctgggtcccccatttcctgggactagcctgggtctctcctgtttcctggaaccagcctgggcccccccatttcctgggagcagcccgggtccccacatctcctgggactagcctgggtcccccgtttcctgggactagcctgggtctctcctgtttcctggaaccagcctgggcccccccatttcctgggagcagcctgggtccccacatctcctgggacgggccctcgcaccccagcccagcagccccttttgcaggtcttgtccatggagaagaagctggacttcctggtgaatatctacatgcagcggatgggcatccccccgacagagaccgaggcctactttggggccaaagagccggagccggcgccgccgtaccacagcccggaagacagccgggagcatgtcgacaggcacggctgcattgtcaagatcgtgcgctccagcagctccacgggccagaagaacttctcggcgcccccggccgcgccccctgtccagtgtccgccctccacctcctggcagccacagagccacccgcgccagggccacggcacctcccccgtgggggaccacggctccctggtgcgcatcccgccgccgcctgcccacgagcggtcgctgtccgcctacggcgggggcaaccgcgccagcatggagttcctgcggcaggaggacaccccgggctgcaggccccccgaggggaacctgcgggacagcgacacgtccatctccatcccgtccgtggaccacgaggagctggagcgttccttcagcggcttcagcatctcccagtccaaggagaacctggatgctctcaacagctgctacgcggccgtggcgccttgtgccaaagtcaggccctacattgcggagggagagtcagacaccgactccgacctctgtaccccgtgcgggcccccgccacgctcggccaccggcgagggtccctttggtgacgtgggctgggccgggcccaggaagtgaggcggcgctgggccagtggacccgcccgcggccctcctcagcacggtgcctccgaggttttgaggcgggaaccctctggggcccttttcttacagtaactgagtgtggcgggaagggtgggccctggaggggcccatgtgggctgaaggatgggggctcctggcagtgaccttttacaaaagttattttccaacaggggctggagggctgggcagggccctgtggctccaggagcagcgtgcaggagcaaggctgccctgtccactctgctcagggccgcggccgacatcagcccggtgtgaggaggggcgggagtgatgacggggtgttgccagcgtggcaacaggcggggggttgtctcagccgagcccaggggaggcacaaagggcaggcctgttccctgaggacctgcgcaaagggcgggcctgtttggtgaggacctgcggccttgggtcccggtggggtttccgggcagctacaggcgggtgtggccggccgctgtgcgtggcctctgccttcacacctgacctgcccggcgggctttcctgttccccacctcaggggcgcccaaatacagagctattggttggcgtcttctccctgtaccttctgggatctgagggctctttccatggaagccagccccgaggtggagaccttcgcctgcagccgaggagcgggtggggcctgggaaccaaactggagccagagtggacgtccagccctctggtcttggcctccagagggagggcctggctcacggtggggccagggagccggctccaaagggtcttcaaaaagggggtccttgggggctccagctgcctcgccctggcctttctgtgggtgcgtgagagccagcagcaccccagccttggagaccgggggggcaggaccccaagtcctcccctctctcctgactgccctggccgggtgccggcactgcgagacccacctggtgagcaggcctcacagttcttagccagggccccacctcgcctgtgtcccaccagtgccccgacagacctggggcagggctgggccatgatgcagcgggccaggatagcctccaccgtcagcacagggccgccctccccgcctttccggaggaaaccactcccacctcagcccagctgtgcgccctccctagctctcctgccccctggagctgatggccccttctccactgaccgattccttagcggggcctcttggggtctcgggcctcgggtgcaccgtcccatgcccgtcctgttgtgggcaccgtggcccttggggcaggcggctctaatgcgggagcgagtccctagctccagacttaagaaccagaccccgggagcatctggcatttggcgtgacggcgtcgcaggcgggcctgggctccctggagagtggcctccctgggagtgagcagggctggggtcgtgggcgcaaatactcctgcagagcaagtgcaggggagtcctgggcccgtttctcctccacctgcgttttcagtgcacttggcttggctgggaggtcctgaggccctgaggccagcaggggaaccagtcctgagggagaggactttgaaagcagcatttgagggtcgtacgcccctggctggtgggggtcctggcgctcagggtgttcggggagccatgtctggcgtccattgtggggagctgctgccctggcctctctgcctacccccagcccggccagggcactcccaggccctgtcgccattgaggtgcctccgctgggctgtctcctcacccctccctgtgctggagcctgtcccaaaaaggtgccaactgggaggcctcggaagccactgtccaggctcccactgcctgtctgctctgttcccaaaggcagcgtgtgtggcctcgggccctgcggtggcatgaagcatcccttctggtgtgggcatcgctacgtgttttgggggcagcgtttcacggcggtgcccttgctgtctcccttgggctggctcgagcctggggtccatgtccctttgccgtcccgtcatggggcagggaatccatagcggggcccacaggcaggggtatgagtgcgtcccacccaacgcagcaccagccccggccaccgctccccgtgtccccagttccgtctcagctacctggactccaggaccctggagaagggagacctggcagtggagggaggctgtgctgtgtgtccccctgcaggtgtgaccccgcctgctctttcctcccccgccaggtgtggccccgcctgctctttcctcccccaccagtatggccccacctgctctttcctccccccccaaggtgtggccccacctgttctttcctcccctgccgaggtgtgaccccacctgctctttcctccctcccagtatggccccacctgctctttcctcccccgaggtgaggccccgcctgctctttcctcccatggggccgctgaggcatgagcacctgggcacaggttggggctctgcaggatgaggaagacaggccaatcccttccctcccagaagctggccgcccagcaggagggactgaggccagactcatgtccagcaaggaacgtgtggtgtgtcccctgggaagtctctgggccctgggaagagggaaggtgcacgtcctgggatggttgcggggccctgttttgggagacaaaggggtagagggtctgtcttgggcccccccagactctagcctgagcagtgcagccacctactgccccacctcagagaagtgcagcgggaaggaggctggaggtggtgcggcgctgcctcgggtgtctgcgtgaatgagcgtggccaaggaccagtgccacctcatggcaaagagctcccgcagtgtttgttagagtgcacatccctacgtgcccactggcacacacacgtgctcacatacatgtccgcatacaggcgtacacatgcacgcttgcacacatgcacacagaccacatagcacacatgtgcactgaccacacctgtatagaccatgcacagtacacatacgtgcatacacatgcctgcatacaggcatacacatgcacgcttacatgtacacgtgcacagatcacacacatgcacacacgtgtagctcacacacagtatacacatacacaagtgcacagaccacacacagcactaacacatgcacacacaaagtgcataggccacacagcacatgcacacaggtgcacagaccacacagcacacacaagtgcacagagcacactgcacacatgcacacacacacgcgtgcatgcacactcctcgcacttccagccttggagcccttctgtctctggtctttctctttgaccctgctgagtgtaagctgcctggggaggggctacaaggagtaattgtggctttaggggtcgtggtgatgctggaatgtcaagcgccgtcgtggggtatccgactgtccgggctcctggtccgcagtggcagagcgccaggcagagcccaatcagggtctcgtgctgcccttccctcccacagcctggcagccatccagaggaggggctctaccagatgccaaggtgccccggtgtctgtatgggtgtccggttgggtcctgtgtttggtctgccctggaggtgctgggccctcctgggatgggtggctcagcctcgaatcccaggccccagcccaggcaggtgctgctgcctgttgtggtttcctggcccagcttctccttctccctctgcataaaatcacagtccgtgagtcttccagctgccaccacggctgggacacgctgggggagggctcctcccatgcctcctgcacacagccgtctgagcagggcaggtgcccaacaccccccaccggaggacacgctgcccctcagcgatgcccctaccttttggggggcctcgtctcaagcccccccttggaggctgaaatcaccccaggcactgtgagggcttctccagggggcaccctttgagctgtgggtctgatcaccccaagtcccgcccggaggagaggcacagccagggcgtgtggtttaatgtttgcccccttcggggctggaggtctcagtgtttctagattccagaccctgctgccagagagacctgctgccggagagaaggggaggaggactccagctgggctcggtcccccacagtcagggacccccataaaggacacccccttctctctagaaagagctgggctctcagctatttctagttgcttcccagaagccgaggagcagaaggagctgtgagagctttgcagaaacgcccttgtccccgccctcctgagctatgaatgccgtacagagcagaggctggggcattggcaagatcacaggttgatgctgcacagccccattgacacaaaccctcaaagcagacgtgagagggacggttcacaaagctcggacctgccgtggagggtgcccggcagacgtggcgtgagagggacggctcacgaggcttggacctgctgtggagggtgcccagcagacgtggtgtgagaggaacggctcacgagacttggacctggtggagggtgcccagcagacgtggtgtgagagggacggctcacagggcttggacctgccatggagggtgcccggcagacgtggtgtgagagggatggttcacagggcttggacctgccatggagggtgcccggcagatgtggtgggagagagatggctcatgaggcttggacctgccgtggagggtgcccagcagacgtggtatgagagggatggctcacgaggcttggacctggtggagggtgcccggcagacgtgtgagagggacggttcacaaggcttggacctgccatggagggtgcccagcagacgtggtgtgagagggacagctcacgaggcttggacctgctgtggagggtgcccagcagggggctgagctctgaggggtgggtgctcagtgcacgggtgcccccagtgtcctctgatcctgtccggtgcctcccccaacccccacacccatgcagaactcccaggtcacatgcacgtatgtccagggcatgggggtggcgtgaagaggcctggtcagggcctttaggggctgcaggacggaatggccgcctggggagcctgtgtggctgtgccgggcagccatcctgcattcccacccagcgcgcagtctccacctcggccccagcaaagcgctaagcagccggagagacagccagggcggcttcctgaaggatgtgggatggtggactccggggtcgagggaatacgcaggttcctgtccctccgggagacctagagaagctgcacacccaggagctttccatgacccgggagcatgagtgaatgggggttccagtttgctgaactttgccgtcttgtaagggtgggggctgacggccgaccctgggaggaggtgacatcgccgggggaggttgtgggcaacggtggaggaggagagacgggaggggaccatttgggatggaggggcctcttcagagttttaaaaggcgtttgtggggtggagttgagtgtgctctgggcttggacacttgccgtggtgcccctggctggccgaggagactggctctggccaggggccccgtcctgagaggtcctcagcgtctgactctcggccaggcgccagcaaggaggggccggtccccggggctaccaggcaggcacgtgcacatcgccatcgccacacgccaactccgcctgggttttacaaagtcgttgccttaatgcatgtggacaggaactccctgaggtcgccccatgccccctggctgtgccaggtacggacgccctggaccctgcgaacaggtggggcgggcgaggggcccaagggacgggctccagagacacgcgcagggcaggaggggtctcacggaggggtctcgcactgaggcgcccagagctggtggtcccgctggacgccatccctctgcccgggatccacacggcccacgtgtgcccgccatgcccgcgccccacgccattgcagtctgccatcctctggccgtgacggtggctgcagcttccccatttgcgccgttgcctctggctgtctgcacttttgttcatgctccaaagaacatttcataatgccttcagtaccgacgtacacttctgaccattttgtatgtgtccttgtgccgtagtgaccaggcctttttttggtggatgtgttaccccgcacacttcaatctcaactttgtgcaccgtccattttctagggatagacgcccagggaatgaactctagttttctaacagattagctgagatattaacttactcacacggacaggttgatgccagagccgtaagaatgcgccagtgcgggtttgcgggggacttcgggtgtggggtcctgcggccgcgatggccgtggaaggttctggggatccctgctgccacggggacgagttcggacgccaggtggacctgtgcactcagtaaaacgcagtgattcaacctg

SEQ ID NO: 3-10 - skipped (8 entries)

SEQ ID NO: 11 - DNA, 18 bp, Artificial sequence
  Primer
ggtttgccctgaaggttc

SEQ ID NO: 12 - DNA, 20 bp, Artificial sequence
  Primer
gaggttggtggcgtagaatc

SEQ ID NO: 13 - DNA, 24 bp, Artificial sequence
  Probe
tctcaaagtgcttctgcctgtgct

SEQ ID NO: 14-20 - skipped (7 entries)

SEQ ID NO: 21 - DNA, 20 bp, Artificial sequence
  Synthetic oligonucleotide
ggaggaaagctgaggccacc

SEQ ID NO: 22 - DNA, 20 bp, Artificial sequence
  Synthetic oligonucleotide
cccacgggaaccgacagaca

SEQ ID NO: 23 - DNA, 20 bp, Artificial sequence
  Synthetic oligonucleotide
cgaagaaggagacaccgatg

SEQ ID NO: 24 - DNA, 20 bp, Artificial sequence
  Synthetic oligonucleotide
aggccaccttgaggcctggg

SEQ ID NO: 25 - DNA, 20 bp, Artificial sequence
  Synthetic oligonucleotide
tactgcaggcagcgcgaaga

SEQ ID NO: 26 - DNA, 20 bp, Artificial sequence
  Synthetic oligonucleotide
gaccgctcgtgggcaggcgg

SEQ ID NO: 27 - DNA, 20 bp, Artificial sequence
  Synthetic oligonucleotide
cagcgaccgctcgtgggcag

SEQ ID NO: 28 - DNA, 20 bp, Artificial sequence
  Synthetic oligonucleotide
gcaggcggcggcgggatgcg

SEQ ID NO: 29 - DNA, 20 bp, Artificial sequence
  Synthetic oligonucleotide
tcgtgggcaggcggcggcgg

SEQ ID NO: 30 - DNA, 20 bp, Artificial sequence
  Synthetic oligonucleotide
cgcgaagaaggagacaccga

SEQ ID NO: 31 - DNA, 20 bp, Artificial sequence
  Synthetic oligonucleotide
acagaatctactccaggcaa

SEQ ID NO: 32 - DNA, 20 bp, Artificial sequence
  Synthetic oligonucleotide
aagctgaggccaccttgagg

SEQ ID NO: 33 - DNA, 20 bp, Artificial sequence
  Synthetic oligonucleotide
ttcaagtgtttccacacaca

SEQ ID NO: 34 - DNA, 20 bp, Artificial sequence
  Synthetic oligonucleotide
tcatgttttttccaaaccta

SEQ ID NO: 35 - DNA, 20 bp, Artificial sequence
  Synthetic oligonucleotide
acacacacttcacatcctcg

SEQ ID NO: 36 - DNA, 20 bp, Artificial sequence
  Synthetic oligonucleotide
ccgacagacagacagaaaaa

SEQ ID NO: 37 - DNA, 20 bp, Artificial sequence
  Synthetic oligonucleotide
gtcactttttaaaccgtcaa

SEQ ID NO: 38 - DNA, 20 bp, Artificial sequence
  Synthetic oligonucleotide
ccggagtccaccatcccaca

SEQ ID NO: 39 - DNA, 20 bp, Artificial sequence
  Synthetic oligonucleotide
tggttataatcctttcttct

SEQ ID NO: 40 - DNA, 20 bp, Artificial sequence
  Synthetic oligonucleotide
tctcatttacatttttcgca

SEQ ID NO: 41 - DNA, 20 bp, Artificial sequence
  Synthetic oligonucleotide
aggaaagctgaggccacctt

SEQ ID NO: 42 - DNA, 20 bp, Artificial sequence
  Synthetic oligonucleotide
gtctcatttacatttttcgc

SEQ ID NO: 43 - DNA, 20 bp, Artificial sequence
  Synthetic oligonucleotide
gtcgctgtcccgcaggttcc

SEQ ID NO: 44 - DNA, 20 bp, Artificial sequence
  Synthetic oligonucleotide
gctgaggccaccttgaggcc

SEQ ID NO: 45 - DNA, 20 bp, Artificial sequence
  Synthetic oligonucleotide
cgctgtcccgcaggttcccc

SEQ ID NO: 46 - DNA, 20 bp, Artificial sequence
  Synthetic oligonucleotide
tccgactcttttttccacgc

SEQ ID NO: 47 - DNA, 20 bp, Artificial sequence
  Synthetic oligonucleotide
gtactggttccactcttgaa

SEQ ID NO: 48 - DNA, 20 bp, Artificial sequence
  Synthetic oligonucleotide
ccatgattgctcttcccatt

SEQ ID NO: 49 - DNA, 20 bp, Artificial sequence
  Synthetic oligonucleotide
gtgggcaggcggcggcggga

SEQ ID NO: 50 - DNA, 20 bp, Artificial sequence
  Synthetic oligonucleotide
gcaggcagcgcgaagaagga

SEQ ID NO: 51 - DNA, 20 bp, Artificial sequence
  Synthetic oligonucleotide
acgcgattacctcactcact

SEQ ID NO: 52 - DNA, 20 bp, Artificial sequence
  Synthetic oligonucleotide
ctcccacgggaaccgacaga

SEQ ID NO: 53 - DNA, 20 bp, Artificial sequence
  Synthetic oligonucleotide
acacacattttcagggaccc

SEQ ID NO: 54 - DNA, 20 bp, Artificial sequence
  Synthetic oligonucleotide
gtcccgcaggttcccctcgg

SEQ ID NO: 55 - DNA, 20 bp, Artificial sequence
  Synthetic oligonucleotide
cgggaaccgacagacagaca

SEQ ID NO: 56 - DNA, 20 bp, Artificial sequence
  Synthetic oligonucleotide
gagccatttctcaacacaca

SEQ ID NO: 57 - DNA, 20 bp, Artificial sequence
  Synthetic oligonucleotide
aagaaggagacaccgatgag

SEQ ID NO: 58 - DNA, 20 bp, Artificial sequence
  Synthetic oligonucleotide
gtccgtattttcaaggtggc

SEQ ID NO: 59 - DNA, 20 bp, Artificial sequence
  Synthetic oligonucleotide
ctgtccatgcatttcctacc

SEQ ID NO: 60 - DNA, 20 bp, Artificial sequence
  Synthetic oligonucleotide
gctcgtgggcaggcggcggc

SEQ ID NO: 61 - DNA, 20 bp, Artificial sequence
  Synthetic oligonucleotide
ttgcaatttcctttccagtc

SEQ ID NO: 62 - DNA, 20 bp, Artificial sequence
  Synthetic oligonucleotide
tcatgtccatcttacatcga

SEQ ID NO: 63 - DNA, 20 bp, Artificial sequence
  Synthetic oligonucleotide
ctgtcccgcaggttcccctc

SEQ ID NO: 64 - DNA, 20 bp, Artificial sequence
  Synthetic oligonucleotide
ggaaccgacagacagacaga

SEQ ID NO: 65 - DNA, 20 bp, Artificial sequence
  Synthetic oligonucleotide
gccagatttactctgcaaca

SEQ ID NO: 66 - DNA, 20 bp, Artificial sequence
  Synthetic oligonucleotide
gcacaagtctcacctcaatt

SEQ ID NO: 67 - DNA, 20 bp, Artificial sequence
  Synthetic oligonucleotide
gtacaatgtcttatcactcc

SEQ ID NO: 68 - DNA, 20 bp, Artificial sequence
  Synthetic oligonucleotide
ctgcaggcagcgcgaagaag

SEQ ID NO: 69 - DNA, 20 bp, Artificial sequence
  Synthetic oligonucleotide
gctgctcccacgggaaccga

SEQ ID NO: 70 - DNA, 20 bp, Artificial sequence
  Synthetic oligonucleotide
aaccgacagacagacagaaa

SEQ ID NO: 71 - DNA, 20 bp, Artificial sequence
  Synthetic oligonucleotide
tgaggccaccttgaggcctg

SEQ ID NO: 72 - DNA, 20 bp, Artificial sequence
  Synthetic oligonucleotide
caaccatgctctcctatgca

SEQ ID NO: 73 - DNA, 20 bp, Artificial sequence
  Synthetic oligonucleotide
atggaatgattctcttccgc

SEQ ID NO: 74 - DNA, 20 bp, Artificial sequence
  Synthetic oligonucleotide
ggtcactttttaaaccgtca

SEQ ID NO: 75 - DNA, 20 bp, Artificial sequence
  Synthetic oligonucleotide
gtagcagattcatctcccca

SEQ ID NO: 76 - DNA, 20 bp, Artificial sequence
  Synthetic oligonucleotide
ccatgcatttcctacctgga

SEQ ID NO: 77 - DNA, 20 bp, Artificial sequence
  Synthetic oligonucleotide
gctgactccattgtccctca

SEQ ID NO: 78 - DNA, 20 bp, Artificial sequence
  Synthetic oligonucleotide
gtgtcgctgtcccgcaggtt

SEQ ID NO: 79 - DNA, 20 bp, Artificial sequence
  Synthetic oligonucleotide
caggattatctccatctcaa

SEQ ID NO: 80 - DNA, 20 bp, Artificial sequence
  Synthetic oligonucleotide
ccgctcgtgggcaggcggcg

SEQ ID NO: 81 - DNA, 20 bp, Artificial sequence
  Synthetic oligonucleotide
actcactgatccttcttcaa

SEQ ID NO: 82 - DNA, 20 bp, Artificial sequence
  Synthetic oligonucleotide
atgtcttatcactccagagt

SEQ ID NO: 83 - DNA, 20 bp, Artificial sequence
  Synthetic oligonucleotide
ctcatgttttttccaaacct

SEQ ID NO: 84 - DNA, 20 bp, Artificial sequence
  Synthetic oligonucleotide
tccgtattttcaaggtggct

SEQ ID NO: 85 - DNA, 20 bp, Artificial sequence
  Synthetic oligonucleotide
tggagaccagcattcaacca

SEQ ID NO: 86 - DNA, 20 bp, Artificial sequence
  Synthetic oligonucleotide
cacgggaaccgacagacaga

SEQ ID NO: 87 - DNA, 20 bp, Artificial sequence
  Synthetic oligonucleotide
gggcaggcggcggcgggatg

SEQ ID NO: 88 - DNA, 20 bp, Artificial sequence
  Synthetic oligonucleotide
cgtgactttttctcagccct

SEQ ID NO: 89 - DNA, 20 bp, Artificial sequence
  Synthetic oligonucleotide
gcagcgcgaagaaggagaca

SEQ ID NO: 90 - DNA, 20 bp, Artificial sequence
  Synthetic oligonucleotide
tgctcccacgggaaccgaca

SEQ ID NO: 91 - DNA, 20 bp, Artificial sequence
  Synthetic oligonucleotide
aggcagcgcgaagaaggaga

SEQ ID NO: 92 - DNA, 20 bp, Artificial sequence
  Synthetic oligonucleotide
gcgaccgctcgtgggcaggc

SEQ ID NO: 93 - DNA, 20 bp, Artificial sequence
  Synthetic oligonucleotide
acacgatctttcaaactgac

SEQ ID NO: 94 - DNA, 20 bp, Artificial sequence
  Synthetic oligonucleotide
tgagaacatttttacagcca

SEQ ID NO: 95 - DNA, 20 bp, Artificial sequence
  Synthetic oligonucleotide
agcgcgaagaaggagacacc

SEQ ID NO: 96 - DNA, 20 bp, Artificial sequence
  Synthetic oligonucleotide
gaaagctgaggccaccttga

SEQ ID NO: 97 - DNA, 20 bp, Artificial sequence
  Synthetic oligonucleotide
gccttgcaatttcctttcca

SEQ ID NO: 98 - DNA, 20 bp, Artificial sequence
  Synthetic oligonucleotide
cgacagaatctactccaggc

SEQ ID NO: 99 - DNA, 20 bp, Artificial sequence
  Synthetic oligonucleotide
gattgactccactgtgtaac

SEQ ID NO: 100 - DNA, 20 bp, Artificial sequence
  Synthetic oligonucleotide
attgactccactgtgtaact

SEQ ID NO: 101 - DNA, 20 bp, Artificial sequence
  Synthetic oligonucleotide
ttgactccactgtgtaacta

SEQ ID NO: 102 - DNA, 20 bp, Artificial sequence
  Synthetic oligonucleotide
gactccactgtgtaactaag

SEQ ID NO: 103 - DNA, 20 bp, Artificial sequence
  Synthetic oligonucleotide
ctccactgtgtaactaagca

SEQ ID NO: 104 - DNA, 20 bp, Artificial sequence
  Synthetic oligonucleotide
ggagggggggaggctaccgg

SEQ ID NO: 105 - DNA, 20 bp, Artificial sequence
  Synthetic oligonucleotide
tccactgtgtaactaagcag

SEQ ID NO: 106 - DNA, 20 bp, Artificial sequence
  Synthetic oligonucleotide
ggccagaggcccctaaaacc

SEQ ID NO: 107 - DNA, 20 bp, Artificial sequence
  Synthetic oligonucleotide
gccagaggcccctaaaaccc

SEQ ID NO: 108 - DNA, 20 bp, Artificial sequence
  Synthetic oligonucleotide
ccagaggcccctaaaaccct

SEQ ID NO: 109 - DNA, 20 bp, Artificial sequence
  Synthetic oligonucleotide
cagaggcccctaaaaccctg

SEQ ID NO: 110 - DNA, 20 bp, Artificial sequence
  Synthetic oligonucleotide
agaggcccctaaaaccctgg

SEQ ID NO: 111 - DNA, 20 bp, Artificial sequence
  Synthetic oligonucleotide
gaggcccctaaaaccctgga

SEQ ID NO: 112 - DNA, 20 bp, Artificial sequence
  Synthetic oligonucleotide
aggcccctaaaaccctggac

SEQ ID NO: 113 - DNA, 20 bp, Artificial sequence
  Synthetic oligonucleotide
ggcccctaaaaccctggacc

SEQ ID NO: 114 - DNA, 20 bp, Artificial sequence
  Synthetic oligonucleotide
gcccctaaaaccctggaccc

SEQ ID NO: 115 - DNA, 20 bp, Artificial sequence
  Synthetic oligonucleotide
gagggggggaggctaccggg

SEQ ID NO: 116 - DNA, 20 bp, Artificial sequence
  Synthetic oligonucleotide
cccctaaaaccctggacccc

SEQ ID NO: 117 - DNA, 20 bp, Artificial sequence
  Synthetic oligonucleotide
tggcgcaaaagccaggatgg

SEQ ID NO: 118 - DNA, 20 bp, Artificial sequence
  Synthetic oligonucleotide
gcgcaaaagccaggatggca

SEQ ID NO: 119 - DNA, 20 bp, Artificial sequence
  Synthetic oligonucleotide
cgcaaaagccaggatggcac

SEQ ID NO: 120 - DNA, 20 bp, Artificial sequence
  Synthetic oligonucleotide
gcaaaagccaggatggcaca

SEQ ID NO: 121 - DNA, 20 bp, Artificial sequence
  Synthetic oligonucleotide
aaaagccaggatggcacagc

SEQ ID NO: 122 - DNA, 20 bp, Artificial sequence
  Synthetic oligonucleotide
aagccaggatggcacagcga

SEQ ID NO: 123 - DNA, 20 bp, Artificial sequence
  Synthetic oligonucleotide
agggcccagagcactgttgc

SEQ ID NO: 124 - DNA, 20 bp, Artificial sequence
  Synthetic oligonucleotide
agccaggatggcacagcgag

SEQ ID NO: 125 - DNA, 20 bp, Artificial sequence
  Synthetic oligonucleotide
cgggccagaggacatggggc

SEQ ID NO: 126 - DNA, 20 bp, Artificial sequence
  Synthetic oligonucleotide
ggccagaggacatggggcca

SEQ ID NO: 127 - DNA, 20 bp, Artificial sequence
  Synthetic oligonucleotide
gccagaggacatggggccat

SEQ ID NO: 128 - DNA, 20 bp, Artificial sequence
  Synthetic oligonucleotide
ccagaggacatggggccatc

SEQ ID NO: 129 - DNA, 20 bp, Artificial sequence
  Synthetic oligonucleotide
agaggacatggggccatcag

SEQ ID NO: 130 - DNA, 20 bp, Artificial sequence
  Synthetic oligonucleotide
aggacatggggccatcaggc

SEQ ID NO: 131 - DNA, 20 bp, Artificial sequence
  Synthetic oligonucleotide
ggacatggggccatcaggcc

SEQ ID NO: 132 - DNA, 20 bp, Artificial sequence
  Synthetic oligonucleotide
acggagggtctgctaagggc

SEQ ID NO: 133 - DNA, 20 bp, Artificial sequence
  Synthetic oligonucleotide
ggagggtctgctaagggcac

SEQ ID NO: 134 - DNA, 20 bp, Artificial sequence
  Synthetic oligonucleotide
gagggtctgctaagggcaca

SEQ ID NO: 135 - DNA, 20 bp, Artificial sequence
  Synthetic oligonucleotide
agggtctgctaagggcacac

SEQ ID NO: 136 - DNA, 20 bp, Artificial sequence
  Synthetic oligonucleotide
ggtctgctaagggcacactc

SEQ ID NO: 137 - DNA, 20 bp, Artificial sequence
  Synthetic oligonucleotide
tctgctaagggcacactcaa

SEQ ID NO: 138 - DNA, 20 bp, Artificial sequence
  Synthetic oligonucleotide
ggcccagagcactgttgctg

SEQ ID NO: 139 - DNA, 20 bp, Artificial sequence
  Synthetic oligonucleotide
ctgctaagggcacactcaag

SEQ ID NO: 140 - DNA, 20 bp, Artificial sequence
  Synthetic oligonucleotide
caggaggcagccgttccctg

SEQ ID NO: 141 - DNA, 20 bp, Artificial sequence
  Synthetic oligonucleotide
ggaggcagccgttccctgag

SEQ ID NO: 142 - DNA, 20 bp, Artificial sequence
  Synthetic oligonucleotide
gaggcagccgttccctgagc

SEQ ID NO: 143 - DNA, 20 bp, Artificial sequence
  Synthetic oligonucleotide
aggcagccgttccctgagcc

SEQ ID NO: 144 - DNA, 20 bp, Artificial sequence
  Synthetic oligonucleotide
ggcagccgttccctgagcct

SEQ ID NO: 145 - DNA, 20 bp, Artificial sequence
  Synthetic oligonucleotide
gcagccgttccctgagcctc

SEQ ID NO: 146 - DNA, 20 bp, Artificial sequence
  Synthetic oligonucleotide
agccgttccctgagcctcgc

SEQ ID NO: 147 - DNA, 20 bp, Artificial sequence
  Synthetic oligonucleotide
gcccagagcactgttgctgg

SEQ ID NO: 148 - DNA, 20 bp, Artificial sequence
  Synthetic oligonucleotide
gccgttccctgagcctcgcc

SEQ ID NO: 149 - DNA, 20 bp, Artificial sequence
  Synthetic oligonucleotide
cccagagcactgttgctgga

SEQ ID NO: 150 - DNA, 20 bp, Artificial sequence
  Synthetic oligonucleotide
cagagcactgttgctggagc

SEQ ID NO: 151 - DNA, 20 bp, Artificial sequence
  Synthetic oligonucleotide
gagcactgttgctggagcct

SEQ ID NO: 152 - DNA, 20 bp, Artificial sequence
  Synthetic oligonucleotide
aaatgggggggcccaggctg

SEQ ID NO: 153 - DNA, 20 bp, Artificial sequence
  Synthetic oligonucleotide
agcactgttgctggagcctg

SEQ ID NO: 154 - DNA, 20 bp, Artificial sequence
  Synthetic oligonucleotide
ctctctttctccaccgcacg

SEQ ID NO: 155 - DNA, 20 bp, Artificial sequence
  Synthetic oligonucleotide
tctctttctccaccgcacga

SEQ ID NO: 156 - DNA, 20 bp, Artificial sequence
  Synthetic oligonucleotide
ctctttctccaccgcacgag

SEQ ID NO: 157 - DNA, 20 bp, Artificial sequence
  Synthetic oligonucleotide
tctttctccaccgcacgagc

SEQ ID NO: 158 - DNA, 20 bp, Artificial sequence
  Synthetic oligonucleotide
ctttctccaccgcacgagcc

SEQ ID NO: 159 - DNA, 20 bp, Artificial sequence
  Synthetic oligonucleotide
tttctccaccgcacgagcca

SEQ ID NO: 160 - DNA, 20 bp, Artificial sequence
  Synthetic oligonucleotide
ttctccaccgcacgagccat

SEQ ID NO: 161 - DNA, 20 bp, Artificial sequence
  Synthetic oligonucleotide
ctccaccgcacgagccattt

SEQ ID NO: 162 - DNA, 20 bp, Artificial sequence
  Synthetic oligonucleotide
tccaccgcacgagccatttc

SEQ ID NO: 163 - DNA, 20 bp, Artificial sequence
  Synthetic oligonucleotide
ccgctcaggcaggcctcagg

SEQ ID NO: 164 - DNA, 20 bp, Artificial sequence
  Synthetic oligonucleotide
gctcaggcaggcctcagggc

SEQ ID NO: 165 - DNA, 20 bp, Artificial sequence
  Synthetic oligonucleotide
ctcaggcaggcctcagggca

SEQ ID NO: 166 - DNA, 20 bp, Artificial sequence
  Synthetic oligonucleotide
tcaggcaggcctcagggcaa

SEQ ID NO: 167 - DNA, 20 bp, Artificial sequence
  Synthetic oligonucleotide
aggcaggcctcagggcaagt

SEQ ID NO: 168 - DNA, 20 bp, Artificial sequence
  Synthetic oligonucleotide
gcaggcctcagggcaagtgg

SEQ ID NO: 169 - DNA, 20 bp, Artificial sequence
  Synthetic oligonucleotide
atgggggggcccaggctggt

SEQ ID NO: 170 - DNA, 20 bp, Artificial sequence
  Synthetic oligonucleotide
caggcctcagggcaagtggg

SEQ ID NO: 171 - DNA, 20 bp, Artificial sequence
  Synthetic oligonucleotide
cagcttgaggaatgggcatc

SEQ ID NO: 172 - DNA, 20 bp, Artificial sequence
  Synthetic oligonucleotide
gcttgaggaatgggcatcag

SEQ ID NO: 173 - DNA, 20 bp, Artificial sequence
  Synthetic oligonucleotide
cttgaggaatgggcatcaga

SEQ ID NO: 174 - DNA, 20 bp, Artificial sequence
  Synthetic oligonucleotide
ttgaggaatgggcatcagat

SEQ ID NO: 175 - DNA, 20 bp, Artificial sequence
  Synthetic oligonucleotide
gaggaatgggcatcagattc

SEQ ID NO: 176 - DNA, 20 bp, Artificial sequence
  Synthetic oligonucleotide
ggaatgggcatcagattccc

SEQ ID NO: 177 - DNA, 20 bp, Artificial sequence
  Synthetic oligonucleotide
tctgcacccagctgctctcg

SEQ ID NO: 178 - DNA, 20 bp, Artificial sequence
  Synthetic oligonucleotide
tctgcatcccaccgaggatg

SEQ ID NO: 179 - DNA, 20 bp, Artificial sequence
  Synthetic oligonucleotide
tcctaaggtggctccgacca

SEQ ID NO: 180 - DNA, 20 bp, Artificial sequence
  Synthetic oligonucleotide
ctaaggtggctccgaccagg

SEQ ID NO: 181 - DNA, 20 bp, Artificial sequence
  Synthetic oligonucleotide
taaggtggctccgaccaggc

SEQ ID NO: 182 - DNA, 20 bp, Artificial sequence
  Synthetic oligonucleotide
aaggtggctccgaccaggca

SEQ ID NO: 183 - DNA, 20 bp, Artificial sequence
  Synthetic oligonucleotide
ggtggctccgaccaggcatc

SEQ ID NO: 184 - DNA, 20 bp, Artificial sequence
  Synthetic oligonucleotide
tggctccgaccaggcatctc

SEQ ID NO: 185 - DNA, 20 bp, Artificial sequence
  Synthetic oligonucleotide
ggaaatgggggggcccaggc

SEQ ID NO: 186 - DNA, 20 bp, Artificial sequence
  Synthetic oligonucleotide
ctgcacccagctgctctcga

SEQ ID NO: 187 - DNA, 20 bp, Artificial sequence
  Synthetic oligonucleotide
ggctccgaccaggcatctca

SEQ ID NO: 188 - DNA, 20 bp, Artificial sequence
  Synthetic oligonucleotide
tccaggcctccgcctacaaa

SEQ ID NO: 189 - DNA, 20 bp, Artificial sequence
  Synthetic oligonucleotide
ccaggcctccgcctacaaac

SEQ ID NO: 190 - DNA, 20 bp, Artificial sequence
  Synthetic oligonucleotide
caggcctccgcctacaaacc

SEQ ID NO: 191 - DNA, 20 bp, Artificial sequence
  Synthetic oligonucleotide
aggcctccgcctacaaaccg

SEQ ID NO: 192 - DNA, 20 bp, Artificial sequence
  Synthetic oligonucleotide
ggcctccgcctacaaaccgt

SEQ ID NO: 193 - DNA, 20 bp, Artificial sequence
  Synthetic oligonucleotide
cctccgcctacaaaccgtgg

SEQ ID NO: 194 - DNA, 20 bp, Artificial sequence
  Synthetic oligonucleotide
ctccgcctacaaaccgtggt

SEQ ID NO: 195 - DNA, 20 bp, Artificial sequence
  Synthetic oligonucleotide
tccgcctacaaaccgtggtg

SEQ ID NO: 196 - DNA, 20 bp, Artificial sequence
  Synthetic oligonucleotide
ggcgggagggagggggggag

SEQ ID NO: 197 - DNA, 20 bp, Artificial sequence
  Synthetic oligonucleotide
ccgcctacaaaccgtggtgt

SEQ ID NO: 198 - DNA, 20 bp, Artificial sequence
  Synthetic oligonucleotide
gggaagaggactcgacgcag

SEQ ID NO: 199 - DNA, 20 bp, Artificial sequence
  Synthetic oligonucleotide
gaagaggactcgacgcagga

SEQ ID NO: 200 - DNA, 20 bp, Artificial sequence
  Synthetic oligonucleotide
aagaggactcgacgcaggag

SEQ ID NO: 201 - DNA, 20 bp, Artificial sequence
  Synthetic oligonucleotide
agaggactcgacgcaggagg

SEQ ID NO: 202 - DNA, 20 bp, Artificial sequence
  Synthetic oligonucleotide
aggactcgacgcaggagggg

SEQ ID NO: 203 - DNA, 20 bp, Artificial sequence
  Synthetic oligonucleotide
gactcgacgcaggaggggtc

SEQ ID NO: 204 - DNA, 20 bp, Artificial sequence
  Synthetic oligonucleotide
actcgacgcaggaggggtct

SEQ ID NO: 205 - DNA, 20 bp, Artificial sequence
  Synthetic oligonucleotide
ggagggaaggcgtcaccacc

SEQ ID NO: 206 - DNA, 20 bp, Artificial sequence
  Synthetic oligonucleotide
agggaaggcgtcaccacctc

SEQ ID NO: 207 - DNA, 20 bp, Artificial sequence
  Synthetic oligonucleotide
gggaaggcgtcaccacctca

SEQ ID NO: 208 - DNA, 20 bp, Artificial sequence
  Synthetic oligonucleotide
ggaaggcgtcaccacctcac

SEQ ID NO: 209 - DNA, 20 bp, Artificial sequence
  Synthetic oligonucleotide
gaaggcgtcaccacctcacc

SEQ ID NO: 210 - DNA, 20 bp, Artificial sequence
  Synthetic oligonucleotide
aaggcgtcaccacctcaccg

SEQ ID NO: 211 - DNA, 20 bp, Artificial sequence
  Synthetic oligonucleotide
aggcgtcaccacctcaccgc

SEQ ID NO: 212 - DNA, 20 bp, Artificial sequence
  Synthetic oligonucleotide
ggcgtcaccacctcaccgcc

SEQ ID NO: 213 - DNA, 20 bp, Artificial sequence
  Synthetic oligonucleotide
cgggagggagggggggaggc

SEQ ID NO: 214 - DNA, 20 bp, Artificial sequence
  Synthetic oligonucleotide
gcgtcaccacctcaccgcca

SEQ ID NO: 215 - DNA, 20 bp, Artificial sequence
  Synthetic oligonucleotide
ctggccctcgaggccgctgc

SEQ ID NO: 216 - DNA, 20 bp, Artificial sequence
  Synthetic oligonucleotide
ggccctcgaggccgctgcag

SEQ ID NO: 217 - DNA, 20 bp, Artificial sequence
  Synthetic oligonucleotide
gccctcgaggccgctgcagt

SEQ ID NO: 218 - DNA, 20 bp, Artificial sequence
  Synthetic oligonucleotide
ccctcgaggccgctgcagtg

SEQ ID NO: 219 - DNA, 20 bp, Artificial sequence
  Synthetic oligonucleotide
ctcgaggccgctgcagtgcc

SEQ ID NO: 220 - DNA, 20 bp, Artificial sequence
  Synthetic oligonucleotide
cgaggccgctgcagtgcctc

SEQ ID NO: 221 - DNA, 20 bp, Artificial sequence
  Synthetic oligonucleotide
gggagggagggggggaggct

SEQ ID NO: 222 - DNA, 20 bp, Artificial sequence
  Synthetic oligonucleotide
gaggccgctgcagtgcctca

SEQ ID NO: 223 - DNA, 20 bp, Artificial sequence
  Synthetic oligonucleotide
agcagcatcctgagccactc

SEQ ID NO: 224 - DNA, 20 bp, Artificial sequence
  Synthetic oligonucleotide
gcagcatcctgagccactcc

SEQ ID NO: 225 - DNA, 20 bp, Artificial sequence
  Synthetic oligonucleotide
cagcatcctgagccactccc

SEQ ID NO: 226 - DNA, 20 bp, Artificial sequence
  Synthetic oligonucleotide
agcatcctgagccactccct

SEQ ID NO: 227 - DNA, 20 bp, Artificial sequence
  Synthetic oligonucleotide
gcatcctgagccactccctc

SEQ ID NO: 228 - DNA, 20 bp, Artificial sequence
  Synthetic oligonucleotide
atcctgagccactccctcct

SEQ ID NO: 229 - DNA, 20 bp, Artificial sequence
  Synthetic oligonucleotide
cctgagccactccctcctcc

SEQ ID NO: 230 - DNA, 20 bp, Artificial sequence
  Synthetic oligonucleotide
ggagggagggggggaggcta

SEQ ID NO: 231 - DNA, 20 bp, Artificial sequence
  Synthetic oligonucleotide
ctgagccactccctcctcca

SEQ ID NO: 232 - DNA, 20 bp, Artificial sequence
  Synthetic oligonucleotide
cgccttgtgctggccgcctc

SEQ ID NO: 233 - DNA, 20 bp, Artificial sequence
  Synthetic oligonucleotide
ccttgtgctggccgcctccc

SEQ ID NO: 234 - DNA, 20 bp, Artificial sequence
  Synthetic oligonucleotide
cttgtgctggccgcctccct

SEQ ID NO: 235 - DNA, 20 bp, Artificial sequence
  Synthetic oligonucleotide
ttgtgctggccgcctccctc

SEQ ID NO: 236 - DNA, 20 bp, Artificial sequence
  Synthetic oligonucleotide
gtgctggccgcctccctcac

SEQ ID NO: 237 - DNA, 20 bp, Artificial sequence
  Synthetic oligonucleotide
gctggccgcctccctcacac

SEQ ID NO: 238 - DNA, 20 bp, Artificial sequence
  Synthetic oligonucleotide
ctggccgcctccctcacacc

SEQ ID NO: 239 - DNA, 20 bp, Artificial sequence
  Synthetic oligonucleotide
ggatgctgggaacacgccgg

SEQ ID NO: 240 - DNA, 20 bp, Artificial sequence
  Synthetic oligonucleotide
atgctgggaacacgccggaa

SEQ ID NO: 241 - DNA, 20 bp, Artificial sequence
  Synthetic oligonucleotide
tgctgggaacacgccggaag

SEQ ID NO: 242 - DNA, 20 bp, Artificial sequence
  Synthetic oligonucleotide
gctgggaacacgccggaagc

SEQ ID NO: 243 - DNA, 20 bp, Artificial sequence
  Synthetic oligonucleotide
tgggaacacgccggaagcca

SEQ ID NO: 244 - DNA, 20 bp, Artificial sequence
  Synthetic oligonucleotide
ggaacacgccggaagccacg

SEQ ID NO: 245 - DNA, 20 bp, Artificial sequence
  Synthetic oligonucleotide
agggagggggggaggctacc

SEQ ID NO: 246 - DNA, 20 bp, Artificial sequence
  Synthetic oligonucleotide
gaacacgccggaagccacga

SEQ ID NO: 247 - DNA, 20 bp, Artificial sequence
  Synthetic oligonucleotide
atatccagggcgccggcacc

SEQ ID NO: 248 - DNA, 20 bp, Artificial sequence
  Synthetic oligonucleotide
atccagggcgccggcacctc

SEQ ID NO: 249 - DNA, 20 bp, Artificial sequence
  Synthetic oligonucleotide
tccagggcgccggcacctct

SEQ ID NO: 250 - DNA, 20 bp, Artificial sequence
  Synthetic oligonucleotide
ccagggcgccggcacctctg

SEQ ID NO: 251 - DNA, 20 bp, Artificial sequence
  Synthetic oligonucleotide
agggcgccggcacctctgga

SEQ ID NO: 252 - DNA, 20 bp, Artificial sequence
  Synthetic oligonucleotide
ggcgccggcacctctggaga

SEQ ID NO: 253 - DNA, 20 bp, Artificial sequence
  Synthetic oligonucleotide
gcgccggcacctctggagag

SEQ ID NO: 254 - DNA, 20 bp, Artificial sequence
  Synthetic oligonucleotide
gagattgactccactgtgta

SEQ ID NO: 255 - DNA, 20 bp, Artificial sequence
  Synthetic oligonucleotide
ccaccatcatcaccaccacc

SEQ ID NO: 256 - DNA, 20 bp, Artificial sequence
  Synthetic oligonucleotide
caccatcatcaccaccacca

SEQ ID NO: 257 - DNA, 20 bp, Artificial sequence
  Synthetic oligonucleotide
accatcatcaccaccaccat

SEQ ID NO: 258 - DNA, 20 bp, Artificial sequence
  Synthetic oligonucleotide
catcatcaccaccaccatca

SEQ ID NO: 259 - DNA, 20 bp, Artificial sequence
  Synthetic oligonucleotide
tcatcaccaccaccatcacc

SEQ ID NO: 260 - DNA, 20 bp, Artificial sequence
  Synthetic oligonucleotide
aggaaatgggggggcccagg

SEQ ID NO: 261 - DNA, 20 bp, Artificial sequence
  Synthetic oligonucleotide
cacccgtgtcttagcccttt

SEQ ID NO: 262 - DNA, 20 bp, Artificial sequence
  Synthetic oligonucleotide
catcaccaccaccatcaccg

SEQ ID NO: 263 - DNA, 20 bp, Artificial sequence
  Synthetic oligonucleotide
agcagggctggagaatcttg

SEQ ID NO: 264 - DNA, 20 bp, Artificial sequence
  Synthetic oligonucleotide
cagggctggagaatcttggg

SEQ ID NO: 265 - DNA, 20 bp, Artificial sequence
  Synthetic oligonucleotide
agggctggagaatcttggga

SEQ ID NO: 266 - DNA, 20 bp, Artificial sequence
  Synthetic oligonucleotide
gggctggagaatcttgggac

SEQ ID NO: 267 - DNA, 20 bp, Artificial sequence
  Synthetic oligonucleotide
gctggagaatcttgggactg

SEQ ID NO: 268 - DNA, 20 bp, Artificial sequence
  Synthetic oligonucleotide
tggagaatcttgggactgtt

SEQ ID NO: 269 - DNA, 20 bp, Artificial sequence
  Synthetic oligonucleotide
cccacccatctgcacccagc

SEQ ID NO: 270 - DNA, 20 bp, Artificial sequence
  Synthetic oligonucleotide
ggagaatcttgggactgttg

SEQ ID NO: 271 - DNA, 20 bp, Artificial sequence
  Synthetic oligonucleotide
tcaggccccaggaaggacat

SEQ ID NO: 272 - DNA, 20 bp, Artificial sequence
  Synthetic oligonucleotide
aggccccaggaaggacatta

SEQ ID NO: 273 - DNA, 20 bp, Artificial sequence
  Synthetic oligonucleotide
ggccccaggaaggacattac

SEQ ID NO: 274 - DNA, 20 bp, Artificial sequence
  Synthetic oligonucleotide
gccccaggaaggacattact

SEQ ID NO: 275 - DNA, 20 bp, Artificial sequence
  Synthetic oligonucleotide
cccaggaaggacattactat

SEQ ID NO: 276 - DNA, 20 bp, Artificial sequence
  Synthetic oligonucleotide
caggaaggacattactatcg

SEQ ID NO: 277 - DNA, 20 bp, Artificial sequence
  Synthetic oligonucleotide
aggaaggacattactatcgt

SEQ ID NO: 278 - DNA, 20 bp, Artificial sequence
  Synthetic oligonucleotide
gccccaggtaactgcaaagg

SEQ ID NO: 279 - DNA, 20 bp, Artificial sequence
  Synthetic oligonucleotide
cccaggtaactgcaaagggg

SEQ ID NO: 280 - DNA, 20 bp, Artificial sequence
  Synthetic oligonucleotide
ccaggtaactgcaaaggggg

SEQ ID NO: 281 - DNA, 20 bp, Artificial sequence
  Synthetic oligonucleotide
caggtaactgcaaagggggc

SEQ ID NO: 282 - DNA, 20 bp, Artificial sequence
  Synthetic oligonucleotide
ggtaactgcaaagggggcca

SEQ ID NO: 283 - DNA, 20 bp, Artificial sequence
  Synthetic oligonucleotide
taactgcaaagggggccaca

SEQ ID NO: 284 - DNA, 20 bp, Artificial sequence
  Synthetic oligonucleotide
cacccatctgcacccagctg

SEQ ID NO: 285 - DNA, 20 bp, Artificial sequence
  Synthetic oligonucleotide
aactgcaaagggggccacag

SEQ ID NO: 286 - DNA, 20 bp, Artificial sequence
  Synthetic oligonucleotide
accagagctcgctcgggcca

SEQ ID NO: 287 - DNA, 20 bp, Artificial sequence
  Synthetic oligonucleotide
cagagctcgctcgggccaga

SEQ ID NO: 288 - DNA, 20 bp, Artificial sequence
  Synthetic oligonucleotide
agagctcgctcgggccagag

SEQ ID NO: 289 - DNA, 20 bp, Artificial sequence
  Synthetic oligonucleotide
gagctcgctcgggccagagg

SEQ ID NO: 290 - DNA, 20 bp, Artificial sequence
  Synthetic oligonucleotide
gctcgctcgggccagagggc

SEQ ID NO: 291 - DNA, 20 bp, Artificial sequence
  Synthetic oligonucleotide
tcgctcgggccagagggcac

SEQ ID NO: 292 - DNA, 20 bp, Artificial sequence
  Synthetic oligonucleotide
acccatctgcacccagctgc

SEQ ID NO: 293 - DNA, 20 bp, Artificial sequence
  Synthetic oligonucleotide
cgctcgggccagagggcacc

SEQ ID NO: 294 - DNA, 20 bp, Artificial sequence
  Synthetic oligonucleotide
ctctcgttccaaccgtgctc

SEQ ID NO: 295 - DNA, 20 bp, Artificial sequence
  Synthetic oligonucleotide
ctcgttccaaccgtgctcag

SEQ ID NO: 296 - DNA, 20 bp, Artificial sequence
  Synthetic oligonucleotide
tcgttccaaccgtgctcaga

SEQ ID NO: 297 - DNA, 20 bp, Artificial sequence
  Synthetic oligonucleotide
cgttccaaccgtgctcagaa

SEQ ID NO: 298 - DNA, 20 bp, Artificial sequence
  Synthetic oligonucleotide
ttccaaccgtgctcagaacc

SEQ ID NO: 299 - DNA, 20 bp, Artificial sequence
  Synthetic oligonucleotide
ccaaccgtgctcagaaccag

SEQ ID NO: 300 - DNA, 20 bp, Artificial sequence
  Synthetic oligonucleotide
cccatctgcacccagctgct

SEQ ID NO: 301 - DNA, 20 bp, Artificial sequence
  Synthetic oligonucleotide
caaccgtgctcagaaccagg

SEQ ID NO: 302 - DNA, 20 bp, Artificial sequence
  Synthetic oligonucleotide
ggcaccacacgaccgccacg

SEQ ID NO: 303 - DNA, 20 bp, Artificial sequence
  Synthetic oligonucleotide
caccacacgaccgccacgga

SEQ ID NO: 304 - DNA, 20 bp, Artificial sequence
  Synthetic oligonucleotide
accacacgaccgccacggag

SEQ ID NO: 305 - DNA, 20 bp, Artificial sequence
  Synthetic oligonucleotide
ccacacgaccgccacggagc

SEQ ID NO: 306 - DNA, 20 bp, Artificial sequence
  Synthetic oligonucleotide
acacgaccgccacggagcct

SEQ ID NO: 307 - DNA, 20 bp, Artificial sequence
  Synthetic oligonucleotide
acgaccgccacggagcctgt

SEQ ID NO: 308 - DNA, 20 bp, Artificial sequence
  Synthetic oligonucleotide
ccatctgcacccagctgctc

SEQ ID NO: 309 - DNA, 20 bp, Artificial sequence
  Synthetic oligonucleotide
cgaccgccacggagcctgtg

SEQ ID NO: 310 - DNA, 20 bp, Artificial sequence
  Synthetic oligonucleotide
ttttaaagcgtctcatgtga

SEQ ID NO: 311 - DNA, 20 bp, Artificial sequence
  Synthetic oligonucleotide
ttaaagcgtctcatgtgaaa

SEQ ID NO: 312 - DNA, 20 bp, Artificial sequence
  Synthetic oligonucleotide
taaagcgtctcatgtgaaaa

SEQ ID NO: 313 - DNA, 20 bp, Artificial sequence
  Synthetic oligonucleotide
aaagcgtctcatgtgaaaac

SEQ ID NO: 314 - DNA, 20 bp, Artificial sequence
  Synthetic oligonucleotide
agcgtctcatgtgaaaactg

SEQ ID NO: 315 - DNA, 20 bp, Artificial sequence
  Synthetic oligonucleotide
cgtctcatgtgaaaactgtc

SEQ ID NO: 316 - DNA, 20 bp, Artificial sequence
  Synthetic oligonucleotide
catctgcacccagctgctct

SEQ ID NO: 317 - DNA, 20 bp, Artificial sequence
  Synthetic oligonucleotide
gtctcatgtgaaaactgtca

SEQ ID NO: 318 - DNA, 20 bp, Artificial sequence
  Synthetic oligonucleotide
cacagggggagaaggggaag

SEQ ID NO: 319 - DNA, 20 bp, Artificial sequence
  Synthetic oligonucleotide
cagggggagaaggggaaggg

SEQ ID NO: 320 - DNA, 20 bp, Artificial sequence
  Synthetic oligonucleotide
agggggagaaggggaagggc

SEQ ID NO: 321 - DNA, 20 bp, Artificial sequence
  Synthetic oligonucleotide
gggggagaaggggaagggca

SEQ ID NO: 322 - DNA, 20 bp, Artificial sequence
  Synthetic oligonucleotide
gggagaaggggaagggcatt

SEQ ID NO: 323 - DNA, 20 bp, Artificial sequence
  Synthetic oligonucleotide
gagaaggggaagggcattcc

SEQ ID NO: 324 - DNA, 20 bp, Artificial sequence
  Synthetic oligonucleotide
atctgcacccagctgctctc

SEQ ID NO: 325 - DNA, 20 bp, Artificial sequence
  Synthetic oligonucleotide
agaaggggaagggcattccc

SEQ ID NO: 326 - DNA, 20 bp, Artificial sequence
  Synthetic oligonucleotide
gcaggcgtatctgcatccca

SEQ ID NO: 327 - DNA, 20 bp, Artificial sequence
  Synthetic oligonucleotide
aggcgtatctgcatcccacc

SEQ ID NO: 328 - DNA, 20 bp, Artificial sequence
  Synthetic oligonucleotide
ggcgtatctgcatcccaccg

SEQ ID NO: 329 - DNA, 20 bp, Artificial sequence
  Synthetic oligonucleotide
gcgtatctgcatcccaccga

SEQ ID NO: 330 - DNA, 20 bp, Artificial sequence
  Synthetic oligonucleotide
gtatctgcatcccaccgagg

SEQ ID NO: 331 - DNA, 20 bp, Artificial sequence
  Synthetic oligonucleotide
tatctgcatcccaccgagga

SEQ ID NO: 332 - DNA, 20 bp, Artificial sequence
  Synthetic oligonucleotide
atctgcatcccaccgaggat

SEQ ID NO: 333 - DNA, 20 bp, Artificial sequence
  Synthetic oligonucleotide
tggccgcatcacttcagtct

SEQ ID NO: 334 - DNA, 20 bp, Artificial sequence
  Synthetic oligonucleotide
gccgcatcacttcagtctct

SEQ ID NO: 335 - DNA, 20 bp, Artificial sequence
  Synthetic oligonucleotide
cgcatcacttcagtctctct

SEQ ID NO: 336 - DNA, 20 bp, Artificial sequence
  Synthetic oligonucleotide
caggaaatgggggggcccag

SEQ ID NO: 337 - DNA, 20 bp, Artificial sequence
  Synthetic oligonucleotide
ggcagtgggtgccaggacag

SEQ ID NO: 338 - DNA, 20 bp, Artificial sequence
  Synthetic oligonucleotide
gcatcacttcagtctctctc

SEQ ID NO: 339 - DNA, 20 bp, Artificial sequence
  Synthetic oligonucleotide
tgggacactttgtcgtacga

SEQ ID NO: 340 - DNA, 20 bp, Artificial sequence
  Synthetic oligonucleotide
ggacactttgtcgtacgacc

SEQ ID NO: 341 - DNA, 20 bp, Artificial sequence
  Synthetic oligonucleotide
gacactttgtcgtacgaccc

SEQ ID NO: 342 - DNA, 20 bp, Artificial sequence
  Synthetic oligonucleotide
acactttgtcgtacgaccca

SEQ ID NO: 343 - DNA, 20 bp, Artificial sequence
  Synthetic oligonucleotide
actttgtcgtacgacccacg

SEQ ID NO: 344 - DNA, 20 bp, Artificial sequence
  Synthetic oligonucleotide
tttgtcgtacgacccacgcg

SEQ ID NO: 345 - DNA, 20 bp, Artificial sequence
  Synthetic oligonucleotide
agccaggtccacccgtgtct

SEQ ID NO: 346 - DNA, 20 bp, Artificial sequence
  Synthetic oligonucleotide
ttgtcgtacgacccacgcgc

SEQ ID NO: 347 - DNA, 20 bp, Artificial sequence
  Synthetic oligonucleotide
ggccacgtcctggtctctcc

SEQ ID NO: 348 - DNA, 20 bp, Artificial sequence
  Synthetic oligonucleotide
ccacgtcctggtctctcctc

SEQ ID NO: 349 - DNA, 20 bp, Artificial sequence
  Synthetic oligonucleotide
cacgtcctggtctctcctct

SEQ ID NO: 350 - DNA, 20 bp, Artificial sequence
  Synthetic oligonucleotide
acgtcctggtctctcctctg

SEQ ID NO: 351 - DNA, 20 bp, Artificial sequence
  Synthetic oligonucleotide
gtcctggtctctcctctgcg

SEQ ID NO: 352 - DNA, 20 bp, Artificial sequence
  Synthetic oligonucleotide
tcctggtctctcctctgcgt

SEQ ID NO: 353 - DNA, 20 bp, Artificial sequence
  Synthetic oligonucleotide
cctggtctctcctctgcgtt

SEQ ID NO: 354 - DNA, 20 bp, Artificial sequence
  Synthetic oligonucleotide
gccaggtccacccgtgtctt

SEQ ID NO: 355 - DNA, 20 bp, Artificial sequence
  Synthetic oligonucleotide
ctggtctctcctctgcgttg

SEQ ID NO: 356 - DNA, 20 bp, Artificial sequence
  Synthetic oligonucleotide
cttcagcgccagcctcctgg

SEQ ID NO: 357 - DNA, 20 bp, Artificial sequence
  Synthetic oligonucleotide
tcagcgccagcctcctggcc

SEQ ID NO: 358 - DNA, 20 bp, Artificial sequence
  Synthetic oligonucleotide
cagcgccagcctcctggccc

SEQ ID NO: 359 - DNA, 20 bp, Artificial sequence
  Synthetic oligonucleotide
agcgccagcctcctggcccc

SEQ ID NO: 360 - DNA, 20 bp, Artificial sequence
  Synthetic oligonucleotide
cgccagcctcctggccccag

SEQ ID NO: 361 - DNA, 20 bp, Artificial sequence
  Synthetic oligonucleotide
ccagcctcctggccccaggg

SEQ ID NO: 362 - DNA, 20 bp, Artificial sequence
  Synthetic oligonucleotide
ccaggtccacccgtgtctta

SEQ ID NO: 363 - DNA, 20 bp, Artificial sequence
  Synthetic oligonucleotide
cagcctcctggccccagggg

SEQ ID NO: 364 - DNA, 20 bp, Artificial sequence
  Synthetic oligonucleotide
ctgccctgggctgagagagg

SEQ ID NO: 365 - DNA, 20 bp, Artificial sequence
  Synthetic oligonucleotide
gccctgggctgagagaggtc

SEQ ID NO: 366 - DNA, 20 bp, Artificial sequence
  Synthetic oligonucleotide
ccctgggctgagagaggtca

SEQ ID NO: 367 - DNA, 20 bp, Artificial sequence
  Synthetic oligonucleotide
cctgggctgagagaggtcac

SEQ ID NO: 368 - DNA, 20 bp, Artificial sequence
  Synthetic oligonucleotide
tgggctgagagaggtcactt

SEQ ID NO: 369 - DNA, 20 bp, Artificial sequence
  Synthetic oligonucleotide
ggctgagagaggtcactttg

SEQ ID NO: 370 - DNA, 20 bp, Artificial sequence
  Synthetic oligonucleotide
caggtccacccgtgtcttag

SEQ ID NO: 371 - DNA, 20 bp, Artificial sequence
  Synthetic oligonucleotide
gctgagagaggtcactttgc

SEQ ID NO: 372 - DNA, 20 bp, Artificial sequence
  Synthetic oligonucleotide
tcacaggacctgctagcact

SEQ ID NO: 373 - DNA, 20 bp, Artificial sequence
  Synthetic oligonucleotide
acaggacctgctagcactgc

SEQ ID NO: 374 - DNA, 20 bp, Artificial sequence
  Synthetic oligonucleotide
caggacctgctagcactgcc

SEQ ID NO: 375 - DNA, 20 bp, Artificial sequence
  Synthetic oligonucleotide
aggacctgctagcactgcct

SEQ ID NO: 376 - DNA, 20 bp, Artificial sequence
  Synthetic oligonucleotide
gacctgctagcactgcctcg

SEQ ID NO: 377 - DNA, 20 bp, Artificial sequence
  Synthetic oligonucleotide
cctgctagcactgcctcgac

SEQ ID NO: 378 - DNA, 20 bp, Artificial sequence
  Synthetic oligonucleotide
aggtccacccgtgtcttagc

SEQ ID NO: 379 - DNA, 20 bp, Artificial sequence
  Synthetic oligonucleotide
ctgctagcactgcctcgaca

SEQ ID NO: 380 - DNA, 20 bp, Artificial sequence
  Synthetic oligonucleotide
tgaagcaggtggatcgcctg

SEQ ID NO: 381 - DNA, 20 bp, Artificial sequence
  Synthetic oligonucleotide
aagcaggtggatcgcctgag

SEQ ID NO: 382 - DNA, 20 bp, Artificial sequence
  Synthetic oligonucleotide
agcaggtggatcgcctgagc

SEQ ID NO: 383 - DNA, 20 bp, Artificial sequence
  Synthetic oligonucleotide
gcaggtggatcgcctgagcc

SEQ ID NO: 384 - DNA, 20 bp, Artificial sequence
  Synthetic oligonucleotide
aggtggatcgcctgagccca

SEQ ID NO: 385 - DNA, 20 bp, Artificial sequence
  Synthetic oligonucleotide
gtggatcgcctgagcccagg

SEQ ID NO: 386 - DNA, 20 bp, Artificial sequence
  Synthetic oligonucleotide
tggatcgcctgagcccagga

SEQ ID NO: 387 - DNA, 20 bp, Artificial sequence
  Synthetic oligonucleotide
ccaagggtcccggtgagccc

SEQ ID NO: 388 - DNA, 20 bp, Artificial sequence
  Synthetic oligonucleotide
aagggtcccggtgagcccag

SEQ ID NO: 389 - DNA, 20 bp, Artificial sequence
  Synthetic oligonucleotide
agggtcccggtgagcccagg

SEQ ID NO: 390 - DNA, 20 bp, Artificial sequence
  Synthetic oligonucleotide
gggtcccggtgagcccagga

SEQ ID NO: 391 - DNA, 20 bp, Artificial sequence
  Synthetic oligonucleotide
gtcccggtgagcccaggagc

SEQ ID NO: 392 - DNA, 20 bp, Artificial sequence
  Synthetic oligonucleotide
cccggtgagcccaggagcac

SEQ ID NO: 393 - DNA, 20 bp, Artificial sequence
  Synthetic oligonucleotide
gtccacccgtgtcttagccc

SEQ ID NO: 394 - DNA, 20 bp, Artificial sequence
  Synthetic oligonucleotide
ccggtgagcccaggagcacc

SEQ ID NO: 395 - DNA, 20 bp, Artificial sequence
  Synthetic oligonucleotide
tgggggccttggctgccacc

SEQ ID NO: 396 - DNA, 20 bp, Artificial sequence
  Synthetic oligonucleotide
ggggccttggctgccaccac

SEQ ID NO: 397 - DNA, 20 bp, Artificial sequence
  Synthetic oligonucleotide
gggccttggctgccaccacc

SEQ ID NO: 398 - DNA, 20 bp, Artificial sequence
  Synthetic oligonucleotide
ggccttggctgccaccacca

SEQ ID NO: 399 - DNA, 20 bp, Artificial sequence
  Synthetic oligonucleotide
ccttggctgccaccaccacg

SEQ ID NO: 400 - DNA, 20 bp, Artificial sequence
  Synthetic oligonucleotide
ttggctgccaccaccacgcg

SEQ ID NO: 401 - DNA, 20 bp, Artificial sequence
  Synthetic oligonucleotide
tggctgccaccaccacgcgg

SEQ ID NO: 402 - DNA, 20 bp, Artificial sequence
  Synthetic oligonucleotide
gcggggctaggcccggggtc

SEQ ID NO: 403 - DNA, 20 bp, Artificial sequence
  Synthetic oligonucleotide
ggggctaggcccggggtctg

SEQ ID NO: 404 - DNA, 20 bp, Artificial sequence
  Synthetic oligonucleotide
gggctaggcccggggtctgg

SEQ ID NO: 405 - DNA, 20 bp, Artificial sequence
  Synthetic oligonucleotide
ggctaggcccggggtctggt

SEQ ID NO: 406 - DNA, 20 bp, Artificial sequence
  Synthetic oligonucleotide
ctaggcccggggtctggtct

SEQ ID NO: 407 - DNA, 20 bp, Artificial sequence
  Synthetic oligonucleotide
aggcccggggtctggtctca

SEQ ID NO: 408 - DNA, 20 bp, Artificial sequence
  Synthetic oligonucleotide
ccacccgtgtcttagccctt

SEQ ID NO: 409 - DNA, 20 bp, Artificial sequence
  Synthetic oligonucleotide
ggcccggggtctggtctcag

SEQ ID NO: 410 - DNA, 20 bp, Artificial sequence
  Synthetic oligonucleotide
caccaccatcatcaccacca

SEQ ID NO: 411 - DNA, 20 bp, Artificial sequence
  Synthetic oligonucleotide
ggcgctgcaggcacagcccc

SEQ ID NO: 412 - DNA, 20 bp, Artificial sequence
  Synthetic oligonucleotide
cgctgcaggcacagcccccc

SEQ ID NO: 413 - DNA, 20 bp, Artificial sequence
  Synthetic oligonucleotide
ctgcaggcacagccccccaa

SEQ ID NO: 414 - DNA, 20 bp, Artificial sequence
  Synthetic oligonucleotide
tgcacagagcagctgtcagt

SEQ ID NO: 415 - DNA, 20 bp, Artificial sequence
  Synthetic oligonucleotide
tgcaggcacagccccccaag

SEQ ID NO: 416 - DNA, 20 bp, Artificial sequence
  Synthetic oligonucleotide
ggagggggagcccgaacagg

SEQ ID NO: 417 - DNA, 20 bp, Artificial sequence
  Synthetic oligonucleotide
agggggagcccgaacaggca

SEQ ID NO: 418 - DNA, 20 bp, Artificial sequence
  Synthetic oligonucleotide
gggggagcccgaacaggcac

SEQ ID NO: 419 - DNA, 20 bp, Artificial sequence
  Synthetic oligonucleotide
ggggagcccgaacaggcacg

SEQ ID NO: 420 - DNA, 20 bp, Artificial sequence
  Synthetic oligonucleotide
ggagcccgaacaggcacggg

SEQ ID NO: 421 - DNA, 20 bp, Artificial sequence
  Synthetic oligonucleotide
agcccgaacaggcacggggg

SEQ ID NO: 422 - DNA, 20 bp, Artificial sequence
  Synthetic oligonucleotide
cgaccagggggcagtgggtg

SEQ ID NO: 423 - DNA, 20 bp, Artificial sequence
  Synthetic oligonucleotide
gcccgaacaggcacggggga

SEQ ID NO: 424 - DNA, 20 bp, Artificial sequence
  Synthetic oligonucleotide
ggggcaggaggcagtgagat

SEQ ID NO: 425 - DNA, 20 bp, Artificial sequence
  Synthetic oligonucleotide
ggcaggaggcagtgagatgg

SEQ ID NO: 426 - DNA, 20 bp, Artificial sequence
  Synthetic oligonucleotide
gcaggaggcagtgagatggg

SEQ ID NO: 427 - DNA, 20 bp, Artificial sequence
  Synthetic oligonucleotide
caggaggcagtgagatgggg

SEQ ID NO: 428 - DNA, 20 bp, Artificial sequence
  Synthetic oligonucleotide
ggaggcagtgagatggggtg

SEQ ID NO: 429 - DNA, 20 bp, Artificial sequence
  Synthetic oligonucleotide
aggcagtgagatggggtgtc

SEQ ID NO: 430 - DNA, 20 bp, Artificial sequence
  Synthetic oligonucleotide
ggcagtgagatggggtgtcg

SEQ ID NO: 431 - DNA, 20 bp, Artificial sequence
  Synthetic oligonucleotide
ctcctcactcccaccgcacc

SEQ ID NO: 432 - DNA, 20 bp, Artificial sequence
  Synthetic oligonucleotide
cctcactcccaccgcacccc

SEQ ID NO: 433 - DNA, 20 bp, Artificial sequence
  Synthetic oligonucleotide
ctcactcccaccgcacccca

SEQ ID NO: 434 - DNA, 20 bp, Artificial sequence
  Synthetic oligonucleotide
tcactcccaccgcaccccag

SEQ ID NO: 435 - DNA, 20 bp, Artificial sequence
  Synthetic oligonucleotide
actcccaccgcaccccaggc

SEQ ID NO: 436 - DNA, 20 bp, Artificial sequence
  Synthetic oligonucleotide
tcccaccgcaccccaggcac

SEQ ID NO: 437 - DNA, 20 bp, Artificial sequence
  Synthetic oligonucleotide
accagggggcagtgggtgcc

SEQ ID NO: 438 - DNA, 20 bp, Artificial sequence
  Synthetic oligonucleotide
cccaccgcaccccaggcacc

SEQ ID NO: 439 - DNA, 20 bp, Artificial sequence
  Synthetic oligonucleotide
atacaaaaattagccgggtg

SEQ ID NO: 440 - DNA, 20 bp, Artificial sequence
  Synthetic oligonucleotide
acaaaaattagccgggtgtg

SEQ ID NO: 441 - DNA, 20 bp, Artificial sequence
  Synthetic oligonucleotide
caaaaattagccgggtgtgg

SEQ ID NO: 442 - DNA, 20 bp, Artificial sequence
  Synthetic oligonucleotide
aaaaattagccgggtgtggt

SEQ ID NO: 443 - DNA, 20 bp, Artificial sequence
  Synthetic oligonucleotide
aaattagccgggtgtggtgg

SEQ ID NO: 444 - DNA, 20 bp, Artificial sequence
  Synthetic oligonucleotide
attagccgggtgtggtggca

SEQ ID NO: 445 - DNA, 20 bp, Artificial sequence
  Synthetic oligonucleotide
ccagggggcagtgggtgcca

SEQ ID NO: 446 - DNA, 20 bp, Artificial sequence
  Synthetic oligonucleotide
ttagccgggtgtggtggcag

SEQ ID NO: 447 - DNA, 20 bp, Artificial sequence
  Synthetic oligonucleotide
ctgccctgaatgcctttttt

SEQ ID NO: 448 - DNA, 20 bp, Artificial sequence
  Synthetic oligonucleotide
gccctgaatgcctttttttt

SEQ ID NO: 449 - DNA, 20 bp, Artificial sequence
  Synthetic oligonucleotide
ccctgaatgccttttttttt

SEQ ID NO: 450 - DNA, 20 bp, Artificial sequence
  Synthetic oligonucleotide
cctgaatgcctttttttttt

SEQ ID NO: 451 - DNA, 20 bp, Artificial sequence
  Synthetic oligonucleotide
tgaatgcctttttttttttt

SEQ ID NO: 452 - DNA, 20 bp, Artificial sequence
  Synthetic oligonucleotide
aatgcctttttttttttttt

SEQ ID NO: 453 - DNA, 20 bp, Artificial sequence
  Synthetic oligonucleotide
cagggggcagtgggtgccag

SEQ ID NO: 454 - DNA, 20 bp, Artificial sequence
  Synthetic oligonucleotide
atgccttttttttttttttt

SEQ ID NO: 455 - DNA, 20 bp, Artificial sequence
  Synthetic oligonucleotide
gaggtcaggagcttcagacc

SEQ ID NO: 456 - DNA, 20 bp, Artificial sequence
  Synthetic oligonucleotide
ggtcaggagcttcagaccag

SEQ ID NO: 457 - DNA, 20 bp, Artificial sequence
  Synthetic oligonucleotide
gtcaggagcttcagaccagc

SEQ ID NO: 458 - DNA, 20 bp, Artificial sequence
  Synthetic oligonucleotide
tcaggagcttcagaccagcc

SEQ ID NO: 459 - DNA, 20 bp, Artificial sequence
  Synthetic oligonucleotide
aggagcttcagaccagcctg

SEQ ID NO: 460 - DNA, 20 bp, Artificial sequence
  Synthetic oligonucleotide
gagcttcagaccagcctggc

SEQ ID NO: 461 - DNA, 20 bp, Artificial sequence
  Synthetic oligonucleotide
agcttcagaccagcctggcc

SEQ ID NO: 462 - DNA, 20 bp, Artificial sequence
  Synthetic oligonucleotide
gtcctccacgcatgaacctg

SEQ ID NO: 463 - DNA, 20 bp, Artificial sequence
  Synthetic oligonucleotide
cctccacgcatgaacctgtg

SEQ ID NO: 464 - DNA, 20 bp, Artificial sequence
  Synthetic oligonucleotide
ctccacgcatgaacctgtga

SEQ ID NO: 465 - DNA, 20 bp, Artificial sequence
  Synthetic oligonucleotide
tccacgcatgaacctgtgat

SEQ ID NO: 466 - DNA, 20 bp, Artificial sequence
  Synthetic oligonucleotide
ccacgcatgaacctgtgatt

SEQ ID NO: 467 - DNA, 20 bp, Artificial sequence
  Synthetic oligonucleotide
cacgcatgaacctgtgattc

SEQ ID NO: 468 - DNA, 20 bp, Artificial sequence
  Synthetic oligonucleotide
cgcatgaacctgtgattcta

SEQ ID NO: 469 - DNA, 20 bp, Artificial sequence
  Synthetic oligonucleotide
gggggcagtgggtgccagga

SEQ ID NO: 470 - DNA, 20 bp, Artificial sequence
  Synthetic oligonucleotide
gcatgaacctgtgattctag

SEQ ID NO: 471 - DNA, 20 bp, Artificial sequence
  Synthetic oligonucleotide
ggttctgatgtcacacccgc

SEQ ID NO: 472 - DNA, 20 bp, Artificial sequence
  Synthetic oligonucleotide
ttctgatgtcacacccgcgc

SEQ ID NO: 473 - DNA, 20 bp, Artificial sequence
  Synthetic oligonucleotide
tctgatgtcacacccgcgct

SEQ ID NO: 474 - DNA, 20 bp, Artificial sequence
  Synthetic oligonucleotide
ctgatgtcacacccgcgctt

SEQ ID NO: 475 - DNA, 20 bp, Artificial sequence
  Synthetic oligonucleotide
gatgtcacacccgcgcttgt

SEQ ID NO: 476 - DNA, 20 bp, Artificial sequence
  Synthetic oligonucleotide
tgtcacacccgcgcttgtag

SEQ ID NO: 477 - DNA, 20 bp, Artificial sequence
  Synthetic oligonucleotide
gtcacacccgcgcttgtagg

SEQ ID NO: 478 - DNA, 20 bp, Artificial sequence
  Synthetic oligonucleotide
cttgtaggcgtttgccacag

SEQ ID NO: 479 - DNA, 20 bp, Artificial sequence
  Synthetic oligonucleotide
tgtaggcgtttgccacagcc

SEQ ID NO: 480 - DNA, 20 bp, Artificial sequence
  Synthetic oligonucleotide
gtaggcgtttgccacagccg

SEQ ID NO: 481 - DNA, 20 bp, Artificial sequence
  Synthetic oligonucleotide
taggcgtttgccacagccgc

SEQ ID NO: 482 - DNA, 20 bp, Artificial sequence
  Synthetic oligonucleotide
ggcgtttgccacagccgcac

SEQ ID NO: 483 - DNA, 20 bp, Artificial sequence
  Synthetic oligonucleotide
cgtttgccacagccgcacgc

SEQ ID NO: 484 - DNA, 20 bp, Artificial sequence
  Synthetic oligonucleotide
gggcagtgggtgccaggaca

SEQ ID NO: 485 - DNA, 20 bp, Artificial sequence
  Synthetic oligonucleotide
gtttgccacagccgcacgcg

SEQ ID NO: 486 - DNA, 20 bp, Artificial sequence
  Synthetic oligonucleotide
cttgtggccgcatcacttca

SEQ ID NO: 487 - DNA, 20 bp, Artificial sequence
  Synthetic oligonucleotide
tgtggccgcatcacttcagt

SEQ ID NO: 488 - DNA, 20 bp, Artificial sequence
  Synthetic oligonucleotide
gtggccgcatcacttcagtc

SEQ ID NO: 489 - DNA, 20 bp, Artificial sequence
  Synthetic oligonucleotide
cccaggaaatgggggggccc

SEQ ID NO: 490 - DNA, 20 bp, Artificial sequence
  Synthetic oligonucleotide
tgggggggcccaggctggtt

SEQ ID NO: 491 - DNA, 20 bp, Artificial sequence
  Synthetic oligonucleotide
gaatgggcatcagattccca

SEQ ID NO: 492 - DNA, 20 bp, Artificial sequence
  Synthetic oligonucleotide
gcacctgagagcgcgccatc

SEQ ID NO: 493 - DNA, 20 bp, Artificial sequence
  Synthetic oligonucleotide
acctgagagcgcgccatccc

SEQ ID NO: 494 - DNA, 20 bp, Artificial sequence
  Synthetic oligonucleotide
cctgagagcgcgccatcccc

SEQ ID NO: 495 - DNA, 20 bp, Artificial sequence
  Synthetic oligonucleotide
ctgagagcgcgccatcccca

SEQ ID NO: 496 - DNA, 20 bp, Artificial sequence
  Synthetic oligonucleotide
gagagcgcgccatccccacc

SEQ ID NO: 497 - DNA, 20 bp, Artificial sequence
  Synthetic oligonucleotide
gagcgcgccatccccacccg

SEQ ID NO: 498 - DNA, 20 bp, Artificial sequence
  Synthetic oligonucleotide
tgaggggtctgcacagagca

SEQ ID NO: 499 - DNA, 20 bp, Artificial sequence
  Synthetic oligonucleotide
agagcgcgccatccccaccc

SEQ ID NO: 500 - DNA, 20 bp, Artificial sequence
  Synthetic oligonucleotide
agcgcgccatccccacccgg

SEQ ID NO: 501 - DNA, 20 bp, Artificial sequence
  Synthetic oligonucleotide
cccctgcccagctggtgtcc

SEQ ID NO: 502 - DNA, 20 bp, Artificial sequence
  Synthetic oligonucleotide
cctgcccagctggtgtccgg

SEQ ID NO: 503 - DNA, 20 bp, Artificial sequence
  Synthetic oligonucleotide
ctgcccagctggtgtccgga

SEQ ID NO: 504 - DNA, 20 bp, Artificial sequence
  Synthetic oligonucleotide
tgcccagctggtgtccggac

SEQ ID NO: 505 - DNA, 20 bp, Artificial sequence
  Synthetic oligonucleotide
cccagctggtgtccggacca

SEQ ID NO: 506 - DNA, 20 bp, Artificial sequence
  Synthetic oligonucleotide
cagctggtgtccggaccaac

SEQ ID NO: 507 - DNA, 20 bp, Artificial sequence
  Synthetic oligonucleotide
agctggtgtccggaccaacg

SEQ ID NO: 508 - DNA, 20 bp, Artificial sequence
  Synthetic oligonucleotide
tagatgctccggacacgcag

SEQ ID NO: 509 - DNA, 20 bp, Artificial sequence
  Synthetic oligonucleotide
gatgctccggacacgcagga

SEQ ID NO: 510 - DNA, 20 bp, Artificial sequence
  Synthetic oligonucleotide
atgctccggacacgcaggac

SEQ ID NO: 511 - DNA, 20 bp, Artificial sequence
  Synthetic oligonucleotide
tgctccggacacgcaggaca

SEQ ID NO: 512 - DNA, 20 bp, Artificial sequence
  Synthetic oligonucleotide
ctccggacacgcaggacagc

SEQ ID NO: 513 - DNA, 20 bp, Artificial sequence
  Synthetic oligonucleotide
ccggacacgcaggacagctg

SEQ ID NO: 514 - DNA, 20 bp, Artificial sequence
  Synthetic oligonucleotide
aggggtctgcacagagcagc

SEQ ID NO: 515 - DNA, 20 bp, Artificial sequence
  Synthetic oligonucleotide
cggacacgcaggacagctgc

SEQ ID NO: 516 - DNA, 20 bp, Artificial sequence
  Synthetic oligonucleotide
gcaggccatgccccaggcag

SEQ ID NO: 517 - DNA, 20 bp, Artificial sequence
  Synthetic oligonucleotide
aggccatgccccaggcagcc

SEQ ID NO: 518 - DNA, 20 bp, Artificial sequence
  Synthetic oligonucleotide
ggccatgccccaggcagccc

SEQ ID NO: 519 - DNA, 20 bp, Artificial sequence
  Synthetic oligonucleotide
gccatgccccaggcagccca

SEQ ID NO: 520 - DNA, 20 bp, Artificial sequence
  Synthetic oligonucleotide
catgccccaggcagcccaca

SEQ ID NO: 521 - DNA, 20 bp, Artificial sequence
  Synthetic oligonucleotide
tgccccaggcagcccacacg

SEQ ID NO: 522 - DNA, 20 bp, Artificial sequence
  Synthetic oligonucleotide
ggggtctgcacagagcagct

SEQ ID NO: 523 - DNA, 20 bp, Artificial sequence
  Synthetic oligonucleotide
gccccaggcagcccacacgc

SEQ ID NO: 524 - DNA, 20 bp, Artificial sequence
  Synthetic oligonucleotide
gcccacacgcccgtctccct

SEQ ID NO: 525 - DNA, 20 bp, Artificial sequence
  Synthetic oligonucleotide
ccacacgcccgtctccctct

SEQ ID NO: 526 - DNA, 20 bp, Artificial sequence
  Synthetic oligonucleotide
cacacgcccgtctccctctc

SEQ ID NO: 527 - DNA, 20 bp, Artificial sequence
  Synthetic oligonucleotide
acacgcccgtctccctctct

SEQ ID NO: 528 - DNA, 20 bp, Artificial sequence
  Synthetic oligonucleotide
acgcccgtctccctctctgg

SEQ ID NO: 529 - DNA, 20 bp, Artificial sequence
  Synthetic oligonucleotide
cgcccgtctccctctctgga

SEQ ID NO: 530 - DNA, 20 bp, Artificial sequence
  Synthetic oligonucleotide
gcccgtctccctctctggag

SEQ ID NO: 531 - DNA, 20 bp, Artificial sequence
  Synthetic oligonucleotide
gggtctgcacagagcagctg

SEQ ID NO: 532 - DNA, 20 bp, Artificial sequence
  Synthetic oligonucleotide
cccgtctccctctctggagc

SEQ ID NO: 533 - DNA, 20 bp, Artificial sequence
  Synthetic oligonucleotide
gctgcctcccgcggtcgagg

SEQ ID NO: 534 - DNA, 20 bp, Artificial sequence
  Synthetic oligonucleotide
tgcctcccgcggtcgaggca

SEQ ID NO: 535 - DNA, 20 bp, Artificial sequence
  Synthetic oligonucleotide
gcctcccgcggtcgaggcag

SEQ ID NO: 536 - DNA, 20 bp, Artificial sequence
  Synthetic oligonucleotide
cctcccgcggtcgaggcagc

SEQ ID NO: 537 - DNA, 20 bp, Artificial sequence
  Synthetic oligonucleotide
tcccgcggtcgaggcagcca

SEQ ID NO: 538 - DNA, 20 bp, Artificial sequence
  Synthetic oligonucleotide
ccgcggtcgaggcagccagg

SEQ ID NO: 539 - DNA, 20 bp, Artificial sequence
  Synthetic oligonucleotide
cgcggtcgaggcagccagga

SEQ ID NO: 540 - DNA, 20 bp, Artificial sequence
  Synthetic oligonucleotide
cacttctcaaactcggtgcc

SEQ ID NO: 541 - DNA, 20 bp, Artificial sequence
  Synthetic oligonucleotide
acttctcaaactcggtgccc

SEQ ID NO: 542 - DNA, 20 bp, Artificial sequence
  Synthetic oligonucleotide
cttctcaaactcggtgcccc

SEQ ID NO: 543 - DNA, 20 bp, Artificial sequence
  Synthetic oligonucleotide
ttctcaaactcggtgcccca

SEQ ID NO: 544 - DNA, 20 bp, Artificial sequence
  Synthetic oligonucleotide
tctcaaactcggtgccccag

SEQ ID NO: 545 - DNA, 20 bp, Artificial sequence
  Synthetic oligonucleotide
tcaaactcggtgccccagga

SEQ ID NO: 546 - DNA, 20 bp, Artificial sequence
  Synthetic oligonucleotide
aaactcggtgccccaggaac

SEQ ID NO: 547 - DNA, 20 bp, Artificial sequence
  Synthetic oligonucleotide
gtctgcacagagcagctgtc

SEQ ID NO: 548 - DNA, 20 bp, Artificial sequence
  Synthetic oligonucleotide
aactcggtgccccaggaacc

SEQ ID NO: 549 - DNA, 20 bp, Artificial sequence
  Synthetic oligonucleotide
cacgaacgcgacggtgggac

SEQ ID NO: 550 - DNA, 20 bp, Artificial sequence
  Synthetic oligonucleotide
cgaacgcgacggtgggacga

SEQ ID NO: 551 - DNA, 20 bp, Artificial sequence
  Synthetic oligonucleotide
gaacgcgacggtgggacgag

SEQ ID NO: 552 - DNA, 20 bp, Artificial sequence
  Synthetic oligonucleotide
aacgcgacggtgggacgagg

SEQ ID NO: 553 - DNA, 20 bp, Artificial sequence
  Synthetic oligonucleotide
cgcgacggtgggacgaggca

SEQ ID NO: 554 - DNA, 20 bp, Artificial sequence
  Synthetic oligonucleotide
cgacggtgggacgaggcagg

SEQ ID NO: 555 - DNA, 20 bp, Artificial sequence
  Synthetic oligonucleotide
gacggtgggacgaggcagga

SEQ ID NO: 556 - DNA, 20 bp, Artificial sequence
  Synthetic oligonucleotide
ttttaacagccacatgtggc

SEQ ID NO: 557 - DNA, 20 bp, Artificial sequence
  Synthetic oligonucleotide
ttaacagccacatgtggctg

SEQ ID NO: 558 - DNA, 20 bp, Artificial sequence
  Synthetic oligonucleotide
taacagccacatgtggctgg

SEQ ID NO: 559 - DNA, 20 bp, Artificial sequence
  Synthetic oligonucleotide
aacagccacatgtggctggc

SEQ ID NO: 560 - DNA, 20 bp, Artificial sequence
  Synthetic oligonucleotide
cagccacatgtggctggcag

SEQ ID NO: 561 - DNA, 20 bp, Artificial sequence
  Synthetic oligonucleotide
gccacatgtggctggcagct

SEQ ID NO: 562 - DNA, 20 bp, Artificial sequence
  Synthetic oligonucleotide
ctgcacagagcagctgtcag

SEQ ID NO: 563 - DNA, 20 bp, Artificial sequence
  Synthetic oligonucleotide
ccacatgtggctggcagctc

SEQ ID NO: 564 - DNA, 20 bp, Artificial sequence
  Synthetic oligonucleotide
gggaggcgctgcaggcacag

SEQ ID NO: 565 - DNA, 20 bp, Artificial sequence
  Synthetic oligonucleotide
gaggcgctgcaggcacagcc

SEQ ID NO: 566 - DNA, 20 bp, Artificial sequence
  Synthetic oligonucleotide
aggcgctgcaggcacagccc

SEQ ID NO: 567 - DNA, 20 bp, Artificial sequence
  Synthetic oligonucleotide
atggcagactgcaatggcgt

SEQ ID NO: 568 - DNA, 20 bp, Artificial sequence
  Synthetic oligonucleotide
ctgtctgagctggggactct

SEQ ID NO: 569 - DNA, 20 bp, Artificial sequence
  Synthetic oligonucleotide
gtcagcgtgatctgtgggac

SEQ ID NO: 570 - DNA, 20 bp, Artificial sequence
  Synthetic oligonucleotide
ccgcaggttcccctcggggg

SEQ ID NO: 571 - DNA, 20 bp, Artificial sequence
  Synthetic oligonucleotide
ctccccaggcggccattccg

SEQ ID NO: 572 - DNA, 20 bp, Artificial sequence
  Synthetic oligonucleotide
gggactctatctgggctagg

SEQ ID NO: 573 - DNA, 20 bp, Artificial sequence
  Synthetic oligonucleotide
gccacaccttggggggggag

SEQ ID NO: 574 - DNA, 20 bp, Artificial sequence
  Synthetic oligonucleotide
gtaactgcaaagggggccac

SEQ ID NO: 575 - DNA, 20 bp, Artificial sequence
  Synthetic oligonucleotide
gcagctaggcctggggccca

SEQ ID NO: 576 - DNA, 20 bp, Artificial sequence
  Synthetic oligonucleotide
attaccaccaccattaccac

SEQ ID NO: 577 - DNA, 20 bp, Artificial sequence
  Synthetic oligonucleotide
ctccgggagacccctctgct

SEQ ID NO: 578 - DNA, 20 bp, Artificial sequence
  Synthetic oligonucleotide
tctgagctggcagggctgcc

SEQ ID NO: 579 - DNA, 20 bp, Artificial sequence
  Synthetic oligonucleotide
cctgtctgagctgggaggcc

SEQ ID NO: 580 - DNA, 20 bp, Artificial sequence
  Synthetic oligonucleotide
cacatcaccatcgccaccat

SEQ ID NO: 581 - DNA, 20 bp, Artificial sequence
  Synthetic oligonucleotide
gagctggggactctatctgg

SEQ ID NO: 582 - DNA, 20 bp, Artificial sequence
  Synthetic oligonucleotide
caccctccacagcaggtcca

SEQ ID NO: 583 - DNA, 20 bp, Artificial sequence
  Synthetic oligonucleotide
ggaccttcggagggcccagg

SEQ ID NO: 584 - DNA, 20 bp, Artificial sequence
  Synthetic oligonucleotide
ggatgaggggaggacggcgg

SEQ ID NO: 585 - DNA, 20 bp, Artificial sequence
  Synthetic oligonucleotide
accatttgcagcggcaaagt

SEQ ID NO: 586 - DNA, 20 bp, Artificial sequence
  Synthetic oligonucleotide
cgttgaatcccggcggccat

SEQ ID NO: 587 - DNA, 20 bp, Artificial sequence
  Synthetic oligonucleotide
tgcggggctaggcccggggt

SEQ ID NO: 588 - DNA, 20 bp, Artificial sequence
  Synthetic oligonucleotide
gagaatcttgggactgttga

SEQ ID NO: 589 - DNA, 20 bp, Artificial sequence
  Synthetic oligonucleotide
ctccaccctcagggtaggat

SEQ ID NO: 590 - DNA, 20 bp, Artificial sequence
  Synthetic oligonucleotide
aggctggcagctaggcctgg

SEQ ID NO: 591 - DNA, 20 bp, Artificial sequence
  Synthetic oligonucleotide
gggaaggctccaccctctgg

SEQ ID NO: 592 - DNA, 20 bp, Artificial sequence
  Synthetic oligonucleotide
cctccgggagacccctctgc

SEQ ID NO: 593 - DNA, 20 bp, Artificial sequence
  Synthetic oligonucleotide
agacagagggatcgaggcct

SEQ ID NO: 594 - DNA, 20 bp, Artificial sequence
  Synthetic oligonucleotide
cacaggctccccaggcggcc

SEQ ID NO: 595 - DNA, 20 bp, Artificial sequence
  Synthetic oligonucleotide
cccccggcgatgtcacctcc

SEQ ID NO: 596 - DNA, 20 bp, Artificial sequence
  Synthetic oligonucleotide
gaggacggcgggaagacgat

SEQ ID NO: 597 - DNA, 20 bp, Artificial sequence
  Synthetic oligonucleotide
ccctgcctcagacagagcca

SEQ ID NO: 598 - DNA, 20 bp, Artificial sequence
  Synthetic oligonucleotide
aaccttcctctgcctcagtt

SEQ ID NO: 599 - DNA, 20 bp, Artificial sequence
  Synthetic oligonucleotide
ctcagacagagccagggttg

SEQ ID NO: 600 - DNA, 20 bp, Artificial sequence
  Synthetic oligonucleotide
cctcagggaaggccacaccc

SEQ ID NO: 601 - DNA, 20 bp, Artificial sequence
  Synthetic oligonucleotide
ccctcggcaaacgccgtccc

SEQ ID NO: 602 - DNA, 20 bp, Artificial sequence
  Synthetic oligonucleotide
cacagcctgggccccggggc

SEQ ID NO: 603 - DNA, 20 bp, Artificial sequence
  Synthetic oligonucleotide
ctgtccaagctgggggactc

SEQ ID NO: 604 - DNA, 20 bp, Artificial sequence
  Synthetic oligonucleotide
cagaggatggcagactgcaa

SEQ ID NO: 605 - DNA, 20 bp, Artificial sequence
  Synthetic oligonucleotide
tcaccacctccaccatcacc

SEQ ID NO: 606 - DNA, 20 bp, Artificial sequence
  Synthetic oligonucleotide
gcctcacctcgggggaggaa

SEQ ID NO: 607 - DNA, 20 bp, Artificial sequence
  Synthetic oligonucleotide
ctcacagcattccagctcgc

SEQ ID NO: 608 - DNA, 20 bp, Artificial sequence
  Synthetic oligonucleotide
tgggcaccctccacagcagg

SEQ ID NO: 609 - DNA, 20 bp, Artificial sequence
  Synthetic oligonucleotide
aggccccatccacagggaag

SEQ ID NO: 610 - DNA, 20 bp, Artificial sequence
  Synthetic oligonucleotide
ctcctgccctcggcaaacgc

SEQ ID NO: 611 - DNA, 20 bp, Artificial sequence
  Synthetic oligonucleotide
ggcaggtccgagctttgtga

SEQ ID NO: 612 - DNA, 20 bp, Artificial sequence
  Synthetic oligonucleotide
ctggcactgggcttccttcc

SEQ ID NO: 613 - DNA, 20 bp, Artificial sequence
  Synthetic oligonucleotide
tcgcacggccagcccaccgt

SEQ ID NO: 614 - DNA, 20 bp, Artificial sequence
  Synthetic oligonucleotide
agtggctcgcacggccagcc

SEQ ID NO: 615 - DNA, 20 bp, Artificial sequence
  Synthetic oligonucleotide
gccagagagtacacggcggc

SEQ ID NO: 616 - DNA, 20 bp, Artificial sequence
  Synthetic oligonucleotide
gggaggaggctgcatctacc

SEQ ID NO: 617 - DNA, 20 bp, Artificial sequence
  Synthetic oligonucleotide
gctgccctgtccaagctggg

SEQ ID NO: 618 - DNA, 20 bp, Artificial sequence
  Synthetic oligonucleotide
ggaaggccacacccacaggg

SEQ ID NO: 619 - DNA, 20 bp, Artificial sequence
  Synthetic oligonucleotide
cctcagggtaggatccaccc

SEQ ID NO: 620 - DNA, 20 bp, Artificial sequence
  Synthetic oligonucleotide
cagggaaggccccatccaca

SEQ ID NO: 621 - DNA, 20 bp, Artificial sequence
  Synthetic oligonucleotide
ccgcagctggcactgggctt

SEQ ID NO: 622 - DNA, 20 bp, Artificial sequence
  Synthetic oligonucleotide
ctgggccccggggcacgctc

SEQ ID NO: 623 - DNA, 20 bp, Artificial sequence
  Synthetic oligonucleotide
agggaggaggctgcatctac

SEQ ID NO: 624 - DNA, 20 bp, Artificial sequence
  Synthetic oligonucleotide
atccggggttactttcctgc

SEQ ID NO: 625 - DNA, 20 bp, Artificial sequence
  Synthetic oligonucleotide
ctccacggcaggtccgagct

SEQ ID NO: 626 - DNA, 20 bp, Artificial sequence
  Synthetic oligonucleotide
tggggggggaggaaagagca

SEQ ID NO: 627 - DNA, 20 bp, Artificial sequence
  Synthetic oligonucleotide
gagtacacggcggcagggct

SEQ ID NO: 628 - DNA, 20 bp, Artificial sequence
  Synthetic oligonucleotide
gcattccagctcgcttcagg

SEQ ID NO: 629 - DNA, 20 bp, Artificial sequence
  Synthetic oligonucleotide
tcccggtgagcccaggagca

SEQ ID NO: 630 - DNA, 20 bp, Artificial sequence
  Synthetic oligonucleotide
ccaccatcatcaccacctcc

SEQ ID NO: 631 - DNA, 20 bp, Artificial sequence
  Synthetic oligonucleotide
ttacaagacggcaaagttca

SEQ ID NO: 632 - DNA, 20 bp, Artificial sequence
  Synthetic oligonucleotide
ataaacagacagagggatcg

SEQ ID NO: 633 - DNA, 20 bp, Artificial sequence
  Synthetic oligonucleotide
caatggtggtcagcgtgatc

SEQ ID NO: 634 - DNA, 20 bp, Artificial sequence
  Synthetic oligonucleotide
ggcggggcctcacctcgggg

SEQ ID NO: 635 - DNA, 20 bp, Artificial sequence
  Synthetic oligonucleotide
tcaagtggaccttcggaggg

SEQ ID NO: 636 - DNA, 20 bp, Artificial sequence
  Synthetic oligonucleotide
ccatcgccaccatcatcacc

SEQ ID NO: 637 - DNA, 20 bp, Artificial sequence
  Synthetic oligonucleotide
ccacccttacaagacggcaa

SEQ ID NO: 638 - DNA, 20 bp, Artificial sequence
  Synthetic oligonucleotide
ggtggtcagcgtgatctgtg

SEQ ID NO: 639 - DNA, 20 bp, Artificial sequence
  Synthetic oligonucleotide
tcactcaaccttcctctgcc

SEQ ID NO: 640 - DNA, 20 bp, Artificial sequence
  Synthetic oligonucleotide
ctggcagggctgccctgtct

SEQ ID NO: 641 - DNA, 20 bp, Artificial sequence
  Synthetic oligonucleotide
tcccggcggccatcacgtga

SEQ ID NO: 642 - DNA, 20 bp, Artificial sequence
  Synthetic oligonucleotide
ggttactttcctgcccccca

SEQ ID NO: 643 - DNA, 20 bp, Artificial sequence
  Synthetic oligonucleotide
tgagctgggaggccctgtct

SEQ ID NO: 644 - DNA, 20 bp, Artificial sequence
  Synthetic oligonucleotide
tgcagcggcaaagtcctagc

SEQ ID NO: 645 - DNA, 20 bp, Artificial sequence
  Synthetic oligonucleotide
agagtacacggcggcagggc

SEQ ID NO: 646 - DNA, 20 bp, Artificial sequence
  Synthetic oligonucleotide
gctggcactgggcttccttc

SEQ ID NO: 647 - DNA, 20 bp, Artificial sequence
  Synthetic oligonucleotide
tcccccggcgatgtcacctc

SEQ ID NO: 648 - DNA, 20 bp, Artificial sequence
  Synthetic oligonucleotide
cctgtccaagctgggggact

SEQ ID NO: 649 - DNA, 20 bp, Artificial sequence
  Synthetic oligonucleotide
agacggcaaagttcagcaaa

SEQ ID NO: 650 - DNA, 20 bp, Artificial sequence
  Synthetic oligonucleotide
cagggaaggctccaccctct

SEQ ID NO: 651 - DNA, 20 bp, Artificial sequence
  Synthetic oligonucleotide
cctgggccccggggcacgct

SEQ ID NO: 652 - DNA, 20 bp, Artificial sequence
  Synthetic oligonucleotide
ggcctcacctcgggggagga

SEQ ID NO: 653 - DNA, 20 bp, Artificial sequence
  Synthetic oligonucleotide
ctgagctgggaggccctgtc

SEQ ID NO: 654 - DNA, 20 bp, Artificial sequence
  Synthetic oligonucleotide
ctgcatctaccccaggcagc

SEQ ID NO: 655 - DNA, 20 bp, Artificial sequence
  Synthetic oligonucleotide
tgcatctaccccaggcagca

SEQ ID NO: 656 - DNA, 20 bp, Artificial sequence
  Synthetic oligonucleotide
accaccatcatcaccacctc

SEQ ID NO: 657 - DNA, 20 bp, Artificial sequence
  Synthetic oligonucleotide
ggaaggacattactatcgtc

SEQ ID NO: 658 - DNA, 20 bp, Artificial sequence
  Synthetic oligonucleotide
ggtcagcgtgatctgtggga

SEQ ID NO: 659 - DNA, 20 bp, Artificial sequence
  Synthetic oligonucleotide
caggcggccattccgtcctg

SEQ ID NO: 660 - DNA, 20 bp, Artificial sequence
  Synthetic oligonucleotide
ctcaagtggaccttcggagg

SEQ ID NO: 661 - DNA, 20 bp, Artificial sequence
  Synthetic oligonucleotide
caggctggcagctaggcctg

SEQ ID NO: 662 - DNA, 20 bp, Artificial sequence
  Synthetic oligonucleotide
cctcacagcattccagctcg

SEQ ID NO: 663 - DNA, 20 bp, Artificial sequence
  Synthetic oligonucleotide
tgagctggggactctatctg

SEQ ID NO: 664 - DNA, 20 bp, Artificial sequence
  Synthetic oligonucleotide
ggcgatgtcacctcctccca

SEQ ID NO: 665 - DNA, 20 bp, Artificial sequence
  Synthetic oligonucleotide
cctcagacagagccagggtt

SEQ ID NO: 666 - DNA, 20 bp, Artificial sequence
  Synthetic oligonucleotide
cccacctccgggagacccct

SEQ ID NO: 667 - DNA, 20 bp, Artificial sequence
  Synthetic oligonucleotide
ccaatggtggtcagcgtgat

SEQ ID NO: 668 - DNA, 20 bp, Artificial sequence
  Synthetic oligonucleotide
ccctcagggtaggatccacc

SEQ ID NO: 669 - DNA, 20 bp, Artificial sequence
  Synthetic oligonucleotide
gggaaggccacacccacagg

SEQ ID NO: 670 - DNA, 20 bp, Artificial sequence
  Synthetic oligonucleotide
tccgcagctggcactgggct

SEQ ID NO: 671 - DNA, 20 bp, Artificial sequence
  Synthetic oligonucleotide
agctgccctgtccaagctgg

SEQ ID NO: 672 - DNA, 20 bp, Artificial sequence
  Synthetic oligonucleotide
aaggccccatccacagggaa

SEQ ID NO: 673 - DNA, 20 bp, Artificial sequence
  Synthetic oligonucleotide
ggggactctatctgggctag

SEQ ID NO: 674 - DNA, 20 bp, Artificial sequence
  Synthetic oligonucleotide
cattaccaccaccattacca

SEQ ID NO: 675 - DNA, 20 bp, Artificial sequence
  Synthetic oligonucleotide
ggccagagagtacacggcgg

SEQ ID NO: 676 - DNA, 20 bp, Artificial sequence
  Synthetic oligonucleotide
tctgagctgggaggccctgt

SEQ ID NO: 677 - DNA, 20 bp, Artificial sequence
  Synthetic oligonucleotide
gcaccctccacagcaggtcc

SEQ ID NO: 678 - DNA, 20 bp, Artificial sequence
  Synthetic oligonucleotide
cggcaggtccgagctttgtg

SEQ ID NO: 679 - DNA, 20 bp, Artificial sequence
  Synthetic oligonucleotide
aatcccggcggccatcacgt

SEQ ID NO: 680 - DNA, 20 bp, Artificial sequence
  Synthetic oligonucleotide
cccctgcctcagacagagcc

SEQ ID NO: 681 - DNA, 20 bp, Artificial sequence
  Synthetic oligonucleotide
cagacagagggatcgaggcc

SEQ ID NO: 682 - DNA, 20 bp, Artificial sequence
  Synthetic oligonucleotide
ccctcagggaaggccacacc

SEQ ID NO: 683 - DNA, 20 bp, Artificial sequence
  Synthetic oligonucleotide
tcacatcaccatcgccacca

SEQ ID NO: 684 - DNA, 20 bp, Artificial sequence
  Synthetic oligonucleotide
ggcagctaggcctggggccc

SEQ ID NO: 685 - DNA, 20 bp, Artificial sequence
  Synthetic oligonucleotide
gataaacagacagagggatc

SEQ ID NO: 686 - DNA, 20 bp, Artificial sequence
  Synthetic oligonucleotide
aggtaactgcaaagggggcc

SEQ ID NO: 687 - DNA, 20 bp, Artificial sequence
  Synthetic oligonucleotide
cagtggctcgcacggccagc

SEQ ID NO: 688 - DNA, 20 bp, Artificial sequence
  Synthetic oligonucleotide
ttggggggggaggaaagagc

SEQ ID NO: 689 - DNA, 20 bp, Artificial sequence
  Synthetic oligonucleotide
gctggcagggctgccctgtc

SEQ ID NO: 690 - DNA, 20 bp, Artificial sequence
  Synthetic oligonucleotide
cttacaagacggcaaagttc

SEQ ID NO: 691 - DNA, 20 bp, Artificial sequence
  Synthetic oligonucleotide
atcaccacctccaccatcac

SEQ ID NO: 692 - DNA, 20 bp, Artificial sequence
  Synthetic oligonucleotide
ggctgccaccaccacgcgga

SEQ ID NO: 693 - DNA, 20 bp, Artificial sequence
  Synthetic oligonucleotide
caccatttgcagcggcaaag

SEQ ID NO: 694 - DNA, 20 bp, Artificial sequence
  Synthetic oligonucleotide
accatcgccaccatcatcac

SEQ ID NO: 695 - DNA, 20 bp, Artificial sequence
  Synthetic oligonucleotide
aggatgaggggaggacggcg

SEQ ID NO: 696 - DNA, 20 bp, Artificial sequence
  Synthetic oligonucleotide
tcagggaaggccccatccac

SEQ ID NO: 697 - DNA, 20 bp, Artificial sequence
  Synthetic oligonucleotide
gtccgagctttgtgaaccgt

SEQ ID NO: 698 - DNA, 20 bp, Artificial sequence
  Synthetic oligonucleotide
agcattccagctcgcttcag

SEQ ID NO: 699 - DNA, 20 bp, Artificial sequence
  Synthetic oligonucleotide
caagggtcccggtgagccca

SEQ ID NO: 700 - DNA, 20 bp, Artificial sequence
  Synthetic oligonucleotide
gatggcagactgcaatggcg

SEQ ID NO: 701 - DNA, 20 bp, Artificial sequence
  Synthetic oligonucleotide
caaccttcctctgcctcagt

SEQ ID NO: 702 - DNA, 20 bp, Artificial sequence
  Synthetic oligonucleotide
tggtggtcagcgtgatctgt

SEQ ID NO: 703 - DNA, 20 bp, Artificial sequence
  Synthetic oligonucleotide
tccacagcaggtccaagcct

SEQ ID NO: 704 - DNA, 20 bp, Artificial sequence
  Synthetic oligonucleotide
tggaccttcggagggcccag

SEQ ID NO: 705 - DNA, 20 bp, Artificial sequence
  Synthetic oligonucleotide
ccccggggcacgctcagctc

SEQ ID NO: 706 - DNA, 20 bp, Artificial sequence
  Synthetic oligonucleotide
gctccccaggcggccattcc

SEQ ID NO: 707 - DNA, 20 bp, Artificial sequence
  Synthetic oligonucleotide
cctcctgccctcggcaaacg

SEQ ID NO: 708 - DNA, 20 bp, Artificial sequence
  Synthetic oligonucleotide
gtctgagctggcagggctgc

SEQ ID NO: 709 - DNA, 20 bp, Artificial sequence
  Synthetic oligonucleotide
gggttactttcctgcccccc

SEQ ID NO: 710 - DNA, 20 bp, Artificial sequence
  Synthetic oligonucleotide
ggccacaccttgggggggga

SEQ ID NO: 711 - DNA, 20 bp, Artificial sequence
  Synthetic oligonucleotide
gctccaccctcagggtagga

SEQ ID NO: 712 - DNA, 20 bp, Artificial sequence
  Synthetic oligonucleotide
ccagaggatggcagactgca

SEQ ID NO: 713 - DNA, 20 bp, Artificial sequence
  Synthetic oligonucleotide
cccgcaggttcccctcgggg

SEQ ID NO: 714 - DNA, 20 bp, Artificial sequence
  Synthetic oligonucleotide
acgttgaatcccggcggcca

SEQ ID NO: 715 - DNA, 20 bp, Artificial sequence
  Synthetic oligonucleotide
ttgcagcggcaaagtcctag

SEQ ID NO: 716 - DNA, 20 bp, Artificial sequence
  Synthetic oligonucleotide
ctcgcacggccagcccaccg

SEQ ID NO: 717 - DNA, 20 bp, Artificial sequence
  Synthetic oligonucleotide
ctcactcaaccttcctctgc

SEQ ID NO: 718 - DNA, 20 bp, Artificial sequence
  Synthetic oligonucleotide
ctggagaatcttgggactgt

SEQ ID NO: 719 - DNA, 20 bp, Artificial sequence
  Synthetic oligonucleotide
ggaggacggcgggaagacga

SEQ ID NO: 720 - DNA, 20 bp, Artificial sequence
  Synthetic oligonucleotide
acctccgggagacccctctg

SEQ ID NO: 721 - DNA, 20 bp, Artificial sequence
  Synthetic oligonucleotide
gatccggggttactttcctg

SEQ ID NO: 722 - DNA, 20 bp, Artificial sequence
  Synthetic oligonucleotide
gccctcggcaaacgccgtcc

SEQ ID NO: 723 - DNA, 20 bp, Artificial sequence
  Synthetic oligonucleotide
cccatccacagggaaggctc

SEQ ID NO: 724 - DNA, 20 bp, Artificial sequence
  Synthetic oligonucleotide
gaaggccccatccacaggga

SEQ ID NO: 725 - DNA, 20 bp, Artificial sequence
  Synthetic oligonucleotide
gctcactcaaccttcctctg

SEQ ID NO: 726 - DNA, 20 bp, Artificial sequence
  Synthetic oligonucleotide
agctggcagggctgccctgt

SEQ ID NO: 727 - DNA, 20 bp, Artificial sequence
  Synthetic oligonucleotide
ccaggaaggacattactatc

SEQ ID NO: 728 - DNA, 20 bp, Artificial sequence
  Synthetic oligonucleotide
ccctgtccaagctgggggac

SEQ ID NO: 729 - DNA, 20 bp, Artificial sequence
  Synthetic oligonucleotide
cttggctgccaccaccacgc

SEQ ID NO: 730 - DNA, 20 bp, Artificial sequence
  Synthetic oligonucleotide
acagacagagggatcgaggc

SEQ ID NO: 731 - DNA, 20 bp, Artificial sequence
  Synthetic oligonucleotide
tcagggaaggctccaccctc

SEQ ID NO: 732 - DNA, 20 bp, Artificial sequence
  Synthetic oligonucleotide
accctcagggtaggatccac

SEQ ID NO: 733 - DNA, 20 bp, Artificial sequence
  Synthetic oligonucleotide
ccccaggtaactgcaaaggg

SEQ ID NO: 734 - DNA, 20 bp, Artificial sequence
  Synthetic oligonucleotide
ccctcacagcattccagctc

SEQ ID NO: 735 - DNA, 20 bp, Artificial sequence
  Synthetic oligonucleotide
ccaggctggcagctaggcct

SEQ ID NO: 736 - DNA, 20 bp, Artificial sequence
  Synthetic oligonucleotide
cctcccccggcgatgtcacc

SEQ ID NO: 737 - DNA, 20 bp, Artificial sequence
  Synthetic oligonucleotide
ccaggcggccattccgtcct

SEQ ID NO: 738 - DNA, 20 bp, Artificial sequence
  Synthetic oligonucleotide
acggcaggtccgagctttgt

SEQ ID NO: 739 - DNA, 20 bp, Artificial sequence
  Synthetic oligonucleotide
ggctggagaatcttgggact

SEQ ID NO: 740 - DNA, 20 bp, Artificial sequence
  Synthetic oligonucleotide
gccagaggatggcagactgc

SEQ ID NO: 741 - DNA, 20 bp, Artificial sequence
  Synthetic oligonucleotide
cggcgatgtcacctcctccc

SEQ ID NO: 742 - DNA, 20 bp, Artificial sequence
  Synthetic oligonucleotide
tcccgcaggttcccctcggg

SEQ ID NO: 743 - DNA, 20 bp, Artificial sequence
  Synthetic oligonucleotide
agggaaggctccaccctctg

SEQ ID NO: 744 - DNA, 20 bp, Artificial sequence
  Synthetic oligonucleotide
acagtggctcgcacggccag

SEQ ID NO: 745 - DNA, 20 bp, Artificial sequence
  Synthetic oligonucleotide
ggatggcagactgcaatggc

SEQ ID NO: 746 - DNA, 20 bp, Artificial sequence
  Synthetic oligonucleotide
gagctgccctgtccaagctg

SEQ ID NO: 747 - DNA, 20 bp, Artificial sequence
  Synthetic oligonucleotide
gagagtacacggcggcaggg

SEQ ID NO: 748 - DNA, 20 bp, Artificial sequence
  Synthetic oligonucleotide
ctgggaggccctgtctgagc

SEQ ID NO: 749 - DNA, 20 bp, Artificial sequence
  Synthetic oligonucleotide
agggaaggccacacccacag

SEQ ID NO: 750 - DNA, 20 bp, Artificial sequence
  Synthetic oligonucleotide
tggcagctaggcctggggcc

SEQ ID NO: 751 - DNA, 20 bp, Artificial sequence
  Synthetic oligonucleotide
tcaaccttcctctgcctcag

SEQ ID NO: 752 - DNA, 20 bp, Artificial sequence
  Synthetic oligonucleotide
caccatcgccaccatcatca

SEQ ID NO: 753 - DNA, 20 bp, Artificial sequence
  Synthetic oligonucleotide
atcacatcaccatcgccacc

SEQ ID NO: 754 - DNA, 20 bp, Artificial sequence
  Synthetic oligonucleotide
gctgcatctaccccaggcag

SEQ ID NO: 755 - DNA, 20 bp, Artificial sequence
  Synthetic oligonucleotide
ccttacaagacggcaaagtt

SEQ ID NO: 756 - DNA, 20 bp, Artificial sequence
  Synthetic oligonucleotide
cagcattccagctcgcttca

SEQ ID NO: 757 - DNA, 20 bp, Artificial sequence
  Synthetic oligonucleotide
ctgggcaccctccacagcag

SEQ ID NO: 758 - DNA, 20 bp, Artificial sequence
  Synthetic oligonucleotide
gggcctcacctcgggggagg

SEQ ID NO: 759 - DNA, 20 bp, Artificial sequence
  Synthetic oligonucleotide
tcatcaccacctccaccatc

SEQ ID NO: 760 - DNA, 20 bp, Artificial sequence
  Synthetic oligonucleotide
tttgcagcggcaaagtccta

SEQ ID NO: 761 - DNA, 20 bp, Artificial sequence
  Synthetic oligonucleotide
ccctcctgccctcggcaaac

SEQ ID NO: 762 - DNA, 20 bp, Artificial sequence
  Synthetic oligonucleotide
gcccggggtctggtctcagc

SEQ ID NO: 763 - DNA, 20 bp, Artificial sequence
  Synthetic oligonucleotide
ggggttactttcctgccccc

SEQ ID NO: 764 - DNA, 20 bp, Artificial sequence
  Synthetic oligonucleotide
cctatctcaacaacaacaaa

SEQ ID NO: 765 - DNA, 20 bp, Artificial sequence
  Synthetic oligonucleotide
gcctgggccccggggcacgc

SEQ ID NO: 766 - DNA, 20 bp, Artificial sequence
  Synthetic oligonucleotide
ctccacagcaggtccaagcc

SEQ ID NO: 767 - DNA, 20 bp, Artificial sequence
  Synthetic oligonucleotide
agctggcactgggcttcctt

SEQ ID NO: 768 - DNA, 20 bp, Artificial sequence
  Synthetic oligonucleotide
tgatccggggttactttcct

SEQ ID NO: 769 - DNA, 20 bp, Artificial sequence
  Synthetic oligonucleotide
tgccctcggcaaacgccgtc

SEQ ID NO: 770 - DNA, 20 bp, Artificial sequence
  Synthetic oligonucleotide
gctcgcacggccagcccacc

SEQ ID NO: 771 - DNA, 20 bp, Artificial sequence
  Synthetic oligonucleotide
gaatcccggcggccatcacg

SEQ ID NO: 772 - DNA, 20 bp, Artificial sequence
  Synthetic oligonucleotide
cacgttgaatcccggcggcc

SEQ ID NO: 773 - DNA, 20 bp, Artificial sequence
  Synthetic oligonucleotide
tgtctgagctgggaggccct

SEQ ID NO: 774 - DNA, 20 bp, Artificial sequence
  Synthetic oligonucleotide
agggtaggatccacccacag

SEQ ID NO: 775 - DNA, 20 bp, Artificial sequence
  Synthetic oligonucleotide
gtccgcagctggcactgggc

SEQ ID NO: 776 - DNA, 20 bp, Artificial sequence
  Synthetic oligonucleotide
ccattaccaccaccattacc

SEQ ID NO: 777 - DNA, 20 bp, Artificial sequence
  Synthetic oligonucleotide
gcctcagacagagccagggt

SEQ ID NO: 778 - DNA, 20 bp, Artificial sequence
  Synthetic oligonucleotide
cacctccgggagacccctct

SEQ ID NO: 779 - DNA, 20 bp, Artificial sequence
  Synthetic oligonucleotide
ggctccccaggcggccattc

SEQ ID NO: 780 - DNA, 20 bp, Artificial sequence
  Synthetic oligonucleotide
gggccacaccttgggggggg

SEQ ID NO: 781 - DNA, 20 bp, Artificial sequence
  Synthetic oligonucleotide
gccccggggcacgctcagct

SEQ ID NO: 782 - DNA, 20 bp, Artificial sequence
  Synthetic oligonucleotide
aagacggcaaagttcagcaa

SEQ ID NO: 783 - DNA, 20 bp, Artificial sequence
  Synthetic oligonucleotide
gggaggacggcgggaagacg

SEQ ID NO: 784 - DNA, 20 bp, Artificial sequence
  Synthetic oligonucleotide
agataaacagacagagggat

SEQ ID NO: 785 - DNA, 20 bp, Artificial sequence
  Synthetic oligonucleotide
gcccctgcctcagacagagc

SEQ ID NO: 786 - DNA, 20 bp, Artificial sequence
  Synthetic oligonucleotide
tgtctgagctggcagggctg

SEQ ID NO: 787 - DNA, 20 bp, Artificial sequence
  Synthetic oligonucleotide
ggagacccctctgctcacgg

SEQ ID NO: 788 - DNA, 20 bp, Artificial sequence
  Synthetic oligonucleotide
acacggcggcagggctcacg

SEQ ID NO: 789 - DNA, 20 bp, Artificial sequence
  Synthetic oligonucleotide
tggggactctatctgggcta

SEQ ID NO: 790 - DNA, 20 bp, Artificial sequence
  Synthetic oligonucleotide
cgcctacaaaccgtggtgtc

SEQ ID NO: 791 - DNA, 20 bp, Artificial sequence
  Synthetic oligonucleotide
gcaccatttgcagcggcaaa

SEQ ID NO: 792 - DNA, 20 bp, Artificial sequence
  Synthetic oligonucleotide
gtggaccttcggagggccca

SEQ ID NO: 793 - DNA, 20 bp, Artificial sequence
  Synthetic oligonucleotide
ctgagctggggactctatct

SEQ ID NO: 794 - DNA, 20 bp, Artificial sequence
  Synthetic oligonucleotide
cctcaagtggaccttcggag

SEQ ID NO: 795 - DNA, 20 bp, Artificial sequence
  Synthetic oligonucleotide
caccttggggggggaggaaa

SEQ ID NO: 796 - DNA, 20 bp, Artificial sequence
  Synthetic oligonucleotide
aggctgcatctaccccaggc

SEQ ID NO: 797 - DNA, 20 bp, Artificial sequence
  Synthetic oligonucleotide
cccaagggtcccggtgagcc

SEQ ID NO: 798 - DNA, 20 bp, Artificial sequence
  Synthetic oligonucleotide
ggtccgagctttgtgaaccg

SEQ ID NO: 799 - DNA, 20 bp, Artificial sequence
  Synthetic oligonucleotide
ttccagctcgcttcaggaag

SEQ ID NO: 800 - DNA, 20 bp, Artificial sequence
  Synthetic oligonucleotide
agcccctgcctcagacagag

SEQ ID NO: 801 - DNA, 20 bp, Artificial sequence
  Synthetic oligonucleotide
gccctgtccaagctggggga

SEQ ID NO: 802 - DNA, 20 bp, Artificial sequence
  Synthetic oligonucleotide
tctgagctggggactctatc

SEQ ID NO: 803 - DNA, 20 bp, Artificial sequence
  Synthetic oligonucleotide
acctcaagtggaccttcgga

SEQ ID NO: 804 - DNA, 20 bp, Artificial sequence
  Synthetic oligonucleotide
tgaatcccggcggccatcac

SEQ ID NO: 805 - DNA, 20 bp, Artificial sequence
  Synthetic oligonucleotide
cctccacagcaggtccaagc

SEQ ID NO: 806 - DNA, 20 bp, Artificial sequence
  Synthetic oligonucleotide
caccctcagggtaggatcca

SEQ ID NO: 807 - DNA, 20 bp, Artificial sequence
  Synthetic oligonucleotide
gaggaggctgcatctacccc

SEQ ID NO: 808 - DNA, 20 bp, Artificial sequence
  Synthetic oligonucleotide
ccacagcaggtccaagcctc

SEQ ID NO: 809 - DNA, 20 bp, Artificial sequence
  Synthetic oligonucleotide
ggagctgccctgtccaagct

SEQ ID NO: 810 - DNA, 20 bp, Artificial sequence
  Synthetic oligonucleotide
ccccaggaaggacattacta

SEQ ID NO: 811 - DNA, 20 bp, Artificial sequence
  Synthetic oligonucleotide
aacagacagagggatcgagg

SEQ ID NO: 812 - DNA, 20 bp, Artificial sequence
  Synthetic oligonucleotide
agcctgggccccggggcacg

SEQ ID NO: 813 - DNA, 20 bp, Artificial sequence
  Synthetic oligonucleotide
aacagtggctcgcacggcca

SEQ ID NO: 814 - DNA, 20 bp, Artificial sequence
  Synthetic oligonucleotide
acagcattccagctcgcttc

SEQ ID NO: 815 - DNA, 20 bp, Artificial sequence
  Synthetic oligonucleotide
agagagtacacggcggcagg

SEQ ID NO: 816 - DNA, 20 bp, Artificial sequence
  Synthetic oligonucleotide
acacgttgaatcccggcggc

SEQ ID NO: 817 - DNA, 20 bp, Artificial sequence
  Synthetic oligonucleotide
ggctgcatctaccccaggca

SEQ ID NO: 818 - DNA, 20 bp, Artificial sequence
  Synthetic oligonucleotide
cggggttactttcctgcccc

SEQ ID NO: 819 - DNA, 20 bp, Artificial sequence
  Synthetic oligonucleotide
cagggtaggatccacccaca

SEQ ID NO: 820 - DNA, 20 bp, Artificial sequence
  Synthetic oligonucleotide
gcagggctggagaatcttgg

SEQ ID NO: 821 - DNA, 20 bp, Artificial sequence
  Synthetic oligonucleotide
acaccttggggggggaggaa

SEQ ID NO: 822 - DNA, 20 bp, Artificial sequence
  Synthetic oligonucleotide
ggggcctcacctcgggggag

SEQ ID NO: 823 - DNA, 20 bp, Artificial sequence
  Synthetic oligonucleotide
tccctatctcaacaacaaca

SEQ ID NO: 824 - DNA, 20 bp, Artificial sequence
  Synthetic oligonucleotide
catcacatcaccatcgccac

SEQ ID NO: 825 - DNA, 20 bp, Artificial sequence
  Synthetic oligonucleotide
atcatcaccacctccaccat

SEQ ID NO: 826 - DNA, 20 bp, Artificial sequence
  Synthetic oligonucleotide
gcactgggcttccttcctgc

SEQ ID NO: 827 - DNA, 20 bp, Artificial sequence
  Synthetic oligonucleotide
cccaggcggccattccgtcc

SEQ ID NO: 828 - DNA, 20 bp, Artificial sequence
  Synthetic oligonucleotide
tcaccatcgccaccatcatc

SEQ ID NO: 829 - DNA, 20 bp, Artificial sequence
  Synthetic oligonucleotide
ggggaggacggcgggaagac

SEQ ID NO: 830 - DNA, 20 bp, Artificial sequence
  Synthetic oligonucleotide
tagataaacagacagaggga

SEQ ID NO: 831 - DNA, 20 bp, Artificial sequence
  Synthetic oligonucleotide
taggcccggggtctggtctc

SEQ ID NO: 832 - DNA, 20 bp, Artificial sequence
  Synthetic oligonucleotide
gccttggctgccaccaccac

SEQ ID NO: 833 - DNA, 20 bp, Artificial sequence
  Synthetic oligonucleotide
cccaggctggcagctaggcc

SEQ ID NO: 834 - DNA, 20 bp, Artificial sequence
  Synthetic oligonucleotide
ccacctccgggagacccctc

SEQ ID NO: 835 - DNA, 20 bp, Artificial sequence
  Synthetic oligonucleotide
caagacggcaaagttcagca

SEQ ID NO: 836 - DNA, 20 bp, Artificial sequence
  Synthetic oligonucleotide
agcaccatttgcagcggcaa

SEQ ID NO: 837 - DNA, 20 bp, Artificial sequence
  Synthetic oligonucleotide
gagctggcagggctgccctg

SEQ ID NO: 838 - DNA, 20 bp, Artificial sequence
  Synthetic oligonucleotide
gtctgagctgggaggccctg

SEQ ID NO: 839 - DNA, 20 bp, Artificial sequence
  Synthetic oligonucleotide
cccttacaagacggcaaagt

SEQ ID NO: 840 - DNA, 20 bp, Artificial sequence
  Synthetic oligonucleotide
acctcgggggaggaaagagc

SEQ ID NO: 841 - DNA, 20 bp, Artificial sequence
  Synthetic oligonucleotide
atgtctgagctggcagggct

SEQ ID NO: 842 - DNA, 20 bp, Artificial sequence
  Synthetic oligonucleotide
atggtggtcagcgtgatctg

SEQ ID NO: 843 - DNA, 20 bp, Artificial sequence
  Synthetic oligonucleotide
cacggcaggtccgagctttg

SEQ ID NO: 844 - DNA, 20 bp, Artificial sequence
  Synthetic oligonucleotide
acctcccccggcgatgtcac

SEQ ID NO: 845 - DNA, 20 bp, Artificial sequence
  Synthetic oligonucleotide
ggccagaggatggcagactg

SEQ ID NO: 846 - DNA, 20 bp, Artificial sequence
  Synthetic oligonucleotide
agctcactcaaccttcctct

SEQ ID NO: 847 - DNA, 20 bp, Artificial sequence
  Synthetic oligonucleotide
gggagacccctctgctcacg

SEQ ID NO: 848 - DNA, 20 bp, Artificial sequence
  Synthetic oligonucleotide
cagcgtgatctgtgggaccg

SEQ ID NO: 849 - DNA, 20 bp, Artificial sequence
  Synthetic oligonucleotide
ctgccctcggcaaacgccgt

SEQ ID NO: 850 - DNA, 20 bp, Artificial sequence
  Synthetic oligonucleotide
ctggggactctatctgggct

SEQ ID NO: 851 - DNA, 20 bp, Artificial sequence
  Synthetic oligonucleotide
ggccccaggtaactgcaaag

SEQ ID NO: 852 - DNA, 20 bp, Artificial sequence
  Synthetic oligonucleotide
ggccacacccacagggaagg

SEQ ID NO: 853 - DNA, 20 bp, Artificial sequence
  Synthetic oligonucleotide
tggtcagcgtgatctgtggg

SEQ ID NO: 854 - DNA, 20 bp, Artificial sequence
  Synthetic oligonucleotide
gtgatccggggttactttcc

SEQ ID NO: 855 - DNA, 20 bp, Artificial sequence
  Synthetic oligonucleotide
tgtcccgcaggttcccctcg

SEQ ID NO: 856 - DNA, 20 bp, Artificial sequence
  Synthetic oligonucleotide
agtggaccttcggagggccc

SEQ ID NO: 857 - DNA, 20 bp, Artificial sequence
  Synthetic oligonucleotide
aggatggcagactgcaatgg

SEQ ID NO: 858 - DNA, 20 bp, Artificial sequence
  Synthetic oligonucleotide
ggaaggccccatccacaggg

SEQ ID NO: 859 - DNA, 20 bp, Artificial sequence
  Synthetic oligonucleotide
aggctccccaggcggccatt

SEQ ID NO: 860 - DNA, 20 bp, Artificial sequence
  Synthetic oligonucleotide
cccctcctgccctcggcaaa

SEQ ID NO: 861 - DNA, 20 bp, Artificial sequence
  Synthetic oligonucleotide
caggttcccctcggggggcc

SEQ ID NO: 862 - DNA, 20 bp, Artificial sequence
  Synthetic oligonucleotide
tacacggcggcagggctcac

SEQ ID NO: 863 - DNA, 20 bp, Artificial sequence
  Synthetic oligonucleotide
tgcctcagacagagccaggg

SEQ ID NO: 864 - DNA, 20 bp, Artificial sequence
  Synthetic oligonucleotide
ccggcgatgtcacctcctcc

SEQ ID NO: 865 - DNA, 20 bp, Artificial sequence
  Synthetic oligonucleotide
ggccccggggcacgctcagc

SEQ ID NO: 866 - DNA, 20 bp, Artificial sequence
  Synthetic oligonucleotide
atttgcagcggcaaagtcct

SEQ ID NO: 867 - DNA, 20 bp, Artificial sequence
  Synthetic oligonucleotide
ccccatccacagggaaggct

SEQ ID NO: 868 - DNA, 20 bp, Artificial sequence
  Synthetic oligonucleotide
agctgggaggccctgtctga

SEQ ID NO: 869 - DNA, 20 bp, Artificial sequence
  Synthetic oligonucleotide
ctggcagctaggcctggggc

SEQ ID NO: 870 - DNA, 20 bp, Artificial sequence
  Synthetic oligonucleotide
ctcaaccttcctctgcctca

SEQ ID NO: 871 - DNA, 20 bp, Artificial sequence
  Synthetic oligonucleotide
ggctcgcacggccagcccac

SEQ ID NO: 872 - DNA, 20 bp, Artificial sequence
  Synthetic oligonucleotide
cagggaaggccacacccaca

SEQ ID NO: 873 - DNA, 20 bp, Artificial sequence
  Synthetic oligonucleotide
aggtccgagctttgtgaacc

SEQ ID NO: 874 - DNA, 20 bp, Artificial sequence
  Synthetic oligonucleotide
accattaccaccaccattac

SEQ ID NO: 875 - DNA, 20 bp, Artificial sequence
  Synthetic oligonucleotide
cagctggcactgggcttcct

SEQ ID NO: 876 - DNA, 20 bp, Artificial sequence
  Synthetic oligonucleotide
gcctccgcctacaaaccgtg

SEQ ID NO: 877 - DNA, 20 bp, Artificial sequence
  Synthetic oligonucleotide
gtccaagctgggggactcta

SEQ ID NO: 878 - DNA, 20 bp, Artificial sequence
  Synthetic oligonucleotide
caggtccgagctttgtgaac

SEQ ID NO: 879 - DNA, 20 bp, Artificial sequence
  Synthetic oligonucleotide
aagtggaccttcggagggcc

SEQ ID NO: 880 - DNA, 20 bp, Artificial sequence
  Synthetic oligonucleotide
aaacagacagagggatcgag

SEQ ID NO: 881 - DNA, 20 bp, Artificial sequence
  Synthetic oligonucleotide
gctggcagctaggcctgggg

SEQ ID NO: 882 - DNA, 20 bp, Artificial sequence
  Synthetic oligonucleotide
attccagctcgcttcaggaa

SEQ ID NO: 883 - DNA, 20 bp, Artificial sequence
  Synthetic oligonucleotide
aacctcccccggcgatgtca

SEQ ID NO: 884 - DNA, 20 bp, Artificial sequence
  Synthetic oligonucleotide
tcccctcctgccctcggcaa

SEQ ID NO: 885 - DNA, 20 bp, Artificial sequence
  Synthetic oligonucleotide
ttccaggcctccgcctacaa

SEQ ID NO: 886 - DNA, 20 bp, Artificial sequence
  Synthetic oligonucleotide
aggaggctgcatctacccca

SEQ ID NO: 887 - DNA, 20 bp, Artificial sequence
  Synthetic oligonucleotide
gctggggactctatctgggc

SEQ ID NO: 888 - DNA, 20 bp, Artificial sequence
  Synthetic oligonucleotide
cacagcattccagctcgctt

SEQ ID NO: 889 - DNA, 20 bp, Artificial sequence
  Synthetic oligonucleotide
ctgcctcagacagagccagg

SEQ ID NO: 890 - DNA, 20 bp, Artificial sequence
  Synthetic oligonucleotide
aatggtggtcagcgtgatct

SEQ ID NO: 891 - DNA, 20 bp, Artificial sequence
  Synthetic oligonucleotide
acccttacaagacggcaaag

SEQ ID NO: 892 - DNA, 20 bp, Artificial sequence
  Synthetic oligonucleotide
ccatcatcaccacctccacc

SEQ ID NO: 893 - DNA, 20 bp, Artificial sequence
  Synthetic oligonucleotide
gggaaggccccatccacagg

SEQ ID NO: 894 - DNA, 20 bp, Artificial sequence
  Synthetic oligonucleotide
aacctcaagtggaccttcgg

SEQ ID NO: 895 - DNA, 20 bp, Artificial sequence
  Synthetic oligonucleotide
cccggcgatgtcacctcctc

SEQ ID NO: 896 - DNA, 20 bp, Artificial sequence
  Synthetic oligonucleotide
tgccctgtccaagctggggg

SEQ ID NO: 897 - DNA, 20 bp, Artificial sequence
  Synthetic oligonucleotide
gtggtcagcgtgatctgtgg

SEQ ID NO: 898 - DNA, 20 bp, Artificial sequence
  Synthetic oligonucleotide
gagctgggaggccctgtctg

SEQ ID NO: 899 - DNA, 20 bp, Artificial sequence
  Synthetic oligonucleotide
acaagacggcaaagttcagc

SEQ ID NO: 900 - DNA, 20 bp, Artificial sequence
  Synthetic oligonucleotide
gctgtcccgcaggttcccct

SEQ ID NO: 901 - DNA, 20 bp, Artificial sequence
  Synthetic oligonucleotide
ggcactgggcttccttcctg

SEQ ID NO: 902 - DNA, 20 bp, Artificial sequence
  Synthetic oligonucleotide
gccccatccacagggaaggc

SEQ ID NO: 903 - DNA, 20 bp, Artificial sequence
  Synthetic oligonucleotide
gagctcactcaaccttcctc

SEQ ID NO: 904 - DNA, 20 bp, Artificial sequence
  Synthetic oligonucleotide
catgtctgagctggcagggc

SEQ ID NO: 905 - DNA, 20 bp, Artificial sequence
  Synthetic oligonucleotide
ctcacctcgggggaggaaag

SEQ ID NO: 906 - DNA, 20 bp, Artificial sequence
  Synthetic oligonucleotide
taccaccaccattaccacca

SEQ ID NO: 907 - DNA, 20 bp, Artificial sequence
  Synthetic oligonucleotide
tcagggtaggatccacccac

SEQ ID NO: 908 - DNA, 20 bp, Artificial sequence
  Synthetic oligonucleotide
gggggccttggctgccacca

SEQ ID NO: 909 - DNA, 20 bp, Artificial sequence
  Synthetic oligonucleotide
tgagctggcagggctgccct

SEQ ID NO: 910 - DNA, 20 bp, Artificial sequence
  Synthetic oligonucleotide
ccaccctcagggtaggatcc

SEQ ID NO: 911 - DNA, 20 bp, Artificial sequence
  Synthetic oligonucleotide
gggaggccctgtctgagctg

SEQ ID NO: 912 - DNA, 20 bp, Artificial sequence
  Synthetic oligonucleotide
tggctcgcacggccagccca

SEQ ID NO: 913 - DNA, 20 bp, Artificial sequence
  Synthetic oligonucleotide
ggaggaggctgcatctaccc

SEQ ID NO: 914 - DNA, 20 bp, Artificial sequence
  Synthetic oligonucleotide
gcaggttcccctcggggggc

SEQ ID NO: 915 - DNA, 20 bp, Artificial sequence
  Synthetic oligonucleotide
cagagagtacacggcggcag

SEQ ID NO: 916 - DNA, 20 bp, Artificial sequence
  Synthetic oligonucleotide
ccggggttactttcctgccc

SEQ ID NO: 917 - DNA, 20 bp, Artificial sequence
  Synthetic oligonucleotide
gtacacggcggcagggctca

SEQ ID NO: 918 - DNA, 20 bp, Artificial sequence
  Synthetic oligonucleotide
ggaggccctgtctgagctgg

SEQ ID NO: 919 - DNA, 20 bp, Artificial sequence
  Synthetic oligonucleotide
caacagtggctcgcacggcc

SEQ ID NO: 920 - DNA, 20 bp, Artificial sequence
  Synthetic oligonucleotide
gtctgagctggggactctat

SEQ ID NO: 921 - DNA, 20 bp, Artificial sequence
  Synthetic oligonucleotide
cggggcctcacctcggggga

SEQ ID NO: 922 - DNA, 20 bp, Artificial sequence
  Synthetic oligonucleotide
ccctccacagcaggtccaag

SEQ ID NO: 923 - DNA, 20 bp, Artificial sequence
  Synthetic oligonucleotide
caggccccaggaaggacatt

SEQ ID NO: 924 - DNA, 20 bp, Artificial sequence
  Synthetic oligonucleotide
ggcaccctccacagcaggtc

SEQ ID NO: 925 - DNA, 20 bp, Artificial sequence
  Synthetic oligonucleotide
caggctccccaggcggccat

SEQ ID NO: 926 - DNA, 20 bp, Artificial sequence
  Synthetic oligonucleotide
cggccagaggatggcagact

SEQ ID NO: 927 - DNA, 20 bp, Artificial sequence
  Synthetic oligonucleotide
ccccaggcggccattccgtc

SEQ ID NO: 928 - DNA, 20 bp, Artificial sequence
  Synthetic oligonucleotide
ccacggcaggtccgagcttt

SEQ ID NO: 929 - DNA, 20 bp, Artificial sequence
  Synthetic oligonucleotide
ttagataaacagacagaggg

SEQ ID NO: 930 - DNA, 20 bp, Artificial sequence
  Synthetic oligonucleotide
gaggatggcagactgcaatg

SEQ ID NO: 931 - DNA, 20 bp, Artificial sequence
  Synthetic oligonucleotide
ttactttcctgccccccacg

SEQ ID NO: 932 - DNA, 20 bp, Artificial sequence
  Synthetic oligonucleotide
cagcctgggccccggggcac

SEQ ID NO: 933 - DNA, 20 bp, Artificial sequence
  Synthetic oligonucleotide
ctcagggaaggctccaccct

SEQ ID NO: 934 - DNA, 20 bp, Artificial sequence
  Synthetic oligonucleotide
tcagcgtgatctgtgggacc

SEQ ID NO: 935 - DNA, 20 bp, Artificial sequence
  Synthetic oligonucleotide
gggccccggggcacgctcag

SEQ ID NO: 936 - DNA, 20 bp, Artificial sequence
  Synthetic oligonucleotide
gctaggcccggggtctggtc

SEQ ID NO: 937 - DNA, 20 bp, Artificial sequence
  Synthetic oligonucleotide
tgaggggaggacggcgggaa

SEQ ID NO: 938 - DNA, 20 bp, Artificial sequence
  Synthetic oligonucleotide
ccccacctccgggagacccc

SEQ ID NO: 939 - DNA, 20 bp, Artificial sequence
  Synthetic oligonucleotide
cagcaccatttgcagcggca

SEQ ID NO: 940 - DNA, 20 bp, Artificial sequence
  Synthetic oligonucleotide
gcagctggcactgggcttcc

SEQ ID NO: 941 - DNA, 20 bp, Artificial sequence
  Synthetic oligonucleotide
cgggagacccctctgctcac

SEQ ID NO: 942 - DNA, 20 bp, Artificial sequence
  Synthetic oligonucleotide
tcagggaaggccacacccac

SEQ ID NO: 943 - DNA, 20 bp, Artificial sequence
  Synthetic oligonucleotide
catttgcagcggcaaagtcc

SEQ ID NO: 944 - DNA, 20 bp, Artificial sequence
  Synthetic oligonucleotide
cagcagggctggagaatctt

SEQ ID NO: 945 - DNA, 20 bp, Artificial sequence
  Synthetic oligonucleotide
cctgccctcggcaaacgccg

SEQ ID NO: 946 - DNA, 20 bp, Artificial sequence
  Synthetic oligonucleotide
cacacgttgaatcccggcgg

SEQ ID NO: 947 - DNA, 20 bp, Artificial sequence
  Synthetic oligonucleotide
gcccaggctggcagctaggc

SEQ ID NO: 948 - DNA, 20 bp, Artificial sequence
  Synthetic oligonucleotide
cacaccttggggggggagga

SEQ ID NO: 949 - DNA, 20 bp, Artificial sequence
  Synthetic oligonucleotide
ttgaatcccggcggccatca

SEQ ID NO: 950 - DNA, 20 bp, Artificial sequence
  Synthetic oligonucleotide
caccattaccaccaccatta

SEQ ID NO: 951 - DNA, 20 bp, Artificial sequence
  Synthetic oligonucleotide
cagcccctgcctcagacaga

SEQ ID NO: 952 - DNA, 20 bp, Artificial sequence
  Synthetic oligonucleotide
atcaccatcgccaccatcat

SEQ ID NO: 953 - DNA, 20 bp, Artificial sequence
  Synthetic oligonucleotide
actcaaccttcctctgcctc

SEQ ID NO: 954 - DNA, 20 bp, Artificial sequence
  Synthetic oligonucleotide
aggccacacccacagggaag

SEQ ID NO: 955 - DNA, 20 bp, Artificial sequence
  Synthetic oligonucleotide
cctgcctcagacagagccag

SEQ ID NO: 956 - DNA, 20 bp, Artificial sequence
  Synthetic oligonucleotide
gcggggcctcacctcggggg

SEQ ID NO: 957 - DNA, 20 bp, Artificial sequence
  Synthetic oligonucleotide
ctgtctgagctgggaggccc

SEQ ID NO: 958 - DNA, 20 bp, Artificial sequence
  Synthetic oligonucleotide
agggaaggccccatccacag

SEQ ID NO: 959 - DNA, 20 bp, Artificial sequence
  Synthetic oligonucleotide
gtggctcgcacggccagccc

SEQ ID NO: 960 - DNA, 20 bp, Artificial sequence
  Synthetic oligonucleotide
gaggctgcatctaccccagg

SEQ ID NO: 961 - DNA, 20 bp, Artificial sequence
  Synthetic oligonucleotide
tgtccaagctgggggactct

SEQ ID NO: 962 - DNA, 20 bp, Artificial sequence
  Synthetic oligonucleotide
ctcagggaaggccacaccca

SEQ ID NO: 963 - DNA, 20 bp, Artificial sequence
  Synthetic oligonucleotide
cctcggcaaacgccgtccct

SEQ ID NO: 964 - DNA, 20 bp, Artificial sequence
  Synthetic oligonucleotide
aaacctcaagtggaccttcg

SEQ ID NO: 965 - DNA, 20 bp, Artificial sequence
  Synthetic oligonucleotide
ccatttgcagcggcaaagtc

SEQ ID NO: 966 - DNA, 20 bp, Artificial sequence
  Synthetic oligonucleotide
tcagacagagccagggttgg

SEQ ID NO: 967 - DNA, 20 bp, Artificial sequence
  Synthetic oligonucleotide
tggcactgggcttccttcct

SEQ ID NO: 968 - DNA, 20 bp, Artificial sequence
  Synthetic oligonucleotide
ctcagggtaggatccaccca

SEQ ID NO: 969 - DNA, 20 bp, Artificial sequence
  Synthetic oligonucleotide
gctgggaggccctgtctgag

SEQ ID NO: 970 - DNA, 20 bp, Artificial sequence
  Synthetic oligonucleotide
catcgccaccatcatcacca

SEQ ID NO: 971 - DNA, 20 bp, Artificial sequence
  Synthetic oligonucleotide
ggaggctgcatctaccccag

SEQ ID NO: 972 - DNA, 20 bp, Artificial sequence
  Synthetic oligonucleotide
acagcctgggccccggggca

SEQ ID NO: 973 - DNA, 20 bp, Artificial sequence
  Synthetic oligonucleotide
tcaccattaccaccaccatt

SEQ ID NO: 974 - DNA, 20 bp, Artificial sequence
  Synthetic oligonucleotide
cagctaggcctggggcccac

SEQ ID NO: 975 - DNA, 20 bp, Artificial sequence
  Synthetic oligonucleotide
tcgctgtcccgcaggttccc

SEQ ID NO: 976 - DNA, 20 bp, Artificial sequence
  Synthetic oligonucleotide
gggcaccctccacagcaggt

SEQ ID NO: 977 - DNA, 20 bp, Artificial sequence
  Synthetic oligonucleotide
ccacaccttggggggggagg

SEQ ID NO: 978 - DNA, 20 bp, Artificial sequence
  Synthetic oligonucleotide
tcacagcattccagctcgct

SEQ ID NO: 979 - DNA, 20 bp, Artificial sequence
  Synthetic oligonucleotide
tgggccccggggcacgctca

SEQ ID NO: 980 - DNA, 20 bp, Artificial sequence
  Synthetic oligonucleotide
agaggatggcagactgcaat

SEQ ID NO: 981 - DNA, 20 bp, Artificial sequence
  Synthetic oligonucleotide
tccccaggcggccattccgt

SEQ ID NO: 982 - DNA, 20 bp, Artificial sequence
  Synthetic oligonucleotide
cactcaaccttcctctgcct

SEQ ID NO: 983 - DNA, 20 bp, Artificial sequence
  Synthetic oligonucleotide
acggccagaggatggcagac

SEQ ID NO: 984 - DNA, 20 bp, Artificial sequence
  Synthetic oligonucleotide
acaggctccccaggcggcca

SEQ ID NO: 985 - DNA, 20 bp, Artificial sequence
  Synthetic oligonucleotide
cccacaacctcccccggcga

SEQ ID NO: 986 - DNA, 20 bp, Artificial sequence
  Synthetic oligonucleotide
gtcaggccccaggaaggaca

SEQ ID NO: 987 - DNA, 20 bp, Artificial sequence
  Synthetic oligonucleotide
gttgaatcccggcggccatc

SEQ ID NO: 988 - DNA, 20 bp, Artificial sequence
  Synthetic oligonucleotide
cgcagctggcactgggcttc

SEQ ID NO: 989 - DNA, 20 bp, Artificial sequence
  Synthetic oligonucleotide
cattccagctcgcttcagga

SEQ ID NO: 990 - DNA, 20 bp, Artificial sequence
  Synthetic oligonucleotide
gaaggccacacccacaggga

SEQ ID NO: 991 - DNA, 20 bp, Artificial sequence
  Synthetic oligonucleotide
gcagcggcaaagtcctagca

SEQ ID NO: 992 - DNA, 20 bp, Artificial sequence
  Synthetic oligonucleotide
tcctgccctcggcaaacgcc

SEQ ID NO: 993 - DNA, 20 bp, Artificial sequence
  Synthetic oligonucleotide
ggccccatccacagggaagg

SEQ ID NO: 994 - DNA, 20 bp, Artificial sequence
  Synthetic oligonucleotide
cgcaggttcccctcgggggg

SEQ ID NO: 995 - DNA, 20 bp, Artificial sequence
  Synthetic oligonucleotide
ctgagctggcagggctgccc

SEQ ID NO: 996 - DNA, 20 bp, Artificial sequence
  Synthetic oligonucleotide
ggaaggctccaccctctggg

SEQ ID NO: 997 - DNA, 20 bp, Artificial sequence
  Synthetic oligonucleotide
agctggggactctatctggg

SEQ ID NO: 998 - DNA, 20 bp, Artificial sequence
  Synthetic oligonucleotide
ccccggcgatgtcacctcct

SEQ ID NO: 999 - DNA, 20 bp, Artificial sequence
  Synthetic oligonucleotide
cggtgagcccaggagcaccc

SEQ ID NO: 1000 - DNA, 20 bp, Artificial sequence
  Synthetic oligonucleotide
actgcaaagggggccacagt

SEQ ID NO: 1001 - DNA, 20 bp, Artificial sequence
  Synthetic oligonucleotide
caagtggaccttcggagggc

SEQ ID NO: 1002 - DNA, 20 bp, Artificial sequence
  Synthetic oligonucleotide
cctcacctcgggggaggaaa

SEQ ID NO: 1003 - DNA, 20 bp, Artificial sequence
  Synthetic oligonucleotide
tgggaggccctgtctgagct

SEQ ID NO: 1004 - DNA, 20 bp, Artificial sequence
  Synthetic oligonucleotide
accatcatcaccacctccac

SEQ ID NO: 1005 - DNA, 20 bp, Artificial sequence
  Synthetic oligonucleotide
ggagctcactcaaccttcct

SEQ ID NO: 1006 - DNA, 20 bp, Artificial sequence
  Synthetic oligonucleotide
gcaggtccgagctttgtgaa

SEQ ID NO: 1007 - DNA, 20 bp, Artificial sequence
  Synthetic oligonucleotide
aggacggcgggaagacgatg

SEQ ID NO: 1008 - DNA, 20 bp, Artificial sequence
  Synthetic oligonucleotide
tccacggcaggtccgagctt

SEQ ID NO: 1009 - DNA, 20 bp, Artificial sequence
  Synthetic oligonucleotide
tccggggttactttcctgcc

SEQ ID NO: 1010 - DNA, 20 bp, Artificial sequence
  Synthetic oligonucleotide
accctccacagcaggtccaa

SEQ ID NO: 1011 - DNA, 20 bp, Artificial sequence
  Synthetic oligonucleotide
ttaccaccaccattaccacc

SEQ ID NO: 1012 - DNA, 20 bp, Artificial sequence
  Synthetic oligonucleotide
ccatgtctgagctggcaggg

SEQ ID NO: 1013 - DNA, 20 bp, Artificial sequence
  Synthetic oligonucleotide
tgtctgagctggggactcta

SEQ ID NO: 1014 - DNA, 20 bp, Artificial sequence
  Synthetic oligonucleotide
ctgggggccttggctgccac

SEQ ID NO: 1015 - DNA, 20 bp, Artificial sequence
  Synthetic oligonucleotide
taaacagacagagggatcga

SEQ ID NO: 1016 - DNA, 20 bp, Artificial sequence
  Synthetic oligonucleotide
tccaccctcagggtaggatc

SEQ ID NO: 1017 - DNA, 20 bp, Artificial sequence
  Synthetic oligonucleotide
cgcacggccagcccaccgtg

SEQ ID NO: 1018 - DNA, 20 bp, Artificial sequence
  Synthetic oligonucleotide
cggggctaggcccggggtct

SEQ ID NO: 1019 - DNA, 20 bp, Artificial sequence
  Synthetic oligonucleotide
atgaggggaggacggcggga

SEQ ID NO: 1020 - DNA, 20 bp, Artificial sequence
  Synthetic oligonucleotide
acatcaccatcgccaccatc

SEQ ID NO: 1021 - DNA, 20 bp, Artificial sequence
  Synthetic oligonucleotide
tccgggagacccctctgctc

SEQ ID NO: 1022 - DNA, 20 bp, Artificial sequence
  Synthetic oligonucleotide
tacaagacggcaaagttcag

SEQ ID NO: 1023 - DNA, 20 bp, Artificial sequence
  Synthetic oligonucleotide
gttactttcctgccccccac

SEQ ID NO: 1024 - DNA, 20 bp, Artificial sequence
  Synthetic oligonucleotide
agtacacggcggcagggctc

SEQ ID NO: 1025 - DNA, 20 bp, Artificial sequence
  Synthetic oligonucleotide
cacccttacaagacggcaaa

SEQ ID NO: 1026 - DNA, 20 bp, Artificial sequence
  Synthetic oligonucleotide
ccgggagacccctctgctca

SEQ ID NO: 1027 - DNA, 20 bp, Artificial sequence
  Synthetic oligonucleotide
ctgccctgtccaagctgggg

SEQ ID NO: 1028 - DNA, 20 bp, Artificial sequence
  Synthetic oligonucleotide
ccagagagtacacggcggca

SEQ ID NO: 1029 - DNA, 20 bp, Artificial sequence
  Synthetic oligonucleotide
ggctggcagctaggcctggg